(19) United States
(12) Patent Application Publication
(10) Pub. No.: US 2024/0400544 A1
(43) Pub. Date: Dec. 5, 2024

(54) N-(HYDROXYALKYL (HETERO)ARYL) TETRAHYDROFURAN CARBOXAMIDE ANALOGS AS MODULATORS OF SODIUM CHANNELS

(71) Applicant: VERTEX PHARMACEUTICALS INCORPORATED, Boston, MA (US)

(72) Inventors: Elizabeth Mary BECK, Abingdon, Oxfordshire (GB); Robert PULLIN, Oxford, Oxfordshire (GB); Gorka ETXEBARRIA JARDI, Badalona (ES); Dean STAMOS, Lexington, MA (US); Yvonne SCHMIDT, San Diego, CA (US); Joseph PONTILLO, San Diego, CA (US); Stephen Andrew THOMSON, Durham, NC (US); David Matthew SHAW, Oxford, Oxfordshire (GB); Nadia M. AHMAD, Hayes, Middlesex (GB); Lidio Marx CARVALHO MEIRELES, San Marcos, CA (US); Sarah SKERRATT, Cambridge, Cambridgeshire (GB); Sara S. HADIDA RUAH, La Jolla, CA (US); Timothy Donald NEUBERT, San Diego, CA (US); Dennis James HURLEY, San Marcos, CA (US); Alexander KINTZER, Carlsbad, CA (US); Steven John DURRANT, Headington, Oxford (GB); Christopher WRAY, Berkshire (GB); Anisa Nizarali VIRANI, Thatcham, Berkshire (GB); Kiri NORTH, Oxford, Oxfordshire (GB); Reece JACQUES, Oxford, Oxfordshire (GB); Mark Thomas Miller, Rancho Santa Fe, CA (US); Jinglan ZHOU, San Diego, CA (US); Ronald Marcellus KNEGTEL, Abingdon, Oxfordshire (GB); Ewa Iwona CHUDYK, Wantage, Oxfordshire (GB); Joanne Louise PINDER, Didcot, Oxfordshire (GB); Bruno Artur SOUSA, Reading, Berkshire (GB); James DODD, Wallingford, Oxfordshire (GB); Michael Edward O'DONNELL, Abingdon, Oxfordshire (GB); Bhairavi GALAN, Abingdon, Oxfordshire (GB); Rebecca E. STEVENS, Gloucester, Gloucestershire (GB)

(73) Assignee: VERTEX PHARMACEUTICALS INCORPORATED, Boston, MA (US)

(21) Appl. No.: 18/566,282

(22) PCT Filed: Jun. 3, 2022
(86) PCT No.: PCT/US2022/032202

§ 371 Date: Dec. 1, 2023

Related U.S. Application Data
(60) Provisional application No. 63/196,970, filed on Jun. 4, 2021.

(51) Int. Cl. C07D 413/14; A61K 31/4355; A61K 31/443; A61K 31/506; A61K 31/5377; C07D 405/12; C07D 405/14; C07D 491/04

(52) U.S. Cl. CPC C07D 413/14; A61K 31/4355; A61K 31/443; A61K 31/506; A61K 31/5377; C07D 405/12; C07D 405/14; C07D 491/04

(57) ABSTRACT

Compounds of formula (I) and pharmaceutically acceptable salts thereof, useful as inhibitors of sodium channels are provided. Also provided are pharmaceutical compositions comprising the compounds or pharmaceutically acceptable salts and methods of using the compounds, pharmaceutically acceptable salts, and pharmaceutical compositions in the treatment of various disorders, including pain.

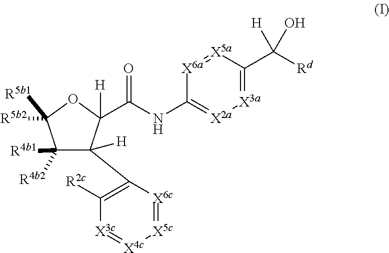

### CROSS-REFERENCE TO RELATED APPLICATIONS

This application claims the benefit of U.S. Provisional Application No. 63/196,970, filed Jun. 4, 2021, which is incorporated by reference herein in its entirety.

### BACKGROUND

Pain is a protective mechanism that allows healthy animals to avoid tissue damage and to prevent further damage to injured tissue. Nonetheless there are many conditions where pain persists beyond its usefulness, or where patients would benefit from inhibition of pain. Neuropathic pain is a form of chronic pain caused by an injury to the sensory nerves (Dieleman, J. P., et al., Incidence rates and treatment of neuropathic pain conditions in the general population. Pain, 2008. 137(3): p. 681-8). Neuropathic pain can be divided into two categories, pain caused by generalized metabolic damage to the nerve and pain caused by a discrete nerve injury. The metabolic neuropathies include post-herpetic neuropathy, diabetic neuropathy, and drug-induced neuropathy. Discrete nerve injury indications include post-amputation pain, post-surgical nerve injury pain, and nerve entrapment injuries like neuropathic back pain. Neuropathic pain is a major cause of disability worldwide, negatively affecting patient's sleep, mood, and functionality. Clin. Ther., 2018 40(6): p. 828-49.

Voltage-gated sodium channels (Na_{V}s) are involved in pain signaling. Na_{V}s mediate the rapid upstroke of the action potential of many excitable cell types (e.g. neurons, skeletal myocytes, cardiac myocytes), and thus are involved in the initiation of electrical signaling in those cells (Hille, Bertil, Ion Channels of Excitable Membranes, Third ed. (Sinauer Associates, Inc., Sunderland, MA, 2001)). Support for the assertion that Na_{V}s play a critical and central role in pain signaling arises from (1) evaluation of the role Na_{V}s plays in normal physiology, (2) pathological states arising from mutations in the Nav1.8 gene (SCN10A). (3) preclinical work in animal models, and (4) pharmacology of known Nav1.8-modulating agents. In addition, because Nav1.8 expression is restricted to peripheral neurons, particularly those that sense pain (e.g., the dorsal root ganglia), Nav1.8 inhibitors are less likely to be associated with the side effects commonly observed with other sodium channel modulators and the abuse liability associated with opioid therapies. Therefore, targeting the underlying biology of pain through selective Nav1.8 inhibition represents a novel approach to analgesic drug development that has the potential to address an urgent unmet need for safe and effective acute and chronic pain therapies. (Rush, A. M. and T. R. Cummins, Painful Research: Identification of a Small-Molecule Inhibitor that Selectively Targets Na_{V}1.8 Sodium Channels. Mol. Interv., 2007. 7(4): p. 192-5); England, S., Voltage-gated sodium channels: the search for subtype-selective analgesics. Expert Opin. Investig. Drugs 17 (12), p. 1849-64 (2008); Krafte, D. S. and Bannon, A. W., Sodium channels and nociception: recent concepts and therapeutic opportunities. Curr. Opin. Pharmacol. 8 (1), p. 50-56 (2008)). Because of the role Na_{V}s play in the initiation and propagation of neuronal signals, antagonists that reduce Na_{V} currents can prevent or reduce neural signaling and Na_{V} channels have been considered likely targets to reduce pain in conditions where hyper-excitability is observed (Chahine, M., Chatelier, A., Babich, O., and Krupp, J. J., Voltage-gated sodium channels in neurological disorders. CNS Neurol. Disord. Drug Targets 7 (2), p. 144-58 (2008)). Several clinically useful analgesics have been identified as inhibitors of Na_{V} channels. The local anesthetic drugs such as lidocaine block pain by inhibiting Na_{V} channels, and other compounds, such as carbamazepine, lamotrigine, and tricyclic antidepressants that have proven effective at reducing pain have also been suggested to act by sodium channel inhibition (Soderpalm, B., Anticonvulsants: aspects of their mechanisms of action. Eur. J Pain 6 Suppl. A, p. 3-9 (2002); Wang, G. K., Mitchell, J., and Wang, S. Y., Block of persistent late Na⁺ currents by antidepressant sertraline and paroxetine. J. Membr. Biol. 222 (2), p. 79-90 (2008)).

The Na_{V}s form a subfamily of the voltage-gated ion channel super-family and comprises 9 isoforms, designated Na_{V}1.1-Na_{V}1.9. The tissue localizations of the nine isoforms vary. Na_{V}1.4 is the primary sodium channel of skeletal muscle, and Na_{V}1.5 is the primary sodium channel of cardiac myocytes. Na_{V}s 1.7, 1.8, and 1.9 are primarily localized to the peripheral nervous system, while Na_{V}s 1.1, 1.2, 1.3, and 1.6 are neuronal channels found in both the central and peripheral nervous systems. The functional behaviors of the nine isoforms are similar but distinct in the specifics of their voltage-dependent and kinetic behavior (Catterall, W. A., Goldin, A. L., and Waxman, S. G., International Union of Pharmacology. XLVII. Nomenclature and structure-function relationships of voltage-gated sodium channels. Pharmacol. Rev. 57 (4), p. 397 (2005)).

Upon their discovery, Na_{V}1.8 channels were identified as likely targets for analgesia (Akopian, A. N., L. Sivilotti, and J. N. Wood, A tetrodotoxin-resistant voltage-gated sodium channel expressed by sensory neurons. Nature, 1996. 379(6562): p. 257-62). Since then, Na_{V}1.8 has been shown to be a carrier of the sodium current that maintains action potential firing in small dorsal root ganglia (DRG) neurons (Blair, N. T. and B. P. Bean, Roles of tetrodotoxin (TTX)-sensitive Na+ current, TTX-resistant Na⁺current, and Ca²⁺ current in the action potentials of nociceptive sensory neurons. J. Neurosci., 2002. 22(23): p. 10277-90). Na_{V}1.8 is involved in spontaneous firing in damaged neurons, like those that drive neuropathic pain (Roza, C., et al., The tetrodotoxin-resistant Na⁺ channel Na_{V}1.8 is essential for the expression of spontaneous activity in damaged sensory axons of mice. J. Physiol., 2003. 550(Pt 3): p. 921-6; Jarvis, M. F., et al., A-803467, a potent and selective Na_{V}1.8 sodium channel blocker, attenuates neuropathic and inflammatory pain in the rat. Proc. Natl. Acad. Sci. USA, 2007. 104(20): p. 8520-5; Joshi, S. K., et al., Involvement of the TTX-resistant sodium channel Na_{V}1.8 in inflammatory and neuropathic, but not post-operative, pain states. Pain, 2006. 123(1-2): pp. 75-82; Lai, J., et al., Inhibition of neuropathic pain by decreased expression of the tetrodotoxin-resistant sodium channel, Na_{V}1.8. Pain, 2002. 95(1-2): p. 143-52; Dong, X. W., et al., Small interfering RNA-mediated selective knockdown of Na_{V}1.8 tetrodotoxin-resistant sodium channel reverses mechanical allodynia in neuropathic rats. Neuroscience, 2007. 146(2): p. 812-21; Huang, H. L., et al., Proteomic profiling of neuromas reveals alterations in protein composition and local protein synthesis in hyper-excitable nerves. Mol. Pain, 2008. 4: p. 33; Black, J. A., et al., Multiple sodium channel isoforms and mitogen-activated protein kinases are present in painful human neuromas. Ann. Neurol., 2008. 64(6): p. 644-53; Coward, K., et al., Immunolocalization of SNS/PN3 and NaN/SNS2 sodium channels in human pain states. Pain, 2000. 85(1-2): p. 41-50; Yiangou, Y., et al., SNS/PN3 and SNS2/NaN sodium channel-like immunoreactivity in human adult and neonate injured sensory nerves. FEBS Lett., 2000. 467(2-3): p. 249-52; Ruangsri, S., et al., Relationship of axonal voltage-gated sodium channel 1.8 (Na_{V}1.8) mRNA accumulation to sciatic nerve injury-induced painful neuropathy in rats. J. Biol. Chem. 286(46): p. 39836-47). The small DRG neurons where Na_{V}1.8 is expressed include the nociceptors involved in pain signaling. Na_{V}1.8 mediates large amplitude action potentials in small neurons of the dorsal root ganglia (Blair, N. T. and B. P. Bean, Roles of tetrodotoxin (TTX)-sensitive Na⁺ current, TTX-resistant Na⁺ current, and Ca²⁺ current in the action potentials of nociceptive sensory neurons. J. Neurosci., 2002. 22(23): p. 10277-90). Na_{V}1.8 is necessary for rapid repetitive action potentials in nociceptors and for spontaneous activity of damaged neurons (Choi, J. S. and S. G. Waxman, Physiological interactions between Na_{V}1.7 and Na_{V}1.8 sodium channels: a computer simulation study. J. Neurophysiol. 106(6): p. 3173-84; Renganathan, M., T. R. Cummins, and S. G. Waxman, Contribution of Na(_{V})1.8 sodium channels to action potential electrogenesis in DRG neurons. J. Neurophysiol., 2001. 86(2): p. 629-40; Roza, C., et al., The tetrodotoxin-resistant Na⁺ channel Na_{V}1.8 is essential for the expression of spontaneous activity in damaged sensory axons of mice. J. Physiol., 2003. 550(Pt 3): p. 921-6). In depolarized or damaged DRG neurons, Na_{V}1.8 appears to be a driver of hyper-excitability (Rush, A. M., et al., A single sodium channel mutation produces hyper- or hypoexcitability in different types of neurons. Proc. Natl. Acad. Sci. USA, 2006. 103(21): p. 8245-50). In some animal pain models, Na_{V}1.8 mRNA expression levels have been shown to increase in the DRG (Sun, W., et al., Reduced conduction failure of the main axon of polymodal nociceptive C-fibers contributes to painful diabetic neuropathy in rats. Brain, 135(Pt 2): p. 359-75; Strickland, I. T., et al., Changes in the expression of Na_{V}1.7, Na_{V}1.8 and Na_{V}1.9 in a distinct population of dorsal root ganglia innervating the rat knee joint in a model of chronic inflammatory joint pain. Eur. J. Pain, 2008. 12(5): p. 564-72; Qiu, F., et al., Increased expression of tetrodotoxin-resistant sodium channels Na_{V}1.8 and Na_{V}1.9 within dorsal root ganglia in a rat model of bone cancer pain. Neurosci. Lett., 512(2): p. 61-6).

The inventors have discovered that some voltage-gated sodium channel inhibitors have limitations as therapeutic agents due to, for example, a poor therapeutic window (e.g., due to a lack of Na_{V} isoform selectivity, low potency, and/or other reasons). Accordingly, there remains a need to develop selective voltage-gated sodium channel inhibitors, such as selective Na_{V}1.8 inhibitors.

### SUMMARY

In one aspect, the invention relates to a compound described herein, or a pharmaceutically acceptable salt thereof.

In another aspect, the invention relates to a pharmaceutical composition comprising the compound, or a pharmaceutically acceptable salt thereof, and one or more pharmaceutically acceptable carriers or vehicles.

In still another aspect, the invention relates to a method of inhibiting a voltage gated sodium channel in a subject by administering the compound, pharmaceutically acceptable salt, or pharmaceutical composition to the subject.

In yet another aspect, the invention relates to a method of treating or lessening the severity in a subject of a variety of diseases, disorders, or conditions, including, but not limited to, chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, and cardiac arrhythmia, by administering the compound, pharmaceutically acceptable salt, or pharmaceutical composition to the subject.

### BRIEF DESCRIPTION OF THE DRAWINGS

FIG. 1 depicts an XRPD pattern characteristic of amorphous Compound 17.

FIG. 2 depicts an XRPD pattern characteristic of amorphous Compound 25.

### DETAILED DESCRIPTION

In one aspect, the invention relates to a compound of formula (I)

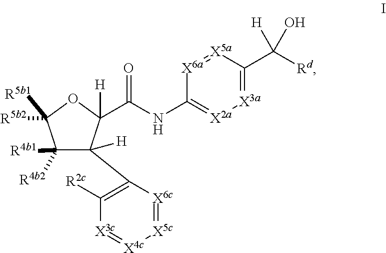

or a pharmaceutically acceptable salt thereof, wherein:
X^{2a} is N, N⁺—O⁻, or C—R^{2a};
X^{3a} is N or N⁺—O⁻;
X^{5a} is N, N⁺—O⁻, or C—R^{5a};
X^{6a} is N, N⁺—O⁻, or C—R^{6a};
R^{d} is (CH₂)ₙ(CHR^{e})ₙ(CH₂)ₚH;
m, n, and p are each independently 0 or 1;
R^{e} is H, OH, halo, C₁-C₆ alkoxy, or C₁-C₆ haloalkoxy;
R^{2a} and R^{6a} are each independently H, halo, C₁-C₆ alkyl, or C₁-C₆ haloalkyl;
R^{5a} is H, halo, CH₂OH, C₁-C₆ alkyl, C₁-C₆ haloalkyl, or R^{5a} and R^{d} form a CH₂CH₂ chain linking the C atoms to which R^{5a} and R^{d} are attached, wherein the CH₂ group that is bound to the C atom to which R^{5a} is attached may be replaced with O;
R^{4b1} and R^{4b2} are each independently H, C₁-C₆ alkyl, C₃-C₆ cycloalkyl, or C₁-C₆ haloalkyl;
R^{5b1} and R^{5b2} are each independently H, C₁-C₆ alkyl, C₃-C₆ cycloalkyl, or C₁-C₆ haloalkyl;
X^{3c} is N or C—R^{3c};
X^{4c} is N or C—R^{4c};
X^{5c} is N or C—R^{5c};
X^{6c} is N or C—R^{6c};
R^{2c} is H, OH, halo, C₁-C₆ alkyl, C₂-C₆ alkenyl, C₁-C₆ haloalkyl, C₁-C₆ alkoxy, C₁-C₆ haloalkoxy, O—CH₂—C(R^{2c1})(R^{2c2})(R^{2c3}), O—CH(R^{2c4})(R^{2c5}), or -L¹-L²-(C₃-C₆ cycloalkyl), wherein said cycloalkyl is optionally substituted with 1-2 halo;
R^{2c3} and R^{2c2} are each independently H or C₁-C₆ alkyl, or R^{2c1} and R^{2c2} together with the C atom to which they are attached form C═O;
R^{2c3} is OH, C₁-C₆ alkoxy, C₁-C₆ haloalkoxy, or N(R^{2c6})(R^{2c7}); or R^{2c2} and R^{2c3} together with the C atom to which they are attached form a 3-7 membered heterocycloalkyl;
R^{2c4} and R^{2c5} together with the C atom to which they are attached form a 3-7 membered heterocycloalkyl;
R^{2c6} and R^{2c7} are each C₁-C₆ alkyl, or R^{2c6} and R^{2c7} together with the N atom to which they are attached form a 3-8 membered heterocycloalkyl;
L¹ is a bond or O;
L² is a bond or C₁-C₆ alkylene;
R^{3c} is H, halo, C₁-C₆ alkyl, or C₁-C₆ haloalkyl; or X^{3c} is C—R^{3c}, and R^{2c} and R^{3c}, together with the carbon atoms to which they are attached, form a ring of formula:

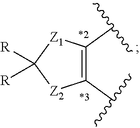

Z₁ and Z₂ are each independently O or CH₂;
each R is independently H or halo;
R^{4c} is H, halo, C₁-C₆ alkyl, C₁-C₆ haloalkyl, C₁-C₆ alkoxy, or C₁-C₆ haloalkoxy;
R^{5c} is H, halo, C₁-C₆ alkyl, or C₁-C₆ haloalkyl; and
R^{6c} is H, halo, C₁-C₆ alkyl, or C₁-C₆ haloalkyl; provided that no more than two of X^{2a}, X^{3a}, X^{5a}, and X^{6a} are N or N⁺—O⁻; and provided that no more than one of X^{3c}, X^{4c}, X^{5c}, and X^{6c} is N; and
provided that:
   R^{5a} and R^{d} form a CH₂CH₂ chain linking the C atoms to which R^{5a} and R^{d} are attached, wherein the CH₂ group that is bound to the C atom to which R^{5a} is attached may be replaced with O; or
R^{2c} is O—CH₂—C(R^{2c1})(R^{2c2})(R^{2c3}) or O—CH(R^{2c4})(R^{2c5}).

For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001, the entire contents of which are hereby incorporated by reference.

As used herein, the term “compounds of the invention” refers to the compounds of formula (I), and all of the embodiments thereof (e.g., formulas (I-A), (I-A-1), (I-A-2), (I-A-3), (I-B), (I-B-1), (I-B-2), (I-B-3), (I-C), (I-C-1), (I-C-2), (I-C-3), (I-D), (I-D-1), (I-D-2), (I-D-3), and (I-D-4), etc.), as described herein, and to the compounds identified in Table A.

As described herein, the compounds of the invention comprise multiple variable groups (e.g., R^{5b1}, X^{3a}, R^{d}, etc.). As one of ordinary skill in the art will recognize, combinations of groups envisioned by this invention are those combinations that result in the formation of stable or chemically feasible compounds. The term “stable,” in this context, refers to compounds that are not substantially altered when subjected to conditions that allow for their production, detection, and optionally their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The chemical structures depicted herein are intended to be understood as they would be understood by one of ordinary skill in the art. For example, with respect to formulas (I), (I-A), (I-B), (I-C), and (I-D), X^{2a} and X^{3a} are connected by a single bond, X^{5a} and X^{6a} are connected by a double bond, and X^{4c} and X^{5c} are connected by a single bond, even though the bonds between these groups may be obscured by the atom labels in the chemical structures. For example, using a different style, formula (I) can be drawn as follows to show the bonds in question:

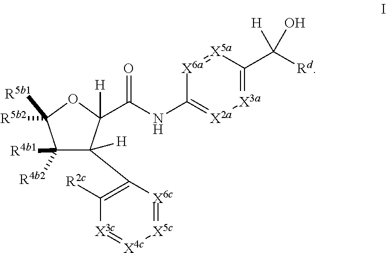

Moreover, a substituent depicted as “CF₃” or “F₃C” in a chemical structure refers to a trifluoromethyl substituent, regardless of which depiction appears in the chemical structure.

As used herein, the term “halo” means F, Cl, Br or I.

As used herein, the term “alkyl” refers to a straight or branched hydrocarbon chain radical group consisting solely of carbon and hydrogen atoms, containing no unsaturation, and having the specified number of carbon atoms, which is attached to the rest of the molecule by a single bond. For example, a “C₁-C₆ alkyl” group is an alkyl group having between one and six carbon atoms.

As used herein, the term “alkenyl” refers to a straight or branched hydrocarbon chain radical group consisting solely of carbon and hydrogen atoms, containing one or more carbon-carbon double bonds, and having the specified number of carbon atoms, which is attached to the rest of the molecule by a single bond. For example, a “C₂-C₆ alkenyl” group is an alkenyl group having between two and six carbon atoms.

As used herein, the term “cycloalkyl” refers to a stable, non-aromatic, mono- or bicyclic (fused, bridged, or spiro) saturated hydrocarbon radical consisting solely of carbon and hydrogen atoms, having the specified number of carbon ring atoms, and which is attached to the rest of the molecule by a single bond. For example, a “C₃-C₈ cycloalkyl” group is a cycloalkyl group having between three and eight carbon atoms.

As used herein, the term “haloalkyl” refers to an alkyl group having the specified number of carbon atoms, wherein one or more of the hydrogen atoms of the alkyl group are replaced by halo groups. For example, a “C₁-C₆ haloalkyl” group is an alkyl group having between one and six carbon atoms, wherein one or more of the hydrogen atoms of the alkyl group are replaced by halo groups.

As used herein, the term “alkoxy” refers to a radical of the formula —ORₐ, where Rₐ is an alkyl group having the specified number of carbon atoms. For example, a “C₁-C₆ alkoxy” group is a radical of the formula —ORₐ where Rₐ is an alkyl group having the between one and six carbon atoms.

As used herein, the term “haloalkoxy” refers to an alkoxy group having the specified number of carbon atoms, wherein one or more of the hydrogen atoms of the of the alkyl group are replaced by halo groups.

As used herein, the term “alkylene” refers to a divalent, straight or branched hydrocarbon chain radical group consisting solely of carbon and hydrogen atoms, containing no unsaturation, and having the specified number of carbon atoms, which is attached to the rest of the molecule by two single bonds. For example, a “C₁-C₆ alkylene” group is an alkylene group having between one and six carbon atoms.

As used herein, the term “optionally substituted” refers to a group that is either unsubstituted or substituted with the subsequently identified substituents. For example, a group that is “optionally substituted with 1-2 halo” is either unsubstituted, substituted with 1 halo group, or substituted with 2 halo groups.

As used herein, labels such as “*2” and “*3”, such as those shown in the following structure, designate designate the atoms to which the corresponding R groups (in this case, the R^{2c} and R^{3c} groups, respectively) are attached.

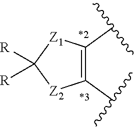

Unless otherwise specified, the compounds of the invention, whether identified by chemical name or chemical structure, include all stereoisomers (e.g., enantiomers and diastereomers), double bond isomers (e.g., (Z) and (E)), conformational isomers, and tautomers of the compounds identified by the chemical names and chemical structures provided herein. In addition, single stereoisomers, double bond isomers, conformational isomers, and tautomers as well as mixtures of stereoisomers, double bond isomers, conformational isomers, and tautomers are within the scope of the invention.

As used herein, in any chemical structure or formula, a non-bold, straight bond attached to a stereocenter of a compound, such as in

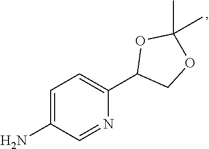

denotes that the configuration of the stereocenter is unspecified. The compound may have any configuration, or a mixture of configurations, at the stereocenter.

As used herein, in any chemical structure or formula, a bold or hashed straight bond attached to a stereocenter of a compound, such as in

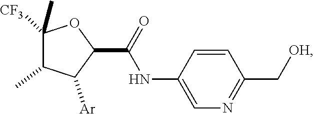

denotes the relative stereochemistry of the stereocenter, relative to other stereocenter(s) to which bold or hashed straight bonds are attached.

As used herein, in any chemical structure or formula, a bold or hashed wedge bond attached to a stereocenter of a compound, such as in

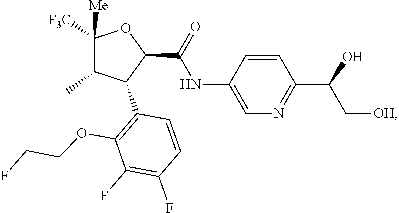 [SMILES: C[C@H]1[C@@H](c2ccc(F)c(F)c2OCCF)[C@H](C(=O)Nc2ccc([C@@H](O)CO)nc2)O[C@@]1(C)C(F)(F)F]

denotes the absolute stereochemistry of the stereocenter, as well as the relative stereochemistry of the stereocenter, relative to other stereocenter(s) to which bold or hashed wedge bonds are attached.

As used herein, the prefix “rac-,” when used in connection with a chiral compound, refers to a racemic mixture of the compound. In a compound bearing the “rac-” prefix, the (R)- and (S)-designators in the chemical name reflect the relative stereochemistry of the compound.

As used herein, the prefix “rel-,” when used in connection with a chiral compound, refers to a single enantiomer of unknown absolute configuration. In a compound bearing the “rel-” prefix, the (R)- and (S)-designators in the chemical name reflect the relative stereochemistry of the compound, but do not necessarily reflect the absolute stereochemistry of the compound. Where the relative stereochemistry of a given stereocenter is unknown, no stereochemical designator is provided. In some instances, the absolute configuration of some stereocenters is known, while only the relative configuration of the other stereocenters is known. In these instances, the stereochemical designators associated with the stereocenters of known absolute configuration are marked with an asterisk (*), e.g., (R*)- and (S*)-, while the stereochemical designators associated with stereocenters of unknown absolute configuration are not so marked. The unmarked stereochemical designators associated with the stereocenters of unknown absolute configuration reflect the relative stereochemistry of those stereocenters with respect to other stereocenters of unknown absolute configuration, but do not necessarily reflect the relative stereochemistry with respect to the stereocenters of known absolute configuration.

As used herein, the term “compound,” when referring to the compounds of the invention, refers to a collection of molecules having identical chemical structures, except that there may be isotopic variation among the constituent atoms of the molecules. The term “compound” includes such a collection of molecules without regard to the purity of a given sample containing the collection of molecules. Thus, the term “compound” includes such a collection of molecules in pure form, in a mixture (e.g., solution, suspension, colloid, or pharmaceutical composition, or dosage form) with one or more other substances, or in the form of a hydrate, solvate, or co-crystal.

As used herein, the term “amorphous” refers to a solid material having no long-range order in the position of its molecules. Amorphous solids are generally glasses or supercooled liquids in which the molecules are arranged in a random manner so that there is no well-defined arrangement, e.g., molecular packing, and no long-range order. Amorphous solids are generally rather isotropic, i.e., exhibit similar properties in all directions and do not have definite melting points. Instead, they typically exhibit a glass transition temperature which marks a transition from glassy amorphous state to supercooled liquid amorphous state upon heating. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. See US 2004/0006237 for a comparison of XRPDs of an amorphous material and crystalline material. In some embodiments, a solid material may comprise an amorphous compound, and the material may, for example, be characterized by a lack of sharp characteristic crystalline peak(s) in its XRPD spectrum (i.e., the material is not crystalline, but is amorphous, as determined by XRPD). Instead, one or several broad peaks (e.g., halos) may appear in the XRPD pattern of the material. See US 2004/0006237 for a representative comparison of XRPDs of an amorphous material and crystalline material. A solid material, comprising an amorphous compound, may be characterized by, for example, a wider temperature range for the melting of the solid material, as compared to the range for the melting of a pure crystalline solid. Other techniques, such as, for example, solid state NMR may also be used to characterize crystalline or amorphous forms.

In the specification and claims, unless otherwise specified, any atom not specifically designated as a particular isotope in any compound of the invention is meant to represent any stable isotope of the specified element. In the Examples, where an atom is not specifically designated as a particular isotope in any compound of the invention, no effort was made to enrich that atom in a particular isotope, and therefore a person of ordinary skill in the art would understand that such atom likely was present at approximately the natural abundance isotopic composition of the specified element.

As used herein, the term “stable,” when referring to an isotope, means that the isotope is not known to undergo spontaneous radioactive decay. Stable isotopes include, but are not limited to, the isotopes for which no decay mode is identified in V. S. Shirley & C. M. Lederer, Isotopes Project, Nuclear Science Division, Lawrence Berkeley Laboratory, Table of Nuclides (January 1980).

As used herein in the specification and claims, “H” refers to hydrogen and includes any stable isotope of hydrogen, namely ¹H and D. In the Examples, where an atom is designated as “H,” no effort was made to enrich that atom in a particular isotope of hydrogen, and therefore a person of ordinary skill in the art would understand that such hydrogen atom likely was present at approximately the natural abundance isotopic composition of hydrogen.

As used herein, “¹H” refers to protium. Where an atom in a compound of the invention, or a pharmaceutically acceptable salt thereof, is designated as protium, protium is present at the specified position at at least the natural abundance concentration of protium.

As used herein, “D,” “d,” and “²H” refer to deuterium.

In some embodiments, the compounds of the invention, and pharmaceutically acceptable salts thereof, include each constituent atom at approximately the natural abundance isotopic composition of the specified element.

In some embodiments, the compounds of the invention, and pharmaceutically acceptable salts thereof, include one or more atoms having an atomic mass or mass number which differs from the atomic mass or mass number of the most abundant isotope of the specified element (“isotope-labeled” compounds and salts). Examples of stable isotopes which are commercially available and suitable for the invention include without limitation isotopes of hydrogen, carbon, nitrogen, oxygen, and phosphorus, for example ²H, ¹³C, ¹⁵N, ¹⁸O, ¹⁷O, and ³¹P, respectively.

The isotope-labeled compounds and salts can be used in a number of beneficial ways, including as medicaments. In some embodiments, the isotope-labeled compounds and salts are deuterium (2H)-labeled. Deuterium (²H)-labeled compounds and salts are therapeutically useful with potential therapeutic advantages over the non-²H-labeled compounds. In general, deuterium (²H)-labeled compounds and salts can have higher metabolic stability as compared to those that are not isotope-labeled owing to the kinetic isotope effect described below. Higher metabolic stability translates directly into an increased in vivo half-life or lower dosages, which under most circumstances would represent a preferred embodiment of the present invention. The isotope-labeled compounds and salts can usually be prepared by carrying out the procedures disclosed in the synthesis schemes, the examples and the related description, replacing a non-isotope-labeled reactant by a readily available isotope-labeled reactant.

The deuterium (²H)-labeled compounds and salts can manipulate the rate of oxidative metabolism of the compound by way of the primary kinetic isotope effect. The primary kinetic isotope effect is a change of the rate for a chemical reaction that results from exchange of isotopic nuclei, which in turn is caused by the change in ground state energies of the covalent bonds involved in the reaction. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For example, if deuterium is bonded to a carbon atom at a non-exchangeable position, rate differences of k_{H}/k_{D}=2-7 are typical. For a further discussion, see S. L. Harbeson and R. D. Tung, Deuterium In Drug Discovery and Development, Ann. Rep. Med. Chem. 2011, 46, 403-417, incorporated in its entirety herein by reference.

The concentration of an isotope (e.g., deuterium) incorporated at a given position of an isotope-labeled compound of the invention, or a pharmaceutically acceptable salt thereof, may be defined by the isotopic enrichment factor. The term “isotopic enrichment factor,” as used herein, means the ratio between the abundance of an isotope at a given position in an isotope-labeled compound (or salt) and the natural abundance of the isotope.

Where an atom in a compound of the invention, or a pharmaceutically acceptable salt thereof, is designated as deuterium, such compound (or salt) has an isotopic enrichment factor for such atom of at least 3000 (˜45% deuterium incorporation). In some embodiments, the isotopic enrichment factor is at least 3500 (˜52.5% deuterium incorporation), at least 4000 (˜60% deuterium incorporation), at least 4500 (˜67.5% deuterium incorporation), at least 5000 (˜75% deuterium incorporation), at least 5500 (˜82.5% deuterium incorporation), at least 6000 (˜90% deuterium incorporation), at least 6333.3 (˜95% deuterium incorporation), at least 6466.7 (˜97% deuterium incorporation), at least 6600 (˜99% deuterium incorporation), or at least 6633.3 (˜99.5% deuterium incorporation).

In some embodiments, the invention relates to a compound of formula (I-A)

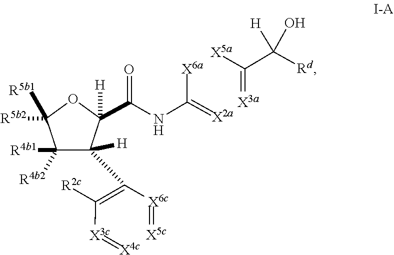

or a pharmaceutically acceptable salt thereof, wherein X^{2a}, X^{3a}, X^{5a}, X^{6a}, R^{d}, R^{4b1}, R^{4b2}, R^{5b1}, R^{5b2}, X^{3c}, X^{4c}, X^{5c}, X^{6c}, and R^{2c} are defined as set forth above in connection with formula (I).

In some embodiments, the invention relates to a compound of formula (I-A-1)

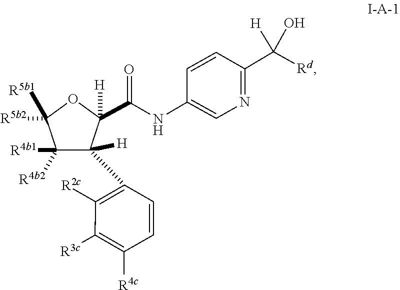

or a pharmaceutically acceptable salt thereof, wherein R^{d}, R^{4b1}, R^{4b2}, R^{5b1}, R^{5b2}, R^{2c}, R^{3c}, and R^{4c} are defined as set forth above in connection with formula (I).

In some embodiments, the invention relates to a compound of formula (I-A-2)

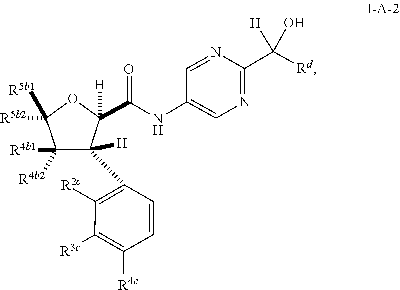

or a pharmaceutically acceptable salt thereof, wherein R^{d}, R^{4b1}, R^{4b2}, R^{5b1}, R^{5b2}, R^{2c}, R^{3c}, and R^{4c} are defined as set forth above in connection with formula (I).

In some embodiments, the invention relates to a compound of formula (I-A-3)

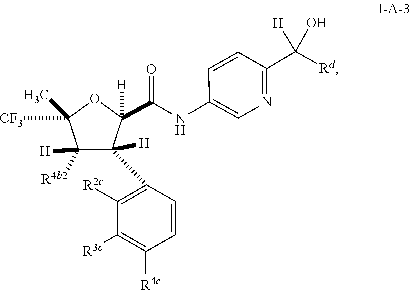

or a pharmaceutically acceptable salt thereof, wherein R^{d}, R^{4b2}, R^{2c}, R^{3c}, and R^{4c} are defined as set forth above in connection with formula (I).

In some embodiments, the invention relates to a compound of formula (I-B)

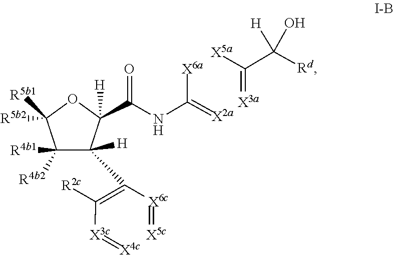

or a pharmaceutically acceptable salt thereof, wherein X^{2a}, X^{3a}, X^{5a}, X^{6a}, R^{d}, R^{4b1}, R^{4b2}, R^{5b1}, R^{5b2}, X^{3c}, X^{4c}X^{5c}, X^{6c}, and R^{2c} are defined as set forth above in connection with formula (I).

In some embodiments, the invention relates to a compound of formula (I-B-1)

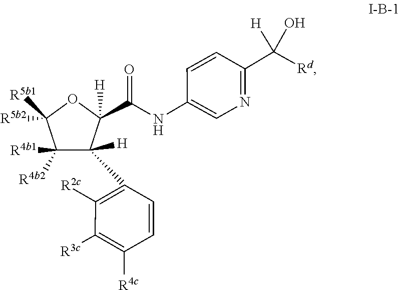

or a pharmaceutically acceptable salt thereof, wherein R^{d}, R^{4b1}, R^{4b2}, R^{5b1}, R^{5b2}, R^{2c}, R^{3c}, and R^{4c} are defined as set forth above in connection with formula (I).

In some embodiments, the invention relates to a compound of formula (I-B-2)

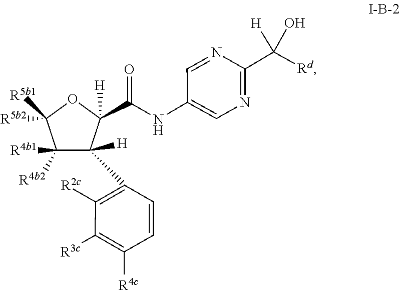

or a pharmaceutically acceptable salt thereof, wherein R^{d}, R^{4b1}, R^{4b2}, R^{5b1}, R^{5b2}, R^{2c}, R^{3c}, and R^{4c} are defined as set forth above in connection with formula (I).

In some embodiments, the invention relates to a compound of formula (I-B-3)

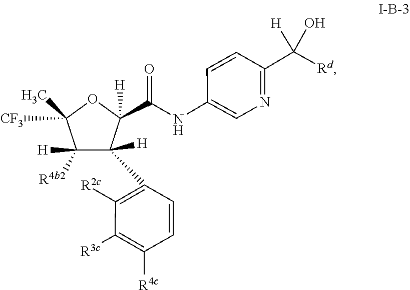

or a pharmaceutically acceptable salt thereof, wherein R^{d}, R^{4b2}, R^{2c}, R^{3c}, and R^{4c} are defined as set forth above in connection with formula (I).

In some embodiments, the invention relates to a compound of formula (I-C)

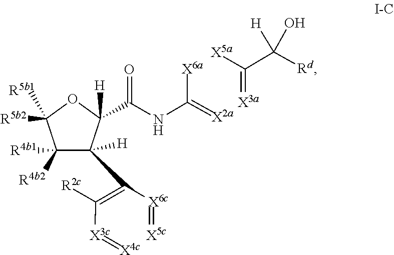

or a pharmaceutically acceptable salt thereof, wherein X^{2a}, X^{3a}, X^{5a}, X^{6a}, R^{d}, R^{4b1}, R^{4b2}, R^{5b1}, R^{5b2}, X^{3c}, X^{4c}X^{5c}, X^{6c}, and R^{2c} are defined as set forth above in connection with formula (I).

In some embodiments, the invention relates to a compound of formula (I-C-1)

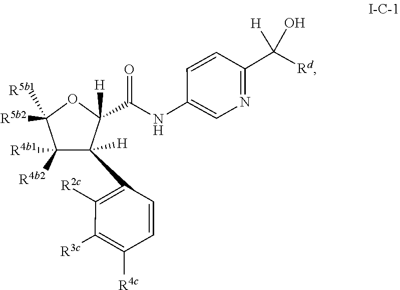

or a pharmaceutically acceptable salt thereof, wherein R^{d}, R^{4b1}, R^{4b2}, R^{5b1}, R^{5b2}, R^{2c}, R^{3c}, and R^{4c} are defined as set forth above in connection with formula (I).

In some embodiments, the invention relates to a compound of formula (I-C-2)

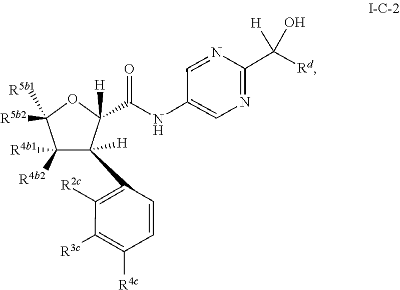

or a pharmaceutically acceptable salt thereof, wherein R^{d}, R^{4b1}, R^{4b2}, R^{5b1}, R^{5b2}, R^{2c}, R^{3c}, and R^{4c} are defined as set forth above in connection with formula (I).

In some embodiments, the invention relates to a compound of formula (I-C-3)

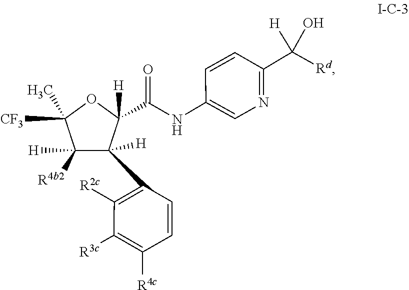

or a pharmaceutically acceptable salt thereof, wherein R^{d}, R^{4b2}, R^{2c}, R^{3c}, and R^{4c} are defined as set forth above in connection with formula (I).

In some embodiments, the invention relates to a compound of formula (I-D)

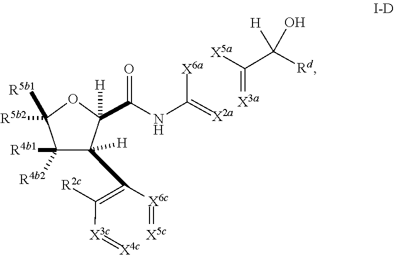

or a pharmaceutically acceptable salt thereof, wherein X^{2a}, X^{3a}, X^{5a}, X^{6a}, R^{d}, R^{4b1}, R^{4b2}, R^{5b1}, R^{5b2}, X^{3c}, X^{4c} X^{5c}, X^{6c}, and R^{2c} are defined as set forth above in connection with formula (I).

In some embodiments, the invention relates to a compound of formula (I-D-1)

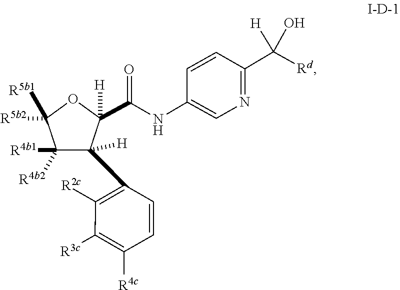

or a pharmaceutically acceptable salt thereof, wherein R^{d}, R^{4b1}, R^{4b2}, R^{5b1}, R^{5b2}, R^{2c}, R^{3c}, and R^{4c} are defined as set forth above in connection with formula (I).

In some embodiments, the invention relates to a compound of formula (I-D-2)

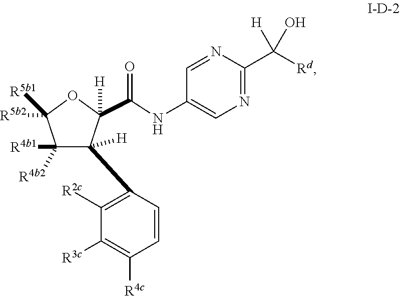

or a pharmaceutically acceptable salt thereof, wherein R^{d}, R^{4b1}, R^{4b2}, R^{5b1}, R^{5b2}, R^{2c}, R^{3c}, and R^{4c} are defined as set forth above in connection with formula (I).

In some embodiments, the invention relates to a compound of formula (I-D-3)

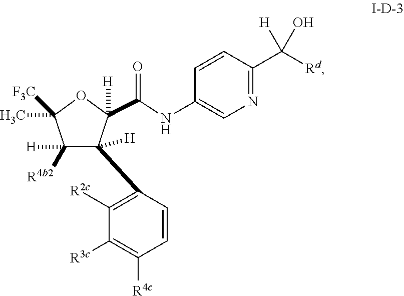

or a pharmaceutically acceptable salt thereof, wherein R^{d}, R^{4b2}, R^{2c}, R^{3c}, and R^{4c} are defined as set forth above in connection with formula (I).

In some embodiments, the invention relates to a compound of formula (I-D-4)

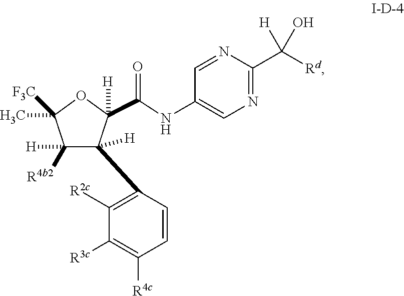

or a pharmaceutically acceptable salt thereof, wherein R^{d}, R^{4b2}, R^{2c}, R^{3c}, and R^{4c} are defined as set forth above in connection with formula (I).

In some embodiments, the invention relates to a compound of any one of formulas (I), (I-A), (I-B), (I-C), and (I-D), or a pharmaceutically acceptable salt thereof, wherein X^{2a} is C—R^{2a}. In other embodiments, X^{2a} is C—R^{2a}, and R^{2a} is H.

In some embodiments, the invention relates to a compound of any one of formulas (I), (I-A), (I-B), (I-C), and (I-D), or a pharmaceutically acceptable salt thereof, wherein X^{3a} is N.

In some embodiments, the invention relates to a compound of any one of formulas (I), (I-A), (I-B), (I-C), and (I-D), or a pharmaceutically acceptable salt thereof, wherein X^{2a} is C—R^{2a}, and R^{2a} is H.

In some embodiments, the invention relates to a compound of any one of formulas (I), (I-A), (I-B), (I-C), and (I-D), or a pharmaceutically acceptable salt thereof, wherein X^{3a} is N.

In some embodiments, the invention relates to a compound of any one of formulas (I), (I-A), (I-B), (I-C), and (I-D), or a pharmaceutically acceptable salt thereof, wherein X^{5a} is N. In other embodiments, X^{5a} is C—R^{5a}. In other embodiments, X^{5a} is C—R^{5a}, and R^{5a} is H.

In some embodiments, the invention relates to a compound of any one of formulas (I), (I-A), (I-B), (I-C), and (I-D), or a pharmaceutically acceptable salt thereof, wherein X^{6a} is C—R^{6a}. In other embodiments, X^{6a} is C—R^{6a}, and R^{6a} is H.

In some embodiments, the invention relates to a compound of any one of formulas (I), (I-A), (I-A-1), (I-A-2), (I-A-3), (I-B), (I-B-1), (I-B-2), (I-B-3), (I-C), (I-C-1), (I-C-2), (I-C-3), (I-D), (I-D-1), (I-D-2), (I-D-3), and (I-D-4), or a pharmaceutically acceptable salt thereof, wherein R^{e} is H. In other embodiments, R^{e} is OH. In other embodiments, R^{e} is C₁-C₆ alkoxy. In other embodiments, R^{e} is methoxy.

In some embodiments, the invention relates to a compound of any one of formulas (I), (I-A), (I-A-1), (I-A-2), (I-A-3), (I-B), (I-B-1), (I-B-2), (I-B-3), (I-C), (I-C-1), (I-C-2), (I-C-3), (I-D), (I-D-1), (I-D-2), (I-D-3), and (I-D-4), or a pharmaceutically acceptable salt thereof, wherein R^{d} is H, CH₂OH, or CH₂OCH₃.

In some embodiments, the invention relates to a compound of any one of formulas (I), (I-A), (I-B), (I-C), and (I-D), or a pharmaceutically acceptable salt thereof, wherein X^{5a} is C—R^{5a}, and R^{5a} and R^{d} form a CH₂CH₂ chain linking the C atoms to which R^{5a} and R^{d} are attached, wherein the CH₂ group that is bound to the C atom to which R^{5a} is attached may be replaced with O.

In some embodiments, the invention relates to a compound of any one of formulas (I), (I-A), (I-A-1), (I-A-2), (I-B), (I-B-1), (I-B-2), (I-C), (I-C-1), (I-C-2), (I-D), (I-D-1), and (I-D-2), or a pharmaceutically acceptable salt thereof, wherein R^{4b1} is H. In other embodiments, R^{4b1} is C₁-C₆ alkyl. In other embodiments, R^{4b1} is CH₃.

In some embodiments, the invention relates to a compound of any one of formulas (I), (I-A), (I-A-1), (I-A-2), (I-A-3), (I-B), (I-B-1), (I-B-2), (I-B-3), (I-C), (I-C-1), (I-C-2), (I-C-3), (I-D), (I-D-1), (I-D-2), (I-D-3), and (I-D-4), or a pharmaceutically acceptable salt thereof, wherein R^{4b2} is H. In other embodiments, R^{4b2} is C₁-C₆ alkyl. In other embodiments, R^{4b2} is CH₃.

In some embodiments, the invention relates to a compound of any one of formulas (I), (I-A), (I-A-1), (I-A-2), (I-B), (I-B-1), (I-B-2), (I-C), (I-C-1), (I-C-2), (I-D), (I-D-1), and (I-D-2), or a pharmaceutically acceptable salt thereof, wherein R^{5b1} is C₁-C₆ alkyl. In other embodiments, R^{5b1} is CH₃. In other embodiments, R^{5b1} is C₁-C₆ haloalkyl. In other embodiments, R^{5b1} is CF₃.

In some embodiments, the invention relates to a compound of any one of formulas (I), (I-A), (I-A-1), (I-A-2), (I-B), (I-B-1), (I-B-2), (I-C), (I-C-1), (I-C-2), (I-D), (I-D-1), and (I-D-2), or a pharmaceutically acceptable salt thereof, wherein R^{5b2} is C₁-C₆ alkyl. In other embodiments, R^{5b2} is CH₃. In other embodiments, R^{5b2} is C₁-C₆ haloalkyl. In other embodiments, R^{5b2} is CF₃.

In some embodiments, the invention relates to a compound of any one of formulas (I), (I-A), (I-A-1), (I-A-2), (I-A-3), (I-B), (I-B-1), (I-B-2), (I-B-3), (I-C), (I-C-1), (I-C-2), (I-C-3), (I-D), (I-D-1), (I-D-2), (I-D-3), and (I-D-4), or a pharmaceutically acceptable salt thereof, wherein R^{2c} is C₁-C₆ alkoxy. In other embodiments, R^{2c} is O—CH₂—C(R^{2c1})(R^{2c2})(R^{2c3}). In other embodiments, R^{2c} is OCH₂CH₂R^{2c3}. In other embodiments, R^{2c} is OCH₂CH₂N(R^{2c})(R^{2c7}). In other embodiments, R^{2c} is

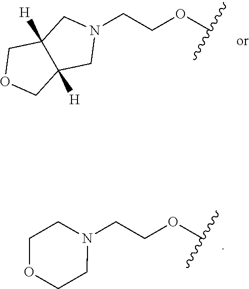 [SMILES: *OCCN1C[C@H]2COC[C@H]2C1 ; *OCCN1CCOCC1]

In other embodiments, R^{2c} is OCH₂CH₂OH, OCH₂CH₂OCH₃, or OCH₂CH₂OCHF₂. In other embodiments, R^{2c} is OCH₂CH(CH₃)R^{2c3}. In other embodiments, R^{2c} is OCH₂CH(CH₃)OCH₃. In other embodiments, R^{2c} is OCH₂C(O)R^{2c3}. In other embodiments, R^{2c} is OCH₂C(O)N(R^{2c6})(R^{2c7}). In other embodiments, R^{2c} is OCH₂C(O)N(CH₃)₂. In other embodiments, R^{2c} is OCH₂CH(R^{2c2})(R^{2c3}). In other embodiments, R^{2c} is

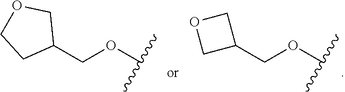 [SMILES: *OCC1CCOC1 ; *OCC1COC1]

In other embodiments, R^{2c} is O—CH(R^{2c4})(R^{2c5}). In other embodiments, R^{2c} is

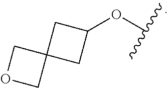 [SMILES: *OC1CC2(COC2)C1]

In some embodiments, the invention relates to a compound of any one of formulas (I), (I-A), (I-B), (I-C), and (I-D), or a pharmaceutically acceptable salt thereof, wherein X^{3c} is C—R^{3c}. In other embodiments, X^{3c} is C—R^{3c}, and R^{3c} is halo. In other embodiments, X^{3c} is C—R^{3c}, and R^{3c} is F. In other embodiments, X^{3c} is C—R^{3c}, and R^{3c} is C₁-C₆ alkyl. In other embodiments, X^{3c} is C—R^{3c}, and R^{3c} is CH₃.

In some embodiments, the invention relates to a compound of any one of formulas (I-A-1), (I-A-2), (I-A-3), (I-B-1), (I-B-2), (I-B-3), (I-C-1), (I-C-2), (I-C-3), (I-D-1), (I-D-2), (I-D-3), and (I-D-4), or a pharmaceutically acceptable salt thereof, wherein R^{3c} is halo. In other embodiments, R^{3c} is F. In other embodiments, R^{3c} is C₁-C₆ alkyl. In other embodiments, R^{3c} is CH₃.

In some embodiments, the invention relates to a compound of any one of formulas (I), (I-A), (I-B), (I-C), and (I-D), or a pharmaceutically acceptable salt thereof, wherein X^{4c} is C—R^{4c}. In other embodiments, X^{4c} is C—R^{4c}, and R^{4c} is halo. In other embodiments, X^{4c} is C—R^{4c}, and R^{4c} is F.

In some embodiments, the invention relates to a compound of any one of formulas (I-A-1), (I-A-2), (I-A-3), (I-B-1), (I-B-2), (I-B-3), (I-C-1), (I-C-2), (I-C-3), (I-D-1), (I-D-2), (I-D-3), and (I-D-4), or a pharmaceutically acceptable salt thereof, wherein R^{4c} is halo. In other embodiments, R^{4c} is F.

In some embodiments, the invention relates to a compound of any one of formulas (I), (I-A), (I-B), (I-C), and (I-D), or a pharmaceutically acceptable salt thereof, wherein X^{5c} is C—R^{5c}. In other embodiments, X^{5c} is C—R^{5c}, and R^{5c} is H.

In some embodiments, the invention relates to a compound of any one of formulas (I), (I-A), (I-B), (I-C), and (I-D), or a pharmaceutically acceptable salt thereof, wherein X^{6c} is C—R^{6c}. In other embodiments, X^{6c} is C—R^{6c}, and R^{6c} is H.

In some embodiments, the invention relates to a compound of any one of formulas (I), (I-A), (I-A-1), (I-A-2), (I-A-3), (I-B), (I-B-1), (I-B-2), (I-B-3), (I-C), (I-C-1), (I-C-2), (I-C-3), (I-D), (I-D-1), (I-D-2), (I-D-3), and (I-D-4), or any embodiment thereof, i.e., the compound in non-salt form.

In some embodiments, the invention relates to a compound selected from Table A, or a pharmaceutically acceptable salt thereof. In other embodiments, the invention relates to a compound selected from Table A, i.e., the compound in non-salt form.

TABLE A
Compound Structures and Names.

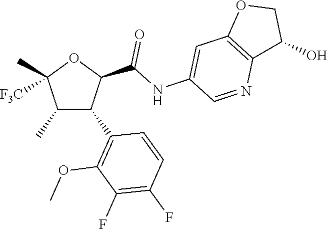 [SMILES: COc1c([C@H]2[C@H](C(=O)Nc3cnc4c(c3)OC[C@@H]4O)O[C@@](C)(C(F)(F)F)[C@H]2C)ccc(F)c1F]

(2R,3S,4S,5R)-3-(3,4-difluoro-2-
methoxyphenyl)-N-((R)-3-hydroxy-2,3-
dihydrofuro[3,2-b]pyridin-6-yl)-4,5-dimethyl-5-
(trifluoromethyl)tetrahydrofuran-2-carboxamide

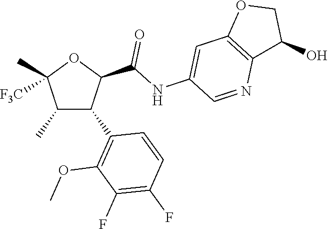 [SMILES: COc1c([C@H]2[C@H](C(=O)Nc3cnc4c(c3)OC[C@H]4O)O[C@@](C)(C(F)(F)F)[C@H]2C)ccc(F)c1F]

(2R,3S,4S,5R)-3-(3,4-difluoro-2-
methoxyphenyl)-N-((S)-3-hydroxy-2,3-
dihydrofuro[3,2-b]pyridin-6-yl)-4,5-dimethyl-5-
(trifluoromethyl)tetrahydrofuran-2-carboxamide

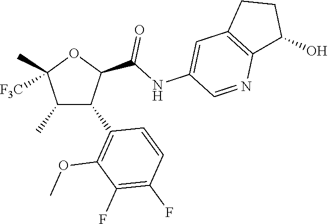 [SMILES: COc1c([C@H]2[C@H](C(=O)Nc3cnc4c(c3)CC[C@@H]4O)O[C@@](C)(C(F)(F)F)[C@H]2C)ccc(F)c1F]

(2R,3S,4S,5R)-3-(3,4-difluoro-2-methoxyphenyl)-
N-((S)-7-hydroxy-6,7-dihydro-5H-
cyclopenta[b]pyridin-3-yl)-4,5-dimethyl-5-
(trifluoromethyl)tetrahydrofuran-2-carboxamide

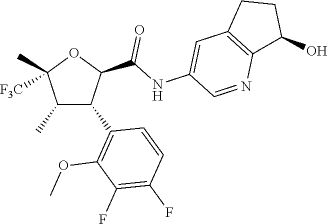 [SMILES: COc1c([C@H]2[C@H](C(=O)Nc3cnc4c(c3)CC[C@H]4O)O[C@@](C)(C(F)(F)F)[C@H]2C)ccc(F)c1F]

(2R,3S,4S,5R)-3-(3,4-difluoro-2-methoxyphenyl)-
N-((R)-7-hydroxy-6,7-dihydro-5H-
cyclopenta[b]pyridin-3-yl)-4,5-dimethyl-5-
(trifluoromethyl)tetrahydrofuran-2-carboxamide

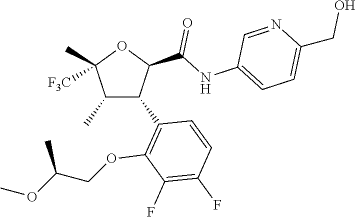 [SMILES: CO[C@@H](C)COc1c([C@H]2[C@H](C(=O)Nc3ccc(CO)nc3)O[C@@](C)(C(F)(F)F)[C@H]2C)ccc(F)c1F]

(2R,3S,4S,5R)-3-(3,4-difluoro-2-((S)-2-
methoxpropoxy)phenyl)-N-(6-
(hydroxymethyl)pyridin-3-yl)-4,5-dimethyl-5-
(trifluoromethyl)tetrahydrofuran-2-carboxamide

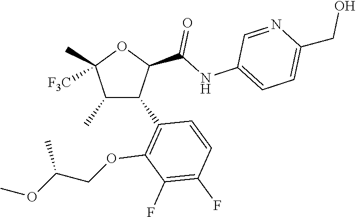 [SMILES: CO[C@H](C)COc1c([C@H]2[C@H](C(=O)Nc3ccc(CO)nc3)O[C@@](C)(C(F)(F)F)[C@H]2C)ccc(F)c1F]

(2R,3S,4S,5R)-3-(3,4-difluoro-2-((R)-2-
methoxypropoxy)phenyl)-N-(6-
(hydroxymethyl)pyridin-3-yl)-4,5-dimethyl-5-
(trifluoromethyl)tetrahydrofuran-2-carboxamide

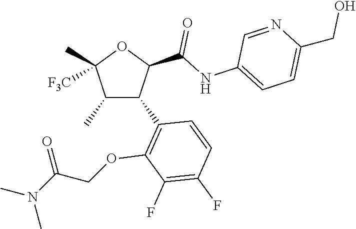 [SMILES: C[C@H]1[C@@H](c2ccc(F)c(F)c2OCC(=O)N(C)C)[C@H](C(=O)Nc2ccc(CO)nc2)O[C@@]1(C)C(F)(F)F]

(2R,3S,4S,5R)-3-(2-(2-(dimethylamino)-2-
oxoethoxy)-3,4-difluorophenyl)-N-(6-
(hydroxymethyl)pyridin-3-yl)-4,5-dimethyl-5-
(trifluoromethyl)tetrahydrofuran-2-
carboxamide

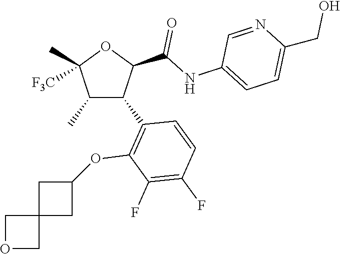 [SMILES: C[C@H]1[C@@H](c2ccc(F)c(F)c2OC2CC3(COC3)C2)[C@H](C(=O)Nc2ccc(CO)nc2)O[C@@]1(C)C(F)(F)F]

(2R,3S,4S,5R)-3-(2-((2-oxaspiro[3.3]heptan-6-
yl)oxy)-3,4-difluorophenyl)-N-(6-
(hydroxymethyl)pyridin-3-yl)-4,5-dimethyl-5-
(trifluoromethyl)tetrahydrofuran-2-carboxamide

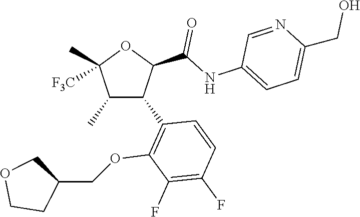 [SMILES: C[C@H]1[C@@H](c2ccc(F)c(F)c2OC[C@H]2CCOC2)[C@H](C(=O)Nc2ccc(CO)nc2)O[C@@]1(C)C(F)(F)F]

(2R,3S,4S,5R)-3-(3,4-difluoro-2-(((S)-
tetrahydrofuran-3-yl)methoxy)phenyl)-N-(6-
(hydroxymethyl)pyridin-3-yl)-4,5-dimethyl-5-
(trifluoromethyl)tetrahydrofuran-2-carboxamide

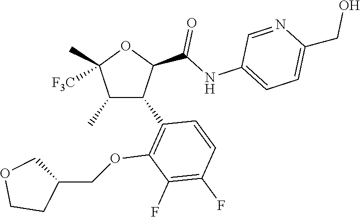 [SMILES: C[C@H]1[C@@H](c2ccc(F)c(F)c2OC[C@@H]2CCOC2)[C@H](C(=O)Nc2ccc(CO)nc2)O[C@@]1(C)C(F)(F)F]

(2R,3S,4S,5R)-3-(3,4-difluoro-2-(((R)-
tetrahydrofuran-3-yl)methoxy)phenyl)-N-(6-
(hydroxymethyl)pyridin-3-yl)-4,5-dimethyl-5-
(trifluoromethyl)tetrahydrofuran-2-carboxamide

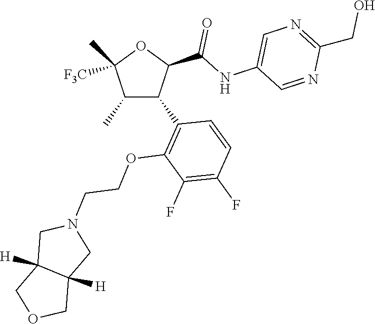 [SMILES: C[C@H]1[C@@H](c2ccc(F)c(F)c2OCCN2C[C@H]3COC[C@H]3C2)[C@H](C(=O)Nc2cnc(CO)nc2)O[C@@]1(C)C(F)(F)F]

(2R,3S,4S,5R)-3-(3,4-difluoro-2-(2-((3aR,6aS)-
tetrahydro-1H-furo[3,4-c]pyrrol-5(3H)-yl)ethoxy)phenyl)-
N-(2-(hydroxymethyl)pyrimidin-5-yl)-4,5-dimethyl-5-
(trifluoromethyl)tetrahydrofuran-2-carboxamide

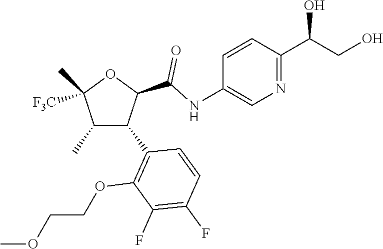 [SMILES: COCCOc1c([C@H]2[C@H](C(=O)Nc3ccc([C@@H](O)CO)nc3)O[C@@](C)(C(F)(F)F)[C@H]2C)ccc(F)c1F]

(2R,3S,4S,5R)-3-(3,4-difluoro-2-(2-
methoxyethoxy)phenyl)-N-(6-((R)-1,2-
dihydroxyethyl)pyridin-3-yl)-4,5-dimethyl-5-
(trifluoromethyl)tetrahydrofuran-2-carboxamide

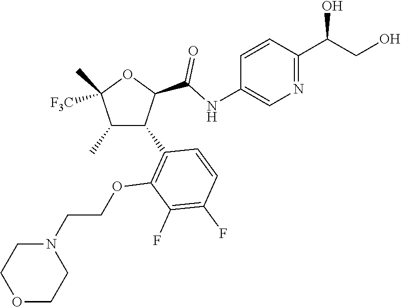 [SMILES: C[C@H]1[C@@H](c2ccc(F)c(F)c2OCCN2CCOCC2)[C@H](C(=O)Nc2ccc([C@@H](O)CO)nc2)O[C@@]1(C)C(F)(F)F]

(2R,3S,4S,5R)-3-(3,4-difluoro-2-(2-
morpholinoethoxy)phenyl)-N-(6-((R)-1,2-
dihydroxyethyl)pyridin-3-yl)-4,5-dimethyl-5-
(trifluoromethyl)tetrahydrofuran-2-carboxamide

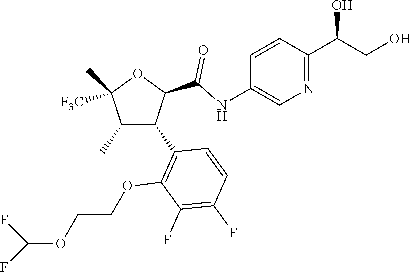 [SMILES: C[C@H]1[C@@H](c2ccc(F)c(F)c2OCCOC(F)F)[C@H](C(=O)Nc2ccc([C@@H](O)CO)nc2)O[C@@]1(C)C(F)(F)F]

(2R,3S,4S,5R)-3-(2-(2-(difluoromethoxy)ethoxy)-
3,4-difluorophenyl)-N-(6-((R)-1,2-
dihydroxyethyl)pyridin-3-yl)-4,5-dimethyl-5-
(trifluoromethyl)tetrahydrofuran-2-carboxamide

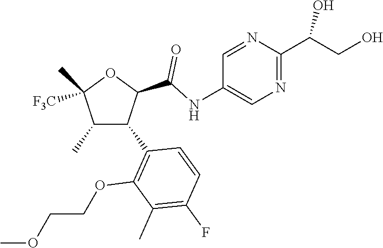 [SMILES: COCCOc1c([C@H]2[C@H](C(=O)Nc3cnc([C@H](O)CO)nc3)O[C@@](C)(C(F)(F)F)[C@H]2C)ccc(F)c1C]

(2R,3S,4S,5R)-N-(2-((S)-1,2-
dihydroxyethyl)pyrimidin-5-yl)-3-(4-fluoro-2-(2-
methoxyethoxy)-3-methylphenyl)-4,5-dimethyl-5-
(trifluoromethyl)tetrahydrofuran-2-carboxamide

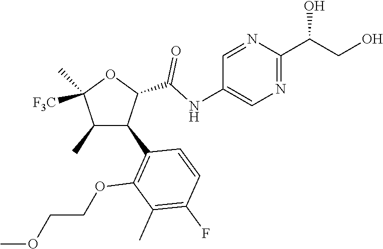 [SMILES: COCCOc1c([C@@H]2[C@@H](C(=O)Nc3cnc([C@H](O)CO)nc3)O[C@](C)(C(F)(F)F)[C@@H]2C)ccc(F)c1C]

(2S,3R,4R,5S)-N-(2-((S)-1,2-
dihydroxyethyl)pyrimidin-5-yl)-3-(4-fluoro-2-(2-
methoxyethoxy)-3-methylphenyl)-4,5-dimethyl-5-
(trifluoromethyl)tetrahydrofuran-2-carboxamide

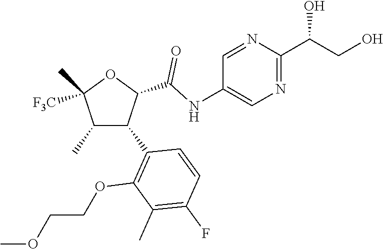 [SMILES: COCCOc1c([C@H]2[C@@H](C(=O)Nc3cnc([C@H](O)CO)nc3)O[C@@](C)(C(F)(F)F)[C@H]2C)ccc(F)c1C]

(2S,3S,4S,5R)-N-(2-((S)-1,2-
dihydroxyethyl)pyrimidin-5-yl)-3-(4-fluoro-2-(2-
methoxyethoxy)-3-methylphenyl)-4,5-dimethyl-5-
(trifluoromethyl)tetrahydrofuran-2-carboxamide

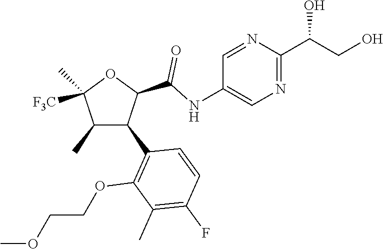 [SMILES: COCCOc1c([C@H]2[C@@H](C)[C@@](C)(C(F)(F)F)O[C@H]2C(=O)Nc2cnc([C@H](O)CO)nc2)ccc(F)c1C]

(2R,3R,4R,5S)-N-(2-((S)-1,2-
dihydroxyethyl)pyrimidin-5-yl)-3-(4-fluoro-2-(2-
methoxyethoxy)-3-methylphenyl)-4,5-dimethyl-5-
(trifluoromethyl)tetrahydrofuran-2-carboxamide

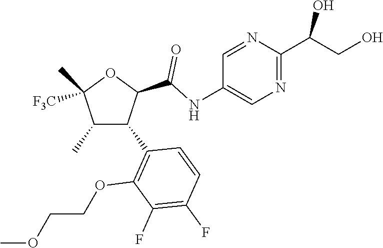 [SMILES: COCCOc1c([C@H]2[C@H](C(=O)Nc3cnc([C@@H](O)CO)nc3)O[C@@](C)(C(F)(F)F)[C@H]2C)ccc(F)c1F]

(2R,3S,4S,5R)-N-(2-((R)-1,2-
dihydroxyethyl)pyrimidin-5-yl)-3-(4-fluoro-2-(2-
methoxyethoxy)-3-methylphenyl)-4,5-dimethyl-5-
(trifluoromethyl)tetrahydrofuran-2-carboxamide

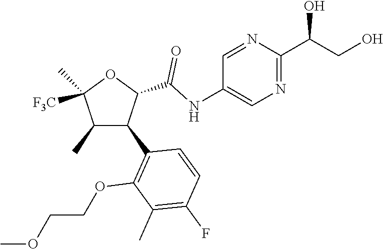 [SMILES: COCCOc1c([C@@H]2[C@@H](C(=O)Nc3cnc([C@@H](O)CO)nc3)O[C@](C)(C(F)(F)F)[C@@H]2C)ccc(F)c1C]

(2S,3R,4R,5S)-N-(2-((R)-1,2-
dihydroxyethyl)pyrimidin-5-yl)-3-(4-fluoro-2-(2-
methoxyethoxy)-3-methylphenyl)-4,5-dimethyl-5-
(trifluoromethyl)tetrahydrofuran-2-carboxamide

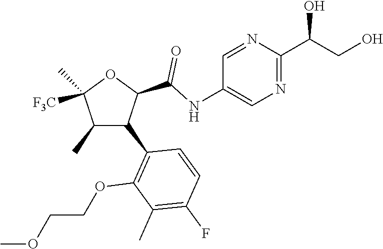

(2R,3R,4R,5S)-N-(2-((R)-1,2-
dihydroxyethyl)pyrimidin-5-yl)-3-(4-fluoro-2-(2-
methoxyethoxy)-3-methylphenyl)-4,5-dimethyl-5-
(trifluoromethyl)tetrahydrofuran-2-carboxamide

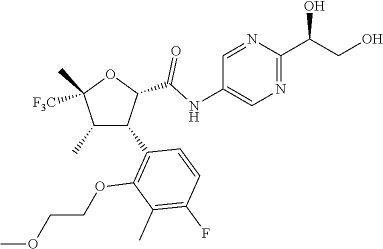 [SMILES: COCCOc1c([C@H]2[C@@H](C(=O)Nc3cnc([C@@H](O)CO)nc3)O[C@@](C)(C(F)(F)F)[C@H]2C)ccc(F)c1C]

(2S,3S,4S,5R)-N-(2-((R)-1,2-
dihydroxyethyl)pyrimidin-5-yl)-3-(4-fluoro-2-(2-
methoxyethoxy)-3-methylphenyl)-4,5-dimethyl-5-
(trifluoromethyl)tetrahydrofuran-2-carboxamide

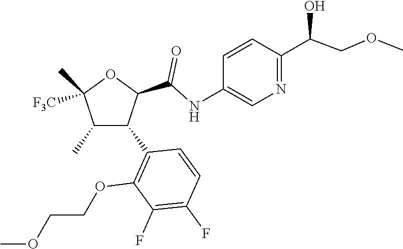

(2R,3S,4S,5R)-3-(3,4-difluoro-2-(2-
methoxyethoxy)phenyl)-N-(6-((R)-1-hydroxy-2-
methoxyethyl)pyridin-3-yl)-4,5-dimethyl-5-
(trifluoromethyl)tetrahydrofuran-2-carboxamide

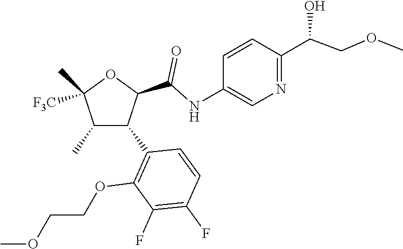

(2R,3S,4S,5R)-3-(3,4-difluoro-2-(2-
methoxyethoxy)phenyl)-N-(6-((S)-1-hydroxy-2-
methoxyethyl)pyridin-3-yl)-4,5-dimethyl-5-
(trifluoromethyl)tetrahydrofuran-2-carboxamide

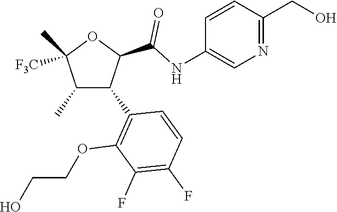 [SMILES: C[C@H]1[C@@H](c2ccc(F)c(F)c2OCCO)[C@H](C(=O)Nc2ccc(CO)nc2)O[C@@]1(C)C(F)(F)F]

(2R,3S,4S,5R)-3-(3,4-difluoro-2-(2-
hydroxyethoxy)phenyl)-N-(6-
(hydroxymethyl)pyridin-3-yl)-4,5-dimethyl-5-
(trifluoromethyl)tetrahydrofuran-2-
carboxamide

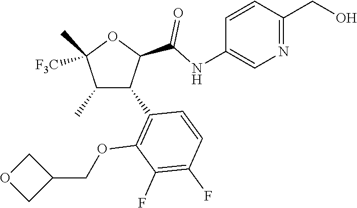 [SMILES: C[C@H]1[C@@H](c2ccc(F)c(F)c2OCC2COC2)[C@H](C(=O)Nc2ccc(CO)nc2)O[C@@]1(C)C(F)(F)F]

(2R,3S,4S,5R)-3-(3,4-difluoro-2-(oxetan-3-
ylmethoxy)phenyl)-N-(6-(hydroxymethyl)pyridin-
3-yl)-4,5-dimethyl-5-
(trifluoromethyl)tetrahydrofuran-2-carboxamide

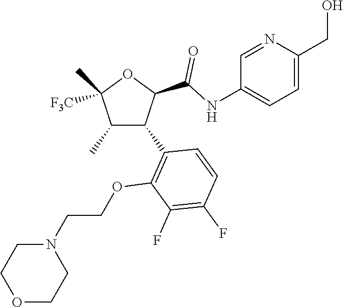 [SMILES: C[C@H]1[C@@H](c2ccc(F)c(F)c2OCCN2CCOCC2)[C@H](C(=O)Nc2ccc(CO)nc2)O[C@@]1(C)C(F)(F)F]

(2R,3S,4S,5R)-3-(3-fluoro-2-(2-
morpholinoethoxy)phenyl)-N-(6-
(hydroxymethyl)pyridin-3-yl)-4,5-dimethyl-5-
(trifluoromethyl)tetrahydrofuran-2-carboxamide

In some embodiments, the invention relates to a compound of formula

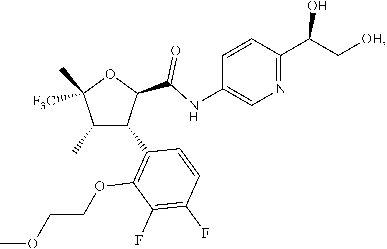 [SMILES: COCCOc1c([C@H]2[C@H](C(=O)Nc3ccc([C@@H](O)CO)nc3)O[C@@](C)(C(F)(F)F)[C@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

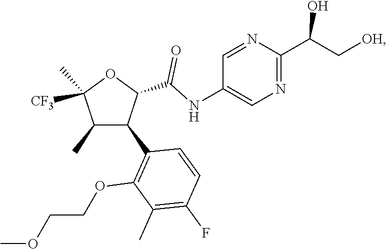 [SMILES: COCCOc1c([C@@H]2[C@@H](C(=O)Nc3cnc([C@@H](O)CO)nc3)O[C@](C)(C(F)(F)F)[C@@H]2C)ccc(F)c1C]

or a pharmaceutically acceptable salt thereof. In other embodiments, the invention relates to the foregoing compound in non-salt form. Such compound is considered to be a “compound of the invention,” as that term is used herein.

In some embodiments the invention relates to a compound of formula

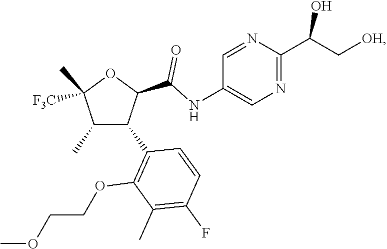 [SMILES: COCCOc1c([C@H]2[C@H](C(=O)Nc3cnc([C@@H](O)CO)nc3)O[C@@](C)(C(F)(F)F)[C@H]2C)ccc(F)c1C]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

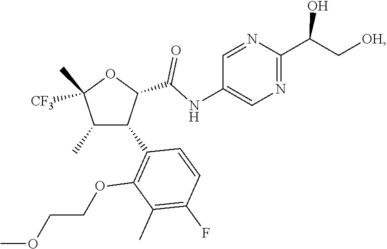 [SMILES: COCCOc1c([C@H]2[C@@H](C(=O)Nc3cnc([C@@H](O)CO)nc3)O[C@@](C)(C(F)(F)F)[C@H]2C)ccc(F)c1C]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

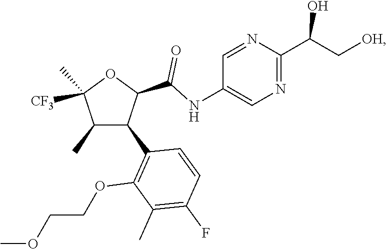 [SMILES: COCCOc1c([C@H]2[C@@H](C)[C@@](C)(C(F)(F)F)O[C@H]2C(=O)Nc2cnc([C@@H](O)CO)nc2)ccc(F)c1C]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

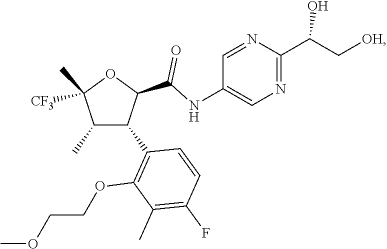 [SMILES: COCCOc1c([C@H]2[C@H](C(=O)Nc3cnc([C@H](O)CO)nc3)O[C@@](C)(C(F)(F)F)[C@H]2C)ccc(F)c1C]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

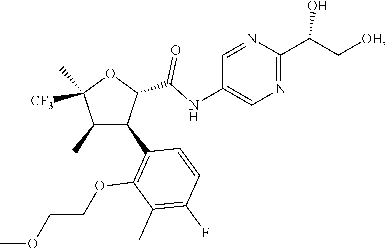 [SMILES: COCCOc1c([C@@H]2[C@@H](C(=O)Nc3cnc([C@H](O)CO)nc3)O[C@](C)(C(F)(F)F)[C@@H]2C)ccc(F)c1C]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

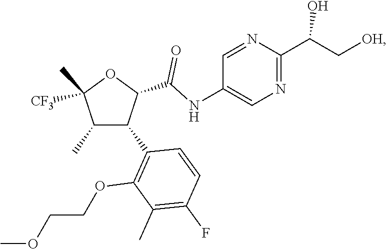 [SMILES: COCCOc1c([C@H]2[C@@H](C(=O)Nc3cnc([C@H](O)CO)nc3)O[C@@](C)(C(F)(F)F)[C@H]2C)ccc(F)c1C]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

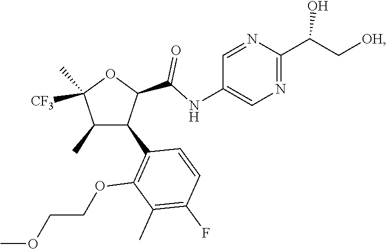 [SMILES: COCCOc1c([C@H]2[C@@H](C)[C@@](C)(C(F)(F)F)O[C@H]2C(=O)Nc2cnc([C@H](O)CO)nc2)ccc(F)c1C]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

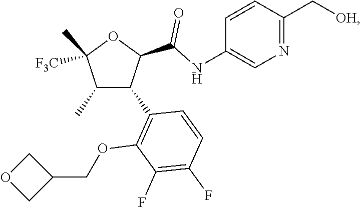 [SMILES: C[C@H]1[C@@H](c2ccc(F)c(F)c2OCC2COC2)[C@H](C(=O)Nc2ccc(CO)nc2)O[C@@]1(C)C(F)(F)F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

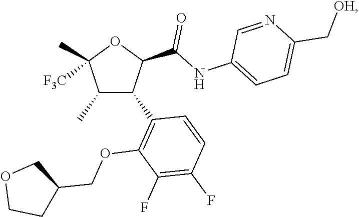 [SMILES: C[C@H]1[C@@H](c2ccc(F)c(F)c2OC[C@H]2CCOC2)[C@H](C(=O)Nc2ccc(CO)nc2)O[C@@]1(C)C(F)(F)F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

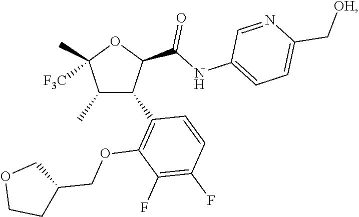 [SMILES: C[C@H]1[C@@H](c2ccc(F)c(F)c2OC[C@@H]2CCOC2)[C@H](C(=O)Nc2ccc(CO)nc2)O[C@@]1(C)C(F)(F)F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

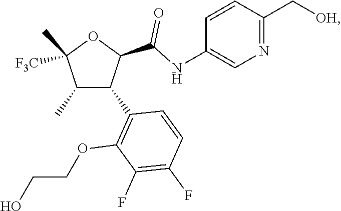 [SMILES: C[C@H]1[C@@H](c2ccc(F)c(F)c2OCCO)[C@H](C(=O)Nc2ccc(CO)nc2)O[C@@]1(C)C(F)(F)F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

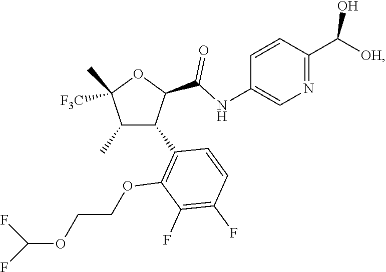

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

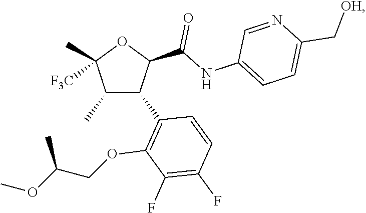 [SMILES: CO[C@@H](C)COc1c([C@H]2[C@H](C(=O)Nc3ccc(CO)nc3)O[C@@](C)(C(F)(F)F)[C@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

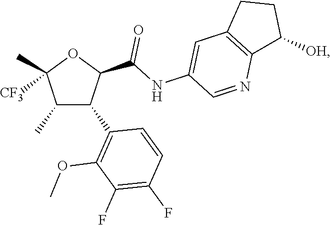

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

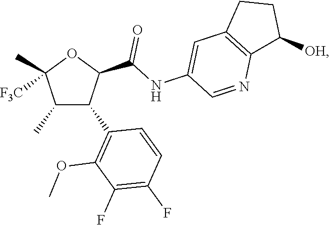 [SMILES: COc1c([C@H]2[C@H](C(=O)Nc3cnc4c(c3)CC[C@H]4O)O[C@@](C)(C(F)(F)F)[C@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

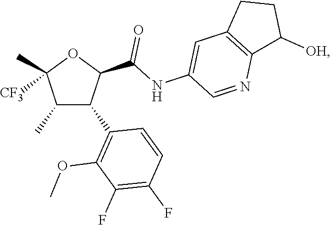

or a pharmaceutically acceptable salt thereof, wherein the compound has the absolute and relative stereochemistry corresponding to the second eluting isomer when the two diastereoisomers of (2R,3S,4S,5R)—N-(7-((tert-butyldimethylsilyl)oxy)-6,7-dihydro-5H-cyclopenta[b]pyridin-3-yl)-3-(3,4-difluoro-2-methoxyphenyl)-4,5-dimethyl-5-(trifluoromethyl)tetrahydrofuran-2-carboxamide are separated by SFC as described in Example 2, Step 2. In other embodiments, the invention relates to the foregoing compound in non-salt form. Such compound is considered to be a “compound of the invention,” as that term is used herein.

### Salts, Compositions, Uses, Formulation, Administration and Additional Agents

### Pharmaceutically Acceptable Salts and Compositions

As discussed herein, the invention provides compounds, and pharmaceutically acceptable salts thereof, that are inhibitors of voltage-gated sodium channels, and, thus, the present compounds, and pharmaceutically acceptable salts thereof, are useful for the treatment of diseases, disorders, and conditions including, but not limited to, chronic pain, gut pain, neuropathic pain, musculoskeletal pain, acute pain, inflammatory pain, cancer pain, idiopathic pain, postsurgical pain (e.g., bunionectomy pain, heriorrhaphy pain or abdominoplasty pain), visceral pain, multiple sclerosis, Charcot-Marie-Tooth syndrome, incontinence, pathological cough, or cardiac arrhythmia. Accordingly, in another aspect of the invention, pharmaceutical compositions are provided, wherein these compositions comprise a compound as described herein, or a pharmaceutically acceptable salt thereof, and optionally comprise a pharmaceutically acceptable carrier, adjuvant or vehicle. In certain embodiments, these compositions optionally further comprise one or more additional therapeutic agents. In some embodiments, the additional therapeutic agent is a sodium channel inhibitor.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” of a compound of this invention includes any non-toxic salt that, upon administration to a recipient, is capable of providing, either directly or indirectly, a compound of this invention or an inhibitorily active metabolite or residue thereof. The salt may be in pure form, in a mixture (e.g., solution, suspension, or colloid) with one or more other substances, or in the form of a hydrate, solvate, or co-crystal. As used herein, the term “inhibitorily active metabolite or residue thereof” means that a metabolite or residue thereof is also an inhibitor of a voltage-gated sodium channel.

Pharmaceutically acceptable salts are well known in the art. For example, S. M. Berge, et al. describe pharmaceutically acceptable salts in detail in J Pharmaceutical Sciences, 1977, 66, 1-19, incorporated herein by reference. Pharmaceutically acceptable salts of the compound of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As described herein, the pharmaceutically acceptable compositions of the invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose, starches such as corn starch and potato starch, cellulose and its derivatives such as sodium carboxymethyl cellulose, ethyl cellulose and cellulose acetate, powdered tragacanth, malt, gelatin, talc, excipients such as cocoa butter and suppository waxes, oils such as peanut oil, cottonseed oil, safflower oil, sesame oil, olive oil, corn oil and soybean oil, glycols, such a propylene glycol or polyethylene glycol, esters such as ethyl oleate and ethyl laurate, agar, buffering agents such as magnesium hydroxide and aluminum hydroxide, alginic acid, pyrogen-free water, isotonic saline, Ringer's solution, ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In another aspect, the invention features a pharmaceutical composition comprising a compound of the invention, or a pharmaceutically acceptable salt thereof, and a pharmaceutically acceptable carrier.

In another aspect, the invention features a pharmaceutical composition comprising a therapeutically effective amount of a compound, or a pharmaceutically acceptable salt thereof, and one or more pharmaceutically acceptable carriers or vehicles.

### Uses of Compounds and Pharmaceutically Acceptable Salts and Compositions

In another aspect, the invention features a method of inhibiting a voltage-gated sodium channel in a subject comprising administering to the subject a compound of the invention or a pharmaceutically acceptable salt thereof or a pharmaceutical composition thereof. In another aspect, the voltage-gated sodium channel is Na_{V}1.8.

In yet another aspect, the invention features a method of treating or lessening the severity in a subject of chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, or cardiac arrhythmia comprising administering an effective amount of a compound of the invention, a pharmaceutically acceptable salt thereof or a pharmaceutical composition thereof.

In yet another aspect, the invention features a method of treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia comprising administering an effective amount of a compound of the invention, a pharmaceutically acceptable salt thereof or a pharmaceutical composition thereof.

In yet another aspect, the invention features a method of treating or lessening the severity in a subject of gut pain, wherein gut pain comprises inflammatory bowel disease pain, Crohn's disease pain or interstitial cystitis pain wherein said method comprises administering an effective amount of a compound of the invention, a pharmaceutically acceptable salt thereof or a pharmaceutical composition thereof.

In yet another aspect, the invention features a method of treating or lessening the severity in a subject of neuropathic pain comprising administering an effective amount of a compound of the invention, a pharmaceutically acceptable salt thereof or a pharmaceutical composition thereof. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy). As used herein, the phrase “idiopathic small-fiber neuropathy” shall be understood to include any small fiber neuropathy.

In yet another aspect, the invention features a method of treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma, traumatic neuroma, Morton's neuroma, nerve entrapment injury, spinal stenosis, carpal tunnel syndrome, radicular pain, sciatica pain, nerve avulsion injury, brachial plexus avulsion injury, complex regional pain syndrome, drug therapy induced neuralgia, cancer chemotherapy induced neuralgia, anti-retroviral therapy induced neuralgia, post spinal cord injury pain, small fiber neuropathy, idiopathic small-fiber neuropathy, idiopathic sensory neuropathy or trigeminal autonomic cephalalgia wherein said method comprises administering an effective amount of a compound of the invention, a pharmaceutically acceptable salt thereof or a pharmaceutical composition thereof.

In yet another aspect, the invention features a method of treating or lessening the severity in a subject of musculoskeletal pain comprising administering an effective amount of a compound of the invention, a pharmaceutically acceptable salt thereof or a pharmaceutical composition thereof. In some aspects, the musculoskeletal pain comprises osteoarthritis pain.

In yet another aspect, the invention features a method of treating or lessening the severity in a subject of musculoskeletal pain, wherein musculoskeletal pain comprises osteoarthritis pain, back pain, cold pain, burn pain or dental pain wherein said method comprises administering an effective amount of a compound of the invention, a pharmaceutically acceptable salt thereof or a pharmaceutical composition thereof.

In yet another aspect, the invention features a method of treating or lessening the severity in a subject of inflammatory pain, wherein inflammatory pain comprises rheumatoid arthritis pain or vulvodynia wherein said method comprises administering an effective amount of a compound of the invention, a pharmaceutically acceptable salt thereof or a pharmaceutical composition thereof.

In yet another aspect, the invention features a method of treating or lessening the severity in a subject of inflammatory pain, wherein inflammatory pain comprises rheumatoid arthritis pain wherein said method comprises administering an effective amount of a compound of the invention, a pharmaceutically acceptable salt thereof or a pharmaceutical composition thereof.

In yet another aspect, the invention features a method of treating or lessening the severity in a subject of idiopathic pain, wherein idiopathic pain comprises fibromyalgia pain wherein said method comprises administering an effective amount of a compound of the invention, a pharmaceutically acceptable salt thereof or a pharmaceutical composition thereof.

In yet another aspect, the invention features a method of treating or lessening the severity in a subject of pathological cough wherein said method comprises administering an effective amount of a compound of the invention, a pharmaceutically acceptable salt thereof or a pharmaceutical composition thereof.

In yet another aspect, the invention features a method of treating or lessening the severity in a subject of acute pain comprising administering an effective amount of a compound of the invention, a pharmaceutically acceptable salt thereof or a pharmaceutical composition thereof. In some aspects, the acute pain comprises acute post-operative pain.

In yet another aspect, the invention features a method of treating or lessening the severity in a subject of postsurgical pain (e.g., joint replacement pain, soft tissue surgery pain, herniorrhaphy pain, bunionectomy pain or abdominoplasty pain) comprising administering an effective amount of a compound of the invention, a pharmaceutically acceptable salt thereof or a pharmaceutical composition thereof.

In yet another aspect, the invention features a method of treating or lessening the severity in a subject of bunionectomy pain comprising administering an effective amount of a compound of the invention, a pharmaceutically acceptable salt thereof or a pharmaceutical composition thereof.

In yet another aspect, the invention features a method of treating or lessening the severity in a subject of herniorrhaphy pain comprising administering an effective amount of a compound of the invention, a pharmaceutically acceptable salt thereof or a pharmaceutical composition thereof.

In yet another aspect, the invention features a method of treating or lessening the severity in a subject of abdominoplasty pain comprising administering an effective amount of a compound of the invention, a pharmaceutically acceptable salt thereof or a pharmaceutical composition thereof.

In yet another aspect, the invention features a method of treating or lessening the severity in a subject of visceral pain comprising administering an effective amount of a compound of the invention, a pharmaceutically acceptable salt thereof or a pharmaceutical composition thereof. In some aspects, the visceral pain comprises visceral pain from abdominoplasty.

In yet another aspect, the invention features a method of treating or lessening the severity in a subject of a neurodegenerative disease comprising administering an effective amount of a compound of the invention, a pharmaceutically acceptable salt thereof or a pharmaceutical composition thereof. In some aspects, the neurodegenerative disease comprises multiple sclerosis. In some aspects, the neurodegenerative disease comprises Pitt Hopkins Syndrome (PTHS).

In yet another aspect, the invention features a method wherein the subject is treated with one or more additional therapeutic agents administered concurrently with, prior to, or subsequent to treatment with an effective amount of the compound, pharmaceutically acceptable salt or pharmaceutical composition. In some embodiments, the additional therapeutic agent is a sodium channel inhibitor.

In another aspect, the invention features a method of inhibiting a voltage-gated sodium channel in a biological sample comprising contacting the biological sample with an effective amount of a compound of the invention, a pharmaceutically acceptable salt thereof or a pharmaceutical composition thereof. In another aspect, the voltage-gated sodium channel is Na_{V}1.8.

In another aspect, the invention features a method of treating or lessening the severity in a subject of acute pain, sub-acute and chronic pain, nociceptive pain, neuropathic pain, inflammatory pain, nociplastic pain, arthritis, migraine, cluster headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility, comprising administering an effective amount of a compound of the invention, a pharmaceutically acceptable salt thereof or a pharmaceutical composition thereof.

In another aspect, the invention features a method of treating or lessening the severity in a subject of femur cancer pain, non-malignant chronic bone pain, rheumatoid arthritis, osteoarthritis, spinal stenosis, neuropathic low back pain, myofascial pain syndrome, fibromyalgia, temporomandibular joint pain, chronic visceral pain, abdominal pain, pancreatic pain, IBS pain, chronic and acute headache pain, migraine, tension headache, cluster headaches, chronic and acute neuropathic pain, post-herpetic neuralgia, diabetic neuropathy, HIV-associated neuropathy, trigeminal neuralgia, Charcot-Marie-Tooth neuropathy, hereditary sensory neuropathy, peripheral nerve injury, painful neuromas, ectopic proximal and distal discharges, radiculopathy, chemotherapy induced neuropathic pain, radiotherapy-induced neuropathic pain, persistent/chronic post-surgical pain (e.g., post amputation, post-thoracotomy, post-cardiac surgery), post-mastectomy pain, central pain, spinal cord injury pain, post-stroke pain, thalamic pain, phantom pain (e.g., following removal of lower extremity, upper extremity, breast), intractable pain, acute pain, acute post-operative pain, acute musculoskeletal pain, joint pain, mechanical low back pain, neck pain, tendonitis, injury pain, exercise pain, acute visceral pain, pyelonephritis, appendicitis, cholecystitis, intestinal obstruction, hernias, chest pain, cardiac pain, pelvic pain, renal colic pain, acute obstetric pain, labor pain, cesarean section pain, acute inflammatory pain, burn pain, trauma pain, acute intermittent pain, endometriosis, acute herpes zoster pain, sickle cell anemia, acute pancreatitis, breakthrough pain, orofacial pain, sinusitis pain, dental pain, multiple sclerosis (MS) pain, pain in depression, leprosy pain, Behcet's disease pain, adiposis dolorosa, phlebitic pain, Guillain-Barre pain, painful legs and moving toes, Haglund syndrome, erythromelalgia pain, Fabry's disease pain, bladder and urogenital disease, urinary incontinence, pathological cough, hyperactive bladder, painful bladder syndrome, interstitial cystitis (IC), prostatitis, complex regional pain syndrome (CRPS), type I, complex regional pain syndrome (CRPS) type II, widespread pain, paroxysmal extreme pain, pruritus, tinnitus, or angina-induced pain, comprising administering an effective amount of a compound of the invention, a pharmaceutically acceptable salt thereof or a pharmaceutical composition thereof.

### Compounds, Pharmaceutically Acceptable Salts, and Compositions for Use

In another aspect, the invention features a compound of the invention, or a pharmaceutically acceptable salt or pharmaceutical composition thereof, for use as a medicament.

In another aspect, the invention features a compound of the invention, or a pharmaceutically acceptable salt or pharmaceutical composition thereof, for use in a method of inhibiting a voltage-gated sodium channel in a subject. In another aspect, the voltage-gated sodium channel is Na_{V}1.8.

In another aspect, the invention features a compound of the invention, or a pharmaceutically acceptable salt or pharmaceutical composition thereof, for use in a method of treating or lessening the severity in a subject of chronic pain, gut pain, neuropathic pain, musculoskeletal pain, acute pain, inflammatory pain, cancer pain, idiopathic pain, postsurgical pain (e.g., herniorrhaphy pain, bunionectomy pain or abdominoplasty pain), visceral pain, multiple sclerosis, Charcot-Marie-Tooth syndrome, incontinence, pathological cough, or cardiac arrhythmia.

In another aspect, the invention features a compound of the invention, or a pharmaceutically acceptable salt or pharmaceutical composition thereof, for use in a method of treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia.

In another aspect, the invention features a compound of the invention, or a pharmaceutically acceptable salt or pharmaceutical composition thereof, for use in a method of treating or lessening the severity in a subject of gut pain, wherein gut pain comprises inflammatory bowel disease pain, Crohn's disease pain or interstitial cystitis pain.

In another aspect, the invention features a compound of the invention, or a pharmaceutically acceptable salt or pharmaceutical composition thereof, for use in a method of treating or lessening the severity in a subject of neuropathic pain. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy). As used herein, the phrase “idiopathic small-fiber neuropathy” shall be understood to include any small fiber neuropathy.

In another aspect, the invention features a compound of the invention, or a pharmaceutically acceptable salt or pharmaceutical composition thereof, for use in a method of treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma, traumatic neuroma, Morton's neuroma, nerve entrapment injury, spinal stenosis, carpal tunnel syndrome, radicular pain, sciatica pain, nerve avulsion injury, brachial plexus avulsion injury, complex regional pain syndrome, drug therapy induced neuralgia, cancer chemotherapy induced neuralgia, anti-retroviral therapy induced neuralgia, post spinal cord injury pain, small fiber neuropathy, idiopathic small-fiber neuropathy, idiopathic sensory neuropathy or trigeminal autonomic cephalalgia.

In another aspect, the invention features a compound of the invention, or a pharmaceutically acceptable salt or pharmaceutical composition thereof, for use in a method of treating or lessening the severity in a subject of musculoskeletal pain. In some aspects, the musculoskeletal pain comprises osteoarthritis pain.

In another aspect, the invention features a compound of the invention, or a pharmaceutically acceptable salt or pharmaceutical composition thereof, for use in a method of treating or lessening the severity in a subject of musculoskeletal pain, wherein musculoskeletal pain comprises osteoarthritis pain, back pain, cold pain, burn pain or dental pain.

In another aspect, the invention features a compound of the invention, or a pharmaceutically acceptable salt or pharmaceutical composition thereof, for use in a method of treating or lessening the severity in a subject of inflammatory pain, wherein inflammatory pain comprises rheumatoid arthritis pain or vulvodynia.

In another aspect, the invention features a compound of the invention, or a pharmaceutically acceptable salt or pharmaceutical composition thereof, for use in a method of treating or lessening the severity in a subject of inflammatory pain, wherein inflammatory pain comprises rheumatoid arthritis pain.

In another aspect, the invention features a compound of the invention, or a pharmaceutically acceptable salt or pharmaceutical composition thereof, for use in a method of treating or lessening the severity in a subject of idiopathic pain, wherein idiopathic pain comprises fibromyalgia pain.

In another aspect, the invention features a compound of the invention, or a pharmaceutically acceptable salt or pharmaceutical composition thereof, for use in a method of treating or lessening the severity in a subject of pathological cough.

In another aspect, the invention features a compound of the invention, or a pharmaceutically acceptable salt or pharmaceutical composition thereof, for use in a method of treating or lessening the severity in a subject of acute pain. In some aspects, the acute pain comprises acute post-operative pain.

In another aspect, the invention features a compound of the invention, or a pharmaceutically acceptable salt or pharmaceutical composition thereof, for use in a method of treating or lessening the severity in a subject of postsurgical pain (e.g., joint replacement pain, soft tissue surgery pain, heriorrhaphy pain, bunionectomy pain or abdominoplasty pain).

In another aspect, the invention features a compound of the invention, or a pharmaceutically acceptable salt or pharmaceutical composition thereof, for use in a method of treating or lessening the severity in a subject of bunionectomy pain.

In another aspect, the invention features a compound of the invention, or a pharmaceutically acceptable salt or pharmaceutical composition thereof, for use in a method of treating or lessening the severity in a subject of heriorrhaphy pain.

In another aspect, the invention features a compound of the invention, or a pharmaceutically acceptable salt or pharmaceutical composition thereof, for use in a method of treating or lessening the severity in a subject of abdominoplasty pain.

In another aspect, the invention features a compound of the invention, or a pharmaceutically acceptable salt or pharmaceutical composition thereof, for use in a method of treating or lessening the severity in a subject of visceral pain. In some aspects, the visceral pain comprises visceral pain from abdominoplasty.

In another aspect, the invention features a compound of the invention, or a pharmaceutically acceptable salt or pharmaceutical composition thereof, for use in a method of treating or lessening the severity in a subject of a neurodegenerative disease. In some aspects, the neurodegenerative disease comprises multiple sclerosis. In some aspects, the neurodegenerative disease comprises Pitt Hopkins Syndrome (PTHS).

In another aspect, the invention features a compound of the invention, or a pharmaceutically acceptable salt or pharmaceutical composition thereof, for use in a method wherein the subject is treated with one or more additional therapeutic agents administered concurrently with, prior to, or subsequent to treatment with an effective amount of the compound, pharmaceutically acceptable salt or pharmaceutical composition. In some embodiments, the additional therapeutic agent is a sodium channel inhibitor.

In another aspect, the invention features a compound of the invention, or a pharmaceutically acceptable salt or pharmaceutical composition thereof, for use in a method of inhibiting a voltage-gated sodium channel in a biological sample comprising contacting the biological sample with an effective amount of a compound of the invention, a pharmaceutically acceptable salt thereof or a pharmaceutical composition thereof. In another aspect, the voltage-gated sodium channel is Na_{V}1.8.

In another aspect, the invention features a compound of the invention, or a pharmaceutically acceptable salt or pharmaceutical composition thereof, for use in a method of treating or lessening the severity in a subject of acute pain, sub-acute and chronic pain, nociceptive pain, neuropathic pain, inflammatory pain, nociplastic pain, arthritis, migraine, cluster headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, heriorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/hemiation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility.

In another aspect, the invention features a compound of the invention, or a pharmaceutically acceptable salt or pharmaceutical composition thereof, for use in a method of treating or lessening the severity in a subject of femur cancer pain, non-malignant chronic bone pain, rheumatoid arthritis, osteoarthritis, spinal stenosis, neuropathic low back pain, myofascial pain syndrome, fibromyalgia, temporomandibular joint pain, chronic visceral pain, abdominal pain, pancreatic pain, IBS pain, chronic and acute headache pain, migraine, tension headache, cluster headaches, chronic and acute neuropathic pain, post-herpetic neuralgia, diabetic neuropathy, HIV-associated neuropathy, trigeminal neuralgia, Charcot-Marie-Tooth neuropathy, hereditary sensory neuropathy, peripheral nerve injury, painful neuromas, ectopic proximal and distal discharges, radiculopathy, chemotherapy induced neuropathic pain, radiotherapy-induced neuropathic pain, persistent/chronic post-surgical pain (e.g., post amputation, post-thoracotomy, post-cardiac surgery), post-mastectomy pain, central pain, spinal cord injury pain, post-stroke pain, thalamic pain, phantom pain (e.g., following removal of lower extremity, upper extremity, breast), intractable pain, acute pain, acute post-operative pain, acute musculoskeletal pain, joint pain, mechanical low back pain, neck pain, tendonitis, injury pain, exercise pain, acute visceral pain, pyelonephritis, appendicitis, cholecystitis, intestinal obstruction, hernias, chest pain, cardiac pain, pelvic pain, renal colic pain, acute obstetric pain, labor pain, cesarean section pain, acute inflammatory pain, burn pain, trauma pain, acute intermittent pain, endometriosis, acute herpes zoster pain, sickle cell anemia, acute pancreatitis, breakthrough pain, orofacial pain, sinusitis pain, dental pain, multiple sclerosis (MS) pain, pain in depression, leprosy pain, Behcet's disease pain, adiposis dolorosa, phlebitic pain, Guillain-Barre pain, painful legs and moving toes, Haglund syndrome, erythromelalgia pain, Fabry's disease pain, bladder and urogenital disease, urinary incontinence, pathological cough, hyperactive bladder, painful bladder syndrome, interstitial cystitis (IC), prostatitis, complex regional pain syndrome (CRPS), type I, complex regional pain syndrome (CRPS) type II, widespread pain, paroxysmal extreme pain, pruritus, tinnitus, or angina-induced pain.

In another aspect, the invention features a compound of the invention, or a pharmaceutically acceptable salt or pharmaceutical composition thereof, for use in a method of treating or lessening the severity in a subject of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbrosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain.

### Manufacture of Medicaments

In another aspect, the invention provides the use of a compound of the invention, or a pharmaceutically acceptable salt or pharmaceutical composition thereof, for the manufacture of a medicament.

In another aspect, the invention provides the use of a compound of the invention, a pharmaceutically acceptable salt thereof, or a pharmaceutical composition thereof for the manufacture of a medicament for use in inhibiting a voltage-gated sodium channel. In another aspect, the voltage-gated sodium channel is Na_{V}1.8.

In yet another aspect, the invention provides the use of a compound of the invention, a pharmaceutically acceptable salt thereof, or a pharmaceutical composition thereof for the manufacture of a medicament for use in treating or lessening the severity in a subject of chronic pain, gut pain, neuropathic pain, musculoskeletal pain, acute pain, inflammatory pain, cancer pain, idiopathic pain, postsurgical pain (e.g., herniorrhaphy pain, bunionectomy pain or abdominoplasty pain), visceral pain, multiple sclerosis, Charcot-Marie-Tooth syndrome, incontinence, pathological cough, or cardiac arrhythmia.

In yet another aspect, the invention provides the use of a compound of the invention, a pharmaceutically acceptable salt thereof, or a pharmaceutical composition thereof for the manufacture of a medicament for use in treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia.

In yet another aspect, the invention provides the use of the compound, pharmaceutically acceptable salt, or pharmaceutical composition described herein for the manufacture of a medicament for use in treating or lessening the severity in a subject of gut pain, wherein gut pain comprises inflammatory bowel disease pain, Crohn's disease pain or interstitial cystitis pain.

In yet another aspect, the invention provides a compound of the invention, a pharmaceutically acceptable salt thereof, or a pharmaceutical composition thereof for the manufacture of a medicament for use in treating or lessening the severity in a subject of neuropathic pain. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy).

In yet another aspect, the invention provides the use of a compound of the invention, a pharmaceutically acceptable salt thereof, or a pharmaceutical composition thereof for the manufacture of a medicament for use in a treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma, traumatic neuroma, Morton's neuroma, nerve entrapment injury, spinal stenosis, carpal tunnel syndrome, radicular pain, sciatica pain, nerve avulsion injury, brachial plexus avulsion injury, complex regional pain syndrome, drug therapy induced neuralgia, cancer chemotherapy induced neuralgia, anti-retroviral therapy induced neuralgia, post spinal cord injury pain, small fiber neuropathy, idiopathic small-fiber neuropathy, idiopathic sensory neuropathy or trigeminal autonomic neuropathy.

In yet another aspect, the invention provides the use of a compound of the invention, a pharmaceutically acceptable salt thereof, or a pharmaceutical composition thereof for the manufacture of a medicament for use in treating or lessening the severity in a subject of musculoskeletal pain. In some aspects the musculoskeletal pain comprises osteoarthritis pain.

In yet another aspect, the invention provides the use of a compound of the invention, a pharmaceutically acceptable salt thereof, or a pharmaceutical composition thereof for the manufacture of a medicament for use in treating or lessening the severity in a subject of musculoskeletal pain, wherein musculoskeletal pain comprises osteoarthritis pain, back pain, cold pain, burn pain or dental pain.

In yet another aspect, the invention provides the use of a compound of the invention, a pharmaceutically acceptable salt thereof, or a pharmaceutical composition thereof for the manufacture of a medicament for use in treating or lessening the severity in a subject of inflammatory pain, wherein inflammatory pain comprises rheumatoid arthritis pain or vulvodynia.

In yet another aspect, the invention provides the use of a compound of the invention, a pharmaceutically acceptable salt thereof, or a pharmaceutical composition thereof for the manufacture of a medicament for use in treating or lessening the severity in a subject of inflammatory pain, wherein inflammatory pain comprises rheumatoid arthritis pain.

In yet another aspect, the invention provides the use of a compound of the invention, a pharmaceutically acceptable salt thereof, or a pharmaceutical composition thereof for the manufacture of a medicament for use in treating or lessening the severity in a subject of idiopathic pain, wherein idiopathic pain comprises fibromyalgia pain.

In yet another aspect, the invention provides the use of a compound of the invention, a pharmaceutically acceptable salt thereof, or a pharmaceutical composition thereof for the manufacture of a medicament for use in treating or lessening the severity in a subject of pathological cough.

In yet another aspect, the invention provides the use of a compound of the invention, a pharmaceutically acceptable salt thereof, or a pharmaceutical composition thereof for the manufacture of a medicament for use in treating or lessening the severity in a subject of acute pain. In some aspects, the acute pain comprises acute post-operative pain.

In yet another aspect, the invention provides the use of a compound of the invention, a pharmaceutically acceptable salt thereof, or a pharmaceutical composition thereof for the manufacture of a medicament for use in treating or lessening the severity in a subject of postsurgical pain (e.g., joint replacement pain, soft tissue surgery pain, herniorrhaphy pain, bunionectomy pain or abdominoplasty pain).

In yet another aspect, the invention provides the use of a compound of the invention, a pharmaceutically acceptable salt thereof, or a pharmaceutical composition thereof for the manufacture of a medicament for use in treating or lessening the severity in a subject of herniorrhaphy pain.

In yet another aspect, the invention provides the use of a compound of the invention, a pharmaceutically acceptable salt thereof, or a pharmaceutical composition thereof for the manufacture of a medicament for use in treating or lessening the severity in a subject of bunionectomy pain.

In yet another aspect, the invention provides the use of a compound of the invention, a pharmaceutically acceptable salt thereof, or a pharmaceutical composition thereof for the manufacture of a medicament for use in treating or lessening the severity in a subject of abdominoplasty pain.

In yet another aspect, the invention provides the use of a compound of the invention, a pharmaceutically acceptable salt thereof, or a pharmaceutical composition thereof for the manufacture of a medicament for use in treating or lessening the severity in a subject of visceral pain. In some aspects, the visceral pain comprises visceral pain from abdominoplasty.

In another aspect, the invention features a compound of the invention, or a pharmaceutically acceptable salt or pharmaceutical composition thereof, for the manufacture of a medicament for use in treating or lessening the severity in a subject of a neurodegenerative disease. In some aspects, the neurodegenerative disease comprises multiple sclerosis. In some aspects, the neurodegenerative disease comprises Pitt Hopkins Syndrome (PTHS).

In yet another aspect, the invention provides the use of a compound of the invention, a pharmaceutically acceptable salt thereof, or a pharmaceutical composition thereof for the manufacture of a medicament for use in combination with one or more additional therapeutic agents administered concurrently with, prior to, or subsequent to treatment with the compound or pharmaceutical composition. In some embodiments, the additional therapeutic agent is a sodium channel inhibitor.

In another aspect, the invention provides the use of a compound of the invention, a pharmaceutically acceptable salt thereof, or a pharmaceutical composition thereof for the manufacture of a medicament for use in treating or lessening the severity of acute pain, sub-acute and chronic pain, nociceptive pain, neuropathic pain, inflammatory pain, nociplastic pain, arthritis, migraine, cluster headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility.

In another aspect, the invention provides the use of a compound of the invention, a pharmaceutically acceptable salt thereof, or a pharmaceutical composition thereof for the manufacture of a medicament for use in treating or lessening the severity of femur cancer pain, non-malignant chronic bone pain, rheumatoid arthritis, osteoarthritis, spinal stenosis, neuropathic low back pain, myofascial pain syndrome, fibromyalgia, temporomandibular joint pain, chronic visceral pain, abdominal pain, pancreatic pain, IBS pain, chronic and acute headache pain, migraine, tension headache, cluster headaches, chronic and acute neuropathic pain, post-herpetic neuralgia, diabetic neuropathy, HIV-associated neuropathy, trigeminal neuralgia, Charcot-Marie-Tooth neuropathy, hereditary sensory neuropathy, peripheral nerve injury, painful neuromas, ectopic proximal and distal discharges, radiculopathy, chemotherapy induced neuropathic pain, radiotherapy-induced neuropathic pain, persistent/chronic post-surgical pain (e.g., post amputation, post-thoracotomy, post-cardiac surgery), post-mastectomy pain, central pain, spinal cord injury pain, post-stroke pain, thalamic pain, phantom pain (e.g., following removal of lower extremity, upper extremity, breast), intractable pain, acute pain, acute post-operative pain, acute musculoskeletal pain, joint pain, mechanical low back pain, neck pain, tendonitis, injury pain, exercise pain, acute visceral pain, pyelonephritis, appendicitis, cholecystitis, intestinal obstruction, hernias, chest pain, cardiac pain, pelvic pain, renal colic pain, acute obstetric pain, labor pain, cesarean section pain, acute inflammatory pain, burn pain, trauma pain, acute intermittent pain, endometriosis, acute herpes zoster pain, sickle cell anemia, acute pancreatitis, breakthrough pain, orofacial pain, sinusitis pain, dental pain, multiple sclerosis (MS) pain, pain in depression, leprosy pain, Behcet's disease pain, adiposis dolorosa, phlebitic pain, Guillain-Barre pain, painful legs and moving toes, Haglund syndrome, erythromelalgia pain, Fabry's disease pain, bladder and urogenital disease, urinary incontinence, pathological cough, hyperactive bladder, painful bladder syndrome, interstitial cystitis (IC), prostatitis, complex regional pain syndrome (CRPS), type I, complex regional pain syndrome (CRPS) type II, widespread pain, paroxysmal extreme pain, pruritus, tinnitus, or angina-induced pain.

In another aspect, the invention provides the use of a compound of the invention, a pharmaceutically acceptable salt thereof, or a pharmaceutical composition thereof for the manufacture of a medicament for use in treating or lessening the severity of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbrosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain.

### Administration of Compounds, Pharmaceutically Acceptable Salts, and Compositions

In certain embodiments of the invention an “effective amount” of a compound of the invention, a pharmaceutically acceptable salt thereof, or a pharmaceutical composition thereof is that amount effective for treating or lessening the severity of one or more of the conditions recited above.

The compounds, salts, and compositions, according to the method of the invention, may be administered using any amount and any route of administration effective for treating or lessening the severity of one or more of the pain or non-pain diseases recited herein. The exact amount required will vary from subject to subject, depending on the species, age, and general condition of the subject, the severity of the condition, the particular agent, its mode of administration, and the like. The compounds, salts, and compositions of the invention are optionally formulated in dosage unit form for ease of administration and uniformity of dosage. The expression “dosage unit form” as used herein refers to a physically discrete unit of agent appropriate for the subject to be treated. It will be understood, however, that the total daily usage of the compounds, salts, and compositions of the invention will be decided by the attending physician within the scope of sound medical judgment. The specific effective dose level for any particular subject or organism will depend upon a variety of factors including the disorder being treated and the severity of the disorder, the activity of the specific compound or salt employed, the specific composition employed, the age, body weight, general health, sex and diet of the subject, the time of administration, route of administration, and rate of excretion of the specific compound or salt employed, the duration of the treatment, drugs used in combination or coincidental with the specific compound or salt employed, and like factors well known in the medical arts. The term “subject” or “patient,” as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the condition being treated. In certain embodiments, the compound, salts, and compositions of the invention may be administered orally or parenterally at dosage levels of about 0.001 mg/kg to about 1000 mg/kg, one or more times a day, effective to obtain the desired therapeutic effect.

Liquid dosage forms for oral administration include, but are not limited to, pharmaceutically acceptable emulsions, microemulsions, solutions, suspensions, syrups and elixirs. In addition to the active compound or salt, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of the compounds of the invention, it is often desirable to slow the absorption of the compounds from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compound or salt of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound or salt is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compound or salt can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound or salt may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound or salt of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the invention contemplates the use of transdermal patches, which have the added advantage of providing controlled delivery of a compound to the body. Such dosage forms are prepared by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As described generally above, the compounds of the invention are useful as inhibitors of voltage-gated sodium channels. In one embodiment, the compounds are inhibitors of Na_{V}1.8 and thus, without wishing to be bound by any particular theory, the compounds, salts, and compositions are particularly useful for treating or lessening the severity of a disease, condition, or disorder where activation or hyperactivity of Na_{V}1.8 is implicated in the disease, condition, or disorder. When activation or hyperactivity of Na_{V}1.8 is implicated in a particular disease, condition, or disorder, the disease, condition, or disorder may also be referred to as a “Na_{V}1.8-mediated disease, condition or disorder.” Accordingly, in another aspect, the invention provides a method for treating or lessening the severity of a disease, condition, or disorder where activation or hyperactivity of Na_{V}1.8 is implicated in the disease state.

The activity of a compound utilized in this invention as an inhibitor of Na_{V}1.8 may be assayed according to methods described generally in International Publication No. WO 2014/120808 A9 and U.S. Publication No. 2014/0213616 A1, both of which are incorporated by reference in their entirety, methods described herein, and other methods known and available to one of ordinary skill in the art.

### Additional Therapeutic Agents

It will also be appreciated that the compounds, salts, and pharmaceutically acceptable compositions of the invention can be employed in combination therapies, that is, the compounds, salts, and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated.” For example, exemplary additional therapeutic agents include, but are not limited to: non-opioid analgesics (indoles such as Etodolac, Indomethacin, Sulindac, Tolmetin, naphthylalkanones such as Nabumetone, oxicams such as Piroxicam, para-aminophenol derivatives, such as Acetaminophen, propionic acids such as Fenoprofen, Flurbiprofen, Ibuprofen, Ketoprofen, Naproxen, Naproxen sodium, Oxaprozin, salicylates such as Aspirin, Choline magnesium trisalicylate, Diflunisal, fenamates such as meclofenamic acid, Mefenamic acid, and pyrazoles such as Phenylbutazone), or opioid (narcotic) agonists (such as Codeine, Fentanyl, Hydromorphone, Levorphanol, Meperidine, Methadone, Morphine, Oxycodone, Oxymorphone, Propoxyphene, Buprenorphine, Butorphanol, Dezocine, Nalbuphine, and Pentazocine). Additionally, nondrug analgesic approaches may be utilized in conjunction with administration of one or more compounds of the invention. For example, anesthesiologic (intraspinal infusion, neural blockade), neurosurgical (neurolysis of CNS pathways), neurostimulatory (transcutaneous electrical nerve stimulation, dorsal column stimulation), physiatric (physical therapy, orthotic devices, diathermy), or psychologic (cognitive methods-hypnosis, biofeedback, or behavioral methods) approaches may also be utilized. Additional appropriate therapeutic agents or approaches are described generally in The Merck Manual, Nineteenth Edition, Ed. Robert S. Porter and Justin L. Kaplan, Merck Sharp &Dohme Corp., a subsidiary of Merck & Co., Inc., 2011, and the Food and Drug Administration website, www.fda.gov, the entire contents of which are hereby incorporated by reference.

In another embodiment, additional appropriate therapeutic agents are selected from the following:
(1) an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine, pentazocine, or difelikefalin;
(2) a nonsteroidal antiinflammatory drug (NSAID), e.g. aspirin, diclofenac, diflunisal, etodolac, fenbufen, fenoprofen, flufenisal, flurbiprofen, ibuprofen (including without limitation intravenous ibuprofen (e.g., Caldolor®)), indomethacin, ketoprofen, ketorolac (including without limitation ketorolac tromethamine (e.g., Toradol®)), meclofenamic acid, mefenamic acid, meloxicam, IV meloxicam (e.g., Anjeso®), nabumetone, naproxen, nimesulide, nitroflurbiprofen, olsalazine, oxaprozin, phenylbutazone, piroxicam, sulfasalazine, sulindac, tolmetin or zomepirac;
(3) a barbiturate sedative, e.g. amobarbital, aprobarbital, butabarbital, butalbital, mephobarbital, metharbital, methohexital, pentobarbital, phenobarbital, secobarbital, talbutal, thiamylal or thiopental;
(4) a benzodiazepine having a sedative action, e.g. chlordiazepoxide, clorazepate, diazepam, flurazepam, lorazepam, oxazepam, temazepam or triazolam;
(5) a histamine (H₁) antagonist having a sedative action, e.g. diphenhydramine, pyrilamine, promethazine, chlorpheniramine or chlorcyclizine;
(6) a sedative such as glutethimide, meprobamate, methaqualone or dichloralphenazone;
(7) a skeletal muscle relaxant, e.g. baclofen, carisoprodol, chlorzoxazone, cyclobenzaprine, methocarbamol or orphenadrine;
(8) an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®), a combination formulation of morphine and dextromethorphan), topiramate, neramexane or perzinfotel including an NR2B antagonist, e.g. ifenprodil, traxoprodil or (−)—(R)-6-{2-[4-(3-fluorophenyl)-4-hydroxy-l-piperidinyl]-1-hydroxyethyl-3,4-dihydro-2(1H)-quinolinone;
(9) an alpha-adrenergic, e.g. doxazosin, tamsulosin, clonidine, guanfacine, dexmedetomidine, modafinil, or 4-amino-6,7-dimethoxy-2-(5-methane-sulfonamido-1, 2,3,4-tetrahydroisoquinolin-2-yl)-5-(2-pyridyl) quinazoline;
(10) a tricyclic antidepressant, e.g. desipramine, imipramine, amitriptyline or nortriptyline;
(11) an anticonvulsant, e.g. carbamazepine (Tegretol®), lamotrigine, topiramate, lacosamide (Vimpat®) or valproate;
(12) a tachykinin (NK) antagonist, particularly an NK-3, NK-2 or NK-1 antagonist, e.g. (alphaR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
(13) a muscarinic antagonist, e.g oxybutynin, tolterodine, propiverine, tropsium chloride, darifenacin, solifenacin, temiverine and ipratropium;
(14) a COX-2 selective inhibitor, e.g. celecoxib, rofecoxib, parecoxib, valdecoxib, deracoxib, etoricoxib, or lumiracoxib;
(15) a coal-tar analgesic, in particular paracetamol;
(16) a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
(17) a vanilloid receptor agonist (e.g. resinferatoxin or civamide) or antagonist (e.g. capsazepine, GRC-15300);
(18) a beta-adrenergic such as propranolol;
(19) a local anesthetic such as mexiletine;
(20) a corticosteroid such as dexamethasone;
(21) a 5-HT receptor agonist or antagonist, particularly a 5-HT_{1B/1D} agonist such as eletriptan, sumatriptan, naratriptan, zolmitriptan or rizatriptan;
(22) a 5-HT_{2A} receptor antagonist such as R(+)-alpha-(2,3-dimethoxy-phenyl)-1-[2-(4-fluorophenylethyl)]-4-piperidinemethanol (MDL-100907);
(23) a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
(24) Tramadol®, Tramadol ER (Ultram ER®), IV Tramadol, Tapentadol ER (Nucynta®);
(25) a PDE5 inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
(26) an alpha-2-delta ligand such as gabapentin (Neurontin®), gabapentin GR (Gralise®), gabapentin, enacarbil (Horizant®), pregabalin (Lyrica®), 3-methyl gabapentin, (1[alpha],3[alpha],5[alpha])(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
(27) a cannabinoid such as KHK-6188;
(28) metabotropic glutamate subtype 1 receptor (mGluR1) antagonist;
(29) a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
(30) a noradrenaline (norepinephrine) reuptake inhibitor, such as maprotiline, lofepramine, mirtazepine, oxaprotiline, fezolamine, tomoxetine, mianserin, bupropion, bupropion metabolite hydroxybupropion, nomifensine and viloxazine (Vivalan®), especially a selective noradrenaline reuptake inhibitor such as reboxetine, in particular (S,S)-reboxetine;
(31) a dual serotonin-noradrenaline reuptake inhibitor, such as venlafaxine, venlafaxine metabolite O-desmethylvenlafaxine, clomipramine, clomipramine metabolite desmethylclomipramine, duloxetine (Cymbalta®), milnacipran and imipramine;
(32) an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-l-(5-thiazolyl)-butyl]thio]-S-chloro-S-pyridinecarbonitrile; 2-[[(1R,3S)-3-amino-4-hydroxy-l-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-l-(5-thiazolyl) butyl]thio]-6-(trifluoromethyl)-3-pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, NXN-462, or guanidinoethyldisulfide;
(33) an acetylcholinesterase inhibitor such as donepezil;
(34) a prostaglandin E2 subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(15)-1-({[5-chloro-2-(3-fluorophenoxy)pyridin-3-yl]carbonyl}amino)ethyl]benzoic acid;
(35) a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870;
(36) a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl)-1,4-benzoquinone (CV-6504);
(37) a sodium channel blocker, such as lidocaine, lidocaine plus tetracaine cream (ZRS-201) or eslicarbazepine acetate;
(38) a Na_{V}1.7 blocker, such as XEN-402, XEN403, TV-45070, PF-05089771, CNV1014802, GDC-0276, RG7893 BIIB-074 (Vixotrigine), BIIB-095, ASP-1807, DSP-3905, OLP-1002, RQ-00432979, FX-301, DWP-1706, DWP-17061, IMB-110, IMB-111, IMB-112 and such as those disclosed in WO2011/140425 (US2011/306607); WO2012/106499 (US2012196869); WO2012/112743 (US2012245136); WO2012/125613 (US2012264749), WO2012/116440 (US2014187533), WO2011026240 (US2012220605), U.S. Pat. Nos. 8,883,840, 8,466,188, WO2013/109521 (US2015005304), WO2020/117626, and CN111217776, the entire contents of each application hereby incorporated by reference;
(38a) a Na_{V}1.7 blocker such as (2-benzylspiro[3,4-dihydropyrrolo[1,2-a]pyrazine-1,4′-piperidine]-1′-yl)-(4-isopropoxy-3-methyl-phenyl)methanone, 2,2,2-trifluoro-1-[1′-[3-methoxy-4-[2-(trifluoromethoxy)ethoxy]benzoyl]-2,4-dimethyl-spiro[3,4-dihydropyrrolo[1,2-a]pyrazine-1,4′-piperidine]-6-yl]ethanone, [8-fluoro-2-methyl-6-(trifluoromethyl)spiro[3,4-dihydropyrrolo[1,2-a]pyrazine-1,4′-piperidine]-1′-yl]-(4-isobutoxy-3-methoxy-phenyl)methanone, 1-(4-benzhydrylpiperazin-1-yl)-3-[2-(3,4-dimethylphenoxy)ethoxy]propan-2-ol, (4-butoxy-3-methoxy-phenyl)-[2-methyl-6-(trifluoromethyl)spiro[3,4-dihydropyrrolo[1,2-a]pyrazine-1,4′-piperidine]-1′-yl]methanone, [8-fluoro-2-methyl-6-(trifluoromethyl)spiro[3,4-dihydropyrrolo[1,2-a]pyrazine-1,4′-piperidine]-1′-yl]-(5-isopropoxy-6-methyl-2-pyridyl)methanone, (4-isopropoxy-3-methyl-phenyl)-[2-methyl-6-(1,1,2,2,2-pentafluoroethyl)spiro[3,4-dihydropyrrolo[1,2-a]pyrazine-1,4′-piperidine]-1′-yl]methanone, 5-[2-methyl-4-[2-methyl-6-(2,2,2-trifluoroacetyl)spiro[3,4-dihydropyrrolo[1,2-a]pyrazine-1,4′-piperidine]-1′-carbonyl]phenyl]pyridine-2-carbonitrile, (4-isopropoxy-3-methyl-phenyl)-[6-(trifluoromethyl)spiro[3,4-dihydro-2H-pyrrolo[1,2-a]pyrazine-1,4′-piperidine]-1′-yl]methanone, 2,2,2-trifluoro-1-[1′-[3-methoxy-4-[2-(trifluoromethoxy)ethoxy]benzoyl]-2-methyl-spiro[3,4-dihydropyrrolo[1,2-a]pyrazine-1,4′-piperidine]-6-yl]ethanone, 2,2,2-trifluoro-1-[1′-(5-isopropoxy-6-methyl-pyridine-2-carbonyl)-3,3-dimethyl-spiro[2,4-dihydropyrrolo[1,2-a]pyrazine-1,4′-piperidine]-6-yl]ethanone, 2,2,2-trifluoro-1-[1′-(5-isopentyloxypyridine-2-carbonyl)-2-methyl-spiro[3,4-dihydropyrrolo[1,2-a]pyrazine-1,4′-piperidine]-6-yl]ethanone, (4-isopropoxy-3-methoxy-phenyl)-[2-methyl-6-(trifluoromethyl)spiro[3,4-dihydropyrrolo[1,2-a]pyrazine-1,4′-piperidine]-1′-yl]methanone, 2,2,2-trifluoro-1-[1′-(5-isopentyloxypyridine-2-carbonyl)-2,4-dimethyl-spiro[3,4-dihydropyrrolo[1,2-a]pyrazine-1,4′-piperidine]-6-yl]ethanone, 1-[(3S)-2,3-dimethyl-1′-[4-(3,3,3-trifluoropropoxymethyl)benzoyl]spiro[3,4-dihydropyrrolo[1,2-a]pyrazine-1,4′-piperidine]-6-yl]-2,2,2-trifluoro-ethanone, [8-fluoro-2-methyl-6-(trifluoromethyl)spiro[3,4-dihydropyrrolo[1,2-a]pyrazine-1,4′-piperidine]-1′-yl]-[3-methoxy-4-[(1R)-1-methylpropoxy]phenyl]methanone, 2,2,2-trifluoro-1-[1′-(5-isopropoxy-6-methyl-pyridine-2-carbonyl)-2,4-dimethyl-spiro[3,4-dihydropyrrolo[1,2-a]pyrazine-1,4′-piperidine]-6-yl]ethanone, 1-[1′-[4-methoxy-3-(trifluoromethyl)benzoyl]-2-methyl-spiro[3,4-dihydropyrrolo[1,2-a]pyrazine-1,4′-piperidine]-6-yl]-2,2-dimethyl-propan-1-one, (4-isopropoxy-3-methyl-phenyl)-[2-methyl-6-(trifluoromethyl)spiro[3,4-dihydropyrrolo[1,2-a]pyrazine-1,4′-piperidine]-1′-yl]methanone, [2-methyl-6-(1-methylcyclopropanecarbonyl)spiro[3,4-dihydropyrrolo[1,2-a]pyrazine-1,4′-piperidine]-1′-yl]-[4-(3,3,3-trifluoropropoxymethyl)phenyl]methanone, 4-bromo-N-(4-bromophenyl)-3-[(1-methyl-2-oxo-4-piperidyl)sulfamoyl]benzamide or (3-chloro-4-isopropoxy-phenyl)-[2-methyl-6-(1,1,2,2,2-pentafluoroethyl)spiro[3,4-dihydropyrrolo[1,2-a]pyrazine-1,4′-piperidine]-1′-yl]methanone.
(39) a Na_{V}1.8 blocker, such as PF-04531083, PF-06372865 and such as those disclosed in WO2008/135826 (US2009048306), WO2006/011050 (US2008312235), WO2013/061205 (US2014296313), US20130303535, WO2013131018, U.S. Pat. No. 8,466,188, WO2013114250 (US2013274243), WO2014/120808 (US2014213616), WO2014/120815 (US2014228371) WO2014/120820 (US2014221435), WO2015/010065 (US20160152561), WO2015/089361 (US20150166589), WO2019/014352 (US20190016671), WO2018/213426, WO2020/146682, WO2020/146612, WO2020/014243, WO2020/014246, WO2020/092187, WO2020/092667 (US2020140411), WO2020/261114, WO2020/140959, WO2020/151728, WO2021/032074, CN112390745, CN111808019, CN112225695, CN112457294, CN112300051, CN112300069, CN112441969, and CN112479996 (WO2021/047622), the entire contents of each application hereby incorporated by reference;
(39a) a Na_{V}1.8 blocker such as 4,5-dichloro-2-(4-fluoro-2-methoxyphenoxy)-N-(2-oxo-1,2-dihydropyridin-4-yl)benzamide, 2-(4-fluoro-2-methoxyphenoxy)-N-(2-oxo-1,2-dihydropyridin-4-yl)-4-(perfluoroethyl)benzamide, 4,5-dichloro-2-(4-fluorophenoxy)-N-(2-oxo-1,2-dihydropyridin-4-yl)benzamide, 4,5-dichloro-2-(3-fluoro-4-methoxyphenoxy)-N-(2-oxo-1,2-dihydropyridin-4-yl)benzamide, 2-(4-fluoro-2-methoxyphenoxy)-N-(2-oxo-1,2-dihydropyridin-4-yl)-5-(trifluoromethyl)benzamide, N-(2-oxo-1,2-dihydropyridin-4-yl)-2-(4-(trifluoromethoxy)phenoxy)-4-(trifluoromethyl)benzamide, 2-(4-fluorophenoxy)-N-(2-oxo-1,2-dihydropyridin-4-yl)-4-(perfluoroethyl)benzamide, 5-chloro-2-(4-fluoro-2-methoxyphenoxy)-N-(2-oxo-1,2-dihydropyridin-4-yl)benzamide, N-(2-oxo-1,2-dihydropyridin-4-yl)-2-(4-(trifluoromethoxy)phenoxy)-5-(trifluoromethyl)benzamide, 2-(4-fluoro-2-methylphenoxy)-N-(2-oxo-1,2-dihydropyridin-4-yl)-5-(trifluoromethyl)benzamide, 2-(2-chloro-4-fluorophenoxy)-N-(2-oxo-1,2-dihydropyridin-4-yl)-5-(trifluoromethyl)benzamide, 5-chloro-2-(4-fluoro-2-methylphenoxy)-N-(2-oxo-1,2-dihydropyridin-4-yl)benzamide, 4-chloro-2-(4-fluoro-2-methylphenoxy)-N-(2-oxo-1,2-dihydropyridin-4-yl)benzamide, 5-chloro-2-(2-chloro-4-fluorophenoxy)-N-(2-oxo-1,2-dihydropyridin-4-yl)benzamide, 2-((5-fluoro-2-hydroxybenzyl)oxy)-N-(2-oxo-1,2-dihydropyridin-4-yl)-4-(trifluoromethyl)benzamide, N-(2-oxo-1,2-dihydropyridin-4-yl)-2-(o-tolyloxy)-5-(trifluoromethyl)benzamide, 2-(2,4-difluorophenoxy)-N-(2-oxo-1,2-dihydropyridin-4-yl)-4-(trifluoromethyl)benzamide, N-(2-oxo-1,2-dihydropyridin-4-yl)-2-(2-(trifluoromethoxy)phenoxy)-5-(trifluoromethyl)benzamide, 2-(4-fluorophenoxy)-N-(2-oxo-1,2-dihydropyridin-4-yl)-5-(trifluoromethyl)benzamide, 2-(4-fluoro-2-methyl-phenoxy)-N-(2-oxo-1H-pyridin-4-yl)-4-(trifluoromethyl)benzamide, [4-[[2-(4-fluoro-2-methyl-phenoxy)-4-(trifluoromethyl)benzoyl]amino]-2-oxo-1-pyridyl]methyl dihydrogen phosphate, 2-(4-fluoro-2-(methyl-d₃)phenoxy)-N-(2-oxo-1,2-dihydropyridin-4-yl)-4-(trifluoromethyl)benzamide, (4-(2-(4-fluoro-2-(methyl-d₃)phenoxy)-4-(trifluoromethyl)benzamido)-2-oxopyridin-1(2H)-yl)methyl dihydrogen phosphate, 3-(4-fluoro-2-methoxyphenoxy)-N-(3-(methylsulfonyl)phenyl)quinoxaline-2-carboxamide, 3-(2-chloro-4-fluorophenoxy)-N-(3-sulfamoylphenyl)quinoxaline-2-carboxamide, 3-(2-chloro-4-methoxyphenoxy)-N-(3-sulfamoylphenyl)quinoxaline-2-carboxamide, 3-(4-chloro-2-methoxyphenoxy)-N-(3-sulfamoylphenyl)quinoxaline-2-carboxamide, 4-(3-(4-(trifluoromethoxy)phenoxy)quinoxaline-2-carboxamido)picolinic acid, 2-(2,4-difluorophenoxy)-N-(3-sulfamoylphenyl)quinoline-3-carboxamide, 2-(4-fluoro-2-methoxyphenoxy)-N-(3-sulfamoylphenyl)quinoline-3-carboxamide, 3-(2,4-difluorophenoxy)-N-(3-sulfamoylphenyl)quinoxaline-2-carboxamide, N-(3-sulfamoylphenyl)-2-(4-(trifluoromethoxy)phenoxy)quinoline-3-carboxamide, N-(3-sulfamoylphenyl)-3-(4-(trifluoromethoxy)phenoxy)quinoxaline-2-carboxamide, 3-(4-chloro-2-methylphenoxy)-N-(3-sulfamoylphenyl)quinoxaline-2-carboxamide, 5-(3-(4-(trifluoromethoxy)phenoxy)quinoxaline-2-carboxamido)picolinic acid, 3-(4-fluoro-2-methoxyphenoxy)-N-(2-oxo-2,3-dihydro-1H-benzo[d]imidazol-5-yl)quinoxaline-2-carboxamide, 3-(4-fluoro-2-methoxyphenoxy)-N-(pyridin-4-yl)quinoxaline-2-carboxamide, 3-(4-fluorophenoxy)-N-(3-sulfamoylphenyl)quinoxaline-2-carboxamide, N-(3-cyanophenyl)-3-(4-fluoro-2-methoxyphenoxy)quinoxaline-2-carboxamide, N-(4-carbamoylphenyl)-3-(4-fluoro-2-methoxyphenoxy)quinoxaline-2-carboxamide, 4-(3-(4-(trifluoromethoxy)phenoxy)quinoxaline-2-carboxamido)benzoic acid, N-(4-cyanophenyl)-3-(4-fluoro-2-methoxyphenoxy)quinoxaline-2-carboxamide, 5-(4,5-dichloro-2-(4-fluoro-2-methoxyphenoxy)benzamido)picolinic acid, 5-(2-(2,4-dimethoxyphenoxy)-4,6-bis(trifluoromethyl)benzamido)picolinic acid, 4-(4,5-dichloro-2-(4-fluoro-2-methoxyphenoxy)benzamido)benzoic acid, 5-(2-(4-fluoro-2-methoxyphenoxy)-4,6-bis(trifluoromethyl)benzamido)picolinic acid, 4-(2-(4-fluoro-2-methoxyphenoxy)-4-(perfluoroethyl)benzamido)benzoic acid, 5-(2-(4-fluoro-2-methoxyphenoxy)-4-(perfluoroethyl)benzamido)picolinic acid, 4-(2-(4-fluoro-2-methylphenoxy)-4-(trifluoromethyl)benzamido)benzoic acid, 5-(4,5-dichloro-2-(4-fluoro-2-methoxyphenoxy)benzamido)picolinic acid, 4-(2-(2-chloro-4-fluorophenoxy)-4-(perfluoroethyl)benzamido)benzoic acid, 4-(2-(4-fluoro-2-methylphenoxy)-4-(perfluoroethyl)benzamido)benzoic acid, 4-(4,5-dichloro-2-(4-(trifluoromethoxy)phenoxy)benzamido)benzoic acid, 4-(4,5-dichloro-2-(4-chloro-2-methylphenoxy)benzamido)benzoic acid, 5-(4-(tert-butyl)-2-(4-fluoro-2-methoxyphenoxy)benzamido)picolinic acid, 5-(4,5-dichloro-2-(4-(trifluoromethoxy)phenoxy)benzamido)picolinic acid, 4-(4,5-dichloro-2-(4-fluoro-2-methylphenoxy)benzamido)benzoic acid, 5-(4,5-dichloro-2-(2,4-dimethoxyphenoxy)benzamido)picolinic acid, 5-(4,5-dichloro-2-(2-chloro-4-fluorophenoxy)benzamido)picolinic acid, 5-(4,5-dichloro-2-(4-fluoro-2-methylphenoxy)benzamido)picolinic acid, 4-(4,5-dichloro-2-(4-chloro-2-methoxyphenoxy)benzamido)benzoic acid, 5-(4,5-dichloro-2-(2,4-difluorophenoxy)benzamido)picolinic acid, 2-(4-fluorophenoxy)-N-(3-sulfamoylphenyl)-5-(trifluoromethyl)benzamide, 2-(4-fluorophenoxy)-N-(3-sulfamoylphenyl)-4-(trifluoromethyl)benzamide, 2-(2-chloro-4-fluorophenoxy)-N-(3-sulfamoylphenyl)-5-(trifluoromethyl)benzamide, 2-(4-fluorophenoxy)-N-(3-sulfamoylphenyl)-4-(trifluoromethyl)benzamide, 2-(2-chloro-4-fluorophenoxy)-N-(3-sulfamoylphenyl)-6-(trifluoromethyl)benzamide, 2-(2-chloro-4-fluorophenoxy)-5-(difluoromethyl)-N-(3-sulfamoylphenyl)benzamide, 2-(4-fluorophenoxy)-4-(perfluoroethyl)-N-(3-sulfamoylphenyl)benzamide, 2-(4-chloro-2-methoxyphenoxy)-4-(perfluoroethyl)-N-(3-sulfamoylphenyl)benzamide, 2-(4-fluoro-2-methoxyphenoxy)-N-(3-sulfamoylphenyl)-5-(trifluoromethyl)benzamide, 5-chloro-2-(4-fluoro-2-methylphenoxy)-N-(3-sulfamoylphenyl)benzamide, 4,5-dichloro-2-(4-fluoro-2-methoxyphenoxy)-N-(3-sulfamoylphenyl)benzamide, 2,4-dichloro-6-(4-chloro-2-methoxyphenoxy)-N-(3-sulfamoylphenyl)benzamide, 2,4-dichloro-6-(4-fluoro-2-methylphenoxy)-N-(3-sulfamoylphenyl)benzamide, 2-(4-fluoro-2-methoxyphenoxy)-N-(3-sulfamoylphenyl)-4,6-bis(trifluoromethyl)benzamide, 2-(4-fluoro-2-methylphenoxy)-N-(3-sulfamoylphenyl)-4,6-bis(trifluoromethyl)benzamide, 5-chloro-2-(2-chloro-4-fluorophenoxy)-N-(3-sulfamoylphenyl)benzamide, 2-(4-fluoro-2-methoxyphenoxy)-N-(3-sulfamoylphenyl)-4-(trifluoromethoxy)benzamide, 2-(4-fluoro-2-methoxyphenoxy)-N-(3-sulfamoylphenyl)-4-(trifluoromethyl)benzamide, 4,5-dichloro-2-(4-fluorophenoxy)-N-(3-sulfamoylphenyl)benzamide, 2-(4-fluoro-2-methoxyphenoxy)-4-(perfluoroethyl)-N-(3-sulfamoylphenyl)benzamide, 5-fluoro-2-(4-fluoro-2-methylphenoxy)-N-(3-sulfamoylphenyl)benzamide, 2-(2-chloro-4-fluorophenoxy)-4-cyano-N-(3-sulfamoylphenyl)benzamide, N-(3-sulfamoylphenyl)-2-(4-(trifluoromethoxy)phenoxy)-4-(trifluoromethyl)benzamide, N-(3-carbamoyl-4-fluoro-phenyl)-2-fluoro-6-[2-(trideuteriomethoxy)-4-(trifluoromethoxy)phenoxy]-3-(trifluoromethyl)benzamide, N-(3-carbamoyl-4-fluoro-phenyl)-2-fluoro-6-[2-methoxy-4-(trifluoromethoxy)phenoxy]-3-(trifluoromethyl)benzamide, N-(3-carbamoyl-4-fluoro-phenyl)-2-fluoro-6-[2-(trideuteriomethoxy)-4-(trifluoromethoxy)phenoxy]-3-(trifluoromethoxy)benzamide, 4-[[2-fluoro-6-[2-methoxy-4-(trifluoromethoxy)phenoxy]-3-(trifluoromethyl)benzoyl]amino]pyridine-2-carboxamide, 4-[[3-chloro-2-fluoro-6-[2-methoxy-4-(trifluoromethoxy)phenoxy]benzoyl]amino]pyridine-2-carboxamide, 4-[[2-fluoro-6-[2-(trideuteriomethoxy)-4-(trifluoromethoxy)phenoxy]-3-(trifluoromethyl)benzoyl]amino]pyridine-2-carboxamide, N-(3-carbamoyl-4-fluoro-phenyl)-3-(difluoromethyl)-2-fluoro-6-[2-methoxy-4-(trifluoromethoxy)phenoxy]benzamide, 4-[[2-fluoro-6-[2-(trideuteriomethoxy)-4-(trifluoromethoxy)phenoxy]-3-(trifluoromethoxy)benzoyl]amino]pyridine-2-carboxamide, N-(3-carbamoyl-4-fluoro-phenyl)-6-[2-chloro-4-(trifluoromethoxy)phenoxy]-2-fluoro-3-(trifluoromethyl)benzamide, N-(3-carbamoyl-4-fluoro-phenyl)-2-fluoro-6-[2-methyl-4-(trifluoromethoxy)phenoxy]-3-(trifluoromethyl)benzamide, N-(3-carbamoyl-4-fluoro-phenyl)-2,3,4-trifluoro-6-[2-methoxy-4-(trifluoromethoxy)phenoxy]benzamide, N-(2-carbamoyl-4-pyridyl)-3-fluoro-5-[2-methoxy-4-(trifluoromethoxy)phenoxy]-2-(trifluoromethyl)pyridine-4-carboxamide, 4-[[6-[2-(difluoromethoxy)-4-(trifluoromethoxy)phenoxy]-2-fluoro-3-(trifluoromethyl)benzoyl]amino]pyridine-2-carboxamide, N-(3-carbamoyl-4-fluoro-phenyl)-6-[3-chloro-4-(trifluoromethoxy)phenoxy]-2-fluoro-3-(trifluoromethyl)benzamide, N-(3-carbamoyl-4-fluoro-phenyl)-2-fluoro-6-[4-(trifluoromethoxy)phenoxy]-3-(trifluoromethyl)benzamide, N-(4-carbamoyl-3-fluoro-phenyl)-2-fluoro-6-[2-methoxy-4-(trifluoromethoxy)phenoxy]-3-(trifluoromethyl)benzamide, 4-[[2-fluoro-6-[2-(trideuteriomethoxy)-4-(trifluoromethoxy)phenoxy]-4-(trifluoromethyl)benzoyl]amino]pyridine-2-carboxamide, N-(3-carbamoyl-4-fluoro-phenyl)-2-fluoro-6-[3-fluoro-4-(trifluoromethoxy)phenoxy]-3-(trifluoromethyl)benzamide, N-(3-carbamoyl-4-fluoro-phenyl)-2-[2-methoxy-4-(trifluoromethoxy)phenoxy]-5-(1,1,2,2,2-pentafluoroethyl)benzamide, 4-[[4-(difluoromethoxy)-2-fluoro-6-[2-methoxy-4-(trifluoromethoxy)phenoxy]benzoyl]amino]pyridine-2-carboxamide, N-(3-carbamoyl-4-fluoro-phenyl)-2-fluoro-6-[2-fluoro-4-(trifluoromethoxy)phenoxy]-3-(trifluoromethyl)benzamide, 4-[[4-cyclopropyl-2-fluoro-6-[2-methoxy-4-(trifluoromethoxy)phenoxy]benzoyl]amino]pyridine-2-carboxamide, N-(3-carbamoyl-4-fluoro-phenyl)-5-fluoro-2-[2-methoxy-4-(trifluoromethoxy)phenoxy]-4-(trifluoromethyl)benzamide, 5-[[2-fluoro-6-[2-(trideuteriomethoxy)-4-(trifluoromethoxy)phenoxy]-3-(trifluoromethyl)benzoyl]amino]pyridine-2-carboxamide, N-(3-carbamoyl-4-fluoro-phenyl)-2-fluoro-6-(4-fluorophenoxy)-3-(trifluoromethyl)benzamide, 4-(2-fluoro-6-(2-methoxy-4-(trifluoromethoxy)phenoxy)-3-(trifluoromethyl)benzamido)picolinamide, or 4-[[2-fluoro-6-[3-fluoro-2-methoxy-4-(trifluoromethoxy)phenoxy]-3-(trifluoromethyl)benzoyl]amino]pyridine-2-carboxamide;
(40) a combined Na_{V}1.7 and Na_{V}1.8 blocker, such as DSP-2230, Lohocla201 or BL-1021;
(41) a 5-HT3 antagonist, such as ondansetron;
(42) a TPRV 1 receptor agonist, such as capsaicin (NeurogesX®, Qutenza®); and the pharmaceutically acceptable salts and solvates thereof;
(43) a nicotinic receptor antagonist, such as varenicline;
(44) an N-type calcium channel antagonist, such as Z-160;
(45) a nerve growth factor antagonist, such as tanezumab;
(46) an endopeptidase stimulant, such as senrebotase;
(47) an angiotensin II antagonist, such as EMA-401;
(48) acetaminophen (including without limitation intravenous acetaminophen (e.g., Ofirmev®));
(49) bupivacaine (including without limitation bupivacaine liposome injectable suspension (e.g., Exparel®) bupivacaine ER (Posimir), bupivacaine collagen (Xaracoll) and transdermal bupivacaine (Eladur®)); and
(50) bupivacaine and meloxicam combination (e.g., HTX-011).

In one embodiment, the additional appropriate therapeutic agents are selected from V-116517, Pregabalin, controlled release Pregabalin, Ezogabine (Potiga®). Ketamine/amitriptyline topical cream (Amiket®), AVP-923, Perampanel (E-2007), Ralfinamide, transdermal bupivacaine (Eladur®), CNV1014802, JNJ-10234094 (Carisbamate), BMS-954561 or ARC-4558.

In another embodiment, the additional appropriate therapeutic agents are selected from N-(6-amino-5-(2,3,5-trichlorophenyl)pyridin-2-yl)acetamide; N-(6-amino-5-(2-chloro-5-methoxyphenyl)pyridin-2-yl)-1-methyl-1H-pyrazole-5-carboxamide; or 3-((4-(4-(trifluoromethoxy)phenyl)-1H-imidazol-2-yl)methyl)oxetan-3-amine.

In another embodiment, the additional therapeutic agent is selected from a GlyT2/5HT2 inhibitor, such as Operanserin (VVZ149), a TRPV modulator such as CA008, CMX-020, NEO6860, FTABS, CNTX4975, MCP101, MDR16523, or MDR652, a EGR1 inhibitor such as Brivoglide (AYX1), an NGF inhibitor such as Tanezumab, Fasinumab, ASP6294, MEDI7352, a Mu opioid agonist such as Cebranopadol, NKTR181 (oxycodegol), a CB-1 agonist such as NEO1940 (AZN1940), an imidazoline 12 agonist such as CR4056 or a p75NTR-Fc modulator such as LEVI-04.

In another embodiment, the additional therapeutic agent is oliceridine or ropivacaine (TLC590).

In another embodiment, the additional therapeutic agent is a Na_{V}1.7 blocker such as ST-2427 or ST-2578 and those disclosed in WO2010129864, WO2015157559, WO2017059385, WO2018183781, WO2018183782, WO2020072835, and WO2022036297 the entire contents of each application hereby incorporated by reference. In some embodiments, the additional therapeutic agent is a Na_{V}1.7 blocker disclosed in WO2020072835. In some embodiments, the additional therapeutic agent is a Na_{V}1.7 blocker disclosed in WO2022036297.

In another embodiment, the additional therapeutic agent is ASP18071, CC-8464, ANP-230, ANP-231, NOC-100, NTX-1175, ASN008, NW3509, AM-6120, AM-8145, AM-0422, BL-017881, NTM-006, Opiranserin (Unafra™), brivoligide, SR419, NRD.E1, LX9211, LY3016859, ISC-17536, NFX-88, LAT-8881, AP-235, NYX 2925, CNTX-6016, S-600918, S-637880, RQ-00434739, KLS-2031, MEDI 7352, or XT-150.

In another embodiment, the additional therapeutic agent is Olinvyk, Zynrelef, Seglentis, Neumentum, Nevakar, HTX-034, CPL-01, ACP-044, HRS-4800, Tarlige, BAY2395840, LY3526318, Eliapixant, TRV045, RTA901, NRD1355-E1, MT-8554, LY3556050, AP-325, tetrodotoxin, Otenaproxesul, CFTX-1554, Funapide, iN1011-N17, JMKX000623, ETX-801, or ACD440.

In another embodiment, the additional therapeutic agent is a compound disclosed in WO2021257490, WO2021257420, WO2021257418, WO2020014246, WO2020092187, WO2020092667, WO2020261114, CN112457294, CN112225695, CN111808019, WO2021032074, WO2020151728, WO2020140959, WO2022037641, WO2022037647, CN112300051, CN112300069, WO2014120808, WO2015089361, WO2019014352, WO2021113627, WO2013086229, WO2013134518, WO2014211173, WO2014201206, WO2016141035, WO2021252818, WO2021252822, and WO2021252820.

In some embodiments, the additional therapeutic agent is a compound disclosed in WO2013086229. In some embodiments, the additional therapeutic agent is a compound disclosed in WO2013134518. In some embodiments, the additional therapeutic agent is a compound disclosed in WO2014211173. In some embodiments, the additional therapeutic agent is a compound disclosed in WO2014201206. In some embodiments, the additional therapeutic agent is a compound disclosed in WO2016141035. In some embodiments, the additional therapeutic agent is a compound disclosed in WO2021252818. In some embodiments, the additional therapeutic agent is a compound disclosed in WO2021252822. In some embodiments, the additional therapeutic agent is a compound disclosed in WO2021252820. In some embodiments, the additional therapeutic agent is a compound disclosed in WO2020072835. In some embodiments, the additional therapeutic agent is a compound disclosed in WO2022036297.

In another embodiment, the additional therapeutic agent is a sodium channel inhibitor (also known as a sodium channel blocker), such as the Na_{V}1.7 and Na_{V}1.8 blockers identified above.

The amount of additional therapeutic agent present in the compositions of this invention may be no more than the amount that would normally be administered in a composition comprising that therapeutic agent as the only active agent. The amount of additional therapeutic agent in the presently disclosed compositions may range from about 10% to 100% of the amount normally present in a composition comprising that agent as the only therapeutically active agent.

The compounds and salts of this invention or pharmaceutically acceptable compositions thereof may also be incorporated into compositions for coating an implantable medical device, such as prostheses, artificial valves, vascular grafts, stents and catheters. Accordingly, the invention, in another aspect, includes a composition for coating an implantable device comprising a compound or salt of the invention as described generally above, and in classes and subclasses herein, and a carrier suitable for coating said implantable device. In still another aspect, the invention includes an implantable device coated with a composition comprising a compound or salt of the invention as described generally above, and in classes and subclasses herein, and a carrier suitable for coating said implantable device. Suitable coatings and the general preparation of coated implantable devices are described in U.S. Pat. Nos. 6,099,562, 5,886,026, and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition.

Another aspect of the invention relates to inhibiting Na_{V}1.8 activity in a biological sample or a subject, which method comprises administering to the subject, or contacting said biological sample with a compound of the invention, a pharmaceutically acceptable salt thereof, or a pharmaceutical composition thereof. The term “biological sample,” as used herein, includes, without limitation, cell cultures or extracts thereof, biopsied material obtained from a mammal or extracts thereof, and blood, saliva, urine, feces, semen, tears, or other body fluids or extracts thereof.

Inhibition of Na_{V}1.8 activity in a biological sample is useful for a variety of purposes that are known to one of skill in the art. Examples of such purposes include, but are not limited to, the study of sodium channels in biological and pathological phenomena, and the comparative evaluation of new sodium channel inhibitors.

### Synthesis of the Compounds of the Invention

The compounds of the invention can be prepared from known materials by the methods described in the Examples, other similar methods, and other methods known to one skilled in the art. As one skilled in the art would appreciate, the functional groups of the intermediate compounds in the methods described below may need to be protected by suitable protecting groups. Protecting groups may be added or removed in accordance with standard techniques, which are well-known to those skilled in the art. The use of protecting groups is described in detail in T. G. M. Wuts et al., Greene's Protective Groups in Organic Synthesis (4th ed. 2006).

### Radiolabeled Analogs of the Compounds of the Invention

In another aspect, the invention relates to radiolabeled analogs of the compounds of the invention. As used herein, the term “radiolabeled analogs of the compounds of the invention” refers to compounds that are identical to the compounds of the invention, as described herein, including all embodiments thereof, except that one or more atoms has been replaced with a radioisotope of the atom present in the compounds of the invention.

As used herein, the term “radioisotope” refers to an isotope of an element that is known to undergo spontaneous radioactive decay. Examples of radioisotopes include ³H, ¹⁴C, ³²P, ³⁵S, ¹⁸F, ³⁶Cl, and the like, as well as the isotopes for which a decay mode is identified in V. S. Shirley & C. M. Lederer, Isotopes Project, Nuclear Science Division, Lawrence Berkeley Laboratory, Table of Nuclides (January 1980).

The radiolabeled analogs can be used in a number of beneficial ways, including in various types of assays, such as substrate tissue distribution assays. For example, tritium (³H)- and/or carbon-14 (¹⁴C)-labeled compounds may be useful for various types of assays, such as substrate tissue distribution assays, due to relatively simple preparation and excellent detectability.

In another aspect, the invention relates to pharmaceutically acceptable salts of the radiolabeled analogs, in accordance with any of the embodiments described herein in connection with the compounds of the invention.

In another aspect, the invention relates to pharmaceutical compositions comprising the radiolabeled analogs, or pharmaceutically acceptable salts thereof, and a pharmaceutically acceptable carrier, adjuvant or vehicle, in accordance with any of the embodiments described herein in connection with the compounds of the invention.

In another aspect, the invention relates to methods of inhibiting voltage-gated sodium channels and methods of treating or lessening the severity of various diseases and disorders, including pain, in a subject comprising administering an effective amount of the radiolabeled analogs, pharmaceutically acceptable salts thereof, and pharmaceutical compositions thereof, in accordance with any of the embodiments described herein in connection with the compounds of the invention.

In another aspect, the invention relates to radiolabeled analogs, pharmaceutically acceptable salts thereof, and pharmaceutical compositions thereof, for use, in accordance with any of the embodiments described herein in connection with the compounds of the invention.

In another aspect, the invention relates to the use of the radiolabeled analogs, or pharmaceutically acceptable salts thereof, and pharmaceutical compositions thereof, for the manufacture of medicaments, in accordance with any of the embodiments described herein in connection with the compounds of the invention.

In another aspect, the radiolabeled analogs, pharmaceutically acceptable salts thereof, and pharmaceutical compositions thereof, can be employed in combination therapies, in accordance with any of the embodiments described herein in connection with the compounds of the invention.

### Enumerated Embodiments

Additional embodiments, features, and advantages of the disclosure will be apparent from the following detailed description and through practice of the disclosure. The compounds and methods of the present disclosure can be described as embodiments in any of the following enumerated clauses. It will be understood that any of the embodiments described herein can be used in connection with any other embodiments described herein to the extent that the embodiments do not contradict one another.
1. A compound of formula (I)

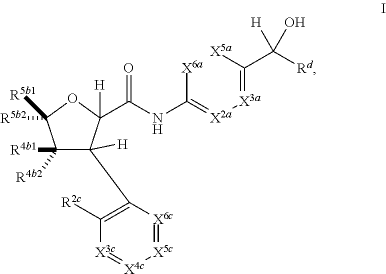

or a pharmaceutically acceptable salt thereof, wherein:
X^{2a} is N, N⁺—O⁻, or C—R^{2a};
X^{3a} is N or N⁺—O⁻;
X^{5a} is N, N⁺—O⁻, or C—R^{5a};
X^{6a} is N, N⁺—O⁻, or C—R^{6a};
R^{d} is (CH₂)ₙ(CHR^{e})ₙ(CH₂)ₚH;
m, n, and p are each independently 0 or 1;
R^{e} is H, OH, halo, C₁-C₆ alkoxy, or C₁-C₆ haloalkoxy;
R^{2a} and R^{6a} are each independently H, halo, C₁-C₆ alkyl, or C₁-C₆ haloalkyl;
R^{5a} is H, halo, CH₂OH, C₁-C₆ alkyl, C₁-C₆ haloalkyl, or R^{5a} and R^{d} form a CH₂CH₂ chain linking the C atoms to which R^{5a} and R^{d} are attached, wherein the CH₂ group that is bound to the C atom to which R^{5a} is attached may be replaced with O;
R^{4b1} and R^{4b2} are each independently H, C₁-C₆ alkyl, C₃-C₆ cycloalkyl, or C₁-C₆ haloalkyl;
R^{5b1} and R² are each independently H, C₁-C₆ alkyl, C₃-C₆ cycloalkyl, or C₁-C₆ haloalkyl;
X^{3c} is N or C—R^{3c};
X^{4c} is N or C—R^{4c};
X^{5c} is N or C—R^{5c};
X^{6c} is N or C—R^{6c};
R^{2c} is H, OH, halo, C₁-C₆ alkyl, C₂-C₆ alkenyl, C₁-C₆ haloalkyl, C₁-C₆ alkoxy, C₁-C₆ haloalkoxy, O—CH₂—C(R^{2c1})(R^{2c2})(R^{2c3}), O—CH(R^{2c4})(R^{2c5}), or -L¹-L²-(C₃-C₆ cycloalkyl), wherein said cycloalkyl is optionally substituted with 1-2 halo;
R^{2c1} and R^{2c2} are each independently H or C₁-C₆ alkyl, or R^{2c1} and R^{2c2} together with the C atom to which they are attached form C═O;
R^{2c3} is OH, C₁-C₆ alkoxy, C₁-C₆ haloalkoxy, or N(R^{2c6})(R^{2c7}); or R^{2c2} and R^{2c3} together with the C atom to which they are attached form a 3-7 membered heterocycloalkyl;
R^{2c4} and R^{2c5} together with the C atom to which they are attached form a 3-7 membered heterocycloalkyl;
R^{2c6} and R^{2c7} are each C₁-C₆ alkyl, or R^{2c6} and R^{2c7} together with the N atom to which they are attached form a 3-8 membered heterocycloalkyl;
L¹ is a bond or O;
L² is a bond or C₁-C₆ alkylene;
R^{3c} is H, halo, C₁-C₆ alkyl, or C₁-C₆ haloalkyl; or X^{3c} is C—R^{3c}, and R^{2c} and R^{3c}, together with the carbon atoms to which they are attached, form a ring of formula:

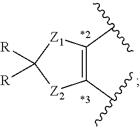

Z₁ and Z₂ are each independently O or CH₂;
each R is independently H or halo;
R^{4c} is H, halo, C₁-C₆ alkyl, C₁-C₆ haloalkyl, C₁-C₆ alkoxy, or C₁-C₆ haloalkoxy;
R^{5c} is H, halo, C₁-C₆ alkyl, or C₁-C₆ haloalkyl; and
R^{6c} is H, halo, C₁-C₆ alkyl, or C₁-C₆ haloalkyl; provided that no more than two of X^{2a}, X^{3a}, X^{5a}, and X^{6a} are N or N⁺—O⁻; and provided that no more than one of X^{3c}, X^{4c}, X^{5c}, and X^{6c} is N; and
provided that:
R^{5a} and R^{d} form a CH₂CH₂ chain linking the C atoms to which R^{5a} and R^{d} are attached, wherein the CH₂ group that is bound to the C atom to which R^{5a} is attached may be replaced with O; or
R^{2c} is O—CH₂—C(R^{2c1})(R^{2c2})(R^{2c3}) or O—CH(R^{2c4})(R^{2c5}).
2. The compound of clause 1, wherein the compound has formula (I-A)

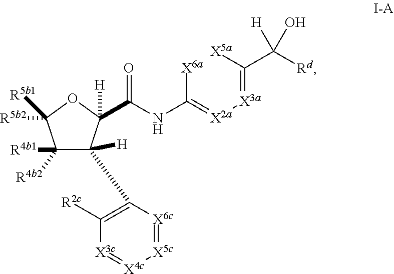

or a pharmaceutically acceptable salt thereof.
3. The compound of clause 1, wherein the compound has formula (I-A-1)

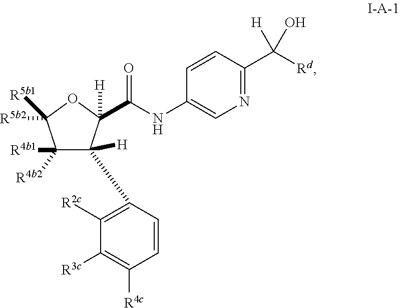

or a pharmaceutically acceptable salt thereof.
4. The compound of clause 1, wherein the compound has formula (I-A-2)

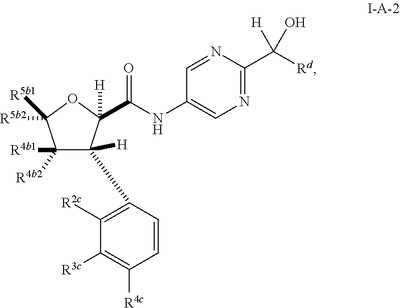

or a pharmaceutically acceptable salt thereof.
5. The compound of clause 1, wherein the compound has formula (I-A-3)

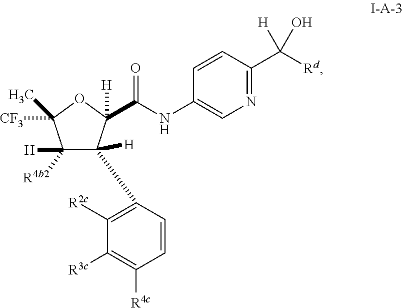

or a pharmaceutically acceptable salt thereof.
6. The compound of clause 1, wherein the compound has formula (I-B)

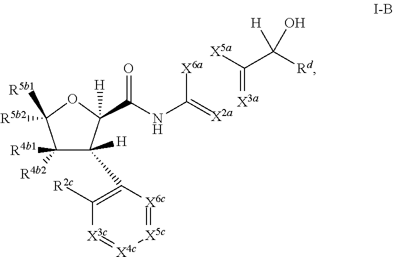

or a pharmaceutically acceptable salt thereof.
7. The compound of clause 1, wherein the compound has formula (I-B-1)

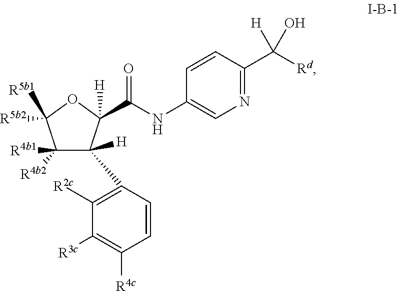

or a pharmaceutically acceptable salt thereof.
8. The compound of clause 1, wherein the compound has formula (I-B-2)

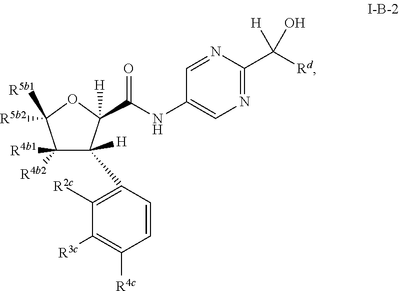

or a pharmaceutically acceptable salt thereof.
9. The compound of clause 1, wherein the compound has formula (I-B-3)

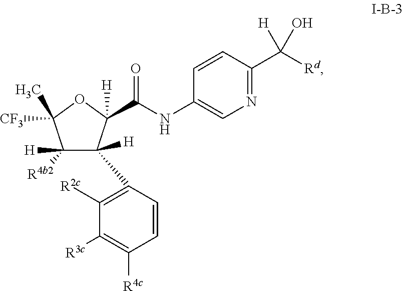

or a pharmaceutically acceptable salt thereof.

10. The compound of clause 1, wherein the compound has formula (I-C)

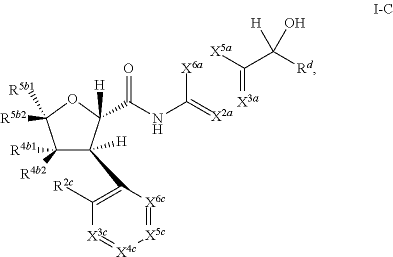

or a pharmaceutically acceptable salt thereof.
11. The compound of clause 1, wherein the compound has formula (I-C-1)

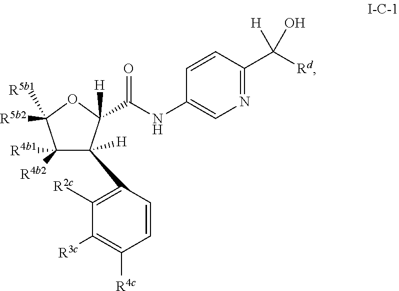

or a pharmaceutically acceptable salt thereof.
12. The compound of clause 1, wherein the compound has formula (I-C-2)

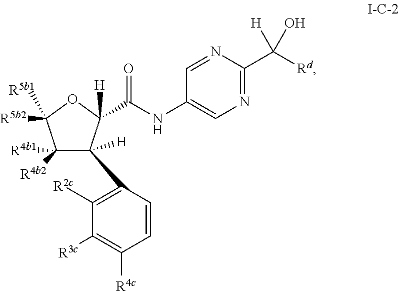

or a pharmaceutically acceptable salt thereof.
13. The compound of clause 1, wherein the compound has formula (I-C-3)

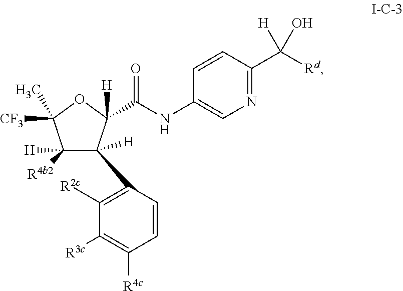

or a pharmaceutically acceptable salt thereof.
14. The compound of clause 1, wherein the compound has formula (I-D)

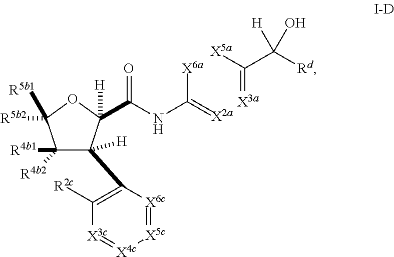

or a pharmaceutically acceptable salt thereof.
15. The compound of clause 1, wherein the compound has formula (I-D-1)

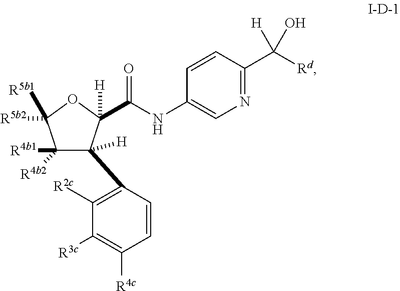

or a pharmaceutically acceptable salt thereof.
16. The compound of clause 1, wherein the compound has formula (I-D-2)

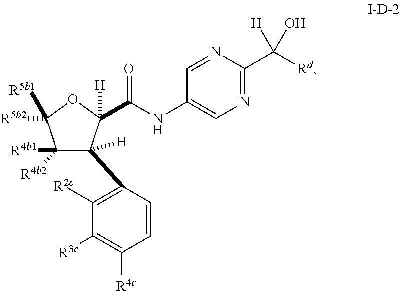

or a pharmaceutically acceptable salt thereof.
17. The compound of clause 1, wherein the compound has formula (I-D-3)

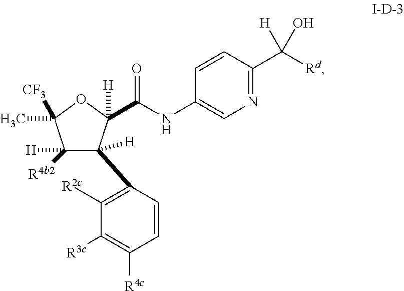

or a pharmaceutically acceptable salt thereof.
18. The compound of clause 1, wherein the compound has formula (I-D-4)

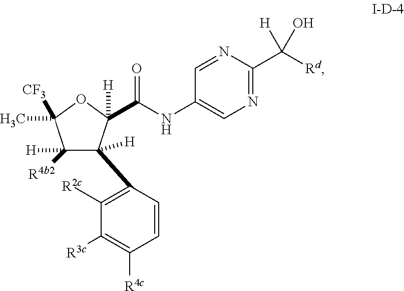

or a pharmaceutically acceptable salt thereof.
19. The compound of any one of clauses 1, 2, 6, 10, or 14, or a pharmaceutically acceptable salt thereof, wherein X^{2a} is C—R^{2a}, and R^{2a} is H.
20. The compound of any one of clauses 1, 2, 6, 10, or 14, or a pharmaceutically acceptable salt thereof, wherein X^{3a} is N.
21. The compound of any one of clauses 1, 2, 6, 10, or 14, or a pharmaceutically acceptable salt thereof, wherein X^{5a} is N.
22. The compound of any one of clauses 1, 2, 6, 10, or 14, or a pharmaceutically acceptable salt thereof, wherein X^{5a} is C—R^{5a}, and R^{5a} is H.
23. The compound of any one of clauses 1, 2, 6, 10, or 14, or a pharmaceutically acceptable salt thereof, wherein X^{6a} is C—R^{6a}, and R^{6a} is H.
24. The compound of any one of clauses 1-23, or a pharmaceutically acceptable salt thereof, wherein R^{e} is H.
25. The compound of any one of clauses 1-23, or a pharmaceutically acceptable salt thereof, wherein R^{e} is OH.
26. The compound of any one of clauses 1-23, or a pharmaceutically acceptable salt thereof, wherein R^{e} is C₁-C₆ alkoxy.
27. The compound of any one of clauses 1-4, 6-8, 10-12, 14-16, or 19-26, or a pharmaceutically acceptable salt thereof, wherein R^{4b1} is H.
28. The compound of any one of clauses 1-4, 6-8, 10-12, 14-16, or 19-26, or a pharmaceutically acceptable salt thereof, wherein R^{4b1} is C₁-C₆ alkyl.
29. The compound of any one of clauses 1-4, 6-8, 10-12, 14-16, or 19-26, or a pharmaceutically acceptable salt thereof, wherein R^{4b1} is CH₃.
30. The compound of any one of clauses 1-29, or a pharmaceutically acceptable salt thereof, wherein R^{4b2} is H.
31. The compound of any one of clauses 1-29, or a pharmaceutically acceptable salt thereof, wherein R^{4b2} is C₁-C₆ alkyl.
32. The compound of any one of clauses 1-29, or a pharmaceutically acceptable salt thereof, wherein R^{4b2} is CH₃.
33. The compound of any one of clauses 1-4, 6-8, 10-12, 14-16, or 19-32, or a pharmaceutically acceptable salt thereof, wherein R^{5b1} is C₁-C₆ alkyl.
34. The compound of clause 33, or a pharmaceutically acceptable salt thereof, wherein R^{5c} is CH₃.
35. The compound of any one of clauses 1-4, 6-8, 10-12, 14-16, or 19-32, or a pharmaceutically acceptable salt thereof, wherein R^{5b1} is C₁-C₆ haloalkyl.
36. The compound of clause 35, or a pharmaceutically acceptable salt thereof, wherein R^{5c} is CF₃.
37. The compound of any one of clauses 1-4, 6-8, 10-12, 14-16, or 19-36, or a pharmaceutically acceptable salt thereof, wherein R^{5b2} is C₁-C₆ alkyl.
38. The compound of any one of clauses 1-4, 6-8, 10-12, 14-16, or 19-36, or a pharmaceutically acceptable salt thereof, wherein R^{5b2} is CH₃.
39. The compound of any one of clauses 1-4, 6-8, 10-12, 14-16, or 19-36, or a pharmaceutically acceptable salt thereof, wherein R^{5b2} is C₁-C₆ haloalkyl.
40. The compound of any one of clauses 1-4, 6-8, 10-12, 14-16, or 19-36, or a pharmaceutically acceptable salt thereof, wherein R^{5b2} is CF₃.
41. The compound of any one of clauses 1-40, or a pharmaceutically acceptable salt thereof, wherein R^{2c} is C₁-C₆ alkoxy.
42. The compound of any one of clauses 1-40, or a pharmaceutically acceptable salt thereof, wherein R^{2c} is O—CH₂—C(R^{2c1})(R^{2c2})(R^{2c3}).
43. The compound of any one of clauses 1-40, or a pharmaceutically acceptable salt thereof, wherein R^{2c} is O—CH(R^{2c4})(R^{2c5}).
44. The compound of any one of clauses 1-2, 6, 10, 14, or 19-43, or a pharmaceutically acceptable salt thereof, wherein X^{3c} is C—R^{3c}, and R^{3c} is halo.
45. The compound of clause 44, or a pharmaceutically acceptable salt thereof, wherein R^{3c} is F.
45. The compound of any one of clauses 1-2, 6, 10, 14, or 19-43, or a pharmaceutically acceptable salt thereof, wherein X^{3c} is C—R^{3c}, and R^{3c} is C₁-C₆ alkyl.
46. The compound of clause 45, or a pharmaceutically acceptable salt thereof, wherein R^{3c} is CH₃.
47. The compound of any one of clauses 1-2, 6, 10, 14, or 19-46, or a pharmaceutically acceptable salt thereof, wherein X^{4c} is C—R^{4c}, and wherein R^{4c} is halo.
48. The compound of clause 45, or a pharmaceutically acceptable salt thereof, wherein wherein R^{4c} is F.
49. The compound of any one of clauses 1-2, 6, 10, 14, or 19-48, or a pharmaceutically acceptable salt thereof, wherein X^{5c} is C—R^{5c}, and wherein R^{5c} is H.
50. The compound of any one of clauses 1-2, 6, 10, 14, or 19-49, or a pharmaceutically acceptable salt thereof, wherein X^{6c} is C—R^{6c}, and wherein R^{6c} is H.
51. A compound selected from Table A, or a pharmaceutically acceptable salt thereof.
52. The compound of any one of clauses 1-51 in non-salt form.
53. A pharmaceutical composition comprising a therapeutically effective amount of the compound of any one of clauses 1-51, or a pharmaceutically acceptable salt thereof, or the compound of clause 52 and one or more pharmaceutically acceptable carriers or vehicles.
54. A pharmaceutical composition comprising the compound of any one of clauses 1-51, or a pharmaceutically acceptable salt thereof, or the compound of clause 52 and one or more pharmaceutically acceptable carriers or vehicles.
55. A method of inhibiting a voltage-gated sodium channel in a subject comprising administering to the subject the compound of any one of clauses 1-51, or a pharmaceutically acceptable salt thereof, the compound of clause 52, or the pharmaceutical composition of clause 53 or 54.
56. The method of clause 55, wherein the voltage-gated sodium channel is Na_{V}1.8.
57. A method of treating or lessening the severity in a subject of chronic pain, gut pain, neuropathic pain, musculoskeletal pain, acute pain, inflammatory pain, cancer pain, idiopathic pain, postsurgical pain, visceral pain, multiple sclerosis, Charcot-Marie-Tooth syndrome, incontinence, pathological cough, or cardiac arrhythmia comprising administering to the subject an effective amount of the compound of any one of clauses 1-51, or a pharmaceutically acceptable salt thereof, the compound of clause 52, or the pharmaceutical composition of clause 53 or 54.
58. The method of clause 57, where the method comprises treating or lessening the severity in the subject of neuropathic pain.
59. The method of clause 58, wherein the neuropathic pain comprises post-herpetic neuralgia.
60. The method of clause 58, wherein the neuropathic pain comprises small-fiber neuropathy.
61. The method of clause 58, wherein the neuropathic pain comprises idiopathic small-fiber neuropathy.
62. The method of clause 58, wherein the neuropathic pain comprises diabetic neuropathy.
63. The method of clause 62, wherein the diabetic neuropathy comprises diabetic peripheral neuropathy.
64. The method of clause 57, wherein the method comprises treating or lessening the severity in the subject of musculoskeletal pain.
65. The method of clause 64, wherein the musculoskeletal pain comprises osteoarthritis pain.
66. The method of clause 57, wherein the method comprises treating or lessening the severity in the subject of acute pain.
67. The method of clause 66, wherein the acute pain comprises acute post-operative pain.
68. The method of clause 57, wherein the method comprises treating or lessening the severity in the subject of postsurgical pain.
69. The method of clause 68, wherein the postsurgical pain comprises bunionectomy pain.
70. The method of clause 68, wherein the postsurgical pain comprises abdominoplasty pain.
71. The method of clause 68, wherein the postsurgical pain comprises herniorrhaphy pain.
72. The method of clause 57, wherein the method comprises treating or lessening the severity in the subject of visceral pain.
73. The method of any one of clauses 55-72, wherein said subject is treated with one or more additional therapeutic agents administered concurrently with, prior to, or subsequent to treatment with the compound, pharmaceutically acceptable salt, or pharmaceutical composition.
74. Use of the compound of any one of clauses 1-51, or a pharmaceutically acceptable salt thereof, the compound of clause 52, or the pharmaceutical composition of clause 53 or 54, as a medicament.

### EXAMPLES

General methods. ¹H NMR spectra were obtained as solutions in an appropriate deuterated solvent such as dimethyl sulfoxide-d₆ (DMSO-d6).

Compound purity, retention time, and electrospray mass spectrometry (ESI-MS) data were determined by LC/MS analysis using Method A or Method B as described below.

Method A. LC/MS analysis was conducted using an Acquity UPLC BEH C₈ column (50×2.1 mm, 1.7 m particle) made by Waters (pn: 186002877) with a (2.1×5 mm, 1.7 μm particle) guard column (pn: 186003978), and a dual gradient run from 2-98% mobile phase B over 4.45 minutes. Mobile phase A=H₂O (10 mM ammonium formate with 0.05% ammonium hydroxide). Mobile phase B=acetonitrile. Flow rate=0.6 mL/min, injection volume=2 μL, and column temperature=45° C.

Method B. LC/MS analysis was conducted using a Waters BEH C18 2.5 μm, 2.1×50 mm, UPLC, 4.6 min run, 2-95% ACN in water (0.1% NH₃ modifier), 0.8 mL/min, 40° C.

X-ray powder diffraction analysis method: X-ray powder diffraction (XRPD) analysis was performed at room temperature in transmission mode using a PANalytical Empyrean system equipped with a sealed tube source and a PIXcel 3D Medipix-3 detector (Malvern PANalytical Inc, Westborough, Massachusetts). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 20 with a step size of 0.0131303° and 49 s per step.

### Abbreviations

Unless otherwise noted, or where the context dictates otherwise, the following abbreviations shall be understood to have the following meanings:

| Abbreviation | Meaning |
|---|---|
| NMR | Nuclear magnetic resonance |
| ESI-MS | Electrospray mass spectrometry |
| LC/MS | Liquid chromatography-mass spectrometry |
| HPLC | High performance liquid chromatography |
| SFC | Supercritical fluid chromatography |
| g | grams |
| mg | milligrams |
| kg | kilograms |
| L | Liter(s) |
| mL | Milliliters |
| μL | Microliters |
| mol | moles |
| mmol | millimoles |
| hr, h | hours |
| min | Minutes |
| MHz | Megahertz |
| Hz | Hertz |
| N | Normal (concentration) |
| M | Molar (concentration) |
| ppm | Parts per million |
| % w/w | Weight-weight concentration |
| DAST | Diethylaminosulfur trifluoride |
| DIEA, DIPEA | N,N-Diisopropyl ethyl amine |
| DMAP | N,N-Dimethylaminopyridine |
| DMF | N,N-Dimethylformamide |
| EDC.HCl | Ethyl carbodiimide hydrochloride |
| EtOH | Ethanol |
| EtOAc | Ethyl acetate |
| HOBt | Hydroxybenzotriazole |
| T3P | Propylphosphonic anhydride, i.e., 2,4,6-tripropyl- |
| | 1,3,5,2,4,6-trioxatriphosphinane 2,4,6-trioxide |
| KOAc | Potassium acetate |
| m-CPBA | meta-chloroperoxybenzoic acid |
| MeOH | Methanol |
| MTBE | Methyl tert-butyl ether |
| NaOH | Sodium hydroxide |
| NBS | N-bromosuccinimide |
| NMP | N-Methylpyrrolidone |
| NMO | N-methylmorpholine N-oxide |
| PPTS | Pyridinium para-toluene sulfonate |
| TBAB | Tetra-n-butylammonium bromide |
| TBAF | Tetra-n-butylammonium fluoride |
| TBSCl | Tert-butyldimethylsilyl chloride |
| TBSOTf | Tert-butyldimethylsilyl trifluoromethanesulfonate |
| THF | Tetrahydrofuran |
| TEA | triethylamine |
| TFA | Trifluoroacetic acid |
| RT | Room temperature |

### Example 1

### rel-(2R*,3S*,4S*,5R*)-3-(3,4-difluoro-2-methoxyphenyl)-N-(3-hydroxy-2,3-dihydrofuro[3,2-b]pyridin-6-yl)-4,5-dimethyl-5-(trifluoromethyl)tetrahydrofuran-2-carboxamide (1) and rel-(2R*,3S*,4S*,5R*)-3-(3,4-difluoro-2-methoxyphenyl)-N-(3-hydroxy-2,3-dihydrofuro[3,2-b]pyridin-6-yl)-4,5-dimethyl-5-(trifluoromethyl)tetrahydrofuran-2-carboxamide (2)

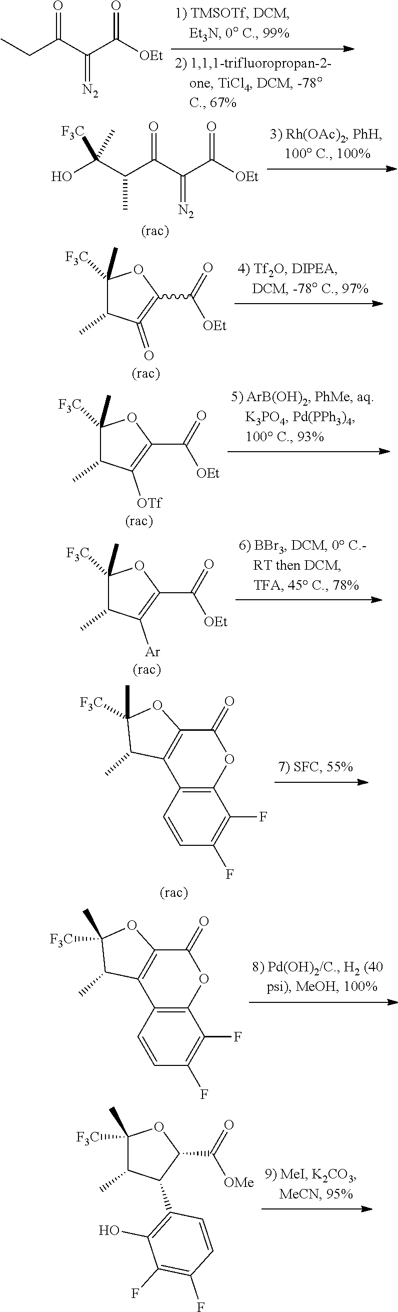

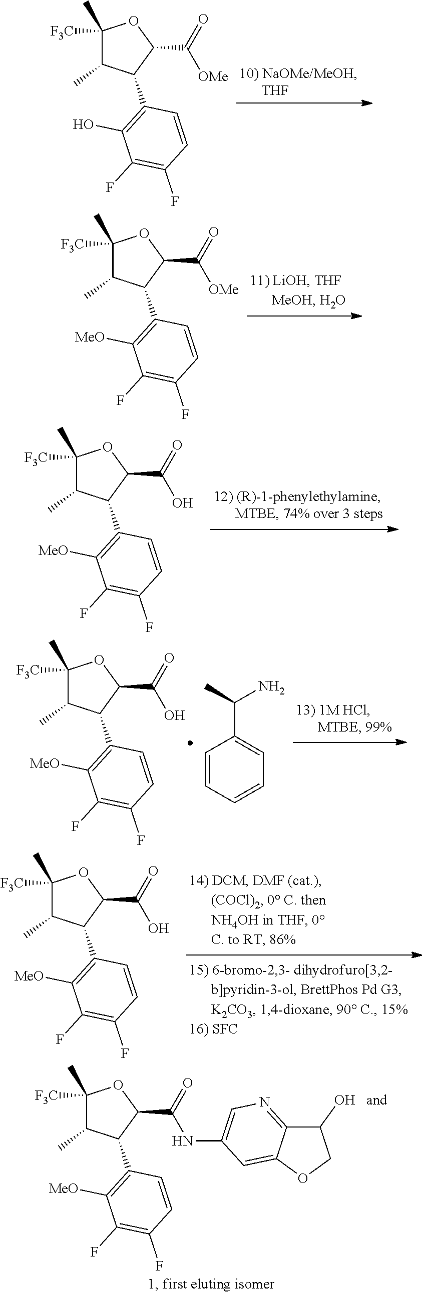

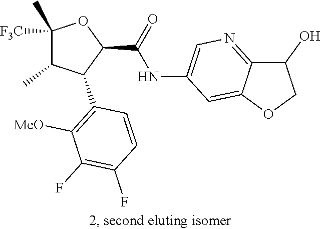 [SMILES: COc1c([C@H]2[C@H](C(=O)Nc3cnc4c(c3)OCC4O)O[C@@](C)(C(F)(F)F)[C@H]2C)ccc(F)c1F]

### Step 1:

NEt₃ (7.7 mL, 55.2 mmol) was added to a solution of ethyl 2-diazo-3-oxo-pentanoate (6.69 g, 39.3 mmol) in DCM (80 mL) with stirring at 0° C. under nitrogen. Trimethylsilyl trifluoromethanesulfonate (8.5 mL, 47.0 mmol) was added dropwise over 5 min and the mixture was stirred at 0° C. for 30 min. The reaction mixture was diluted with pentane (100 mL), the layers separated and the organic phase washed with dilute aqueous sodium bicarbonate (100 mL) and brine (100 mL). The organic layer was dried (MgSO₄), filtered and concentrated in vacuo to give ethyl (Z)-2-diazo-3-trimethylsilyloxy-pent-3-enoate (9.4 g, 99%) as a red oil. ¹H NMR (500 MHz, Chloroform-d) δ 5.33 (q, J=7.0 Hz, 1H), 4.25 (q, J=7.1 Hz, 2H), 1.67 (d, J=7.0 Hz, 3H), 1.29 (t, J=7.1 Hz, 3H), 0.22 (s, 9H) ppm.

### Step 2:

To a solution of 1,1,1-trifluoropropan-2-one (8 mL, 89.4 mmol) in DCM (80 mL) at −78° C. was added TiCl₄ (70 mL of 1 M in DCM, 70.00 mmol) via cannula. A solution of ethyl (Z)-2-diazo-3-trimethylsilyloxy-pent-3-enoate (36.1 g of 31.3% w/w, 46.6 mmol) in DCM (40 mL) was added dropwise over 15 min. After stirring for 100 min, the reaction was quenched with water, allowing the temperature to rise slowly, and extracted with DCM. The combined organic extracts were dried (MgSO₄), filtered, and concentrated in vacuo. Purification by silica gel chromatography (330 g SiO₂, 0 to 20% EtOAc in heptane) gave ethyl rac-(4R,5R)-2-diazo-6,6,6-trifluoro-5-hydroxy-4,5-dimethyl-3-oxohexanoate (8.82 g, 67%) as the main diastereoisomer, which was stored as a solution in toluene. ¹H NMR (500 MHz, Chloroform-d) δ 4.33 (q, J=7.1 Hz, 2H), 4.14 (q, J=7.0 Hz, 1H), 3.98 (s, 1H), 1.43 (q, J=1.2 Hz, 3H), 1.35 (t, J=7.1 Hz, 3H), 1.31 (dq, J=7.0, 1.4 Hz, 3H) ppm. ESI-MS m/z calc. 282.08273, found 283.1 (M+1)⁺; 281.0 (M−1)⁻; Retention time: 0.76 minutes.

### Step 3:

A solution of rhodium tetraacetate (245 mg, 0.55 mmol) in benzene (32 mL) was heated at reflux for 10 min before a solution of ethyl rac-(4R,5R)-2-diazo-6,6,6-trifluoro-5-hydroxy-4,5-dimethyl-3-oxohexanoate (10 g, 35.4 mmol) in benzene (13 mL) was added slowly while refluxing for 60 min. The mixture was concentrated in vacuo to give ethyl rac-(4R,5R)-4,5-dimethyl-3-oxo-5-(trifluoromethyl)tetrahydrofuran-2-carboxylate (9.0 g, 100%) as a green coloured residue, and as a mixture of epimers at the position next to the ester. ¹H NMR (500 MHz, Chloroform-d) δ 4.83-4.57 (m, 1H), 4.38-4.16 (m, 2H), 2.60 (dddd, J=9.3, 8.2, 5.6, 1.4 Hz, 1H), 1.73-1.63 (m, 3H), 1.30 (t, J=7.1 Hz, 3H), 1.24 (ddq, J=6.4, 4.1, 1.9 Hz, 3H) ppm.

### Step 4:

To a solution of ethyl rac-(4R,5R)-4,5-dimethyl-3-oxo-5-(trifluoromethyl)tetrahydrofuran-2-carboxylate (48 g, 188.83 mmol) in DCM (400 mL) cooled to −78° C. was added DIPEA (29.680 g, 40 mL, 229.64 mmol). A solution of trifluoromethylsulfonyl trifluoromethanesulfonate (53.440 g, 32 mL, 189.41 mmol) in DCM (200 mL) was added to the reaction mixture at −78° C. over 1 h. The reaction mixture was stirred at 0° C. for 30 min and then quenched with saturated aqueous NaHCO₃ (100 mL). The organic layer was separated and aqueous layer extracted with DCM (160 mL). The combined organic layers were dried (MgSO₄) and concentrated in vacuo to give ethyl rac-(4R,5R)-2,3-dimethyl-2-(trifluoromethyl)-4-(trifluoromethylsulfonyloxy)-3H-furan-5-carboxylate (71 g, 97%). ¹H NMR (400 MHz, Chloroform-d) δ 4.38-4.32 (m, 2H), 3.29-3.23 (m, 1H), 1.64 (s, 3H), 1.37-1.33 (m, 6H) ppm.

### Step 5:

To stirred a solution of ethyl rac-(4R,5R)-2,3-dimethyl-2-(trifluoromethyl)-4-(trifluoromethylsulfonyloxy)-3H-furan-5-carboxylate (26 g, 67.31 mmol) in toluene (130 mL) was added (3,4-difluoro-2-methoxy-phenyl)boronic acid (14 g, 74.5 mmol) followed by K₃PO₄ (100 mL of 2 M, 200 mmol) and tetrakis(triphenylphosphine)palladium(0) (4 g, 3.46 mmol) under an argon atmosphere. The reaction mixture was heated at 100° C. for 2 h. The reaction mixture was diluted with water and the aqueous layer extracted with EtOAc (2×100 mL). The combined organic layers were concentrated in vacuo. Purification by silica gel chromatography (SiO₂, 0 to 10% EtOAc in heptane) gave ethyl 4-(3,4-difluoro-2-methoxy-phenyl)-2,3-dimethyl-2-(trifluoromethyl)-3H-furan-5-carboxylate (24.4 g, 93%) as a 6:1 diastereomeric mixture, with the major isomer believed to be ethyl rac-(4S,5R)-4-(3,4-difluoro-2-methoxyphenyl)-2,3-dimethyl-2-(trifluoromethyl)-3H-furan-5-carboxylate. Major isomer: ¹H NMR (400 MHz, Chloroform-d) δ 6.88-6.79 (m, 2H), 4.17-4.09 (m, 2H), 3.90 (s, 3H), 3.46 (q, J=7.4 Hz, 1H), 1.67 (s, 3H), 1.12 (t, J=7.4 Hz, 3H), 1.06 (dd, J=5.4, 2.7 Hz, 3H) ppm. Minor isomer: ¹H NMR (400 MHz, Chloroform-d) δ 6.88-6.79 (m, 2H), 4.17-4.09 (m, 2H), 3.88 (s, 3H), 3.76-3.71 (m, 1H), 1.51 (s, 3H), 1.12 (t, J=7.4 Hz, 3H), 0.99 (dd, J=5.4, 2.7 Hz, 3H) ppm. ESI-MS m/z calc. 380.1047, found 381.02 (M+1)⁺.

### Step 6:

To an ice-cooled solution of ethyl rac-(4S,5R)-4-(3,4-difluoro-2-methoxyphenyl)-2,3-dimethyl-2-(trifluoromethyl)-3H-furan-5-carboxylate (110 g, 243.0 mmol) in DCM (360 mL) was added BBr₃ (370 mL of 1 M, 370.0 mmol) dropwise. The mixture was quenched by the addition of water and aqueous sodium bicarbonate solution. The aqueous layer was extracted with DCM and the combined organic extracts were dried (MgSO₄), filtered and concentrated in vacuo. The residue was dissolved in DCM (430 mL) followed by the addition of TFA (40 mL, 519.2 mmol). The reaction mixture was heated to 45° C. Upon reaction completion, the mixture was quenched by addition of aqueous sodium bicarbonate solution and extracted with DCM. The combined organic extracts were dried (MgSO₄), filtered and concentrated in vacuo to give the desired product in a 5:1 mixture of diastereomers. Recrystallization was carried out by solubilizing the crude material in the smallest possible amount of DCM and adding a layer of heptane on top of this solution (liquid-liquid diffusion). After 1 h, 56.5 g (d.r. 97:3 syn:anti) from the first and second crystallization was obtained, and a further 4.6 g (d.r. 96:4 syn:anti) from the third crystallization was obtained. The first to third batches were combined to give 6,7-difluoro-1,2-dimethyl-2-(trifluoromethyl)-1H-furo[2,3-c]chromen-4-one (61 g, 78%), with the major isomer believed to be rac-(1S,2R)-6,7-difluoro-1,2-dimethyl-2-(trifluoromethyl)-1,2-dihydro-4H-furo[2,3-c]chromen-4-one. ESI-MS m/z calc. 320.04718, found 321.5 (M+1); 319.6 (M−1)⁻.

### Step 7:

rac-(1S,2R)-6,7-difluoro-1,2-dimethyl-2-(trifluoromethyl)-1,2-dihydro-4H-furo[2,3-c]chromen-4-one (1348 g, 4.366 mol) was separated by chiral SFC using a (R,R)-Whelk-01 column, 5 m particle size, 15 cm×3 cm from Regis Technologies on a MultiGram III SFC instrument from Berger Instruments to give:

First Eluting Isomers (rt=1.85 min): (1R,2S)-6,7-difluoro-1,2-dimethyl-2-(trifluoromethyl)-1,2-dihydro-4H-furo[2,3-c]chromen-4-one (only an analytical sample was collected). ¹H NMR (400 MHz, DMSO-d₆) δ 7.57 (ddd, J=9.0, 5.5, 2.0 Hz, 1H), 7.51 (ddd, J=10.3, 9.0, 7.0 Hz, 1H), 4.03 (q, J=7.2 Hz, 1H), 1.65 (s, 3H), 1.45 (dt, J=6.9, 2.2 Hz, 3H) ppm. ESI-MS m/z calc. 320.04718, found 321.3 (M+1)⁺; 319.4 (M−1)⁻.

Second Eluting Isomer (rt=2.38 min): (1S,2R)-6,7-Difluoro-1,2-dimethyl-2-(trifluoromethyl)-1,2-dihydro-4H-furo[2,3-c]chromen-4-one (366.99 g, 26%). ¹H NMR (400 MHz, DMSO-d₆) δ 7.57 (ddd, J=9.0, 5.5, 2.0 Hz, 1H), 7.50 (ddd, J=10.3, 9.0, 7.0 Hz, 1H), 4.03 (q, J=7.2 Hz, 1H), 1.65 (s, 3H), 1.45 (dt, J=6.9, 2.2 Hz, 3H) ppm. ESI-MS m/z calc. 320.04518, found 321.4 (M+1)⁺; 319.4 (M−1)⁻.

### Step 8:

A solution of (1S,2R)-6,7-Difluoro-1,2-dimethyl-2-(trifluoromethyl)-1,2-dihydro-4H-furo[2,3-c]chromen-4-one (0.89 kg, 2.78 mol) and palladium hydroxide on carbon (50% wet, 0.39 kg of 20 wt. % loading, 0.278 mol) in MeOH (12 L) was stirred under a 40 psi pressure of hydrogen overnight. An increase in the reaction temperature to 37° C. was observed after reacting overnight and the mixture was cooled to 24° C. The hydrogenation was continued for a total of 48 h. The mixture was filtered through celite, washing with MeOH (20 L) and the filtrates were concentrated in vacuo. The residue was dissolved in toluene (4 L) and concentrated in vacuo, and this process repeated. The residue was dried under vacuum at 40° C. overnight to give methyl (2S,3S,4S,5R)-3-(3,4-difluoro-2-hydroxyphenyl)-4,5-dimethyl-5-(trifluoromethyl)tetrahydrofuran-2-carboxylate (1.0 kg at 91% purity, 100%) as a beige solid. H NMR (400 MHz, DMSO-d₆) 10.20 (br s, 1H), 6.94 (br t, J=7.4 Hz, 1H), 6.79-6.69 (m, 1H), 5.10 (d, J=6.0 Hz, 1H), 4.20 (dd, J=6.1, 8.2 Hz, 1H), 3.43 (s, 3H), 2.94 (quin, J=7.7 Hz, 1H), 1.46 (s, 3H), 0.77 (br d, J=6.8 Hz, 3H) ppm.

### Step 9:

Potassium carbonate (2.0 kg, 14.4 mol) and iodomethane (800 mL, 12.8 mol) were sequentially added to a solution of methyl (2S,3S,4S,5R)-3-(3,4-difluoro-2-hydroxyphenyl)-4,5-dimethyl-5-(trifluoromethyl)tetrahydrofuran-2-carboxylate (1.0 kg, 2.82 mol) in acetonitrile (10 L) under nitrogen at ambient temperature. After stirring overnight, additional iodomethane (120 mL, 2 mmol) was added. After stirring overnight, additional iodomethane (60 mL, 0.85 mmol) was added and the mixture was stirred for a further 3 days. The reaction mixture was diluted with MTBE (30 L), treated with celite (1 kg) and filtered through a bed of celite (1 kg) washing with MTBE (10 L). The filtrate was filtered a second time through celite (1 kg) washing with MTBE (4 L) and the filtrate concentrated in vacuo. The residue was dissolved in toluene (4 L) and concentrated in vacuo, and this process repeated. The residue was dried under vacuum at 40° C. overnight to give methyl (2S,3S,4S,5R)-3-(3,4-difluoro-2-methoxyphenyl)-4,5-dimethyl-5-(trifluoromethyl)tetrahydrofuran-2-carboxylate (0.99 kg at 90% purity, 95%) as a brown solid. ¹H NMR (400 MHz, DMSO-d₆) 7.14-7.00 (m, 2H), 5.14 (d, J=6.0 Hz, 1H), 4.15 (dd, J=6.2, 8.4 Hz, 1H), 3.88 (d, J=1.7 Hz, 3H), 2.97 (quin, J=7.8 Hz, 1H), 1.48 (s, 3H), 0.72 (br d, J=6.6 Hz, 3H) ppm.

### Step 10 and 11:

To a solution of methyl (2S,3S,4S,5R)-3-(3,4-difluoro-2-methoxyphenyl)-4,5-dimethyl-5-(trifluoromethyl) tetrahydrofuran-2-carboxylate (0.98 kg, 2.66 mol) in THF (10 L) was added sodium methoxide (25% w/w in methanol, 65 mL, 0.28 mol) at ambient temperature and the reaction stirred for 5 h. MeOH (1 L), water (1 L) and lithium hydroxide monohydrate (0.168 kg, 4.0 mol) were sequentially added and the mixture was stirred overnight. The reaction mixture was poured into 1M HCl (4.4 L, 4.4 mol) then extracted with MTBE (20 L) and then MTBE (2×5 L). The combined organic extracts were washed with brine (2 L), dried (Na₂SO₄), filtered then treated with activated carbon (50 g, 5% w/w) with stirring for 1 h. The mixture was filtered through celite, washing with MTBE (2×4 L) and the filtrate concentrated in vacuo. The residue was dissolved in toluene (4 L) and concentrated in vacuo, then dissolved in MTBE (4 L) and concentrated in vacuo again to give (2R,3S,4S,5R)-3-(3,4-difluoro-2-methoxyphenyl)-4,5-dimethyl-5-(trifluoromethyl)tetrahydrofuran-2-carboxylic acid (1.06 kg at 77.7% purity) as an amber oil, which was used without further purification.

### Step 12:

(2R,3S,4S,5R)-3-(3,4-difluoro-2-methoxyphenyl)-4,5-dimethyl-5-(trifluoromethyl)tetrahydrofuran-2-carboxylic acid (2.09 kg at 77% purity, 4.54 mol) was dissolved in MTBE (25 L) in a 100-L Chemglass reactor then stirred at 84 rpm at ambient temperature. A mixture of (R)-1-phenylethylamine (0.704 kg, 5.81 mol) and MTBE (2 L) was added to the reactor, followed by additional MTBE to give a total volume of 30 L in the reactor. After 2 h additional MTBE (2 L) was added to the reaction. After a total of 3.5 h the mixture was filtered, washing with MTBE (2 L). The reactor was rinsed with MTBE (4 L), which was used to rinse the solids, which were then compressed and dried on the Buchner funnel for 2 h. The solid product cake was loosened then dried under a stream of nitrogen and under vacuum overnight on the Buchner funnel. The isolated solids were dried in a convection oven at 40° C. for 24 h to give (2R,3S,4S,5R)-3-(3,4-Difluoro-2-methoxyphenyl)-4,5-dimethyl-5-(trifluoromethyl)tetrahydrofuran-2-carboxylic acid (R)-1-phenylethan-1-amine salt (1.86 kg at 95.7% purity, 74% over 3 steps) as an off-white solid. ¹H NMR, 400 MHz, DMSO-d₆) 8.34 (br s, 2H), 7.46-7.41 (m, 2H), 7.36-7.27 (m, 3H), 7.16-7.11 (m, 1H), 7.10-7.03 (m, 1H), 4.58 (d, J=9.9 Hz, 1H), 4.23 (q, J=6.7 Hz, 1H), 3.99 (dd, J=7.8, 9.8 Hz, 1H), 3.90 (d, J=2.0 Hz, 3H), 2.60 (quin, J=7.5 Hz, 1H), 1.50 (s, 3H), 1.40 (d, J=6.7 Hz, 3H), 0.71-0.59 (m, 3H) ppm.

### Step 13:

To a suspension of (2R,3S,4S,5R)-3-(3,4-difluoro-2-methoxy-phenyl)-4,5-dimethyl-5-(trifluoromethyl)tetrahydrofuran-2-carboxylic acid (1R)-1-phenylethanamine salt (10.6 g, 22.29 mmol) in MTBE (250 mL) was added HCl (200 mL of 2 M, 400.0 mmol). The layers were separated and the organic layer was washed with water (200 mL), dried (MgSO₄), filtered and concentrated in vacuo to give (2R,3S,4S,5R)-3-(3,4-difluoro-2-methoxy-phenyl)-4,5-dimethyl-5-(trifluoromethyl)tetrahydrofuran-2-carboxylic acid (8.4 g, 99%) as an oil. ¹H NMR (400 MHz, Chloroform-d) δ 6.96 (ddd, J=7.9, 5.6, 2.0 Hz, 1H), 6.88 (td, J=9.2, 7.3 Hz, 1H), 4.96 (d, J=10.5 Hz, 1H), 4.15 (dd, J=10.5, 8.0 Hz, 1H), 4.02 (d, J=2.8 Hz, 3H), 2.74 (p, J=7.6 Hz, 1H), 1.64 (t, J=1.2 Hz, 3H), 0.79 (dq, J=7.4, 2.3 Hz, 3H) ppm.

### Step 14:

To an ice cold solution of (2R,3S,4S,5R)-3-(3,4-difluoro-2-methoxy-phenyl)-4,5-dimethyl-5-(trifluoromethyl)tetrahydrofuran-2-carboxylic acid (2.50 g, 7.057 mmol) in DCM (12 mL) was added DMF (1 drop) followed by careful addition of oxalyl chloride (1.25 mL, 14.33 mmol). The reaction mixture was warmed to ambient temperature and stirred for 60 minutes. The reaction mixture was concentrated in vacuo and the residue dissolved in DCM (10 mL). This solution was treated with ammonium hydroxide (2.50 mL of 25% w/v, 36.70 mmol) and the reaction mixture was stirred at ambient temperature overnight. The reaction mixture was diluted with DCM (20 mL) and poured over a saturated aqueous NaHCO₃ solution (30 mL). The organic layer was separated and the aqueous layer was extracted with DCM (2×30 mL). The organic layers were combined, washed with brine (50 mL), dried (MgSO₄), filtered and concentrated in vacuo to give a yellow oil. Purification by flash chromatography (SiO₂, 0 to 100% EtOAc in heptane) gave (2R,3S,4S,5R)-3-(3,4-difluoro-2-methoxy-phenyl)-4,5-dimethyl-5-(trifluoromethyl)tetrahydrofuran-2-carboxamide (2.132 g, 86%) as a white solid. ¹H NMR (500 MHz, DMSO-d₆) δ 7.50 (s, 1H), 7.32 (s, 1H), 7.17-7.09 (m, 2H), 4.82 (d, J=10.6 Hz, 1H), 4.04 (dd, J=10.6, 7.5 Hz, 1H), 3.93 (d, J=2.0 Hz, 3H), 2.65 (dq, J=7.5, 7.5 Hz, 1H), 1.55 (s, 3H), 0.67 (d, J=6.3 Hz, 3H) ppm. ¹⁹F NMR (471 MHz, DMSO-d₆) δ −73.5 (s, 3F), −138.67 (s, 1F), −155.48 (s, 1F) ppm. ESI-MS m/z calc. 353.10504, found 354.4 (M+1); 352.4 (M−1)⁻; Retention time: 3.11 minutes.

### Step 15:

To a mixture of (2R,3S,4S,5R)-3-(3,4-difluoro-2-methoxy-phenyl)-4,5-dimethyl-5-(trifluoromethyl)tetrahydrofuran-2-carboxamide (300 mg, 0.849 mmol), BrettPhos Pd G3 (150 mg, 0.165 mmol) and K₂CO₃ (240 mg, 1.73 mmol) under nitrogen was added a solution of 6-bromo-2,3-dihydrofuro[3,2-b]pyridin-3-ol (217 mg, 1.004 mmol) in 1,4-dioxane (5.0 mL). The mixture was sparged with nitrogen for 2 minutes and stirred at 90° C. overnight. The reaction mixture was concentrated in vacuo. Purification by flash chromatography (SiO₂, 15 to 100% EtOAc in hexanes) gave (2R,3S,4S,5R)-3-(3,4-difluoro-2-methoxy-phenyl)-N-(3-hydroxy-2,3-dihydrofuro[3,2-b]pyridin-6-yl)-4,5-dimethyl-5-(trifluoromethyl)tetrahydrofuran-2-carboxamide (63 mg, 15%) as an orange oil. ESI-MS m/z calc. 488.13705, found 489.5 (M+1)⁺; 487.4 (M−1)⁻; Retention time: 3.23 minutes.

### Step 16:

The diastereomers of (2R,3S,4S,5R)-3-(3,4-difluoro-2-methoxyphenyl)-N-(3-hydroxy-2,3-dihydrofuro[2,3-b]pyridin-6-yl)-4,5-dimethyl-5-(trifluoromethyl)tetrahydrofuran-2-carboxamide (62 mg, 0.12 mmol) were separated by chiral SFC using a Chiralpak IC column, 5 m particle size, 25 cm×20 mm from Daicel Corporation on a Prep-100 SFC instrument from Waters:

First Eluting Isomer (rt=1.35 min): rel-(2R*,3S*,4S*,5R*)-3-(3,4-difluoro-2-methoxyphenyl)-N-(3-hydroxy-2,3-dihydrofuro[3,2-b]pyridin-6-yl)-4,5-dimethyl-5-(trifluoromethyl)tetrahydrofuran-2-carboxamide (1, 17 mg, 56%). ¹H NMR (400 MHz, DMSO-d₆) δ 10.41 (s, 1H), 8.27 (d, J=2.0 Hz, 1H), 7.58 (d, J=2.0 Hz, 1H), 7.21-7.10 (m, 2H), 5.78 (d, J=4.6 Hz, 1H), 5.13-5.10 (m, 1H), 5.08 (d, J=10.3 Hz, 1H), 4.62 (dd, J=10.4, 6.9 Hz, 1H), 4.31 (dd, J=10.4, 2.9 Hz, 1H), 4.23 (dd, J=10.3, 7.5 Hz, 1H), 3.94 (d, J=2.2 Hz, 3H), 2.76 (dq, J=7.5, 7.5 Hz, 1H), 1.59 (s, 3H), 0.75-0.70 (m, 3H) ppm. ¹⁹F NMR (471 MHz, DMSO-d₆) δ −73.36 (s, 3F), −138.16 (s, 1F), −154.97 (s, 1F) ppm. ESI-MS m/z calc. 488.13705, found 489.5 (M+1); 487.4 (M−1)⁻; Retention time: 3.23 minutes.

Second Eluting Isomer (rt=1.44 min): rel-(2R*,3S*,4S*,5R*)-3-(3,4-difluoro-2-methoxyphenyl)-N-(3-hydroxy-2,3-dihydrofuro[3,2-b]pyridin-6-yl)-4,5-dimethyl-5-(trifluoromethyl)tetrahydrofuran-2-carboxamide (2, 21 mg, 66%). ¹H NMR (400 MHz, DMSO-d₆) δ 10.43 (s, 1H), 8.27 (d, J=2.0 Hz, 1H), 7.58 (d, J=2.0 Hz, 1H), 7.21-7.11 (m, 2H), 5.80 (s, 1H), 5.12-5.09 (m, 1H), 5.08 (d, J=10.3 Hz, 1H), 4.61 (dd, J=10.4, 6.9 Hz, 1H), 4.31 (dd, J=10.4, 2.9 Hz, 1H), 4.23 (dd, J=10.3, 7.6 Hz, 1H), 3.94 (d, J=2.2 Hz, 3H), 2.76 (dq, J=7.5, 7.5 Hz, 1H), 1.59 (s, 3H), 0.75-0.69 (m, 3H) ppm. ¹⁹F NMR (471 MHz, DMSO-d₆) δ −73.34 (s, 3F), −138.17 (s, 1F), −154.99 (s, 1F) ppm. ESI-MS m/z calc. 488.13705, found 489.5 (M+1)⁺; 487.4 (M−1)⁻; Retention time: 3.23 minutes.

### Example 2

### rel-(2R*,3S*,4S*,5R*)-3-(3,4-difluoro-2-methoxyphenyl)-N-(7-hydroxy-6,7-dihydro-5H-cyclopenta[b]pyridin-3-yl)-4,5-dimethyl-5-(trifluoromethyl)tetrahydrofuran-2-carboxamide (3) and rel-(2R*,3S*,4S*,5R*)-3-(3,4-difluoro-2-methoxyphenyl)-N-(7-hydroxy-6,7-dihydro-5H-cyclopenta[b]pyridin-3-yl)-4,5-dimethyl-5-(trifluoromethyl)tetrahydrofuran-2-carboxamide (4)

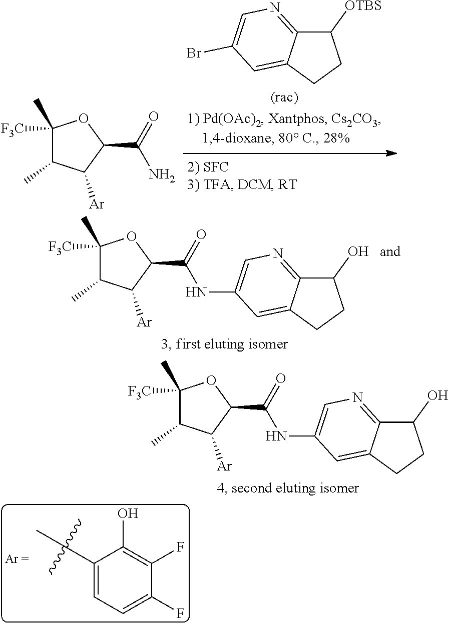

To a solution of (2R,3S,4S,5R)-3-(3,4-difluoro-2-methoxy-phenyl)-4,5-dimethyl-5-(trifluoromethyl)tetrahydrofuran-2-carboxamide (130 mg, 0.368 mmol) in 1,4-dioxane (3.6 mL) was added Xantphos (21 mg, 0.036 mmol), Cs₂CO₃ (240 mg, 0.736 mmol), Pd(OAc)₂ (4.1 mg, 0.018 mmol) and rac-(3-bromo-6,7-dihydro-5H-cyclopenta[b]pyridin-7-yl)oxy-tert-butyl-dimethyl-silane (136 mg, 0.414 mmol). The reaction mixture was degassed with nitrogen/vacuum cycles before sealing and heating at 80° C. overnight. The reaction mixture was diluted with EtOAc (20 mL) and poured over a saturated aqueous NaHCO₃ solution (20 mL). The organic layer was separated and the aqueous layer was extracted with EtOAc (2×20 mL). The organic layers were combined, washed with brine (20 mL), dried (MgSO₄), filtered and concentrated in vacuo to give a yellow oil. Purification by flash chromatography (SiO₂, 0 to 90% EtOAc in heptane) gave (2R,3S,4S,5R)—N-[7-[tert-butyl(dimethyl)silyl]oxy-6,7-dihydro-5H-cyclopenta[b]pyridin-3-yl]-3-(3,4-difluoro-2-methoxy-phenyl)-4,5-dimethyl-5-(trifluoromethyl)tetrahydrofuran-2-carboxamide (63 mg, 28%) as a colourless oil as a mixture of diastereomers at the tert-butyl(dimethyl)silyloxy position. ¹H NMR (500 MHz, Chloroform-d) δ 8.39 (s, 1H), 8.36-8.29 (m, 1H), 8.12-8.04 (m, 1H), 7.11-7.06 (m, 1H), 6.92-6.85 (m, 1H), 5.12 (dd, J=6.8, 4.6 Hz, 1H), 5.01 (dd, J=10.9, 2.5 Hz, 1H), 4.09 (dd, J=10.9, 8.0 Hz, 1H), 3.99 (t, J=2.5 Hz, 3H), 3.03-2.96 (m, 1H), 2.78-2.67 (m, 2H), 2.42-2.34 (m, 1H), 2.05-1.97 (m, 1H), 1.67 (s, 3H), 0.91 (d, J=1.3 Hz, 9H), 0.80-0.77 (m, 3H), 0.18 (s, 3H), 0.13 (s, 3H) ppm. ¹⁹F NMR (471 MHz, DMSO-d₆) δ −74.56 (s, 3F), −137.29 (s, 1F), −154.68 (s, 1F) ppm. ESI-MS m/z calc. 600.24426, found 601.7 (M+1)⁺; 599.7 (M−1)⁻; Retention time: 4.52 minutes.

### Step 2:

The diastereomers of (2R,3S,4S,5R)—N-[7-[tert-butyl(dimethyl)silyl]oxy-6,7-dihydro-5H-cyclopenta[b]pyridin-3-yl]-3-(3,4-difluoro-2-methoxy-phenyl)-4,5-dimethyl-5-(trifluoromethyl)tetrahydrofuran-2-carboxamide (63 mg, 0.105 mmol) were separated by by chiral SFC using a Chiralpak IB column, 5 m particle size, 25 cm×20 mm from Daicel Corporation on a Prep-100 SFC instrument from Waters:

First Eluting Isomer (rt=3.59 min): rel-(2R*,3S*,4S*,5R*)—N-(7-((tert-butyldimethylsilyl)oxy)-6,7-dihydro-5H-cyclopenta[b]pyridin-3-yl)-3-(3,4-difluoro-2-methoxyphenyl)-4,5-dimethyl-5-(trifluoromethyl)tetrahydrofuran-2-carboxamide (20.5 mg, 65%). ESI-MS m/z calc. 600.24426, found 601.2 (M+1)⁺; 599.3 (M−1)⁻; Retention time: 4.44 minutes.

Second Eluting Isomer (rt=4.47 min): rel-(2R*,3S*,4S*,5R*)—N-(7-((tert-butyldimethylsilyl)oxy)-6,7-dihydro-5H-cyclopenta[b]pyridin-3-yl)-3-(3,4-difluoro-2-methoxyphenyl)-4,5-dimethyl-5-(trifluoromethyl)tetrahydrofuran-2-carboxamide (13.5 mg, 43%). ESI-MS m/z calc. 600.24426, found 601.2 (M+1)⁺; 599.3 (M−1)⁻; Retention time: 4.44 minutes.

### Step 3:

TFA (200 μL, 2.596 mmol) was added to a solution of rel-(2R*,3S*,4S*,5R*)—N-(7-((tert-butyldimethylsilyl)oxy)-6,7-dihydro-5H-cyclopenta[b]pyridin-3-yl)-3-(3,4-difluoro-2-methoxyphenyl)-4,5-dimethyl-5-(trifluoromethyl)tetrahydrofuran-2-carboxamide (20.5 mg, 0.03413 mmol, first eluting isomer from step 2) in DCM (1.0 mL) and water (100 μL). The resulting mixture was stirred at ambient temperature over the weekend. The reaction mixture was concentrated in vacuo and azeotroped with MeOH to remove excess TFA. Purification by reverse phase HPLC-MS using a a X-bridge C18 column (150×19 mm, 5 m particle size) from Waters gave rel-(2R*,3S*,4S*,5R*)-3-(3,4-difluoro-2-methoxyphenyl)-N-(7-hydroxy-6,7-dihydro-5H-cyclopenta[b]pyridin-3-yl)-4,5-dimethyl-5-(trifluoromethyl)tetrahydrofuran-2-carboxamide (3, 12.4 mg, 74%). ¹H NMR (500 MHz, DMSO-d₆) δ 10.36 (s, 1H), 8.55 (d, J=2.2 Hz, 1H), 7.93 (d, J=2.2 Hz, 1H), 7.21-7.11 (m, 2H), 5.09 (d, J=10.3 Hz, 1H), 4.89 (dd, J=7.2, 5.1 Hz, 1H), 4.24 (dd, J=10.3, 7.6 Hz, 1H), 3.94 (d, J=2.0 Hz, 3H), 2.95-2.87 (m, 1H), 2.79-2.65 (m, 2H), 2.38-2.28 (m, 1H), 1.87-1.77 (m, 1H), 1.60 (s, 3H), 0.73 (d, J=6.8 Hz, 3H) ppm. ¹⁹F NMR (471 MHz, DMSO-d₆) δ −73.36 (s, 3F), −138.19 (s, 1F), −155.00 (s, 1F) ppm (alcohol OH not observed). ESI-MS m/z calc. 486.1578, found 487.6 (M+1)⁺; 485.5 (M−1)⁻; Retention time: 3.26 minutes.

rel-(2R*,3S*,4S*,5R*)—N-(7-((tert-butyldimethylsilyl)oxy)-6,7-dihydro-5H-cyclopenta[b]pyridin-3-yl)-3-(3,4-difluoro-2-methoxyphenyl)-4,5-dimethyl-5-(trifluoromethyl)tetrahydrofuran-2-carboxamide (13.5 mg, 0.022 mmol, second eluting isomer from step 2) was treated in the same way to give rel-(2R*,3S*,4S*,5R*)-3-(3,4-difluoro-2-methoxyphenyl)-N-(7-hydroxy-6,7-dihydro-5H-cyclopenta[b]pyridin-3-yl)-4,5-dimethyl-5-(trifluoromethyl)tetrahydrofuran-2-carboxamide (4, 12.4 mg, 74%) as a white solid. ¹H NMR (500 MHz, DMSO-d₆) δ 10.32 (s, 1H), 8.54 (d, J=2.2 Hz, 1H), 7.89 (d, J=2.2 Hz, 1H), 7.20-7.11 (m, 2H), 5.25 (d, J=5.5 Hz, 1H), 5.08 (d, J=10.3 Hz, 1H), 4.89-4.84 (m, 1H), 4.23 (dd, J=10.3, 7.7 Hz, 1H), 3.94 (d, J=2.0 Hz, 3H), 2.94-2.86 (m, 1H), 2.79-2.66 (m, 2H), 2.37-2.28 (m, 1H), 1.85-1.77 (m, 1H), 1.60 (s, 3H), 0.73 (d, J=6.3 Hz, 3H) ppm. ¹⁹F NMR (471 MHz, DMSO-d₆) δ −73.36 (s, 3F), −138.20 (s, 1F), −155.00 (s, 1F) ppm. ESI-MS m/z calc. 486.1578, found 487.6 (M+1)⁺; 485.5 (M−1)⁻; Retention time: 3.25 minutes.

### Example 3

### (2R,3S,4S,5R)-3-(3,4-difluoro-2-((S)-2-methoxypropoxy)phenyl)-N-(6-(hydroxymethyl)pyridin-3-yl)-4,5-dimethyl-5-(trifluoromethyl)tetrahydrofuran-2-carboxamide (5)

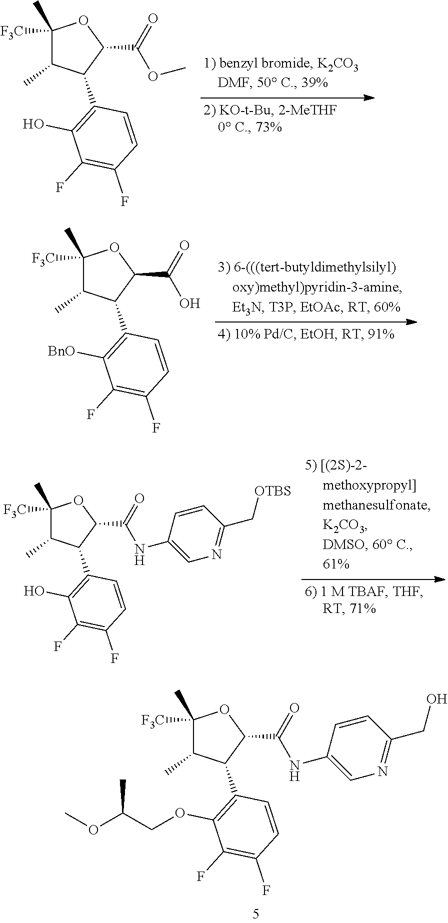

### Step 1:

Benzyl bromide (1.078 g, 0.75 mL, 6.30 mmol) was added to a stirred mixture of methyl (2S,3S,4S,5R)-3-(3,4-difluoro-2-hydroxy-phenyl)-4,5-dimethyl-5-(trifluoromethyl)tetrahydrofuran-2-carboxylate (1.5 g, 4.234 mmol) and K₂CO₃ (760 mg, 5.49 mmol) in DMF (7.5 mL). The reaction mixture was stirred at 50° C. for 3 h then at ambient temperature overnight. The reaction mixture was diluted with water (10 mL) and extracted with diethyl ether (3×10 mL). The combined organic extracts were washed with brine (5 mL), dried (MgSO₄), filtered and concentrated in vacuo. Purification by flash chromatography (SiO₂, 0 to 10% EtOAc in heptane) gave methyl (2S,3S,4S,5R)-3-(2-benzyloxy-3,4-difluoro-phenyl)-4,5-dimethyl-5-(trifluoromethyl)tetrahydrofuran-2-carboxylate (814 mg, 39%) as a yellow oil which solidified to a white solid. ¹H NMR (400 MHz, Chloroform-d) δ 7.51-7.31 (m, 5H), 7.24-7.12 (m, 1H), 6.81 (td, J=9.4, 7.6 Hz, 1H), 5.20 (d, J=11.0 Hz, 1H), 5.02 (d, J=11.4 Hz, 1H), 4.82 (d, J=6.4 Hz, 1H), 4.23 (dd, J=8.5, 6.2 Hz, 1H), 3.52 (s, 3H), 2.76-2.68 (m, 1H), 1.48 (d, J=0.9 Hz, 3H), 0.82 (dd, J=7.3, 1.8 Hz, 3H) ppm. ESI-MS m/z calc. 444.136, found 443.08 (M−1)⁻; Retention time: 1.13 minutes.

### Step 2:

KO-t-Bu (540 mg, 0.59 mL, 4.81 mmol) was added to a solution of methyl (2S,3S,4S,5R)-3-(2-benzyloxy-3,4-difluoro-phenyl)-4,5-dimethyl-5-(trifluoromethyl)tetrahydrofuran-2-carboxylate (0.8 g, 1.620 mmol) in 2-MeTHF (2.5 mL) at 0° C. under argon. The reaction mixture was stirred at 0° C. for 30 min and at ambient temperature for 15 min. The reaction mixture was diluted with diethyl ether (5 mL) and acidified with 1 M HCl (˜4 mL). The organic extracts were washed with brine (5 mL), dried (Na₂SO₄), filtered and concentrated in vacuo to give (2R,3S,4S,5R)-3-(2-benzyloxy-3,4-difluoro-phenyl)-4,5-dimethyl-5-(trifluoromethyl)tetrahydrofuran-2-carboxylic acid (690 mg, 73%) as a yellow oil. ¹H NMR (400 MHz, Chloroform-d) δ 7.46-7.34 (m, 5H), 6.95-6.84 (m, 2H), 5.25-5.22 (m, 1H), 5.09-5.06 (m, 1H), 4.85 (d, J=10.7 Hz, 1H), 3.71 (dt, J=15.0, 7.1 Hz, 1H), 2.50-2.42 (m, 1H), 1.37 (d, J=9.2 Hz, 3H), 0.68-0.66 (m, 3H) ppm; OH acid not observed. ¹⁹F-NMR (376 MHz, Chloroform-d) 6-74.7 (s, 3F), −136.6 (ddd, J=19.8, 9.0, 5.9 Hz, 1F), −152.6 (dd, J=19.9, 6.3 Hz, 1F) ppm. ESI-MS m/z calc. 430.1204, found 429.08 (M−1)⁻; Retention time: 0.58 minutes.

### Step 3:

A 50% T3P solution in EtOAc (1.603 g, 3 mL, 2.51 mmol) was added to a solution of (2R,3S,4S,5R)-3-(2-benzyloxy-3,4-difluoro-phenyl)-4,5-dimethyl-5-(trifluoromethyl)tetrahydrofuran-2-carboxylic acid (600 mg, 1.021 mmol), 6-[[tert-butyl(dimethyl)silyl]oxymethyl]pyridin-3-amine (270 mg, 1.132 mmol) and Et₃N (217.80 mg, 0.3 mL, 2.152 mmol) in EtOAc (8 mL). The reaction mixture was stirred at ambient temperature for 4 h. The reaction mixture was partitioned between EtOAc (50 mL) and water (10 ml). The organic layer was dried (Na₂SO₄), filtrated and concentrated in vacuo to give an oil. Purification by flash chromatography (SiO₂, 0 to 25% EtOAc in heptane) gave (2R,3S,4S,5R)-3-(2-benzyloxy-3,4-difluoro-phenyl)-N-[6-[[tert-butyl(dimethyl)silyl]oxymethyl]-3-pyridyl]-4,5-dimethyl-5-(trifluoromethyl)tetrahydrofuran-2-carboxamide (630 mg, 60%) as a yellow oil. ESI-MS m/z calc. 650.25995, found 651.34 (M+1)⁺; 649.3 (M−1)⁻; Retention time: 3.84 minutes.

### Step 4:

A mixture of (2R,3S,4S,5R)-3-(2-benzyloxy-3,4-difluoro-phenyl)-N-[6-[[tert-butyl(dimethyl)silyl]oxymethyl]-3-pyridyl]-4,5-dimethyl-5-(trifluoromethyl)tetrahydrofuran-2-carboxamide (800 mg, 1.207 mmol) and palladium on carbon (50 mg of 10% wt. loading, 0.469 mmol) in EtOH (20 mL) was stirred under an hydrogen atmosphere (1 atm) at ambient temperature for 5 hours. The reaction mixture was filtered through a 0.45 m cellulose membrane. The filtrate was concentrated in vacuo to give (2R,3S,4S,5R)—N-[6-[[tert-butyl(dimethyl)silyl]oxymethyl]-3-pyridyl]-3-(3,4-difluoro-2-hydroxy-phenyl)-4,5-dimethyl-5-(trifluoromethyl)tetrahydrofuran-2-carboxamide (630 mg, 91%) as a white solid. ¹H NMR (400 MHz, Chloroform-d) δ 8.53 (s, 1H), 8.38 (d, J=5.5 Hz, 1H), 7.67 (d, J=5.5 Hz, 1H), 7.42 (s, 1H), 7.07 (t, J=6.2 Hz, 1H), 6.76 (dd, J=17.2, 9.4 Hz, 1H), 6.38-6.61 (br s, 1H), 5.07 (d, J=10.5 Hz, 1H), 4.82 (s, 2H), 4.15 (dd, J=10.8, 8.0 Hz, 1H), 2.85 (t, J=7.8 Hz, 1H), 1.68 (s, 3H), 0.94 (dd, J=5.4, 1.9 Hz, 9H), 0.83-0.78 (m, 3H), 0.11 (t, J=2.5 Hz, 6H) ppm. ¹⁹F NMR (376 MHz, Chloroform-d) 6-74.5 (s, 3F), −137.9 (d, J=19.4 Hz, 1F), −162.5-−162.5 (m, 1F) ppm. ESI-MS m/z calc. 560.213, found 561.2 (M+1)⁺; 559.1 (M−1)⁻; Retention time: 2.13 minutes.

### Step 5:

[(2S)-2-methoxypropyl] methanesulfonate (40 mg, 0.214 mmol) was added to a suspension of (2R,3S,4S,5R)—N-[6-[[tert-butyl(dimethyl)silyl]oxymethyl]-3-pyridyl]-3-(3,4-difluoro-2-hydroxy-phenyl)-4,5-dimethyl-5-(trifluoromethyl)tetrahydrofuran-2-carboxamide (40 mg, 0.056 mmol) and K₂CO₃ (30 mg, 0.217 mmol) in DMSO (1 mL). The reaction mixture was stirred at 60° C. for 7 h. The reaction mixture was partitioned between EtOAc (20 mL) and water (6 mL). The organic layer was dried (MgSO₄), filtered and concentrated in vacuo to give an oil. Purification by reverse phase chromatography (SiO₂, 10 to 80% water containing 0.1% NH₄OH in MeCN) gave (2R,3S,4S,5R)—N-[6-[[tert-butyl(dimethyl)silyl]oxymethyl]-3-pyridyl]-3-[3,4-difluoro-2-[(2S)-2-methoxypropoxy]phenyl]-4,5-dimethyl-5-(trifluoromethyl)tetrahydrofuran-2-carboxamide (23 mg, 61%) as a colorless oil. ESI-MS m/z calc. 632.2705, found 633.76 (M+1)⁺; 631.66 (M−1)⁻; Retention time: 3.68 minutes.

### Step 6:

A 1M TBAF solution in THF (44.3 mg, 50 μL, 0.16 mmol) was added to a solution of (2R,3S,4S,5R)—N-(6-(((tert-butyldimethylsilyl)oxy)methyl)pyridin-3-yl)-3-(3,4-difluoro-2-((S)-2-methoxypropoxy)phenyl)-4,5-dimethyl-5-(trifluoromethyl)tetrahydrofuran-2-carboxamide (23 mg, 0.0345 mmol) in THF (2 mL) at 0° C. The reaction was stirred at ambient temperature for 2 h. The reaction was partitioned between EtOAc (15 mL) and water (4 mL). The organic layer was dried (MgSO₄), filtered and concentrated in vacuo to give an oil. Purification by reverse phase chromatography (SiO₂, 10 to 80% water containing 0.1% NH₄OH in MeCN) gave (2R,3S,4S,5R)-3-(3,4-difluoro-2-((S)-2-methoxypropoxy)phenyl)-N-(6-(hydroxymethyl)pyridin-3-yl)-4,5-dimethyl-5-(trifluoromethyl)tetrahydrofuran-2-carboxamide (5, 13 mg, 72%) as a white solid. ¹H NMR (400 MHz, Chlorofom-d) δ 8.64 (d, J=2.3 Hz, 1H), 8.57 (s, 1H), 8.16 (dd, J=8.5, 2.5 Hz, 1H), 7.28-7.25 (br s, 1H), 7.12-7.09 (m, 1H), 6.91 (dd, J=16.5, 9.2 Hz, 1H), 5.05 (d, J=11.4 Hz, 1H), 4.74 (s, 2H), 4.32 (dd, J=11.4, 7.3 Hz, 1H), 4.18-4.09 (m, 2H), 3.57 (td, J=6.0, 2.7 Hz, 1H), 3.22 (s, 3H), 2.82 (t, J=7.6 Hz, 1H), 2.02-2.45 (1H), 1.70 (s, 3H), 1.23 (d, J=6.4 Hz, 3H), 0.79-0.77 (m, 3H) ppm. 1 proton hidden under CHCl₃ signal. ¹⁹F NMR (376 MHz, Chlorofom-d) 6-74.4 (d, J=37.5 Hz, 3F), −137.1-−137.3 (m, 1F), −153.7 (dd, J=18.9, 6.0 Hz, 1F) ppm. ESI-MS m/z calc. 518.184, found 519.52 (M+1); 517.4 (M−1)⁻; Retention time: 2.51 minutes.

The following compounds were made using the method described in Example 3, except that different coupling partners were used in step 5:

| Cmpd No. | Compound Name | LC/MS | NMR (shifts in ppm) |
|---|---|---|---|
| 6 | (2R,3S,4S,5R)-3-(3,4-difluoro- | ESI-MS m/z calc. | ¹H NMR (400 MHz, |
| | 2-((R)-2- | 518.184, found 519.53 | Chloroform-d) δ 8.62 (d, |
| | methoxypropoxy)phenyl)-N-(6- | (M + 1)⁺; 517.4 (M − 1)⁻; | J = 2.3 Hz, 1H), 8.50 (s, |
| | (hydroxymethyl)pyridin-3-yl)- | Retention time: 2.49 | 1H), 8.15 (dd, J = 8.5, 2.5 |
| | 4,5-dimethyl-5- | minutes. | Hz, 1H), 7.26-7.24 (m, |
| | (trifluoromethyl)tetrahydrofuran- | | 1H), 7.13-7.09 (m, 1H), |
| | 2-carboxamide | | 6.91 (td, J = 9.2, 7.8 Hz, |
| | | | 1H), 5.01 (d, J = 11.4 Hz, |
| | | | 1H), 4.74 (s, 2H), 4.26-4.19 |
| | | | (m, 2H), 3.93 (ddd, J = |
| | | | 10.4, 6.5, 1.5 Hz, 1H), |
| | | | 3.68 (td, J = 6.4, 3.2 Hz, |
| | | | 1H), 3.33 (s, 3H), 2.86 (t, |
| | | | J = 7.6 Hz, 1H), 2.23-2.51 |
| | | | (br s, 1H), 1.69 (s, 3H), |
| | | | 1.17 (d, J = 6.4 Hz, |
| | | | 3H), 0.80-0.78 (m, 3H) |
| | | | ppm. ¹⁹F NMR (376 |
| | | | MHz, Chlorofom-d) δ −74.5 |
| | | | (s, 3F), −136.4 (qd, |
| | | | J = 9.6, 5.7 Hz, 1F), −153.6 |
| | | | (dd, J = 19.2, 6.0 Hz, 1F) |
| | | | ppm. |
| 7 | (2R,3S,4S,5R)-3-(2-(2- | ESI-MS m/z calc. | ¹H NMR (400 MHz, |
| | (dimethylamino)-2-oxoethoxy)- | 531.1793, found 532.17 | Chloroform-d) δ 8.96-9.36 |
| | 3,4-difluorophenyl)-N-(6- | (M + 1)⁺; 530.0 (M − 1)⁻; | (1H), 8.40 (d, J = 6.0 Hz, |
| | (hydroxymethyl)pyridin-3-yl)- | Retention time: 2.08 | 1H), 7.66 (s, 1H), |
| | 4,5-dimethyl-5- | minutes. | 7.55 (d, J = 5.5 Hz, 1H), |
| | (trifluoromethyl)tetrahydrofuran- | | 7.08-7.04 (m, 1H), 6.92 |
| | 2-carboxamide | | (q, J = 8.7 Hz, 1H), 5.08-5.03 |
| | | | (m, 2H), 4.75 (s, 2H), |
| | | | 4.71 (d, J = 14.7 Hz, |
| | | | 1H), 4.59-4.55 (m, 1H), |
| | | | 2.94 (s, 3H), 2.88 (s, 3H), |
| | | | 2.84 (t, J = 7.8 Hz, 1H), |
| | | | 1.73 (s, 3H), 0.85-0.83 |
| | | | (m, 3H) ppm; NH not |
| | | | observed ¹⁹F NMR (376 |
| | | | MHz, Chlorofom-d) δ −74.5 |
| | | | (s, 3F), −136.6 (s, |
| | | | 1F), −154.0 (d, J = 14.2 |
| | | | Hz, 1F) ppm. |
| 8 | (2R,3S,4S,5R)-3-(2-((2- | ESI-MS m/z calc. | ¹H NMR (301 MHz, |
| | oxaspiro[3.3]heptan-6-yl)oxy)- | 542.184, found 543.54 | Chloroform-d) δ 8.68 (s, |
| | 3,4-difluorophenyl)-N-(6- | (M + 1)⁺; 541.4 (M − 1)⁻; | 1H), 8.59 (s, 1H), 8.21 (d, |
| | (hydroxymethyl)pyridin-3-yl)- | Retention time: 2.36 | J = 8.6 Hz, 1H), 7.30 (s, |
| | 4,5-dimethyl-5- | minutes. | 1H), 7.11-7.07 (m, 1H), |
| | (trifluoromethyl)tetrahydrofuran- | | 6.93-6.84 (m, 1H), 5.02 |
| | 2-carboxamide | | (d, J = 11.0 Hz, 1H), 4.76 |
| | | | (s, 2H), 4.64 (td, J = 9.7, |
| | | | 6.3 Hz, 4H), 4.58-4.48 |
| | | | (m, 1H), 4.07 (dd, J = |
| | | | 10.7, 7.9 Hz, 1H), 2.83-2.67 |
| | | | (m, 3H), 2.65-2.38 |
| | | | (br s, 1H), 2.35 (ddd, J = |
| | | | 22.6, 12.0, 7.0 Hz, 2H), |
| | | | 1.66 (d, J = 14.8 Hz, 3H), |
| | | | 0.77 (dd, J = 7.4, 1.9 Hz, |
| | | | 3H) ppm. ¹⁹F NMR (283 |
| | | | MHz, Chlorofom-d) δ −74.4 |
| | | | (s, 3F), −136.7 (ddd, |
| | | | J = 19.9, 9.4, 5.5 Hz, |
| | | | 1F), −154.4 (dd, J = 19.5, |
| | | | 5.6 Hz, 1F) ppm. |
| 9 | (2R,3S,4S,5R)-3-(3,4-difluoro- | ESI-MS m/z calc. | ¹H NMR (400 MHz, |
| | 2-((tetrahydrofuran-3- | 530.184, found 531.2 | Chloroform-d) δ 8.56 (s, |
| | yl)methoxy)phenyl)-N-(6- | (M + 1)⁺; 529.1 (M − 1)⁻; | 1H), 8.45 (d, J = 6.0 Hz, |
| | (hydroxymethyl)pyridin-3-yl)- | Retention time: 2.41 | 1H), 7.57 (s, 1H), 7.42 (d, |
| | 4,5-dimethyl-5- | minutes. | J = 5.5 Hz, 1H), 7.07 (t, J = |
| | (trifluoromethyl)tetrahydrofuran- | | 2.7 Hz, 1H), 6.91 (d, J = |
| | 2-carboxamide | | 9.6 Hz, 1H), 5.00 (dd, J = |
| | | | 11.2, 2.5 Hz, 1H), 4.73 |
| | | | (s, 2H), 4.22 (t, J = 2.3 |
| | | | Hz, 1H), 4.07-3.96 (m, |
| | | | 2H), 3.89-3.79 (m, 2H), |
| | | | 3.72-3.66 (m, 2H), 2.72 |
| | | | (dd, J = 13.1, 7.6 Hz, |
| | | | 2H), 2.05 (d, J = 7.3 Hz, |
| | | | 1H), 1.67 (s, 4H), 0.80-0.78 |
| | | | (m, 3H) ppm: OH |
| | | | alcohol not observed. |

### Example 4

### (2R,3S,4S,5R)-3-(3,4-difluoro-2-(2-methoxyethoxy)phenyl)-N-(6-((R)-1,2-dihydroxyethyl)pyridin-3-yl)-4,5-dimethyl-5-(trifluoromethyl)tetrahydrofuran-2-carboxamide (10)

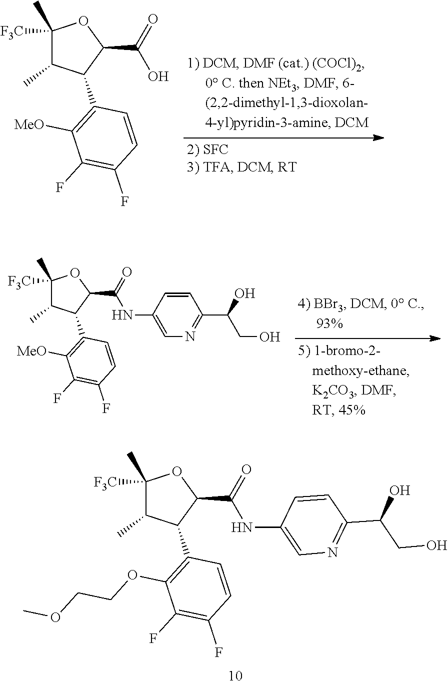 [SMILES: COc1c([C@H]2[C@H](C(=O)O)O[C@@](C)(C(F)(F)F)[C@H]2C)ccc(F)c1F ; COc1c([C@H]2[C@H](C(=O)Nc3ccc([C@@H](O)CO)nc3)O[C@@](C)(C(F)(F)F)[C@H]2C)ccc(F)c1F ; COCCOc1c([C@H]2[C@H](C(=O)Nc3ccc([C@@H](O)CO)nc3)O[C@@](C)(C(F)(F)F)[C@H]2C)ccc(F)c1F]

### Step 1 and 2:

Oxalyl chloride (738 μL, 8.460 mmol) was added dropwise to a solution of (2R,3S,4S,5R)-3-(3,4-difluoro-2-methoxy-phenyl)-4,5-dimethyl-5-(trifluoromethyl)tetrahydrofuran-2-carboxylic acid (1.5 g, 4.234 mmol) and DMF (31 μL, 0.4004 mmol) in dichloromethane (10 mL). After stirring for 30 min at ambient temperature, the solution was concentrated in vacuo. A mixture of rac-6-(2,2-Dimethyl-1,3-dioxolan-4-yl)pyridin-3-amine (904 mg, 4.654 mmol) and Et₃N (706 μL, 5.065 mmol) was added to the residue re-dissolved in dichloromethane (10 mL). The mixture was stirred at ambient temperature for 1 h. The reaction mixture was partitioned between EtOAc (30 mL) and water (30 mL). The aqueous layer was further extracted with EtOAc (50 mL). The combined organic extracts were washed with brine (1×20 mL), dried (MgSO₄), filtered and concentrated in vacuo. Purification by reverse phase preparative HPLC (Waters Sunfire C18, 10 μM, 100 Å column, 0% to 100% MeCN in water containing 0.10% ammonia) gave after freeze-drying a mixture of the 2 diastereoisomers of (2R,3S,4S,5R)-3-(3,4-difluoro-2-methoxyphenyl)-N-(6-(2,2-dimethyl-1,3-dioxolan-4-yl)pyridin-3-yl)-4,5-dimethyl-5-(trifluoromethyl)tetrahydrofuran-2-carboxamide.

The mixture of the 2 diastereoisomers of (2R,3S,4S,5R)-3-(3,4-difluoro-2-methoxyphenyl)-N-(6-(2,2-dimethyl-1,3-dioxolan-4-yl)pyridin-3-yl)-4,5-dimethyl-5-(trifluoromethyl)tetrahydrofuran-2-carboxamide were separated by chiral SFC using a Chiralcel OJ-H column, 5 m particle size, 25 cm×10 mm from Daicel on a Minigram SFC instrument from Berger Instruments:

First Eluting Isomer (rt=2.99 min): (2R,3S,4S,5R)-3-(3,4-difluoro-2-methoxyphenyl)-N-(6-((R)-2,2-dimethyl-1,3-dioxolan-4-yl)pyridin-3-yl)-4,5-dimethyl-5-(trifluoromethyl)tetrahydrofuran-2-carboxamide (700 mg, 60%). ESI-MS m/z calc. 530.184, found 531.2 (M+1)⁺; Retention time: 3.56 minutes.

Second Eluting Isomer (rt=3.63 min): (2R,3S,4S,5R)-3-(3,4-difluoro-2-methoxyphenyl)-N-(6-((S)-2,2-dimethyl-1,3-dioxolan-4-yl)pyridin-3-yl)-4,5-dimethyl-5-(trifluoromethyl)tetrahydrofuran-2-carboxamide (700 mg, 60%). ESI-MS m/z calc. 530.184, found 531.2 (M+1)⁺; Retention time: 3.56 minutes.

### Step 3:

TFA (1.743 mL, 22.62 mmol) was added to a solution of (2R,3S,4S,5R)-3-(3,4-difluoro-2-methoxyphenyl)-N-(6-((R)-2,2-dimethyl-1,3-dioxolan-4-yl)pyridin-3-yl)-4,5-dimethyl-5-(trifluoromethyl)tetrahydrofuran-2-carboxamide (600 mg, 1.112 mmol) (First Eluting Isomer from SFC separation) in DCM (20 mL) and the mixture stirred for 2 hours at ambient temperature. The mixture was concentrated in vacuo and freeze-dried from MeCN and water to give a white solid. Purification by reverse phase preparative HPLC (Waters Sunfire C18, 10 μM, 100 Å column, 0% to 100% MeCN in water containing 0.1% ammonia) gave, after freeze-drying, (2R,3S,4S,5R)-3-(3,4-difluoro-2-methoxyphenyl)-N-(6-((R)-1,2-dihydroxyethyl)pyridin-3-yl)-4,5-dimethyl-5-(trifluoromethyl)tetrahydrofuran-2-carboxamide (304 mg, 55%). ¹H NMR (500 MHz, DMSO-d₆) δ 10.48 (s, 1H), 8.73 (s, 1H), 8.10 (d, J=8.4 Hz, 1H), 7.52 (d, J=8.6 Hz, 1H), 7.23-7.13 (m, 2H), 5.11 (d, J=10.3 Hz, 1H), 4.61 (s, 1H), 4.25 (dd, J=10.3, 7.6 Hz, 1H), 3.95 (d, J=2.1 Hz, 3H), 3.63 (dd, J=11.0, 4.4 Hz, 2H), 3.48 (dd, J=11.0, 6.5 Hz, 2H), 2.77 (p, J=7.6 Hz, 1H), 1.61 (s, 3H), 0.79-0.69 (m, 3H) ppm. ESI-MS m/z calc. 490.1527, found 491.6 (M+1)⁺; Retention time: 2.98 minutes.

### Step 4:

Tribromoborane (500 μL of 1 M solution in DCM, 0.5 mmol) was added to a solution of (2R,3S,4S,5R)-3-(3,4-difluoro-2-methoxy-phenyl)-N-[6-((R)-1,2-dihydroxyethyl)-3-pyridyl]-4,5-dimethyl-5-(trifluoromethyl)tetrahydrofuran-2-carboxamide (80 mg, 0.1631 mmol) in DCM (3 mL) at 0° C. and stirred for 15 min. The reaction mixture was quenched with MeOH (2 mL) then concentrated in vacuo. The residue was dissolved in MeOH (3 mL) and the pH was adjusted to pH 9 with an aqueous 2 M sodium hydroxide solution. The mixture was stirred for 10 minutes. Purification by reverse phase HPLC-MS using an X-bridge C18 column (150×19 mm, 5 m particle size) from Waters gave (2R,3S,4S,5R)-3-(3,4-difluoro-2-hydroxy-phenyl)-N-[6-((R)-1,2-dihydroxyethyl)-3-pyridyl]-4,5-dimethyl-5-(trifluoromethyl)tetrahydrofuran-2-carboxamide (75.5 mg, 93%). ¹H NMR (500 MHz, DMSO-d₆) δ 12.78 (s, 1H), 8.48 (d, J=2.5 Hz, 1H), 7.92 (dd, J=8.5, 2.5 Hz, 1H), 7.36 (d, J=8.5 Hz, 1H), 6.54-6.43 (m, 1H), 5.83 (q, J=8.6 Hz, 1H), 5.72 (d, J=11.3 Hz, 1H), 5.27 (s, 1H), 4.61 (s, 1H), 4.50 (s, 1H), 3.69 (dd, J=11.3, 6.9 Hz, 1H), 3.59 (dd, J=10.5, 5.1 Hz, 1H), 3.47-3.37 (m, 1H), 2.56 (t, J=7.2 Hz, 1H), 1.52 (s, 3H), 0.81-0.76 (m, 4H) ppm. ESI-MS m/z calc. 476.13705, found 477.3 (M+1)⁺; 475.2 (M−1)⁻; Retention time: 2.46 minutes.

### Step 5:

1-Bromo-2-methoxy-ethane (2 μL, 0.021 mmol) was added to a solution of (2R,3S,4S,5R)-3-(3,4-difluoro-2-hydroxy-phenyl)-N-[6-((R)-1,2-dihydroxyethyl)-3-pyridyl]-4,5-dimethyl-5-(trifluoromethyl)tetrahydrofuran-2-carboxamide (10 mg, 0.02 mmol) and K₂CO₃ (3.2 mg, 0.023 mmol) in DMF (400 μL). The mixture was stirred at ambient temperature then at 60° C. Purification by reverse phase HPLC-MS using a a X-bridge C18 column (150×19 mm, 5 m particle size) from Waters gave (2R,3S,4S,5R)-3-(3,4-difluoro-2-(2-methoxyethoxy)phenyl)-N-(6-((R)-1,2-dihydroxyethyl)pyridin-3-yl)-4,5-dimethyl-5-(trifluoromethyl)tetrahydrofuran-2-carboxamide (10, 5.3 mg, 45%). ¹H NMR (500 MHz, DMSO-d₆) δ 10.34 (s, 1H), 8.65 (d, J=2.5 Hz, 1H), 8.00 (dd, J=8.5, 2.5 Hz, 1H), 7.42 (d, J=8.5 Hz, 1H), 7.17 (dd, J=8.6, 4.4 Hz, 2H), 5.33 (s, 1H), 5.11 (d, J=10.7 Hz, 1H), 4.63 (s, 1H), 4.54 (dd, J=6.8, 4.2 Hz, 1H), 4.34 (dd, J=10.8, 7.2 Hz, 1H), 4.30-4.22 (m, 1H), 4.21 (s, 1H), 3.70-3.55 (m, 3H), 3.44 (dd, J=10.9, 6.8 Hz, 1H), 3.29 (s, 3H), 2.84 (t, J=7.4 Hz, 1H), 1.61 (s, 3H), 0.71 (d, J=6.7 Hz, 3H) ppm. ESI-MS m/z calc. 534.17896, found 535.3 (M+1)⁺; 533.0 (M−1)⁻; Retention time: 3.02 minutes.

The following compounds were made using a method similar to that described in Example 4, except that different halides were used in step 5:

| Cmpd No. | Compound Name | LC/MS | NMR (shifts in ppm) |
|---|---|---|---|
| 11 | (2R,3S,4S,5R)-3-(3,4-difluoro- | ESI-MS m/z calc. | ¹H NMR (500 MHz, |
| | 2-(2- | 589.2211, found 590.3 | DMSO-d₆) δ 10.35 (s, |
| | morpholinoethoxy)phenyl)-N- | (M + 1)⁺; 588.3 (M − 1)⁻; | 1H), 8.66 (d, J = 2.4 Hz, |
| | (6-((R)-1,2- | Retention time: 2.91 | 1H), 8.00 (dd, J = 8.6, 2.6 |
| | dihydroxyethyl)pyridin-3-yl)- | minutes. | Hz, 1H), 7.42 (d, J = 8.5 |
| | 4,5-dimethyl-5- | | Hz, 1H), 7.21-7.08 (m, |
| | (trifluoromethyl)tetrahydrofuran- | | 2H), 5.33 (s, 1H), 5.12 (d, |
| | 2-carboxamide | | J = 10.6 Hz, 1H), 4.63 (s, |
| | | | 1H), 4.54 (s, 1H), 4.38-4.26 |
| | | | (m, 2H), 4.22 (ddt, J = |
| | | | 9.8, 6.1, 3.1 Hz, 1H), |
| | | | 3.63 (dt, J = 9.7, 4.4 Hz, |
| | | | 1H), 3.57-3.40 (m, 5H), |
| | | | 2.89 (q, J = 8.2, 7.5 Hz, |
| | | | 1H), 2.65 (t, J = 5.3 Hz, |
| | | | 2H), 2.40 (t, J = 4.7 Hz, |
| | | | 4H), 1.64 (s, 3H), 0.78-0.66 |
| | | | (m, 3H) ppm; 2 × |
| | | | OH alcohol not observed. |
| 12 | (2R,3S,4S,5R)-3-(2-(2- | ESI-MS m/z calc. | ¹H NMR (500 MHz, |
| | (difluoromethoxy)ethoxy)-3,4- | 570.1601, found 571.2 | DMSO-d₆) δ 10.35 (s, |
| | difluorophenyl)-N-(6-((R)-1,2- | (M + 1)⁺; 569.1 (M − 1)⁻; | 1H), 8.64 (dd, J = 2.5, 0.8 |
| | dihydroxyethyl)pyridin-3-yl)- | Retention time: 3.13 | Hz, 1H), 8.00 (dd, J = |
| | 4,5-dimethy1-5- | minutes. | 8.5, 2.5 Hz, 1H), 7.42 (d, |
| | (trifluoromethyl)tetrahydrofuran- | | J = 8.6 Hz, 1H), 7.18 (dd, |
| | 2-carboxamide | | J = 8.5, 4.4 Hz, 2H), 6.76 |
| | | | (t, J = 75.6 Hz, 1H), 5.32 |
| | | | (d, J = 4.9 Hz, 1H), 5.11 |
| | | | (d, J = 10.6 Hz, 1H), 4.63 |
| | | | (t, J = 5.9 Hz, 1H), 4.54 |
| | | | (dt, J = 6.8, 4.5 Hz, 1H), |
| | | | 4.38 (dd, J = 11.5, 4.4 Hz, |
| | | | 1H), 4.35-4.26 (m, 2H), |
| | | | 4.17 (td, J = 3.8, 3.3, 1.9 |
| | | | Hz, 2H), 3.63 (ddd, J = |
| | | | 10.5, 6.1, 4.2 Hz, 1H), |
| | | | 3.44 (ddd, J = 10.9, 6.8, |
| | | | 5.8 Hz, 1H), 2.82 (t, J = |
| | | | 7.4 Hz, 1H), 1.59 (s, 3H), |
| | | | 0.77-0.69 (m, 3H) ppm. |

### Example 5

### rel-(2S,3S,4S,5R)—N-(2-(1,2-dihydroxyethyl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (16) and rel-(2R,3R,4R,5S)—N-(2-(1,2-dihydroxyethyl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (15) and rel-(2S,3R,4R,5S)—N-(2-(1,2-dihydroxyethyl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (13) and rel-(2R,3S,4S,5R)—N-(2-(1,2-dihydroxyethyl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (14)

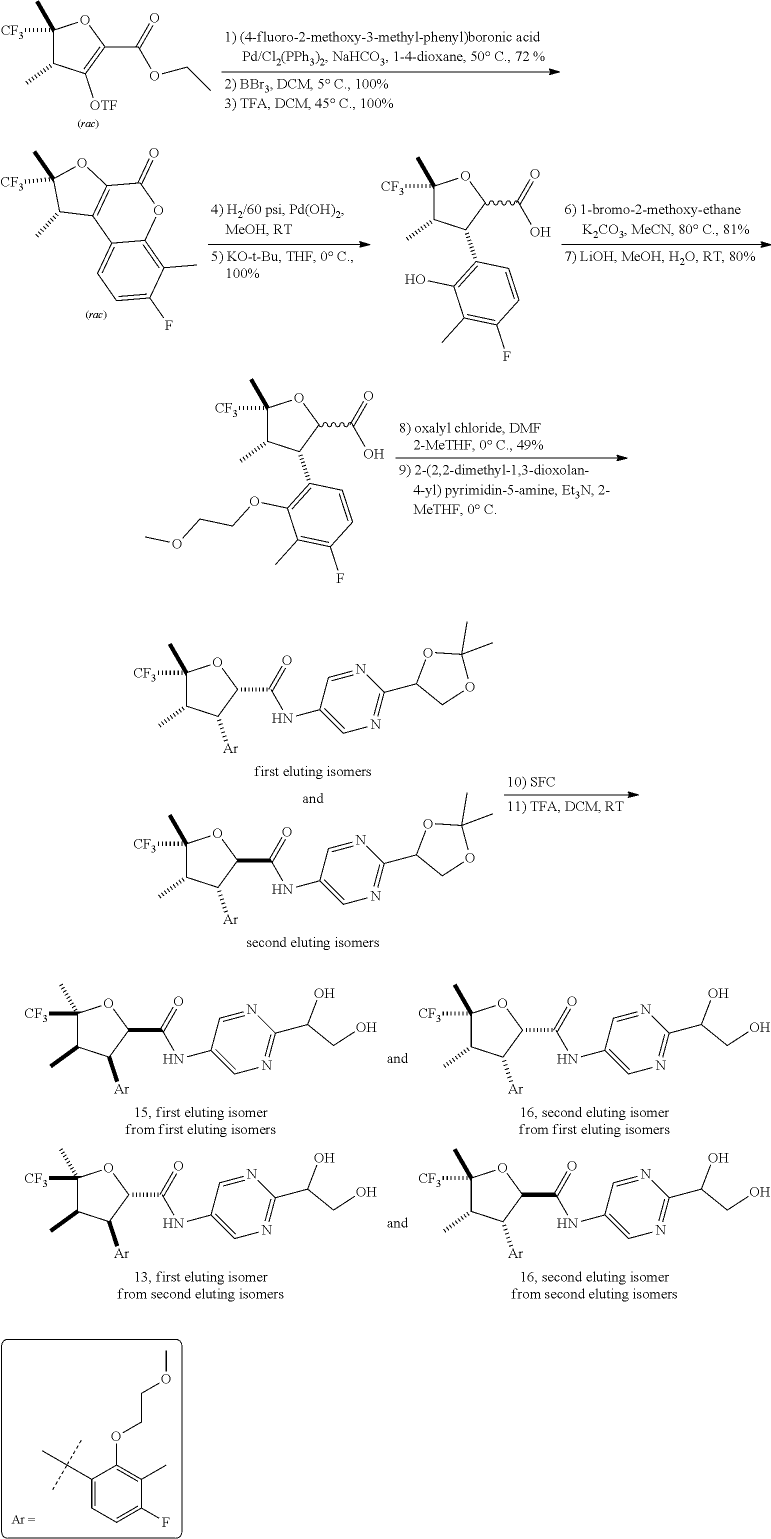

### Step 1:

To a 2-L three-necked round-bottomed flask flanked with a thermometer, was added a mixture of ethyl rac-(4R,5R)-4,5-dimethyl-5-(trifluoromethyl)-3-(((trifluoromethyl)sulfonyl)oxy)-4,5-dihydrofuran-2-carboxylate (39.05 g, 101.1 mmol), (4-fluoro-2-methoxy-3-methyl-phenyl)boronic acid (20.4 g, 110.9 mmol), PdCl₂(PPh₃)₂, (1.4 g, 1.995 mmol) and NaHCO₃ (120 mL) in 1,4-dioxane (400 mL). The orange mixture was heated at 50° C. for 20 min. The reaction mixture was cooled to ambient temperature and diluted with EtOAc (100 mL) and water (100 mL). The layers were separated and the aqueous phase was extracted with EtOAc (4×100 mL). The combined organic extracts were washed with brine (1×50 mL), dried (MgSO₄), filtered and concentrated in vacuo to 100 mL. Charcoal (10 g) was added and the mixture was stirred for 2 h. The mixture was filtered, washing through with EtOAc. The filtrate was concentrated in vacuo to give 50 g of crude product. Purification by flash chromatography (330 g SiO₂, 0 to 35% EtOAc in heptane) gave ethyl rac-(4S,5R)-3-(4-fluoro-2-methoxy-3-methylphenyl)-4,5-dimethyl-5-(trifluoromethyl)-4,5-dihydrofuran-2-carboxylate (27.3 g, 72%) as a pale yellow oil. ¹H NMR (500 MHz, Chloroform-d) δ 6.98-6.88 (m, 1H), 6.81 (t, J=8.7 Hz, 1H), 4.20-4.07 (m, 2H), 3.66 (s, 3H), 3.58-3.49 (m, 1H), 2.21 (d, J=2.1 Hz, 3H), 1.7 (s, 3H), 1.12 (t, J=7.1 Hz, 3H), 1.06 (dq, J=7.2, 2.3 Hz, 3H) ppm. ESI-MS m/z calc. 376.12976, found 377.5 (M+1)⁺; Retention time: 1.09 minutes.

### Step 2:

To a 1-L 3-necked flask flanked with a thermometer, was added ethyl rac-(4S,5R)-3-(4-fluoro-2-methoxy-3-methylphenyl)-4,5-dimethyl-5-(trifluoromethyl)-4,5-dihydrofuran-2-carboxylate (27.35 g, 72.67 mmol) followed by DCM (200 mL). This mixture was cooled to 5° C. in an ice bath. Boron tribromide (112 mL of 1 M solution in DCM, 112.0 mmol) was added over 30 mins at 5° C. and the reaction mixture was stirred for 1 h. Upon completion, the mixture was quenched slowly with water (caution effervescence) (100 mL). A NaHCO₃ solution (100 mL) was added and the mixture was stirred for 30 mins. The aqeuous phase was extracted with DCM (3×50 ml) and the organic layer was washed with NaHCO₃ (5×100 ml). The combined organic layers were dried with MgSO₄, filtered and concentrated in vacuo. To a solution of this solid in EtOAc (100 mL) was added charcoal (15 g) and the mixture was stirred at ambient temperature overnight. The reaction mixture was filtered through celite and the filtrate was concentrated in vacuo to give ethyl rac-(4S,5R)-3-(4-fluoro-2-hydroxy-3-methylphenyl)-4,5-dimethyl-5-(trifluoromethyl)-4,5-dihydrofuran-2-carboxylate (27.7 g, 100%) as a yellow waxy solid. ESI-MS m/z calc. 362.11414, found 363.5 (M+1)⁺; 361.5 (M−1)⁻; Retention time: 0.99 minutes.

### Step 3:

TFA (9.8 mL, 127.2 mmol) was added to a solution of ethyl rac-(4S,5R)-3-(4-fluoro-2-hydroxy-3-methylphenyl)-4,5-dimethyl-5-(trifluoromethyl)-4,5-dihydrofuran-2-carboxylate (27.7 g, 76.45 mmol) in DCM (200 mL) at ambient temperature under stirring. The reaction mixture was heated at reflux and stirred at this temperature for 2.5 hours. The reaction mixture was cooled to ambient temperature and quenched with a saturated aqueous NaHCO₃ solution (100 mL) and the layers were separated. The DCM layer was washed with a saturated aqueous NaHCO₃ solution (4×100 mL). The organic extracts were dried (Na₂SO₄) and concentrated in vacuo to give a waxy solid. This solid was re-dissolved in ethyl acetate (200 mL). Activated charcoal (10 g) was added and the mixture was stirred at ambient temperature overnight. The mixture was filtered through a celite cartridge, washing with ethyl acetate (3×100 ml). The filtrate was concentrated in vacuo to give rac-(1S,2R)-7-fluoro-1,2,6-trimethyl-2-(trifluoromethyl)-1,2-dihydro-4H-furo[2,3-c]chromen-4-one (24.18 g, 100%) as a waxy solid. ESI-MS m z calc. 316.07227, found 317.4 (M+1)⁺; 315.4 (M−1)⁻; Retention time: 0.94 minutes

### Step 4:

A solution of rac-(1S,2R)-7-fluoro-1,2,6-trimethyl-2-(trifluoromethyl)-1,2-dihydro-4H-furo[2,3-c]chromen-4-one (24.77 g, 78.32 mmol) in MeOH (700 mL) was added to 1-L parr hydrogenator flask containing palladium hydroxide on carbon (22 g of 20% wt. loading, 31.33 mmol). The reaction mixture was evacuated and back filled with nitrogen (×3) then evacuated and back filled with hydrogen (×3) and stirred under a hydrogen atmosphere at 60 psi for 116 h. The reaction mixture was filtered through celite, washing with MeOH (1 L) and EtOAc (500 ml). The filtrate was concentrated in vacuo to give methyl rac-(2S,3S,4S,5R)-3-(4-fluoro-2-hydroxy-3-methylphenyl)-4,5-dimethyl-5-(trifluoromethyl)tetrahydrofuran-2-carboxylate (8.896 g, 32%) as an off-white solid. ¹H NMR (500 MHz, Chloroform-d) δ 7.20 (t, J=7.7 Hz, 1H), 6.57 (t, J=8.9 Hz, 1H), 4.88 (d, J=6.4 Hz, 2H), 4.28 (dd, J=8.4, 6.1 Hz, 1H), 3.56 (s, 3H), 2.81 (p, J=7.7 Hz, 1H), 2.14 (d, J=1.7 Hz, 3H), 1.52 (d, J=1.2 Hz, 3H), 0.92 (dq, J=7.4, 1.9 Hz, 3H). ESI-MS m/z calc. 350.11414, found 349.4 (M−1)⁻; Retention time: 0.96 minutes.

### Step 5:

KO-t-Bu (11.40 g, 101.6 mmol) was added to a stirred solution of rac-(2S,3S,4S,5R)-methyl-3-(4-fluoro-2-hydroxy-3-methylphenyl)-4,5-dimethyl-5-(trifluoromethyl)tetrahydrofuran-2-carboxylate (8.896 g, 25.39 mmol) in THF (125 mL) at 0° C. and the reaction mixture was stirred for 15 minutes. 1M HCl (350 mL), brine (100 mL) and DCM (100 mL) were added to the reaction mixture and the layers were separated. The aqueous layer was extracted with DCM (3×100 mL) and the combined organic extracts were dried (MgSO₄), filtered and concentrated in vacuo. The residue was re-dissolved in DCM (71.17 mL) and treated with TFA (26.62 g, 17.99 mL, 233.5 mmol). The reaction was stirred at ambient temperature for 2 h. The solvent was removed in vacuo and the residue azeotroped with DCM (2×50 mL). The residue was partitioned between DCM (100 mL) and water (50 mL) and the layers were separated. The organic layer was washed with water (3×50 mL) and the organic extracts dried (MgSO₄), filtered and concentrated in vacuo to give a mixture of four stereoisomers of 3-(4-fluoro-2-hydroxy-3-methylphenyl)-4,5-dimethyl-5-(trifluoromethyl)tetrahydrofuran-2-carboxylic acid (9.753 g, 100%) as a brown oil ((2S,3S,4S,5R), (2R,3R,4R,5S), (2R,3S,4S,5R), and (2S,3R,4R,5S)). ESI-MS m/z calc. 336.09848, found 335.5 (M−1)⁻; Retention time: 0.56 minutes.

### Step 6:

To a solution of the mixture of stereoisomers of 3-(4-fluoro-2-hydroxy-3-methylphenyl)-4,5-dimethyl-5-(trifluoromethyl)tetrahydrofuran-2-carboxylic acid obtained from Step 5 (1 g, 2.974 mmol) in MeCN (10 mL) was added K₂CO₃ (1.65 g, 11.94 mmol) and 1-bromo-2-methoxy-ethane (1.2 mL, 12.77 mmol) and the mixture was heated at 80° C. overnight. The reaction mixture was cooled down to ambient temperature, diluted with DCM and filtered. The filtrate was concentrated in vacuo to give a mixture of four stereoisomers of 2-methoxyethyl-3-(4-fluoro-2-(2-methoxyethoxy)-3-methylphenyl)-4,5-dimethyl-5-(trifluoromethyl)tetrahydrofuran-2-carboxylate (1.095 g, 81%) as a yellow oil ((2S,3S,4S,5R), (2R,3R,4R,5S), (2R,3S,4S,5R), and (2S,3R,4R,5S)). ESI-MS m/z calc. 452.1822, found 453.6 (M+1); Retention time: 1.04 minutes.

### Step 7:

LiOH (3 mL of 2 M aqueous solution, 6.0 mmol) was added to a solution of the mixture of stereoisomers of 2-methoxyethyl-3-(4-fluoro-2-(2-methoxyethoxy)-3-methylphenyl)-4,5-dimethyl-5-(trifluoromethyl)tetrahydrofuran-2-carboxylate obtained from Step 6 (1.095 g, 2.420 mmol) in MeOH (15 mL)/water (3 mL) and the mixture was stirred at ambient temperature for 15 mins. The reaction was concentrated in vacuo and quenched with 1M HCl. The layers were separated and the aqueous layer was extracted with DCM (2×5 mL). The combined organic extracts were dried by passing through a phase seperation cartridge, filtered and concentrated in vacuo to give a mixture of four stereoisomers of 3-(4-fluoro-2-(2-methoxyethoxy)-3-methylphenyl)-4,5-dimethyl-5-(trifluoromethyl)tetrahydrofuran-2-carboxylic acid (714.6 mg, 60%) ((2S,3S,4S,5R), (2R,3R,4R,5S), (2R,3S,4S,5R), and (2S,3R,4R,5S)). ESI-MS m/z calc. 394.14035, found 395.5 (M+1)⁺; Retention time: 0.47 minutes.

### Step 8:

To an ice-cold solution of the mixture of stereoisomers of 3-(4-fluoro-2-(2-methoxyethoxy)-3-methylphenyl)-4,5-dimethyl-5-(trifluoromethyl)tetrahydrofuran-2-carboxylic acid obtained from Step 7 (400 mg, 0.91 mmol) in 2-MeTHF (8 mL) was added DMF (30 μL of a 0.86 M solution in 2-MeTHF, 0.025 mmol) followed and by oxalyl chloride (170 μL, 1.949 mmol). The mixture was stirred and allowed to warm to ambient temperature over 1 h. The reaction mixture was concentrated in vacuo to give a mixture of four stereoisomers of 3-(4-fluoro-2-(2-methoxyethoxy)-3-methylphenyl)-4,5-dimethyl-5-(trifluoromethyl)tetrahydrofuran-2-carbonyl chloride (410 mg, 49%) ((2S,3S,4S,5R), (2R,3R,4R,5S), (2R,3S,4S,5R), and (2S,3R,4R,5S)). ESI-MS m/z calc. 412.10645, found 409.0 (M+1)⁺; Retention time: 1.07 minutes.

### Step 9:

A solution of the mixture of stereoisomers of 3-(4-fluoro-2-(2-methoxyethoxy)-3-methylphenyl)-4,5-dimethyl-5-(trifluoromethyl)tetrahydrofuran-2-carbonyl chloride obtained from Step 8 (300 mg, 0.3270 mmol) in 2-MeTHF (8 mL) was added to an ice-cold solution of rel-2-(2,2-dimethyl-1,3-dioxolan-4-yl)pyrimidin-5-amine (105 mg, 0.5379 mmol) (Second Eluting Isomer, Intermediate E) and Et₃N (285 μL, 2.045 mmol) in 2-MeTHF (8 mL) and NMP (1 mL). The reaction mixture was allowed to warm to ambient temperature and stirred overnight. The reaction mixture was quenched with water (2 mL) and the layers were separated. The aqueous layer was extracted with EtOAc (2×5 mL). The combined organics extracts were washed with brine (2×5 mL), dried (MgSO₄), filtered and concentrated in vacuo. Purification by flash chromatography (SiO₂, 0 to 25% EtOAc in heptane) gave two isomer mixtures:

### First Eluting Isomers:

(2S,3S,4S,5R)—N-(2-(2,2-dimethyl-1,3-dioxolan-4-yl)pyrimidin-5-yl)-3-(4-fluoro-2-(2-methoxyethoxy)-3-methylphenyl)-4,5-dimethyl-5-(trifluoromethyl)tetrahydrofuran-2-carboxamide and (2R,3R,4R,5S)—N-(2-(2,2-dimethyl-1,3-dioxolan-4-yl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (115.6 mg, 62%). ¹H NMR (500 MHz, Chloroform-d) δ 9.00 (s, 2H), 8.45 (s, 1H), 7.20 (dd, J=8.6, 6.3 Hz, 1H), 6.90 (t, J=8.7 Hz, 1H), 5.28 (t, J=6.7 Hz, 1H), 5.02 (d, J=11.5 Hz, 1H), 4.45 (dd, J=8.3, 6.7 Hz, 1H), 4.38 (dd, J=11.5, 7.5 Hz, 1H), 4.21 (ddd, J=8.3, 6.7, 1.6 Hz, 1H), 4.05 (ddd, J=10.8, 5.1, 2.1 Hz, 1H), 3.85-3.69 (m, 2H), 3.59 (ddd, J=11.0, 5.1, 2.1 Hz, 1H), 3.36 (s, 3H), 2.81 (p, J=7.6 Hz, 1H), 2.23 (d, J=2.0 Hz, 3H), 1.72 (s, 3H), 1.55 (s, 3H), 1.51 (s, 3H), 0.80 (dq, J=7.6, 2.3 Hz, 3H) ppm. ESI-MS m/z calc. 571.2305, found 572.4 (M+1)⁺; 570.3 (M−1)⁻; Retention time: 1.04 minutes.

### Second Eluting Isomers:

(2R,3S,4S,5R)—N-(2-(2,2-dimethyl-1,3-dioxolan-4-yl)pyrimidin-5-yl)-3-(4-fluoro-2-(2-methoxyethoxy)-3-methylphenyl)-4,5-dimethyl-5-(trifluoromethyl)tetrahydrofuran-2-carboxamide and (2S,3R,4R,5S)—N-(2-(2,2-dimethyl-1,3-dioxolan-4-yl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (30 mg, 15%). ¹H NMR (500 MHz, DMSO-d₆) δ 8.81 (d, J=1.0 Hz, 2H), 8.24 (d, J=2.0 Hz, 1H), 7.09 (t, J=7.6 Hz, 1H), 6.64 (t, J=8.7 Hz, 1H), 5.24 (t, J=6.7 Hz, 1H), 4.93 (d, J=6.8 Hz, 1H), 4.61-4.51 (m, 1H), 4.42 (dd, J=8.3, 6.7 Hz, 1H), 4.19-4.09 (m, 1H), 4.00 (t, J=4.4 Hz, 2H), 3.79-3.61 (m, 2H), 3.48 (s, 3H), 2.88 (p, J=7.8 Hz, 1H), 2.27-2.16 (m, 3H), 1.59 (s, 3H), 1.53 (d, J=2.2 Hz, 3H), 1.49 (s, 3H), 0.89-0.79 (m, 3H) ppm. ESI-MS m/z calc. 571.2305, found 572.2 (M+1)⁺; 570.3 (M−1)⁻; Retention time: 1.0 minutes.

### Step 10:

The second eluting isomers from Step 9 were separated by by chiral SFC using a (R,R)-Whelk-O1 column, 5 m particle size, 25 cm×21.2 mm from Regis Technologies on a Minigram SFC instrument from Berger Instruments to give two single isomers of unknown absolute configuration:

First Eluting Isomer (rt=1.88 min): rel-(2S,3R,4R,5S)—N-(2-(2,2-dimethyl-1,3-dioxolan-4-yl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (58 mg, 97%). ESI-MS m/z calc. 571.2305, found 572.0 (M+1)⁺; 570.1 (M−1)⁻; Retention time: 3.58 minutes.

Second Eluting Isomer (rt=3.05 min): rel-(2R,3S,4S,5R)—N-(2-(2,2-dimethyl-1,3-dioxolan-4-yl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (53.5 mg, 88%). ESI-MS m/z calc. 571.2305, found 572.0 (M+1); 570.1 (M−1)⁻; Retention time: 3.57 minutes.

### Step 11:

TFA (390 μL, 5.062 mmol) was added to a stirred solution of rel-(2S,3R,4R,5S)—N-(2-(2,2-dimethyl-1,3-dioxolan-4-yl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (58 mg, 0.1015 mmol, first eluting isomer from Step 10) in DCM (7 mL) and the reaction mixture stirred at ambient temperature for 18 h. The solvent was removed in vacuo and the residue azeotroped with DCM (×2). Purification by reverse phase HPLC-MS using a a X-bridge C18 column (150×19 mm, 5 m particle size) from Waters gave rel-(2S,3R,4R,5S)—N-(2-(1,2-dihydroxyethyl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (13, 31 mg, 57%). ¹H NMR (500 MHz, DMSO-d₆) δ 10.44 (s, 1H), 8.98 (s, 2H), 7.24 (dd, J=8.8, 6.5 Hz, 1H), 6.97 (t, J=8.8 Hz, 1H), 5.16 (d, J=6.0 Hz, 1H), 5.11 (d, J=10.9 Hz, 1H), 4.66-4.50 (m, 2H), 4.37 (dd, J=10.9, 7.3 Hz, 1H), 3.99 (ddd, J=10.8, 5.6, 2.4 Hz, 1H), 3.85 (ddd, J=10.8, 6.6, 2.4 Hz, 1H), 3.77-3.51 (m, 4H), 3.31 (s, 3H), 2.76 (p, J=7.4 Hz, 1H), 2.16 (d, J=1.9 Hz, 3H), 1.64 (s, 3H), 0.75-0.60 (m, 3H) ppm. ESI-MS m/z calc. 531.1992, found 532.3 (M+1)⁺; 530.2 (M−1)⁻; Retention time: 3.03 minutes as a white solid.

rel-(2R,3S,4S,5R)—N-(2-(2,2-dimethyl-1,3-dioxolan-4-yl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (53 mg, 0.09273 mmol, second eluting isomer from Step 10) was treated in the same way to give after reverse phase HPLC-MS chromatography, rel-(2R,3S,4S,5R)—N-(2-(1,2-dihydroxyethyl))pyrimidin-5-yl)-3-(4-fluoro-2-(2-methoxyethoxy)-3-methylphenyl)-4,5-dimethyl-5-(trifluoromethyl)tetrahydrofuran-2-carboxamide (14, 29 mg, 58%). ¹H NMR (500 MHz, DMSO-d₆) δ 10.32 (s, 1H), 8.98 (s, 2H), 7.25 (dd, J=8.7, 6.5 Hz, 1H), 6.97 (t, J=8.8 Hz, 1H), 5.16 (s, 1H), 5.11 (d, J=10.9 Hz, 1H), 4.59 (q, J=7.7, 6.7 Hz, 2H), 4.37 (dd, J=10.9, 7.3 Hz, 1H), 3.99 (ddd, J=10.8, 5.6, 2.4 Hz, 1H), 3.87-3.79 (m, 1H), 3.77-3.55 (m, 4H), 3.32 (s, 3H), 2.76 (p, J=7.4 Hz, 1H), 2.16 (d, J=1.9 Hz, 3H), 1.64 (s, 3H), 0.70 (dd, J=7.3, 2.5 Hz, 3H) ppm. ESI-MS m/z calc. 531.1992, found 532.3 (M+1)⁺; 530.2 (M−1)⁻; Retention time: 3.02 minutes as a white solid.

The following compounds were made using the method described in Example 5, except that the starting material of step 10 was the first eluting isomers from Step 9:

| Cmpd No. | Compound Name | LC/MS | NMR (shifts in ppm) |
|---|---|---|---|
| 15 | rel-(2R,3R,4R,5S)-N-(2-(1,2- | ESI-MS m/z calc. | ¹H NMR (500 MHz, |
| | dihydroxyethyl)pyrimidin-5- | 531.1992, found 532.3 | DMSO-d₆) δ 8.82 (s, 2H), |
| | yl)-3-(4-fluoro-2-(2- | (M + 1)⁺; 530.2 (M − 1)⁻; | 8.27 (s, 1H), 7.14-7.04 |
| | methoxyethoxy)-3- | Retention time: 3.02 | (m, 1H), 6.66 (t, J = 8.7 |
| | methylphenyl)-4,5-dimethyl-5- | minutes. | Hz, 1H), 4.94 (d, J = 6.8 |
| | (trifluoromethyl)tetrahydrofuran- | | Hz, 1H), 4.85 (t, J = 4.6 |
| | 2-carboxamide | | Hz, 1H), 4.59 (dd, J = |
| | (Precursor was the first eluting | | 8.8, 6.9 Hz, 1H), 4.05-3.90 |
| | peak by SFC on Chiralpak IC | | (m, 4H), 3.80-3.68 |
| | column, rt = 1.15 min) | | (m, 2H), 3.49 (s, 3H), |
| | | | 2.89 (p, J = 7.8 Hz, 1H), |
| | | | 2.20 (d, J = 2.1 Hz, 3H), |
| | | | 1.60 (s, 3H), 0.90-0.82 |
| | | | (m, 3H) ppm; alcohols |
| | | | OH not observed. |
| 16 | rel-(2S,3S,4S,5R)-N-(2-(1,2- | ESI-MS m/z calc. | ¹H NMR (500 MHz, |
| | dihydroxyethyl)pyrimidin-5- | 531.1992, found 532.3 | Chloroform-d) δ 8.84 (s, |
| | yl)-3-(4-fluoro-2-(2- | (M + 1)⁺; 530.2 (M − 1)⁻; | 2H), 8.29 (s, 1H), 7.17-7.06 |
| | methoxyethoxy)-3- | Retention time: 3.02 | (m, 1H), 6.68 (t, J = |
| | methylphenyl)-4,5-dimethyl-5- | minutes. | 8.7 Hz, 1H), 4.97 (d, J = |
| | (trifluoromethyl)tetrahydrofuran- | | 6.9 Hz, 1H), 4.87 (d, J = |
| | 2-carboxamide | | 4.3 Hz, 1H), 4.61 (dd, J = |
| | (Precursor was the second | | 8.8, 6.9 Hz, 1H), 4.20 (d, |
| | eluting peak by SFC on | | J = 4.6 Hz, 1H), 4.08-3.89 |
| | Chiralpak IC column, rt = 1.44 | | (m, 4H), 3.84-3.68 |
| | min) | | (m, 2H), 3.51 (s, 3H), |
| | | | 2.90 (dt, J = 17.6, 8.8 Hz, |
| | | | 1H), 2.58 (s, 1H), 2.23 (d, |
| | | | J = 2.1 Hz, 3H), 1.62 (s, |
| | | | 3H), 0.94-0.82 (m, 3H) |
| | | | ppm. |

### Example 6

### rel-(2S,3S,4S,5R)—N-(2-(1,2-dihydroxyethyl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (19) and rel-(2R,3R,4R,5S)—N-(2-(1,2-dihydroxyethyl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (20) and rel-(2R,3S,4S,5R)—N-(2-(1,2-dihydroxyethyl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (17) and rel-(2S,3R,4R,5S)—N-(2-(1,2-dihydroxyethyl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (18)

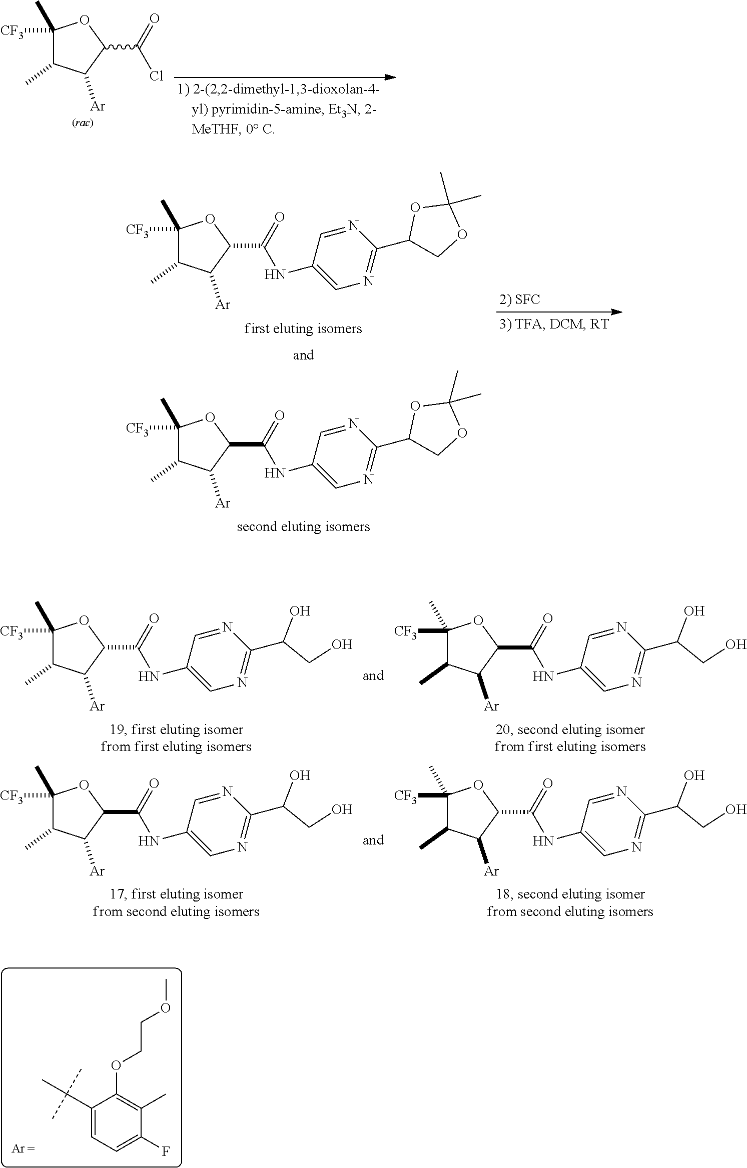

### Step 1:

A solution of the mixture of isomers of 3-(4-fluoro-2-(2-methoxyethoxy)-3-methylphenyl)-4,5-dimethyl-5-(trifluoromethyl)tetrahydrofuran-2-carbonyl chloride obtained from Example 5, Step 8 (300 mg, 0.3270 mmol) in 2-MeTHF (8 mL) was added to an ice-cold solution of rel-2-(2,2-dimethyl-1,3-dioxolan-4-yl)pyrimidin-5-amine (105 mg, 0.5379 mmol) (First Eluting Isomer, Intermediate E) and Et₃N (285 μL, 2.045 mmol) in 2-MeTHF (8 mL) and NMP (1 mL). The reaction mixture was warmed to ambient temperature and stirred overnight. The reaction mixture was quenched with water (2 mL) and the layers were separated. The aqueous layer was extracted with EtOAc (2×5 mL) and the combined organics extracts were washed with brine (2×5 mL), dried (MgSO₄), filtered and concentrated in vacuo. Purification by flash chromatography (SiO₂, 0 to 25% EtOAc in heptane) gave two isomer mixtures:

### First Eluting Isomers:

(2S,3S,4S,5R)—N-(2-(2,2-dimethyl-1,3-dioxolan-4-yl)pyrimidin-5-yl)-3-(4-fluoro-2-(2-methoxyethoxy)-3-methylphenyl)-4,5-dimethyl-5-(trifluoromethyl)tetrahydrofuran-2-carboxamide and (2R,3R,4R,5S)—N-(2-(2,2-dimethyl-1,3-dioxolan-4-yl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (115.6 mg, 62%). ¹H NMR (500 MHz, Chloroform-d) δ 9.00 (s, 2H), 8.45 (s, 1H), 7.20 (dd, J=8.6, 6.3 Hz, 1H), 6.90 (t, J=8.7 Hz, 1H), 5.28 (t, J=6.7 Hz, 1H), 5.02 (d, J=11.5 Hz, 1H), 4.45 (dd, J=8.3, 6.7 Hz, 1H), 4.38 (dd, J=11.5, 7.5 Hz, 1H), 4.21 (ddd, J=8.3, 6.7, 1.6 Hz, 1H), 4.05 (ddd, J=10.8, 5.1, 2.1 Hz, 1H), 3.85-3.69 (m, 2H), 3.59 (ddd, J=11.0, 5.1, 2.1 Hz, 1H), 3.36 (s, 3H), 2.81 (p, J=7.6 Hz, 1H), 2.23 (d, J=2.0 Hz, 3H), 1.72 (s, 3H), 1.55 (s, 3H), 1.51 (s, 3H), 0.80 (dq, J=7.6, 2.3 Hz, 3H) ppm. ESI-MS m/z calc. 571.2305, found 572.4 (M+1)⁺; 570.3 (M−1)⁻; Retention time: 1.04 minutes.

### Second Eluting Isomers:

(2R,3S,4S,5R)—N-(2-(2,2-dimethyl-1,3-dioxolan-4-yl)pyrimidin-5-yl)-3-(4-fluoro-2-(2-methoxyethoxy)-3-methylphenyl)-4,5-dimethyl-5-(trifluoromethyl)tetrahydrofuran-2-carboxamide and (2S,3R,4R,5S)—N-(2-(2,2-dimethyl-1,3-dioxolan-4-yl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (30 mg, 15%). ¹H NMR (500 MHz, DMSO-d₆) δ 8.81 (d, J=1.0 Hz, 2H), 8.24 (d, J=2.0 Hz, 1H), 7.09 (t, J=7.6 Hz, 1H), 6.64 (t, J=8.7 Hz, 1H), 5.24 (t, J=6.7 Hz, 1H), 4.93 (d, J=6.8 Hz, 1H), 4.61-4.51 (m, 1H), 4.42 (dd, J=8.3, 6.7 Hz, 1H), 4.19-4.09 (m, 1H), 4.00 (t, J=4.4 Hz, 2H), 3.79-3.61 (m, 2H), 3.48 (s, 3H), 2.88 (p, J=7.8 Hz, 1H), 2.27-2.16 (m, 3H), 1.59 (s, 3H), 1.53 (d, J=2.2 Hz, 3H), 1.49 (s, 3H), 0.89-0.79 (m, 3H) ppm. ESI-MS m/z calc. 571.2305, found 572.2 (M+1); 570.3 (M−1)⁻; Retention time: 1.0 minutes.

### Step 2:

The second eluting isomers from Step 1 were separated by by chiral SFC using a (R,R)-Whelk-O1 column, 5 m particle size, 25 cm×21.2 mm from Regis Technologies on a Minigram SFC instrument from Berger Instruments to give two single isomers of unknown absolute configuration:

First Eluting Isomer (rt=1.20 min): rel-(2R,3S,4S,5R)—N-(2-(2,2-dimethyl-1,3-dioxolan-4-yl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (15.1 mg, 56%). ESI-MS m/z calc. 571.2305, found 572.2 (M+1)⁺; 570.3 (M−1)⁻; Retention time: 3.37 minutes.

Second Eluting Isomer (rt=1.48 min): rel-(2S,3R,4R,5S)—N-(2-(2,2-dimethyl-1,3-dioxolan-4-yl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (15.3 mg, 56%). ESI-MS m/z calc. 571.2305, found 572.2 (M+1)⁺; 570.3 (M−1)⁻; Retention time: 3.37 minutes.

### Step 3:

TFA (390 μL, 5.062 mmol) was added to a stirred solution of rel-(2R,3S,4S,5R)—N-(2-(2,2-dimethyl-1,3-dioxolan-4-yl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (15 mg, 0.026 mmol, first eluting isomer from Step 2) in DCM (3 mL) and the reaction mixture stirred at ambient temperature for 18 h. The solvent was removed in vacuo and the residue azeotroped with DCM (×2). Purification by reverse phase HPLC-MS using a a X-bridge C18 column (150×19 mm, 5 m particle size) from Waters gave rel-(2R,3S,4S,5R)—N-(2-(1,2-dihydroxyethyl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (17, 43 mg, 58%). ¹H NMR (500 MHz, DMSO-d₆) δ 10.47 (s, 1H), 8.98 (s, 2H), 7.24 (dd, J=8.7, 6.5 Hz, 1H), 6.97 (t, J=8.8 Hz, 1H), 5.17 (d, J=6.0 Hz, 1H), 5.11 (d, J=10.9 Hz, 1H), 4.59 (dt, J=10.6, 5.9 Hz, 2H), 4.37 (dd, J=10.9, 7.3 Hz, 1H), 3.99 (ddd, J=10.9, 5.5, 2.3 Hz, 1H), 3.85 (ddd, J=10.7, 6.6, 2.4 Hz, 1H), 3.75-3.54 (m, 4H), 3.31 (s, 3H), 2.76 (p, J=7.4 Hz, 1H), 2.16 (d, J=2.0 Hz, 3H), 1.64 (s, 3H), 0.76-0.61 (m, 3H) ppm; ESI-MS m/z calc. 531.1992, found 532.3 (M+1)⁺; 530.2 (M−1)⁻; Retention time: 3.02 minutes.

rel-(2S,3R,4R,5S)—N-(2-(2,2-dimethyl-1,3-dioxolan-4-yl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (53 mg, 0.09273 mmol) (Second Eluting Isomer from SFC separation in Step 2) was treated in the same way to give after reverse phase HPLC-MS chromatography, rel-(2S,3R,4R,5S)—N-(2-(1,2-dihydroxyethyl)pyrimidin-5-yl)-3-(4-fluoro-2-(2-methoxyethoxy)-3-methylphenyl)-4,5-dimethyl-5-(trifluoromethyl)tetrahydrofuran-2-carboxamide (18, 45 mg, 16%). ¹H NMR (500 MHz, DMSO-d₆) δ 10.47 (s, 1H), 8.98 (s, 2H), 7.24 (dd, 3=8.7, 6.5 Hz, 1H), 6.97 (t, (=8.8 Hz, 1H), 5.16 (d, t=6.0 Hz, 1H), 5.11 (d, =10.9 Hz, 1H), 4.64-4.50 (m, 2H), 4.37 (dd, =10.9, 7.3 Hz, 1H), 3.99 (ddd, 6=10.9, 5.6, 2.4 Hz, 1H), 3.85 (ddd, J=10.9, 6.6, 2.4 Hz, 1H), 3.79-3.53 (m, 4H), 3.32 (s, 3H), 2.76 (p, J=7.5 Hz, 1H), 2.16 (d, e=2.0 Hz, 3H), 1.64 (s, 3H), 0.77-10.62 (n, 3H) ppm. ESI-MS m/z calc. 531.1992, found 532.3 (M+1; 530.2 (M−1)⁻; Retention time: 3.02 minutes.

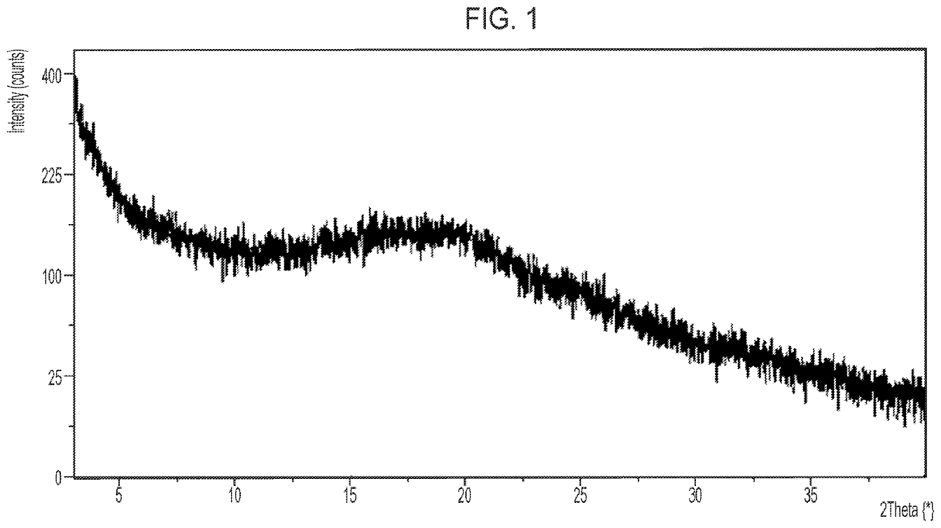

Compound 17 was analyzed by X-ray powder diffraction and determined to be amorphous (see FIG. 1).

The following compounds were made using the method described in Example 6, except that the starting material of step 2 was the first eluting isomers from Step 1:

| Cmpd No. | Compound Name | LC/MS | NMR (shifts in ppm) |
|---|---|---|---|
| 19 | rel-(2S,3S,4S,5R)-N-(2-(1,2- | ESI-MS m/z calc. | ¹H NMR (500 MHz, |
| | dihydroxyethyl)pyrimidin-5- | 531.1992, found 532.3 | DMSO-d₆) δ 10.07 (s, |
| | yl)-3-(4-fluoro-2-(2- | (M + 1)⁺; 530.2 (M − 1)⁻; | 1H), 8.75 (s, 2H), 7.31 (t, |
| | methoxyethoxy)-3- | Retention time: 2.82 | J = 7.9 Hz, 1H), 6.83 (t, J = |
| | methylphenyl)-4,5-dimethyl-5- | minutes. | 8.9 Hz, 1H), 5.14 (d, J = |
| | (trifluoromethyl)tetrahydrofuran- | | 6.1 Hz, 2H), 4.57 (p, J = |
| | 2-carboxamide | | 5.7 Hz, 2H), 4.39-4.30 |
| | (Precursor was the first eluting | | (m, 1H), 3.99-3.84 (m, |
| | peak by SFC on Chiralpak IC | | 2H), 3.76-3.57 (m, 4H), |
| | column, rt = 1.20 min) | | 3.38 (s, 3H), 2.94 (q, J = |
| | | | 7.9 Hz, 1H), 2.13 (d, J = |
| | | | 1.9 Hz, 3H), 1.58 (s, 3H), |
| | | | 0.75 (d, J = 7.5 Hz, 3H) |
| | | | ppm. |
| 20 | rel-(2R,3R,4R,5S)-N-(2-(1,2- | ESI-MS m/z calc. | ¹H NMR (500 MHz, |
| | dihydroxyethyl)pyrimidin-5- | 531.1992, found 532.3 | DMSO-d₆) δ 10.04 (s, |
| | yl)-3-(4-fluoro-2-(2- | (M + 1)⁺; 530.2 (M − 1)⁻; | 1H), 8.75 (s, 2H), 7.31 (t, |
| | methoxyethoxy)-3- | Retention time: 2.83 | J = 7.8 Hz, 1H), 6.83 (t, |
| | methylphenyl)-4,5-dimethyl-5- | minutes. | J = 8.9 Hz, 1H), 5.16-5.08 |
| | (trifluoromethyl)tetrahydrofuran- | | (m, 2H), 4.57 (dt, J = 9.3, |
| | 2-carboxamide | | 5.8 Hz, 2H), 4.38-4.29 |
| | (Precursor was the second | | (m, 1H), 3.91 (qdd, J = |
| | eluting peak by SFC on | | 10.8, 5.8, 3.2 Hz, 2H), |
| | Chiralpak IC column, rt = | | 3.76-3.57 (m, 4H), 3.38 |
| | 1.48 min) | | (s, 3H), 2.95 (p, J = 7.7 |
| | | | Hz, 1H), 2.13 (d, J = 1.9 |
| | | | Hz, 3H), 1.58 (s, 3H), |
| | | | 0.75 (d, J = 7.4 Hz, 3H) |
| | | | ppm. |

### Example 7

### rel-(2R*,3S*,4S*,5R*)-3-(3,4-difluoro-2-(2-methoxyethoxy)phenyl)-N-(6-(1-hydroxy-2-methoxyethyl)pyridin-3-yl)-4,5-dimethyl-5-(trifluoromethyl)tetrahydrofuran-2-carboxamide (21) and rel-(2R*,3S*,4S*,5R*)-3-(3,4-difluoro-2-(2-methoxyethoxy)phenyl)-N-(6-(1-hydroxy-2-methoxyethyl)pyridin-3-yl)-4,5-dimethyl-5-(trifluoromethyl)tetrahydrofuran-2-carboxamide (22)

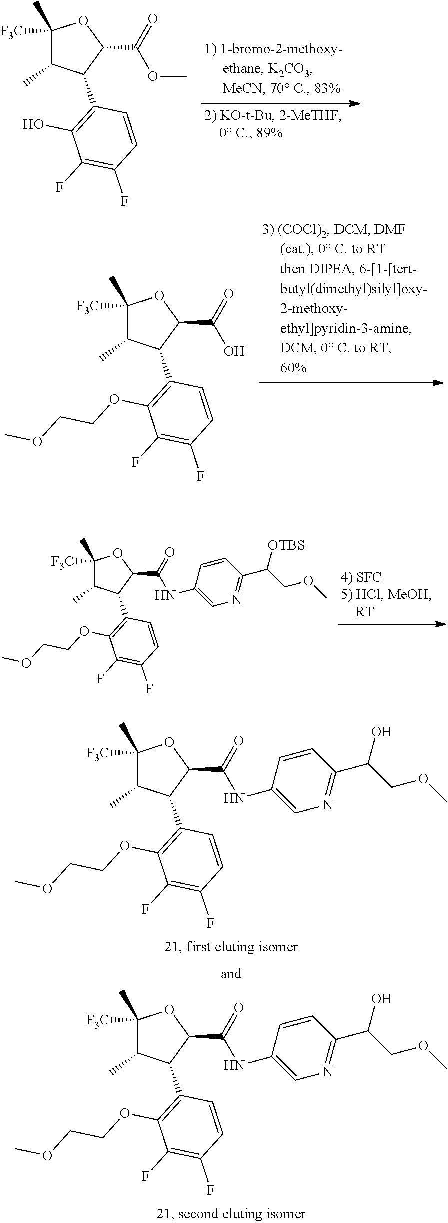

### Step 1:

To a solution of methyl (2S,3S,4S,5R)-3-(3,4-difluoro-2-hydroxy-phenyl)-4,5-dimethyl-5-(trifluoromethyl)tetrahydrofuran-2-carboxylate (3.72 g, 10.50 mmol) and K₂CO₃ (4.4 g, 31.84 mmol) in MeCN (75 mL) was added 1-bromo-2-methoxy-ethane (3 mL, 31.92 mmol) and the reaction mixture was heated at 70° C. for 5 h. The reaction mixture was transferred to a tube and 1-bromo-2-methoxy-ethane (3 mL, 31.92 mmol) was added. The tube was sealed and heated at 90° C. The reaction mixture was concentrated in vacuo and partitioned between water and EtOAc. The aqueous layer was extracted with EtOAc. The combined organics extracts were washed with brine, dried (MgSO₄), filtered and concentrated in vacuo to give 2-methoxyethyl (3S,4S,5R)-3-[3,4-difluoro-2-(2-methoxyethoxy)phenyl]-4,5-dimethyl-5-(trifluoromethyl)tetrahydrofuran-2-carboxylate (4 g, 83%). ESI-MS m/z calc. 456.15714, found 457.2 (M+1)⁺; Retention time: 1.01 minutes.

### Step 2:

KO-t-Bu (2.96 g, 26.38 mmol) was added to a solution of 2-methoxyethyl (3S,4S,5R)-3-[3,4-difluoro-2-(2-methoxyethoxy)phenyl]-4,5-dimethyl-5-(trifluoromethyl)tetrahydrofuran-2-carboxylate (4 g, 8.764 mmol) in 2-MeTHF (100 mL) at 0° C. The reaction mixture was stirred under nitrogen. The reaction mixture was acidified with HCl (15 mL of 2 M, 30.0 mmol) and extracted into EtOAc. The organic layer was separated, dried (MgSO₄), filtered and concentrated in vacuo to give (2R,3S,4S,5R)-3-[3,4-difluoro-2-(2-methoxyethoxy)phenyl]-4,5-dimethyl-5-(trifluoromethyl)tetrahydrofuran-2-carboxylic acid (3.1 g, 89%), which was used as is in the next step. ESI-MS m/z calc. 398.11526, found 397.1 (M+1)⁻; Retention time: 0.59 minutes.

### Step 3:

Oxalyl chloride (350 μL, 4.012 mmol) was added to a solution of (2R,3S,4S,5R)-3-[3,4-difluoro-2-(2-methoxyethoxy)phenyl]-4,5-dimethyl-5-(trifluoromethyl)tetrahydrofuran-2-carboxylic acid (435 mg, 1.092 mmol) and DMF (1 drop) in DCM (5 mL). The reaction mixture was stirred at ambient temperature for 30 minutes. The organic layer was concentrated in vacuo then dissolved in DCM (5 mL) and treated with Et₃N (330 μL, 2.368 mmol) and 6-[1-[tert-butyl(dimethyl)silyl]oxy-2-methoxy-ethyl]pyridin-3-amine (308.3 mg, 1.092 mmol). The reaction mixture was stirred at ambient temperature and then concentrated in vacuo. Purification by reverse phase HPLC-MS using a a X-bridge C18 column (150×19 mm, 5 m particle size) from Waters gave (2R,3S,4S,5R)—N-(6-(1-((tert-butyldimethylsilyl)oxy)-2-methoxyethyl)pyridin-3-yl)-3-(3,4-difluoro-2-(2-methoxyethoxy)phenyl)-4,5-dimethyl-5-(trifluoromethyl)tetrahydrofuran-2-carboxamide as a mixture of diastereomers at the tert-butyl(dimethyl)silyloxy position. ESI-MS m/z calc. 662.3, found 663.4 (M+1)⁺; 661.3 (M−1)⁻; Retention time: 1.28 minutes.

### Step 4:

The diastereomers of (2R,3S,4S,5R)—N-(6-(1-((tert-butyldimethylsilyl)oxy)-2-methoxyethyl)pyridin-3-yl)-3-(3,4-difluoro-2-(2-methoxyethoxy)phenyl)-4,5-dimethyl-5-(trifluoromethyl)tetrahydrofuran-2-carboxamide were separated by chiral SFC using a Chiralpak IB column, 5 m particle size, 25 cm×20 mm from Daicel Corporation on a Prep-100 SFC instrument from Waters:

First Eluting Isomer (rt=2.46 min): rel-(2R*,3S*,4S*,5R*)—N-(6-(1-((tert-butyldimethylsilyl)oxy)-2-methoxyethyl)pyridin-3-yl)-3-(3,4-difluoro-2-(2-methoxyethoxy)phenyl)-4,5-dimethyl-5-(trifluoromethyl)tetrahydrofuran-2-carboxamide. ESI-MS m/z calc. 662.2811, found 663.4 (M+1)⁺; 661.3 (M−1)⁻; Retention time: 4.28 minutes.

Second Eluting Isomer (rt=3.65 min): rel-(2R*,3S*,4S*,5R*)—N-(6-(1-((tert-butyldimethylsilyl)oxy)-2-methoxyethyl)pyridin-3-yl)-3-(3,4-difluoro-2-(2-methoxyethoxy)phenyl)-4,5-dimethyl-5-(trifluoromethyl)tetrahydrofuran-2-carboxamide. ESI-MS m/z calc. 662.2811, found 663.4 (M+1)⁺; 661.4 (M−1)⁻; Retention time: 4.28 minutes.

### Step 5:

Concentrated HCl (900 μl) was added to a solution of rel-(2R*,3S*,4S*,5R*)—N-(6-(1-((tert-butyldimethylsilyl)oxy)-2-methoxyethyl)pyridin-3-yl)-3-(3,4-difluoro-2-(2-methoxyethoxy)phenyl)-4,5-dimethyl-5-(trifluoromethyl)tetrahydrofuran-2-carboxamide (first eluting isomer from Step 4) in MeOH (5 mL). The reaction mixture was stirred at ambient temperature for 15 h and then concentrated in vacuo. Purification by reverse phase HPLC-MS using a a X-bridge C18 column (150×19 mm, 5 m particle size) from Waters gave rel-(2R*,3S*,4S*,5R*)-3-(3,4-difluoro-2-(2-methoxyethoxy)phenyl)-N-(6-(1-hydroxy-2-methoxyethyl)pyridin-3-yl)-4,5-dimethyl-5-(trifluoromethyl)tetrahydrofuran-2-carboxamide (21, 17.8 mg, 5.9%). ¹H NMR (500 MHz, Methanol-d₄) δ 8.71 (dd, J=2.5, 0.8 Hz, 1H), 8.08 (dd, J=8.6, 2.6 Hz, 1H), 7.55 (dt, J=8.7, 0.7 Hz, 1H), 7.18 (ddd, J=8.1, 5.5, 2.1 Hz, 1H), 7.01 (ddd, J=9.9, 8.9, 7.6 Hz, 1H), 5.10 (d, J=10.9 Hz, 1H), 4.47 (dd, J=10.9, 7.6 Hz, 1H), 4.37 (dddd, J=11.1, 5.9, 2.4, 1.1 Hz, 1H), 4.23 (dddd, J=11.2, 6.0, 2.6, 1.2 Hz, 1H), 3.74-3.62 (m, 3H), 3.57 (dd, J=10.1, 7.0 Hz, 1H), 3.37 (s, 3H), 3.36 (s, 3H), 2.88 (q, J=7.5 Hz, 1H), 1.71 (d, J=1.1 Hz, 3H), 0.82 (dq, J=7.4, 2.3 Hz, 3H) ppm. ESI-MS m/z calc. 548.1946, found 549.3 (M+1); 547.2 (M−1)⁻; Retention time: 3.19 minutes.

rel-(2R*,3S*,4S*,5R*)—N-(6-(1-((tert-butyldimethylsilyl)oxy)-2-methoxyethyl)pyridin-3-yl)-3-(3,4-difluoro-2-(2-methoxyethoxy)phenyl)-4,5-dimethyl-5-(trifluoromethyl)tetrahydrofuran-2-carboxamide (second eluting isomer from Step 4) was treated in the same way to give after reverse phase HPLC-MS chromatography rel-(2R*,3S*,4S*,5R*)-3-[3,4-difluoro-2-(2-methoxyethoxy)phenyl]-N-[6-(1-hydroxy-2-methoxy-ethyl)-3-pyridyl]-4,5-dimethyl-5-(trifluoromethyl)tetrahydrofuran-2-carboxamide (22, 19.8 mg, 6.6%). ¹H NMR (500 MHz, Methanol-d₄) δ 8.72 (dd, J=2.6, 0.7 Hz, 1H), 8.07 (dd, J=8.5, 2.5 Hz, 1H), 7.55 (dt, J=8.6, 0.6 Hz, 1H), 7.18 (ddd, J=8.3, 5.6, 2.0 Hz, 1H), 7.01 (ddd, J=9.9, 8.9, 7.6 Hz, 1H), 5.10 (d, J=10.9 Hz, 1H), 4.47 (dd, J=10.9, 7.6 Hz, 1H), 4.37 (dddd, J=11.2, 6.0, 2.5, 1.2 Hz, 1H), 4.23 (dddd, J=11.2, 6.0, 2.5, 1.2 Hz, 1H), 3.73-3.64 (m, 3H), 3.57 (dd, J=10.1, 7.1 Hz, 1H), 3.37 (s, 3H), 3.36 (s, 3H), 2.88 (p, J=7.5 Hz, 1H), 1.71 (d, J=1.2 Hz, 3H), 0.82 (dq, J=7.4, 2.3 Hz, 3H) ppm. ESI-MS m/z calc. 548.1946, found 549.2 (M+1)⁺; 547.2 (M−1)⁻; Retention time: 3.19 minutes.

### Example 8

### (2R,3S,4S,5R)-3-(3,4-difluoro-2-(2-hydroxyethoxy)phenyl)-N-(6-(hydroxymethyl)pyridin-3-yl)-4,5-dimethyl-5-(trifluoromethyl)tetrahydrofuran-2-carboxamide (23)

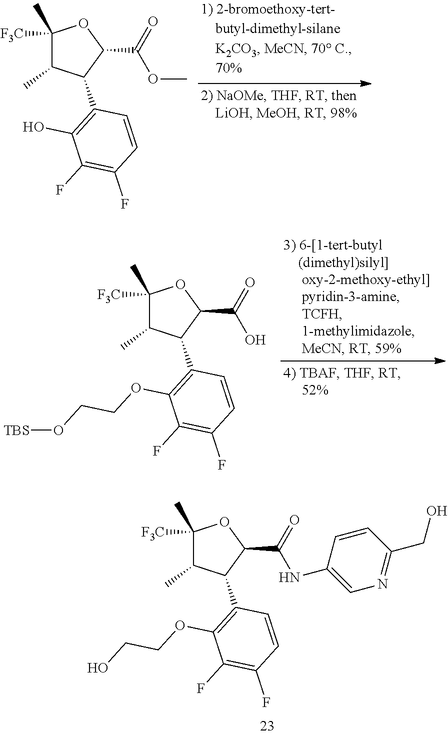

### Step 1:

2-bromoethoxy-tert-butyl-dimethyl-silane (5.5 mL, 25.63 mmol) was added to a mixture of methyl (2S,3S,4S,5R)-3-(3,4-difluoro-2-hydroxy-phenyl)-4,5-dimethyl-5-(trifluoromethyl) tetrahydrofuran-2-carboxylate (3 g, 8.468 mmol) and K₂CO₃ (3.7 g, 26.77 mmol) in MeCN (30 mL). The reaction mixture was heated at −73° C. under nitrogen overnight. More K₂CO₃ (3.7 g, 26.77 mmol) and 2-bromoethoxy-tert-butyl-dimethyl-silane (5.5 mL, 25.63 mmol) were added and the reaction mixture was stirred for 2 days at 70° C. The reaction mixture was cooled to ambient temperature and then filtered through a pre-packed celite cartridge. The filtrate was concentrated in vacuo and then dissolved in MTBE and partitioned between MTBE and water and the organic phase separated. The organic layer was washed further with water (2×) and brine (1×). The organic phase was dried (Na₂SO₄), filtered and concentrated in vacuo to leave a yellow oil 20 g. Purification by flash chromatography (SiO₂, 0 to 25% EtOAc in heptane) gave 2-[tert-butyl(dimethyl)silyl]oxyethyl (2R,3S,4S,5R)-3-[2-[2-[tert-butyl(dimethyl)silyl]oxyethoxy]-3,4-difluoro-phenyl]-4,5-dimethyl-5-(trifluoromethyl)tetrahydrofuran-2-carboxylate (3.9 g, 70%). ¹H NMR (500 MHz, DMSO-d₆) δ 7.18 (ddd, J=8.0, 5.8, 1.8 Hz, 1H), 7.10 (dt, J=9.8, 8.3 Hz, 1H), 5.12 (d, J=10.4 Hz, 1H), 4.25-4.13 (m, 3H), 4.10-4.04 (m, 2H), 3.93-3.86 (m, 2H), 3.70-3.62 (m, 2H), 2.73 (q, J=7.5 Hz, 1H), 1.53 (s, 3H), 0.86 (s, 9H), 0.80 (s, 9H), 0.69 (dt, J=8.5, 4.3 Hz, 3H), 0.06 (s, 6H), −0.02 (d, J=2.2 Hz, 6H) ppm.

### Step 2:

Sodium methanolate (120.5 μL of 25% w/v as a solution in MeOH, 0.55 mmol) was added to a solution of 2-[tert-butyl(dimethyl)silyl]oxyethyl (2R,3S,4S,5R)-3-[2-[2-[tert-butyl(dimethyl)silyl]oxyethoxy]-3,4-difluoro-phenyl]-4,5-dimethyl-5-(trifluoromethyl)tetrahydrofuran-2-carboxylate (3.9 g, 5.93 mmol) in THF (30 mL) at ambient temperature under nitrogen. The reaction mixture was stirred for 5 h. MeOH (30 mL) and LiOH (3.614 mL of 2 M aqueous solution, 7.228 mmol) were added and the reaction mixture was stirred overnight at ambient temperature. The reaction mixture was poured into 1M HCl then extracted with MTBE (2×30 ml). The combined organic layers were washed with brine, dried (Na₂SO₄), filtered and concentrated in vacuo to give (2R,3S,4S,5R)-3-[2-[2-[tert-butyl(dimethyl)silyl]oxyethoxy]-3,4-difluoro-phenyl]-4,5-dimethyl-5-(trifluoromethyl)tetrahydrofuran-2-carboxylic acid (2.89 g, 98%). ESI-MS m/z calc. 498.1861, found 499.6 (M+1)⁺; 497.6 (M−1)⁻; Retention time: 0.82 minutes.

### Step 3:

To a solution of (2R,3S,4S,5R)-3-[2-[2-[tert-butyl(dimethyl)silyl]oxyethoxy]-3,4-difluoro-phenyl]-4,5-dimethyl-5-(trifluoromethyl)tetrahydrofuran-2-carboxylic acid (175 mg, 0.3510 mmol) in MeCN (5 mL) was added [chloro(dimethylamino)methylene]-dimethyl-ammonium (118 mg, 0.4206 mmol), 1-methylimidazole (84 mg, 1.023 mmol) and 6-[[tert-butyl(dimethyl)silyl]oxymethyl]pyridin-3-amine (92 mg, 0.3859 mmol) and the reaction mixture was stirred at ambient temperature overnight. The reaction mixture was poured onto water (15 mL) and EtOAc (15 mL) and the layers separated. The organic layer was washed with brine (5 mL), dried (MgSO₄), filtered and concentrated in vacuo. Purification by flash chromatography (SiO₂, 0 to 25% EtOAc in heptane) gave (2R,3S,4S,5R)-3-[2-[2-[tert-butyl(dimethyl)silyl]oxyethoxy]-3,4-difluoro-phenyl]-N-[6-[[tert-butyl(dimethyl)silyl]oxymethyl]-3-pyridyl]-4,5-dimethyl-5-(trifluoromethyl)tetrahydrofuran-2-carboxamide (150 mg, 59%). ESI-MS m/z calc. 718.3257, found 719.9 (M+1)⁺; Retention time: 1.44 minutes.

### Step 4:

To a solution of (2R,3S,4S,5R)-3-[2-[2-[tert-butyl(dimethyl)silyl]oxyethoxy]-3,4-difluoro-phenyl]-N-[6-[[tert-butyl(dimethyl)silyl]oxymethyl]-3-pyridyl]-4,5-dimethyl-5-(trifluoromethyl) tetrahydrofuran-2-carboxamide (130 mg, 0.180 mmol) in THF (2 mL) was added TBAF (542 μL of 1 M, 0.542 mmol) and the mixture stirred at ambient temperature for 2 h. The reaction mixture was concentrated in vacuo. Purification by reverse phase HPLC-MS using a a X-bridge C18 column (150×19 mm, 5 m particle size) from Waters gave (2R,3S,4S,5R)-3-(3,4-difluoro-2-(2-hydroxyethoxy)phenyl)-N-(6-(hydroxymethyl)pyridin-3-yl)-4,5-dimethyl-5-(trifluoromethyl)tetrahydrofuran-2-carboxamide (23, 48 mg, 52%). ¹H NMR (500 MHz, DMSO-d₆) δ 10.33 (s, 1H), 8.70 (dd, J=2.6, 0.7 Hz, 1H), 8.04 (dd, J=8.5, 2.6 Hz, 1H), 7.44 (dd, J=8.4, 0.9 Hz, 1H), 7.20 (dd, J=8.4, 3.7 Hz, 2H), 5.37 (t, J=5.8 Hz, 1H), 5.13 (d, J=10.8 Hz, 1H), 5.00 (s, 1H), 4.54 (d, J=5.7 Hz, 2H), 4.43 (dd, J=10.8, 7.3 Hz, 1H), 4.25-4.06 (m, 2H), 3.75 (d, J=4.3 Hz, 2H), 3.32 (s, 3H), 2.95 (q, J=7.4 Hz, 1H), 1.65 (s, 3H) ppm. ESI-MS m z calc. 490.1527, found 491.6 (M+1)⁺; 489.6 (M−1)⁻; Retention time: 2.77 minutes.

### Example 9

### (2R,3S,4S,5R)-3-(3,4-difluoro-2-(oxetan-3-ylmethoxy)phenyl)-N-(6-(hydroxymethyl)pyridin-3-yl)-4,5-dimethyl-5-(trifluoromethyl)tetrahydrofuran-2-carboxamide (24)

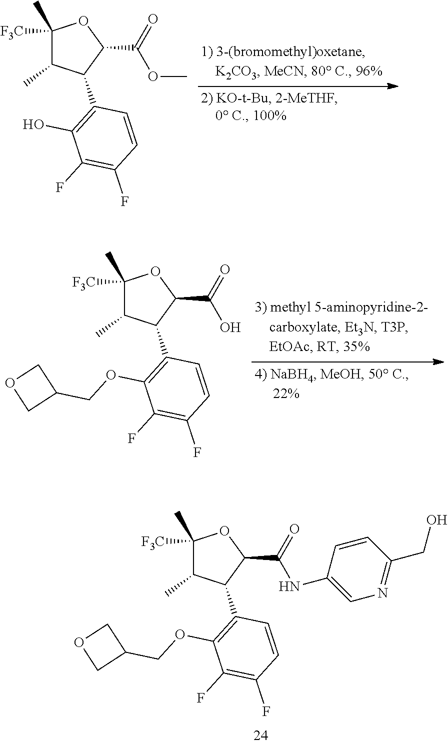 [SMILES: COC(=O)[C@H]1O[C@@](C)(C(F)(F)F)[C@@H](C)[C@H]1c1ccc(F)c(F)c1O ; C[C@H]1[C@@H](c2ccc(F)c(F)c2OCC2COC2)[C@H](C(=O)O)O[C@@]1(C)C(F)(F)F ; C[C@H]1[C@@H](c2ccc(F)c(F)c2OCC2COC2)[C@H](C(=O)Nc2ccc(CO)nc2)O[C@@]1(C)C(F)(F)F]

3-(bromomethyl)oxetane (526 mg, 3.483 mmol) was added to a mixture of methyl (2S,3S,4S,5R)-3-(3,4-difluoro-2-hydroxy-phenyl)-4,5-dimethyl-5-(trifluoromethyl)tetrahydrofuran-2-carboxylate (505 mg, 1.425 mmol) and K₂CO₃ (589 mg, 4.262 mmol) in MeCN (10 mL) and the reaction mixture was stirred at 80° C. for 5 h. A further 100 mg of 3-(bromomethyl)oxetane was added and reaction mixture was stirred at 80° C. overnight. The reaction mixture was partitioned between water and EtOAc. The aqueous layer was extracted with EtOAc. The combined organic layers were washed with brine, dried (MgSO₄), filtered and concentrated in vacuo to give oxetan-3-ylmethyl (3S,4S,5R)-3-[3,4-difluoro-2-(oxetan-3-ylmethoxy)phenyl]-4,5-dimethyl-5-(trifluoromethyl)tetrahydrofuran-2-carboxylate (770 mg, 96%). ESI-MS m/z calc. 480.15714, Retention time: 0.89 minutes.

### Step 2:

KO-t-Bu (328 mg, 2.923 mmol) was added to a solution of oxetan-3-ylmethyl (3S,4S,5R)-3-[3,4-difluoro-2-(oxetan-3-ylmethoxy)phenyl]-4,5-dimethyl-5-(trifluoromethyl)tetrahydrofuran-2-carboxylate (770 mg, 1.362 mmol) in 2-MeTHF (10 mL) cooled to 0° C. and the reaction mixture was stirred for 1 h. The reaction mixture was quenched with diluted HCl solution. The aqueous layer was extracted with EtOAc. The combined organic layers were dried (MgSO₄), filtered and concentrated in vacuo to give (2R,3S,4S,5R)-3-[3,4-difluoro-2-(oxetan-3-ylmethoxy)phenyl]-4,5-dimethyl-5-(trifluoromethyl)tetrahydrofuran-2-carboxylic acid (680 mg, 100%) as a yellow oil. ¹H NMR (400 MHz, Chloroform-d) δ 7.02-6.95 (m, 1H), 6.90 (td, J=9.2, 7.3 Hz, 1H), 4.93 (d, J=10.5 Hz, 1H), 4.89 (ddd, J=7.9, 6.3, 2.4 Hz, 2H), 4.81 (ddd, J=12.4, 7.7, 6.4 Hz, OH), 4.58 (dt, J=11.0, 6.1 Hz, 2H), 4.51 (ddd, J=10.1, 6.2, 2.1 Hz, 1H), 4.29 (ddd, J=10.0, 6.3, 1.6 Hz, 1H), 4.18-4.12 (m, 1H), 3.40 (dt, J=14.1, 7.5 Hz, 1H), 2.70 (p, J=7.6 Hz, 1H), 1.63-1.59 (m, 3H), 0.81-0.72 (m, 3H) ppm. ESI-MS m/z calc. 410.11526, found 409.2 (M−1)⁻; Retention time: 0.56 minutes.

### Step 3:

T3P (450 μL of 50% w/w, 0.755 mmol) and Et₃N (210 μL, 1.5 mmol) were added successively to a solution of (2R,3S,4S,5R)-3-[3,4-difluoro-2-(oxetan-3-ylmethoxy)phenyl]-4,5-dimethyl-5-(trifluoromethyl)tetrahydrofuran-2-carboxylic acid (250 mg, 0.49 mmol) and methyl 5-aminopyridine-2-carboxylate (102 mg, 0.67 mmol) in EtOAc (5 mL). The reaction stirred at ambient temperature for 1.5 h. A further 30 μl of T3P was added and the reaction mixture was stirred at ambient temperature for 1 h. The reaction mixture was partitioned between water and EtOAc. The aqueous layer was extracted with EtOAc. The combined organic layers were washed with brine, dried (MgSO₄), filtered and concentrated in vacuo. Purification by flash chromatography (SiO₂, 0 to 100% EtOAc in heptane) gave methyl 5-[[(2R,3S,4S,5R)-3-[3,4-difluoro-2-(oxetan-3-ylmethoxy)phenyl]-4,5-dimethyl-5-(trifluoromethyl)tetrahydrofuran-2-carbonyl]amino]pyridine-2-carboxylate (95 mg, 35%) as a white solid. H NMR (400 MHz, Chloroform-d) δ 8.68 (dd, J=2.6, 0.7 Hz, 1H), 8.53 (s, 1H), 8.33 (dd, J=8.6, 2.6 Hz, 1H), 8.11 (d, J=8.6 Hz, 1H), 7.11 (ddd, J=8.1, 5.5, 2.1 Hz, 1H), 6.94 (td, J=9.2, 7.5 Hz, 1H), 5.03 (d, J=11.1 Hz, 1H), 4.82 (ddd, J=28.7, 8.0, 6.2 Hz, 2H), 4.61 (t, J=6.0 Hz, 1H), 4.55-4.45 (m, 2H), 4.29 (ddd, J=10.3, 5.4, 2.0 Hz, 1H), 4.24 (dd, J=11.1, 8.0 Hz, 1H), 3.99 (s, 3H), 3.35 (ddd, J=13.7, 7.9, 5.7 Hz, 1H), 2.76 (p, J=7.6 Hz, 1H), 1.69 (d, J=1.2 Hz, 3H), 0.84-0.72 (m, 3H) ppm. ESI-MS m/z calc. 544.16327, found 545.2 (M+1)⁺; 543.2 (M−1)⁻; Retention time: 0.92 minutes.

### Step 4:

NaBH₄ (6 mg, 0.15 mmol) was added to a solution of methyl 5-[[(2R,3S,4S,5R)-3-[3,4-difluoro-2-(oxetan-3-ylmethoxy)phenyl]-4,5-dimethyl-5-(trifluoromethyl)tetrahydrofuran-2-carbonyl]amino]pyridine-2-carboxylate (11 mg, 0.02 mmol) in MeOH (1 mL) and the reaction mixture was stirred at 50° C. for 6 h. The reaction mixture was stirred overnight at 40° C. The reaction mixture was quenched with AcOH and diluted with MeCN, water and MeOH. Purification by reverse phase HPLC-MS using a a X-bridge C18 column (150×19 mm, 5 m particle size) from Waters gave (2R,3S,4S,5R)-3-(3,4-difluoro-2-(oxetan-3-ylmethoxy)phenyl)-N-(6-(hydroxymethyl)pyridin-3-yl)-4,5-dimethyl-5-(trifluoromethyl)tetrahydrofuran-2-carboxamide (Trifluoroacetate salt) (24, 2.9 mg, 22%). ¹H NMR (400 MHz, Methanol-d₄) δ 8.74 (d, J=2.4 Hz, 1H), 8.13 (dd, J=8.5, 2.5 Hz, 1H), 7.54 (d, J=8.6 Hz, 1H), 7.20-7.13 (m, 1H), 7.05-6.96 (m, 1H), 5.09 (d, J=10.6 Hz, 1H), 4.90-4.85 (m, 2H), 4.67 (s, 2H), 4.63 (dt, J=10.6, 6.1 Hz, 2H), 4.50 (ddd, J=10.0, 5.9, 1.8 Hz, 1H), 4.37 (dd, J=10.6, 8.0 Hz, 1H), 4.31 (dd, J=9.6, 6.2 Hz, 1H), 3.51-3.40 (m, 1H), 2.79 (p, J=7.4 Hz, 1H), 1.68-1.64 (m, 3H), 0.86-0.79 (m, 3H) ppm. ESI-MS m/z calc. 516.16833, found 516.9 (M+1); 515.0 (M−1)⁻; Retention time: 3.69 minutes.

### Example 10

### (2R,3S,4S,5R)-3-(3,4-difluoro-2-(2-morpholinoethoxy)phenyl)-N-(6-(hydroxymethyl)pyridin-3-yl)-4,5-dimethyl-5-(trifluoromethyl)tetrahydrofuran-2-carboxamide (25)

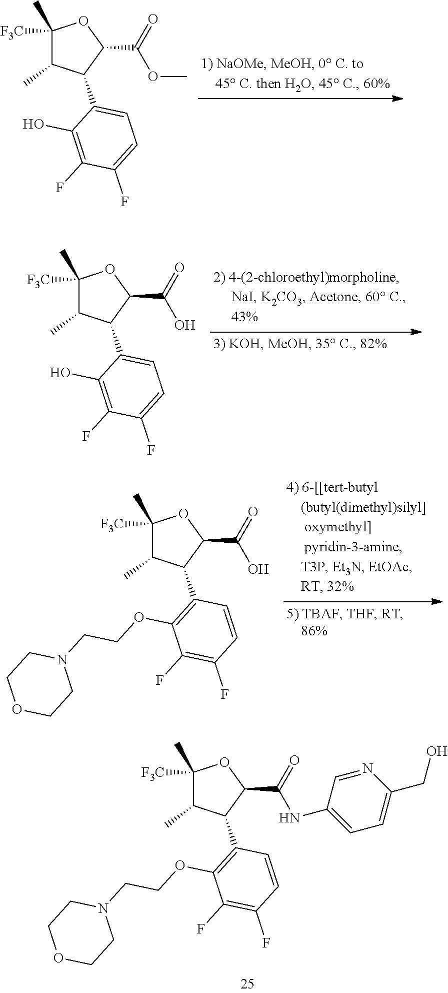 [SMILES: COC(=O)[C@H]1O[C@@](C)(C(F)(F)F)[C@@H](C)[C@H]1c1ccc(F)c(F)c1O ; C[C@H]1[C@@H](c2ccc(F)c(F)c2O)[C@H](C(=O)O)O[C@@]1(C)C(F)(F)F ; C[C@H]1[C@@H](c2ccc(F)c(F)c2OCCN2CCOCC2)[C@H](C(=O)O)O[C@@]1(C)C(F)(F)F ; C[C@H]1[C@@H](c2ccc(F)c(F)c2OCCN2CCOCC2)[C@H](C(=O)Nc2ccc(CO)nc2)O[C@@]1(C)C(F)(F)F]

To a solution of methyl (2S,3S,4S,5R)-3-(3,4-difluoro-2-hydroxy-phenyl)-4,5-dimethyl-5-(trifluoromethyl)tetrahydrofuran-2-carboxylate (5 g, 14.11 mmol) in MeOH (20 mL) cooled on an ice bath was added a solution of NaOMe (9.14 mL of 25% w/v as solution in MeOH, 42.30 mmol) dropwise over 10 mins. The reaction mixture was stirred for 1 h at 0° C. and then stirred at 45° C. overnight. To the reaction mixture was added water (1.52 mL, 84.37 mmol) and the reaction mixture was stirred at 45° C. for 1 h. The reaction mixture was concentrated in vacuo and then partitioned between 2-MeTHF (50 mL) and water (25 mL). The aqueous phase was acidified to pH 1 with HCl and the layers were separated. The aqueous layer was extracted with 2-MeTHF (10 mL) and the combined organic layers were dried (Na₂SO₄), filtered, and concentrated in vacuo to give (2R,3S,4S,5R)-3-(3,4-difluoro-2-hydroxy-phenyl)-4,5-dimethyl-5-(trifluoromethyl)tetrahydrofuran-2-carboxylic acid (3.9 g, 61%). ESI-MS m/z calc. 340.0734, found 339.1 (M−1)⁻; Retention time: 0.46 minutes.

### Step 2:

To a solution of (2R,3S,4S,5R)-3-(3,4-difluoro-2-hydroxy-phenyl)-4,5-dimethyl-5-(trifluoromethyl)tetrahydrofuran-2-carboxylic acid (4 g, 11.76 mmol) in acetone (30 mL) was added 4-(2-chloroethyl)morpholine (Hydrochloride salt) (6.56 g, 35.25 mmol), NaI (1.76 g, 11.74 mmol) and K₂CO₃ (8.12 g, 58.75 mmol) and the reaction mixture was heated to 60° C. for 24 h. The reaction mixture was allowed to cool down and was partitioned between MTBE (100 mL) and water (100 mL). The aqueous layer was further extracted with MTBE (30 mL). The combined organic fractions were washed with brine (1×10 mL), dried (MgSO₄), filtered and concentrated in vacuo. Purification by flash chromatography (SiO₂, 25% MeOH in DCM) gave 2-morpholinoethyl (2R,3S,4S,5R)-3-[3,4-difluoro-2-(2-morpholinoethoxy)phenyl]-4,5-dimethyl-5-(trifluoromethyl)tetrahydrofuran-2-carboxylate (4.8 g, 43%). ESI-MS m/z calc. 566.2415, found 567.3 (M+1)⁺; Retention time: 0.96 minutes.

### Step 3:

KOH (990 mg, 17.65 mmol) was added to solution of 2-morpholinoethyl (2R,3S,4S,5R)-3-[3,4-difluoro-2-(2-morpholinoethoxy)phenyl]-4,5-dimethyl-5-(trifluoromethyl)tetrahydrofuran-2-carboxylate (4 g, 7.060 mmol) in MeOH (20 mL) and the reaction mixture was stirred at 35° C. until completion. Water (20 mL) was added to the reaction mixture and the MeOH was removed in vacuo. The aqueous crude was basified with a 1M NaOH solution to pH 14 and washed with MTBE (20 mL) to remove the ethylmorpholine. The organic phase was extracted with water (×2). The combined aqueous phases were then acidified to pH 4.6 with 6 N HCl and extracted with EtOAc (×3). The combined organic phases were dried (MgSO₄), filtered and concentrated in vacuo to yield (2R,3S,4S,5R)-3-[3,4-difluoro-2-(2-morpholinoethoxy)phenyl]-4,5-dimethyl-5-(trifluoromethyl)tetrahydrofuran-2-carboxylic acid (2.7 g, 82%). ¹H NMR (500 MHz, DMSO-d₆) δ 7.19 (ddd, J=7.9, 5.8, 1.7 Hz, 1H), 7.12 (td, J=9.4, 7.5 Hz, 1H), 5.01 (d, J=10.7 Hz, 1H), 4.39-4.29 (m, 1H), 4.22 (ddd, J=17.1, 10.8, 6.0 Hz, 2H), 3.57 (t, J=4.7 Hz, 4H), 2.83-2.61 (m, 3H), 2.45 (s, 4H), 1.56 (s, 3H), 0.67 (dt, J=7.3, 2.3 Hz, 3H) ppm. ESI-MS m/z calc. 453.1574, found 454.4 (M+1)⁺; 452.2 (M−1)⁻; Retention time: 0.55 minutes.

### Step 4:

To a solution of (2R,3S,4S,5R)-3-[3,4-difluoro-2-(2-morpholinoethoxy)phenyl]-4,5-dimethyl-5-(trifluoromethyl)tetrahydrofuran-2-carboxylic acid (70 mg, 0.149 mmol) in EtOAc (2 mL) was added 6-[[tert-butyl(dimethyl)silyl]oxymethyl]pyridin-3-amine (71 mg, 0.29 mmol), Et₃N (75 μL, 0.53 mmol) and T3P (130 μL, 0.437 mmol) and the reaction mixture was stirred at ambient temperature for 4 h. The reaction mixture was partitioned between EtOAc (10 mL) and water (10 mL). The organic layer was washed again with water (10 mL). The organic phase was washed with brine (1×10 mL), dried (MgSO₄), filtered and concentrated in vacuo to give (2R,3S,4S,5R)—N-[6-[[tert-butyl(dimethyl)silyl]oxymethyl]-3-pyridyl]-3-[3,4-difluoro-2-(2-morpholinoethoxy)phenyl]-4,5-dimethyl-5-(trifluoromethyl)tetrahydrofuran-2-carboxamide (60 mg, 32%). ESI-MS m/z calc. 673.297, found 674.3 (M+1)⁺; 672.4 (M−1)⁻; Retention time: 1.27 minutes.

### Step 5:

TBAF (100 μL of 1 M, 0.1 mmol) was added to a solution of (2R,3S,4S,5R)—N-[6-[[tert-butyl(dimethyl)silyl]oxymethyl]-3-pyridyl]-3-[3,4-difluoro-2-(2-morpholinoethoxy)phenyl]-4,5-dimethyl-5-(trifluoromethyl)tetrahydrofuran-2-carboxamide (80 mg, 0.062 mmol) in THF (2 mL) and the reaction mixture stirred overnight at ambient temperature. The reaction mixture was concentrated in vacuo. Purification by reverse phase HPLC-MS using a a X-bridge C18 column (150×19 mm, 5 m particle size) from Waters gave (2R,3S,4S,5R)-3-(3-fluoro-2-(2-morpholinoethoxy)phenyl)-N-(6-(hydroxymethyl)pyridin-3-yl)-4,5-dimethyl-5-(trifluoromethyl)tetrahydrofuran-2-carboxamide (25, 31 mg, 86%). ¹H NMR (500 MHz, DMSO-d₆) δ 10.35 (s, 1H), 8.67 (d, J=2.5 Hz, 1H), 8.03 (td, J=8.3, 2.5 Hz, 1H), 7.40 (d, J=8.5 Hz, 1H), 7.21-7.09 (m, 2H), 5.35 (s, 1H), 5.12 (d, J=10.6 Hz, 1H), 4.50 (s, 2H), 4.38-4.28 (m, 3H), 4.26-4.18 (m, 2H), 3.57-3.45 (m, 4H), 2.88 (p, J=7.5 Hz, 1H), 2.40 (t, J=4.8 Hz, 4H), 1.64 (s, 3H), 0.74-0.68 (m, 3H) ppm. ESI-MS m/z calc. 559.2106, found 560.3 (M+1)⁺; 558.3 (M−1)⁻; Retention time: 3.0 minutes.

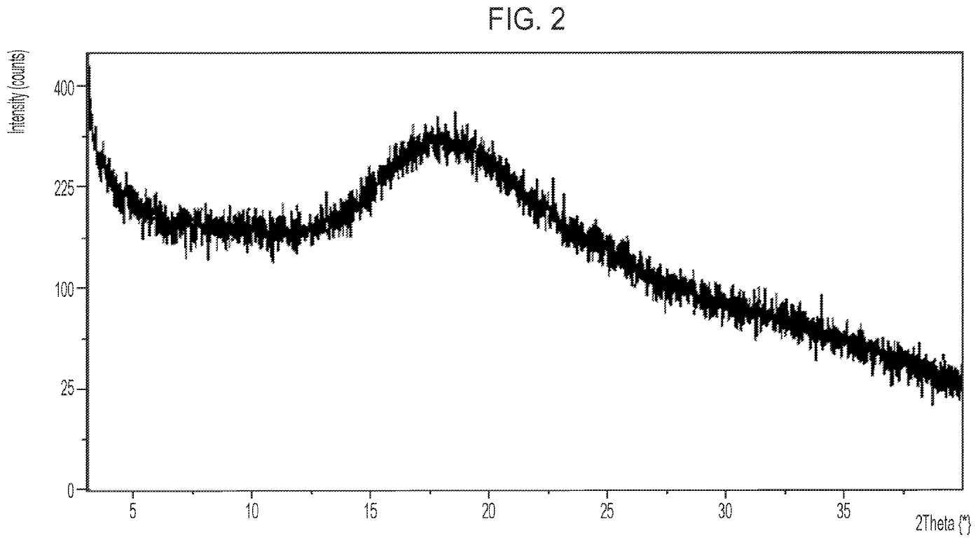

Compound 25 was analyzed by X-ray powder diffraction and determined to be amorphous (see FIG. 2).

### Example 11

### (2R,3S,4S,5R)-3-(3,4-difluoro-2-(2-((3aR,6aS)-tetrahydro-1H-furo[3,4-c]pyrrol-5(3H)-yl)ethoxy)phenyl)-N-(2-(hydroxymethyl)pyrimidin-5-yl)-4,5-dimethyl-5-(trifluoromethyl)tetrahydrofuran-2-carboxamide (26)

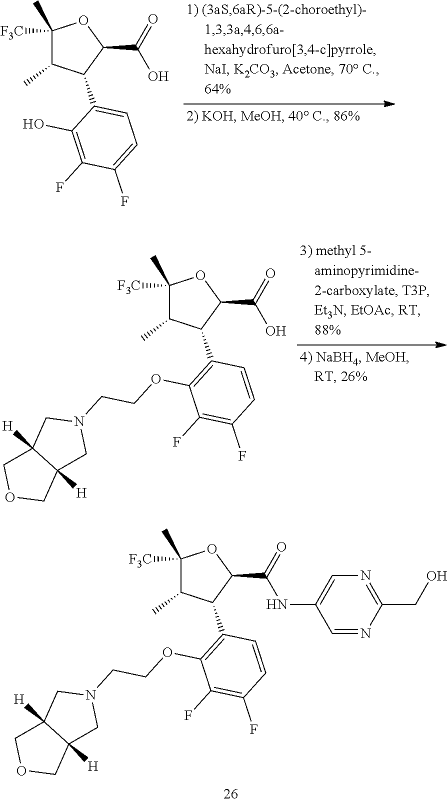 [SMILES: C[C@H]1[C@@H](c2ccc(F)c(F)c2O)[C@H](C(=O)O)O[C@@]1(C)C(F)(F)F ; C[C@H]1[C@@H](c2ccc(F)c(F)c2OCCN2C[C@H]3COC[C@H]3C2)[C@H](C(=O)O)O[C@@]1(C)C(F)(F)F ; C[C@H]1[C@@H](c2ccc(F)c(F)c2OCCN2C[C@H]3COC[C@H]3C2)[C@H](C(=O)Nc2cnc(CO)nc2)O[C@@]1(C)C(F)(F)F]

### Step 1:

NaI (258 mg, 1.721 mmol), K₂CO₃ (952 mg, 6.888 mmol) and (3aS,6aR)-5-(2-chloroethyl)-1,3,3a,4,6,6a-hexahydrofuro[3,4-c]pyrrole (908 mg, 5.169 mmol) were added to a solution of (2R,3S,4S,5R)-3-(3,4-difluoro-2-hydroxy-phenyl)-4,5-dimethyl-5-(trifluoromethyl)tetrahydrofuran-2-carboxylic acid (586 mg, 1.722 mmol) in acetone (9 mL). The reaction mixture was stirred at 70° C. overnight. The reaction mixture was filtered and concentrated in vacuo. Purification by flash chromatography (SiO₂, 0 to 100% heptane 3:1 EtOAc:EtOH with 0.5% NH₄OH) gave 2-[(3aS,6aR)-1,3,3a,4,6,6a-hexahydrofuro[3,4-c]pyrrol-5-yl]ethyl (2R,3S,4S,5R)-3-[2-[2-[(3aS,6aR)-1,3,3a,4,6,6a-hexahydrofuro[3,4-c]pyrrol-5-yl]ethoxy]-3,4-difluoro-phenyl]-4,5-dimethyl-5-(trifluoromethyl)tetrahydrofuran-2-carboxylate (678 mg, 64%) as a pale yellow oil. ¹H NMR (500 MHz, DMSO-d₆) δ 7.23-7.18 (m, 1H), 7.14-7.08 (m, 1H), 5.13 (d, J=10.7 Hz, 1H), 4.29-4.24 (m, 1H), 4.22 (dd, J=10.7, 7.4 Hz, 1H), 4.18-4.12 (m, 2H), 4.11-4.06 (m, 1H), 3.70-3.62 (m, 4H), 3.41-3.37 (m, 2H), 3.25-3.20 (m, 2H), 2.78-2.67 (m, 7H), 2.60-2.54 (m, 2H), 2.47 (t, J=5.5 Hz, 2H), 2.38-2.32 (m, 2H), 2.25-2.19 (m, 3H), 2.16 (dd, J=9.1, 2.8 Hz, 1H), 1.54 (s, 3H), 0.68 (d, J=6.4 Hz, 3H) ppm. ESI-MS m/z calc. 618.2728, found 619.0 (M+1)⁺; Retention time: 3.37 minutes.

### Step 2:

KOH (142 mg, 2.531 mmol) was added to a solution of 2-[(3aS,6aR)-1,3,3a,4,6,6a-hexahydrofuro[3,4-c]pyrrol-5-yl]ethyl (2R,3S,4S,5R)-3-[2-[2-[(3aS,6aR)-1,3,3a,4,6,6a-hexahydrofuro[3,4-c]pyrrol-5-yl]ethoxy]-3,4-difluoro-phenyl]-4,5-dimethyl-5-(trifluoromethyl)tetrahydrofuran-2-carboxylate (640 mg, 1.035 mmol) in MeOH (2.6 mL). The reaction mixture was stirred at 40° C. for 40 minutes. The reaction mixture was diluted with 1M NaOH (50 mL) and extracted with MTBE (50 mL). The organic phase was back-extracted with water (20 mL). The combined aqueous phases were then acidified to pH 5.24 with 0.1 M HCl and the product was extracted with EtOAc (5×50 mL). The combined organic phases were dried (MgSO₄), filtered and concentrated in vacuo to give (2R,3S,4S,5R)-3-[2-[2-[(3aS,6aR)-1,3,3a,4,6,6a-hexahydrofuro[3,4-c]pyrrol-5-yl]ethoxy]-3,4-difluoro-phenyl]-4,5-dimethyl-5-(trifluoromethyl)tetrahydrofuran-2-carboxylic acid (428 mg, 86%) as an off white solid. ¹H NMR (500 MHz, DMSO-d₆) δ 7.21-7.17 (m, 1H), 7.15-7.09 (m, 1H), 4.98 (d, J=10.7 Hz, 1H), 4.31-4.26 (m, 1H), 4.23 (dd, J=10.7, 7.4 Hz, 1H), 4.18-4.13 (m, 1H), 3.65-3.60 (m, 2H), 3.44-3.38 (m, 2H), 2.79-2.69 (m, 5H), 2.27-2.20 (m, 2H), 1.54 (s, 3H), 1.30-1.23 (m, 2H), 0.87 (t, J=6.8 Hz, 1H), 0.67 (d, J=6.4 Hz, 3H) ppm. ESI-MS m/z calc. 479.1731, found 480.0 (M+1)⁺; 478.1 (M−1)⁻; Retention time: 2.25 minutes.

### Step 3:

To a solution of (2R,3S,4S,5R)-3-[2-[2-[(3aS,6aR)-1,3,3a,4,6,6a-hexahydrofuro[3,4-c]pyrrol-5-yl]ethoxy]-3,4-difluoro-phenyl]-4,5-dimethyl-5-(trifluoromethyl)tetrahydrofuran-2-carboxylic acid (100 mg, 0.208 mmol) in EtOAc (1.5 mL) was added methyl 5-aminopyrimidine-2-carboxylate (96 mg, 0.626 mmol) followed by the addition of Et₃N (170 μL, 1.22 mmol). The resulting mixture was cooled to 0° C. and T3P (500 μL, 0.84 mmol) was added dropwise. The reaction mixture was stirred at ambient temperature for 1 h. The reaction mixture was diluted with EtOAc (10 mL) and poured over saturated aqueous NaHCO₃ (15 mL). The organic layer was separated and the aqueous layer was extracted with EtOAc (2×10 mL). The organic layers were combined, washed with brine (20 mL), dried (MgSO₄) and concentrated in vacuo. Purification by flash chromatography (SiO₂, 0 to 100% heptane in 3:1 EtOAc:EtOH with 0.5% NH₄OH) gave methyl 5-[[(2R,3S,4S,5R)-3-[2-[2-[(3aS,6aR)-1,3,3a,4,6,6a-hexahydrofuro[3,4-c]pyrrol-5-yl]ethoxy]-3,4-difluoro-phenyl]-4,5-dimethyl-5-(trifluoromethyl)tetrahydrofuran-2-carbonyl]amino]pyrimidine-2-carboxylate (113 mg, 88%) as a colourless oil. ESI-MS m/z calc. 614.2164, found 615.1 (M+1)⁺; 613.2 (M−1)⁻; Retention time: 0.92 minutes.

### Step 4:

NaBH₄ (20 mg, 0.5286 mmol) was added to a solution of methyl 5-[[(2R,3S,4S,5R)-3-[2-[2-[(3aS,6aR)-1,3,3a,4,6,6a-hexahydrofuro[3,4-c]pyrrol-5-yl]ethoxy]-3,4-difluoro-phenyl]-4,5-dimethyl-5-(trifluoromethyl)tetrahydrofuran-2-carbonyl]amino]pyrimidine-2-carboxylate (111 mg, 0.18 mmol) in MeOH (1.0 mL) cooled to 0° C. The reaction mixture was stirred at ambient temperature for 90 minutes and then quenched with water (2 mL) and stirred for 20 minutes. The reaction mixture was diluted with MeOH (5 mL). Purification by reverse phase HPLC-MS using a a X-bridge C18 column (150×19 mm, 5 m particle size) from Waters gave (2R,3S,4S,5R)-3-(3,4-difluoro-2-(2-((3aR,6aS)-tetrahydro-1H-furo[3,4-c]pyrrol-5(3H)-yl)ethoxy)phenyl)-N-(2-(hydroxymethyl)pyrimidin-5-yl)-4,5-dimethyl-5-(trifluoromethyl)tetrahydrofuran-2-carboxamide (26, 27.6 mg, 26%) as a white solid. ¹H NMR (500 MHz, DMSO-d₆) δ 10.43 (s, 1H), 9.00 (s, 2H), 7.21-7.17 (m, 1H), 7.16-7.10 (m, 1H), 5.25 (t, J=6.3 Hz, 1H), 5.16 (d, J=10.6 Hz, 1H), 4.55 (d, J=6.3 Hz, 2H), 4.35 (dd, J=10.6, 7.4 Hz, 1H), 4.30-4.25 (m, 1H), 4.18-4.13 (m, 1H), 3.57 (dd, J=8.6, 6.3 Hz, 1H), 3.50 (dd, J=8.7, 6.2 Hz, 1H), 3.35 (dd, J=8.9, 3.3 Hz, 1H), 3.27 (dd, J=8.7, 3.2 Hz, 1H), 2.81 (dq, J=7.5, 7.5 Hz, 1H), 2.73-2.67 (m, 3H), 2.66-2.59 (m, 3H), 2.19 (dd, J=8.8, 3.9 Hz, 1H), 2.15 (dd, J=8.5, 3.9 Hz, 1H), 1.63 (s, 3H), 0.70 (d, J=6.6 Hz, 3H) ppm. ESI-MS m/z calc. 586.22144, found 587.5 (M+1)⁺; 585.4 (M−1)⁻; Retention time: 3.04 minutes.

### Preparation of Intermediates

### Intermediate A

### Rac-6-bromo-2,3-dihydrofuro[3,2-b]pyridin-3-ol

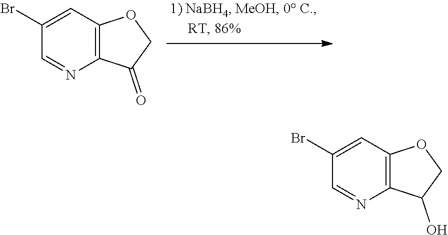 [SMILES: O=C1COc2cc(Br)cnc21 ; OC1COc2cc(Br)cnc21]

NaBH₄ (113 mg, 2.987 mmol) was added portionwise to a suspension of 6-bromofuro[3,2-b]pyridin-3-one (Hydrochloride salt) (300 mg, 1.198 mmol) in MeOH (5.0 mL) cooled to 0° C. The reaction mixture was allowed to warm to ambient temperature and stirred for 2 h. The reaction mixture was poured over water (15 mL) and diluted with EtOAc (15 mL). The organic layer was separated and the aqueous layer was extracted with EtOAc (2×15 mL). The organic layers were combined, washed with brine (20 mL), dried (MgSO₄), filtered and concentrated in vacuo to give rac-6-bromo-2,3-dihydrofuro[3,2-b]pyridin-3-ol (223 mg, 86%) as an orange solid. ¹H NMR (500 MHz, DMSO-d₆) δ 8.21 (d, J=1.9 Hz, 1H), 7.62 (d, J=1.9 Hz, 1H), 5.95 (d, J=5.7 Hz, 1H), 5.16-5.12 (m, 1H), 4.68 (dd, J=10.4, 7.0 Hz, 1H), 4.36 (dd, J=10.4, 3.0 Hz, 1H) ppm. ESI-MS m/z calc. 214.95819, found 216.1 (M+1)⁺; Retention time: 1.57 minutes.

### Intermediate B

### Rac-3-bromo-7-((tert-butyldimethylsilyl)oxy)-6,7-dihydro-5H-cyclopenta[b]pyridine

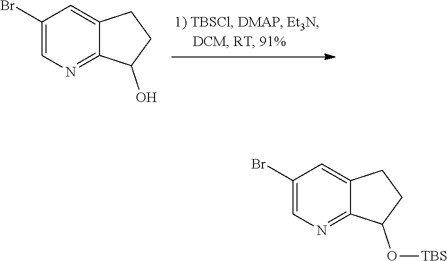

### Step 1:

TBSCl (141 mg, 0.93 mmol) and DMAP (11 mg, 0.09 mmol) were added to a solution of rac-3-bromo-6,7-dihydro-5H-cyclopenta[b]pyridin-7-ol (100 mg, 0.467 mmol) and Et₃N (130 μL, 0.93 mmol) in DCM (2.0 mL). The reaction mixture was left to stir at ambient temperature for 3 h. The reaction mixture was diluted with DCM (20 mL) and poured over a saturated aqueous NaHCO₃ solution (20 mL). The organic layer was separated and the aqueous layer was extracted with DCM (2×15 mL). The organic layers were combined, washed with brine (20 mL), dried (MgSO₄), filtered and concentrated in vacuo. Purification by flash chromatography (SiO₂, 0 to 100% EtOAc in heptane) gave rac-(3-bromo-6,7-dihydro-5H-cyclopenta[b]pyridin-7-yl)oxy-tert-butyl-dimethyl-silane (140.0 mg, 91%) as a colourless oil. ¹H NMR (500 MHz, Chloroform-d) δ 8.50-8.49 (m, 1H), 7.65-7.64 (m, 1H), 5.10 (dd, J=6.9, 4.9 Hz, 1H), 3.07-2.99 (m, 1H), 2.79-2.71 (m, 1H), 2.44-2.37 (m, 1H), 2.06-1.99 (m, 1H), 0.92 (s, 9H), 0.20 (s, 3H), 0.15 (s, 3H) ppm. ESI-MS m/z calc. 327.0654, found 328.4 (M+1)⁺; Retention time: 4.15 minutes.

### Intermediate C

### 6-(((tert-butyldimethylsilyl)oxy)methyl)pyridin-3-amine

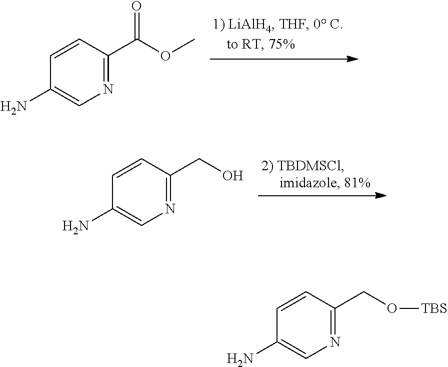

### Step 1:

Lithium aluminium hydride (120 mL of 2 M in toluene, 240 mmol) was added to a stirred suspension of methyl 5-aminopicolinate (21.05 g, 138.35 mmol) in THF (400 mL) at 0° C. under argon. The reaction mixture was stirred at ambient temperature overnight then heated at 90° C. for 6 h. The reaction mixture was then was cooled back down to 0° C. The reaction mixture was quenched by successive addition of water (9.3 mL, dropwise), 15% NaOH in water (9.3 mL) and then more water (28 mL). A white precipitate was filtered off, washing with additional THF (300 mL). The filtrate was concentrated in vacuo to give (5-aminopyridin-2-yl)methanol (16.1 g, 75%) as a brown oil, which was used in the next step without additional purification. ¹H NMR (400 MHz, DMSO-d₆) δ 7.81 (d, J=2.7 Hz, 1H), 7.06 (d, J=8.2 Hz, 1H), 6.89 (dd, J=8.5, 2.5 Hz, 1H), 5.11 (s, 2H), 4.34 (s, 2H) ppm; alcohol OH not observed.

### Step 2:

Imidazole (1.97 g, 28.938 mmol) and tert-butylchlorodimethylsilane (3.41 g, 22.624 mmol) were added to a solution of (5-aminopyridin-2-yl)methanol (3.65 g, 18.641 mmol) in THF (60 mL). The reaction mixture was stirred at ambient temperature for 17 h. The THF layer was decanted off and the oily lower phase was dissolved in water (20 mL) and extracted with EtOAc (2×20 mL). The combined organic phases were washed with brine (10 mL), dried (Na₂SO₄), filtered and concentrated in vacuo. The oily residue (5.8 g) was taken up in a 1:1 mixture of EtOAc and heptane (30 mL). The precipitate was removed by filtration. The filtrate was concentrated in vacuo. Purification by flash chromatography (SiO₂, 25 to 75% EtOAc in heptane) gave 6-(((tert-butyldimethylsilyl)oxy) methyl)pyridin-3-amine (3.92 g, 81%) as a low-melting white solid. ¹H NMR (400 MHz, Chloroform-d) δ 8.00 (d, J=2.7 Hz, 1H), 7.27-7.25 (d, 1H), 7.02 (d, J=2.7 Hz, 1H), 4.72 (s, 2H), 3.82-2.92 (br s, 2H), 0.93 (s, 9H), 0.08 (s, 6H) ppm. ESI-MS m/z calc. 238.1501, found 239.5 (M+1)⁺; Retention time: 0.86 minutes.

### Intermediate D

### [(2S)-2-methoxypropyl] methanesulfonate

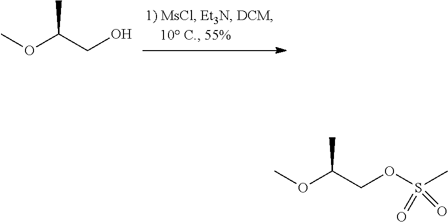

### Step 1:

Mesyl chloride (444 mg, 0.3 mL, 3.876 mmol) was slowly added to a solution of (2S)-2-methoxypropan-1-ol (250 mg, 2.7740 mmol) and Et₃N (435 mg, 0.6 mL, 4.305 mmol) in DCM (4 mL) at 0° C. The reaction mixture was stirred at 10° C. for 1 h and then partitioned between DCM (15 mL) and water (5 mL). The organic layer was dried (MgSO₄), filtered and concentrated in vacuo to give [(2S)-2-methoxypropyl] methanesulfonate (285 mg, 55%) as a yellow oil. ¹H NMR (400 MHz, Chloroform-d) δ 4.21 (dd, J=10.8, 3.4 Hz, 1H), 4.12 (q, J=5.6 Hz, 1H), 3.62 (td, J=6.3, 3.5 Hz, 1H), 3.39 (s, 3H), 3.04 (s, 3H), 1.19 (d, J=6.4 Hz, 3H) ppm.

The following intermediate was prepared using the method described for Intermediate D, Step 1 except that (R)-2-methoxypropan-1-ol was used as the starting material:

| Compound Name | NMR (shifts in ppm) |
|---|---|
| (R)-2-methoxypropyl | ¹H NMR (400 MHz, Chloroform-d) δ 4.21 (dd, |
| methanesulfonate | J = 11.0, 3.2 Hz, 1H), 4.12 (q, J = 5.6 Hz, 1H), |
| | 3.61 (td, J = 6.3, 3.5 Hz, 1H), 3.38 (t, J = 1.8 Hz, |
| | 3H), 3.02 (d, J = 16.5 Hz, 3H), 1.19 (d, J = 6.4 |
| | Hz, 3H) ppm. |

The following intermediate was prepared using the method described for Intermediate D, Step 1 except that 2-oxaspiro[3.3]heptan-6-ol was used as the starting material:

| Compound Name | NMR (shifts in ppm) |
|---|---|
| 2-oxaspiro[3.3]heptan-6-yl | ¹H NMR (400 MHz, Chloroform-d) δ |
| methanesulfonate | 4.86-4.79 (m, 1H), 4.68 (s, 2H), 4.66 (s, |
| | 2H), 2.97 (s, 3H), 2.80-2.75 (m, 2H), |
| | 2.50-2.45 (m, 2H) ppm. |

### Intermediate E

### rel-2-(2,2-dimethyl-1,3-dioxolan-4-yl)pyrimidin-5-amine and rel-2-(2,2-dimethyl-1,3-dioxolan-4-yl)pyrimidin-5-amine

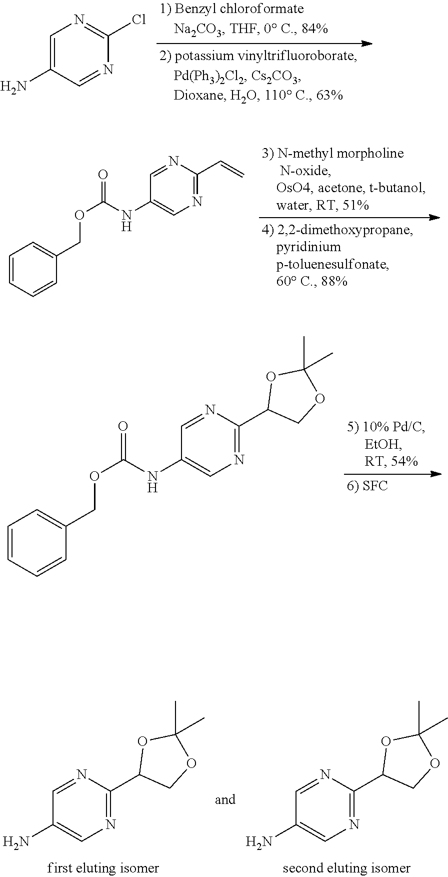 [SMILES: Nc1cnc(Cl)nc1 ; C=Cc1ncc(NC(=O)OCc2ccccc2)cn1 ; CC1(C)OCC(c2ncc(NC(=O)OCc3ccccc3)cn2)O1 ; CC1(C)OCC(c2ncc(N)cn2)O1 ; CC1(C)OCC(c2ncc(N)cn2)O1]

### Step 1:

Benzyl chloroformate (50.820 g, 42 mL, 297.90 mmol) was added via addition funnel to a solution of 2-chloropyrimidin-5-amine (50 g, 385.96 mmol) and Na₂CO₃ (120 g, 1.1322 mol) in THF (1 L) cooled to 0° C. The reaction mixture was stirred at ambient temperature overnight. The reaction mixture was concentrated and diluted with EtOAc (300 mL) and water (500 mL). The layers were separated and the aqueous phase was extracted with EtOAc (3×300 mL). The combined organic layers were dried (Na₂SO₄), filtered and concentrated in vacuo. The residue was triturated with 20% DCM in hexanes. The solid was filtered and dried in vacuo to give benzyl N-(2-chloropyrimidin-5-yl)carbamate (75.9 g, 75%) as a yellowish solid. ESI-MS m/z calc. 263.0462, found 264.1 (M+1)⁺; Retention time: 3.6 minutes.

### Step 2:

A 2-necked round-bottomed flask was charged with a mixture of benzyl N-(2-chloropyrimidin-5-yl)carbamate (10 g, 37.925 mmol), potassium vinyltrifluoroborate (7.6 g, 56.738 mmol) and Cs₂CO₃ (37 g, 113.56 mmol) in 1,4-dioxane (60 mL) and water (60 mL). A reflux condenser was added and the setup was degassed and purged with nitrogen gas. Nitrogen was bubbled through the mixture for 5 mins and then bis(triphenylphosphine)palladium(II) chloride (2.7 g, 3.8467 mmol) was added. The reaction mixture was heated at 110° C. overnight. The reaction mixture was concentrated in vacuo and then partitioned between EtOAc and water. The layers were separated and the aqueous phase was extracted with EtOAc. The combined organic layers were dried (Na₂SO₄), filtered and concentrated in vacuo. Purification by flash chromatography (330 g SiO₂, 0 to 35% EtOAc in heptane) gave benzyl N-(2-vinylpyrimidin-5-yl)carbamate (6.75 g, 63%) as beige coloured solid. ESI-MS m/z calc. 255.1008, found 256.4 (M+1)⁺; Retention time: 2.38 minutes.

### Step 3:

To a solution of benzyl N-(2-vinylpyrimidin-5-yl)carbamate (15.61 g, 61.150 mmol) in acetone (950 mL) and water (150 mL), was added successively N-methyl morpholine N-oxide (8 g, 68.29 mmol) and osmium tetroxide in tert-butanol (14 mL of 2.5% w/w, 1.376 mmol). The reaction mixture was stirred at ambient temperature overnight. The reaction mixture was concentrated in vacuo to remove acetone and the residue was poured over a saturated aqueous NH₄Cl solution (200 mL) and extracted with EtOAc (3×200 mL). The combined organic layer was washed with brine, dried (Na₂SO₄), filtered and concentrated in vacuo. The residue was dissolved in DCM and the precipitate was filtered and concentrated in vacuo to give benzyl N-[2-(1,2-dihydroxyethyl)pyrimidin-5-yl]carbamate (12.73 g, 72%) as grey solid. ESI-MS m/z calc. 289.1063, found 289.8 (M+1)⁺; Retention time: 2.53 minutes.

### Step 4:

Pyridinium p-toluenesulfonate (2.23 g, 8.8738 mmol) was added to a mixture of benzyl N-[2-(1,2-dihydroxyethyl)pyrimidin-5-yl]carbamate (12.72 g, 43.970 mmol) and 2,2-dimethoxypropane (80.465 g, 95 mL, 772.60 mmol) and the reaction mixture was stirred for 24 h at 60° C. The reaction mixture diluted with water (200 mL) and extracted with EtOAc (3×150 mL). The combined organic layers were dried (Na₂SO₄), filtered and concentrated in vacuo. Purification by flash chromatography (80 g SiO₂, 0 to 35% EtOAc in heptane) gave benzyl rac-N-[2-(2,2-dimethyl-1,3-dioxolan-4-yl)pyrimidin-5-yl]carbamate (12.72 g, 88%) as a white solid. ESI-MS m/z calc. 329.1376, found 330.1 (M+1)⁺; Retention time: 2.42 minutes.

### Step 5:

10% Pd/C (4.13 g, 3.88 mol) was added to a solution of benzyl rac-N-[2-(4,4-dimethyl-1,3-dioxolan-2-yl)pyrimidin-5-yl]carbamate (12.72 g, 38.622 mmol) in EtOH (500 mL) under nitrogen. The reaction flask was degassed and purged with nitrogen. The reaction was stirred a under hydrogen atmosphere for 5 h. The reaction mixture was then filtered over a pad of celite and the filtrate was concentrated in vacuo. The white solid was washed with excess diethyl ether, dried in vacuo to give rac-2-(2,2-dimethyl-1,3-dioxolan-4-yl)pyrimidin-5-amine (4.4 g, 54%). ¹H NMR (500 MHz, DMSO-d₆) δ 8.10 (s, 2H), 5.61 (s, 2H), 4.96 (dd, J=7.7, 6.4 Hz, 1H), 4.22 (dd, J=7.9, 6.5 Hz, 1H), 4.07 (t, J=7.8 Hz, 1H), 1.38 (s, 3H), 1.36 (s, 3H). ESI-MS m/z calc. 195.1008, found 196.3 (M+1)⁺; Retention time: 0.7 minutes.

### Step 6:

The enantiomers of rac-2-(2,2-dimethyl-1,3-dioxolan-4-yl)pyrimidin-5-amine (780 mg, 3.996 mmol) were separated by chiral SFC using a Chiralpak IB column, 5 m particle size, 25 cm×20 mm from Daicel Corporation on a Prep-100 SFC instrument from Waters to give two isomers:

First Eluting Isomer (rt=2.01 min): rel-2-(2,2-dimethyl-1,3-dioxolan-4-yl)pyrimidin-5-amine (321.9 mg, 41%). ESI-MS m/z calc. 195.10078, found 196.1 (M+1)⁺; Retention time: 0.35 minutes.

Second Eluting Isomer (rt=2.25 min): rel-2-(2,2-dimethyl-1,3-dioxolan-4-yl)pyrimidin-5-amine (381.3 mg, 49%). ESI-MS m/z calc. 195.10078, found 196.1 (M+1)⁺; Retention time: 0.35 minutes.

### Intermediate F

### Rac-6-(1-((tert-butyldimethylsilyl)oxy)-2-methoxyethyl)pyridin-3-amine

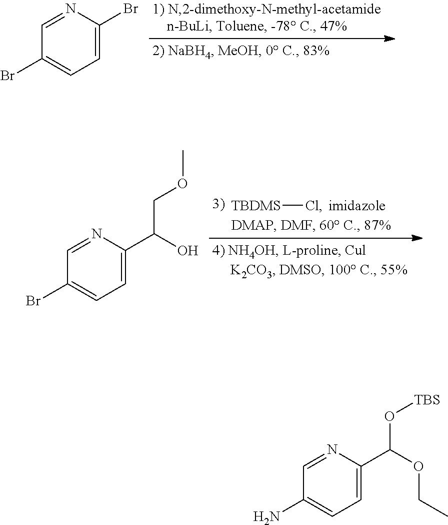

### Step 1:

n-BuLi (50 mL of 2 M, 100.00 mmol) was added to a stirred solution of 2,5-dibromopyridine (20 g, 84.427 mmol) in toluene (600 mL) at −78° C. and the reaction mixture was stirred for 45 min at −78° C. A solution of N,2-dimethoxy-N-methyl-acetamide (13.5 g, 101.39 mmol) in toluene (100 mL) was added to the reaction mixture at −78° C. The mixture was stirred for 30 min at −78° C. The reaction mixture was then poured over a saturated aqueous NH₄Cl solution (300 mL) and extracted with EtOAc (2×500 mL). The combined organic layer was washed with brine (300 mL), dried (Na₂SO₄), filtered and concentrated in vacuo. Purification by flash chromatography (80 g SiO₂, 10 to 15% EtOAc in heptane) gave 1-(5-bromo-2-pyridyl)-2-methoxy-ethanone (9.1 g, 47%). ¹H NMR (400 MHz, DMSO-d₆) δ 8.84 (s, 1H), 8.30-8.28 (m, 1H), 7.88 (d, J=8.3 Hz, 1H), 4.94 (s, 2H), 3.38 (s, 3H) ppm. ESI-MS m/z calc. 228.9738, found 200.0 (M−30)⁺; Retention time: 1.63 minutes.

### Step 2:

Sodium borohydride (1.5 g, 39.648 mmol) was added to a stirred solution of 1-(5-bromo-2-pyridyl)-2-methoxy-ethanone (9 g, 39.120 mmol) in MeOH (100 mL) cooled to 0° C. and the reaction mixture was stirred at ambient temperature for 30 min. The reaction mixture was quenched with water (100 mL) and concentrated in vacuo. The residue was diluted with water (100 mL) and extracted with EtOAc (2×200 mL). The combined extracts were washed with brine (100 mL), dried (Na₂SO₄), filtered and concentrated in vacuo. Purification by flash chromatography (80 g SiO₂, 40 to 60% EtOAc in heptane) gave rac-1-(5-bromo-2-pyridyl)-2-methoxy-ethanol (7.5 g, 83%) as light yellow liquid. ¹H NMR (400 MHz, DMSO-d₆) δ 8.61 (s, 1H), 8.02 (d, J=7.6 Hz, 1H), 7.48 (d, J=7.8 Hz, 1H), 5.64 (s, 1H), 4.71 (s, 1H), 3.61-3.59 (m, 1H), 3.50-3.49 (m, 1H), 3.24 (s, 3H) ppm. ESI-MS m/z calc. 230.9895, found 233.9 (M+2)⁺; Retention time: 1.46 minutes.

### Step 3:

To a stirred solution of rac-1-(5-bromo-2-pyridyl)-2-methoxy-ethanol (7 g, 30.163 mmol) in DMF (50 mL) was added tert-butyldimethylsilyl chloride (7 g, 46.443 mmol), imidazole (6 g, 88.135 mmol) and DMAP (730 mg, 5.9754 mmol) and the reaction mixture was heated at 60° C. for 16 h. The reaction mixture was quenched with ice cold water (500 mL) and extracted with EtOAc (2×300 mL). The combined organic extracts were washed with water (2×100 mL) followed by brine (100 mL), dried (Na₂SO₄), filtered and concentrated in vacuo. Purification by flash chromatography (80 g SiO₂, 5 to 10% EtOAc in heptane) gave rac-[1-(5-bromo-2-pyridyl)-2-methoxy-ethoxy]-tert-butyl-dimethyl-silane (9.1 g, 87%). ¹H NMR (400 MHz, DMSO-d₆) δ 8.61 (s, 1H), 8.63 (d, J=2.2 Hz, 1H), 8.07 (dd, J=2.3 Hz, 8.4 Hz, 1H), 7.45 (d, J=8.4 Hz, 1H), 4.88-4.85 (m, 1H), 3.58-3.54 (m, 1H), 3.48-3.44 (m, 1H), 3.25 (s, 3H), 0.85 (d, J=8.9 Hz, 9H), 0.07 (s, 3H), −0.02 (s, 3H) ppm. ESI-MS m/z calc. 345.076, found 348.0 (M+2)⁺; Retention time: 2.27 minutes.

### Step 4:

To a stirred solution of rac-[1-(5-bromo-2-pyridyl)-2-methoxy-ethoxy]-tert-butyl-dimethyl-silane (9 g, 25.986 mmol) in DMSO (50 mL) in a seal tube was added K₂CO₃ (5.5 g, 39.79 mmol), L-proline (1.2 g, 10.42 mmol), CuI (1 g, 5.25 mmol) and NH₄OH (25%) (2 mL of 25% w/v aqueous solution, 14.2 mmol). The reaction mixture was heated at 100° C. for 16 h. The reaction mixture was then quenched with cold water (500 mL) and extracted with EtOAc (2×200 mL). The combined organic extracts were washed with water (2×100 mL) followed by brine (100 mL), dried (Na₂SO₄), filtered and concentrated in vacuo. Purification by flash chromatography (SiO₂, 5 to 10% EtOAc in heptane) gave rac-6-[1-[tert-butyl(dimethyl)silyl]oxy-2-methoxy-ethyl]pyridin-3-amine (4.07 g, 55%) as brown sticky solid. ¹H NMR (400 MHz, DMSO-d₆) δ 7.83 (d, J=2.5, 1H), 7.10-7.08 (m, 1H), 6.92-6.89 (m, 1H), 5.20-5.18 (m, 2H), 4.72-4.69 (m, 1H), 3.49-3.46 (m, 1H), 3.37-3.33 (m, 1H), 3.25 (s, 3H), 0.84 (s, 9H), 0.02 (s, 3H), −0.07 (s, 3H) ppm. ESI-MS m/z calc. 282.1764, found 283.2 (M+1)⁺; Retention time: 2.21 minutes.

### Example 12

### E-VIPR Assay Detecting and Measuring Na_{V} Inhibition Properties

Sodium ion channels are voltage-dependent proteins that can be activated by inducing membrane voltage changes by applying electric fields. The electrical stimulation instrument and methods of use, referred to as E-VIPR, are described in International Publication No. WO 2002/008748 A3 and C.-J. Huang et al. Characterization of voltage-gated sodium channel blockers by electrical stimulation and fluorescence detection of membrane potential, 24 Nature Biotech. 439-46 (2006), both of which are incorporated by reference in their entirety. The instrument comprises a microtiter plate handler, an optical system for exciting the coumarin dye while simultaneously recording the coumarin and oxonol emissions, a waveform generator, a current- or voltage-controlled amplifier, and parallel electrode pairs that are inserted into assay plate wells. Under integrated computer control, this instrument passes user-programmed electrical stimulus protocols to cells within the wells of the microtiter plate.

16-20 hours prior to running the assay on E-VIPR, HEK cells expressing a truncated form of human Na_{V} 1.8 with full channel activity were seeded into microtiter 384-well plates, pre-coated with matrigel, at a density of 25,000 cells per well. 2.5-5% KIR2.1 Bacmam virus was added to the final cell suspension before seeding into cell plates. HEK cells were grown in Dulbecco's Modified Eagle's Medium (DMEM) supplemented with 10% FBS (Fetal Bovine Serum, qualified; Sigma #F4135), 1% NEAA (Non-Essential Amino Acids, Gibco #11140), 1% HEPES (Gibco #15630), 1% Pen-Strep (Penicillin-Streptomycin; Gibco #15140) and 5 μg/ml Blasticidin (Gibco #R210-01). Cells were expanded in vented cap cell culture flasks, with 90-95% humidity and 5% CO₂.

### Reagents and Stock Solutions:

100 mg/mL Pluronic F-127 (Sigma #P2443), in dry DMSO
Compound Plates: Corning 384-well Polypropylene Round Bottom #3656
Cell Plates: 384-well tissue culture treated plates (Greiner #781091-2B)

2.5-5% KIR 2.1 Bacmam virus (produced in-house), prepared as described in Section 3.3 of J. A. Fornwald et al., Gene Expression in Mammalian Cells Using BacMam, a Modified Baculovirus System, 1350 Methods in Molecular Biology 95-116 (2016), the entire contents of which are incorporated by reference. The concentration used can be dependent on viral titer of each batch.

5 mM DiSBAC₆(3), a voltage sensitive oxonol acceptor (CAS number 169211-44-3; 5-[3-(1,3-dihexylhexahydro-4,6-dioxo-2-thioxo-5-pyrimidinyl)-2-propen-1-ylidene]-1,3-dihexyldihydro-2-thioxo-4,6(1H,5H)-pyrimidinedione), in dry DMSO. The preparation of DiSBAC₆(3) is analogous to that of DiSBAC₄(3) as described in Voltage Sensing by Fluorescence Resonance Energy Transfer in Single Cells, Gonzalez, J. E. and Tsien, R. Y. (1995) Biophys. J. 69, 1272-1280.

5 mM CC2-DMPE, a commercially available membrane-bound coumarin phospholipid FRET donor (ThermoFisher Scientific catalog number K1017, CAS number 393782-57-5; tetradecanoic acid, 1,1′-[(1R)-1-[8-(6-chloro-7-hydroxy-2-oxo-2H-1-benzopyran-3-yl)-3-hydroxy-3-oxido-8-oxo-2,4-dioxa-7-aza-3-phosphaoct-1-yl]-1,2-ethanediyl] ester) was prepared in dry DMSO. See also, Improved indicators of cell membrane potential that use fluorescence resonance energy transfer, Gonzalez, J. E. and Tsien, R. Y. (1997) Chem. Biol. 4, 269-277.

Voltage Assay Background Suppression Compound (VABSC-1) is prepared in H₂O (89-363 mM, range used to maintain solubility)

Human Serum (HS, Millipore #S1P1-01KL, or Sigma SLBR5469V and SLBR5470V as a 50%/50% mixture, for 25% assay final concentration)

### Bath 1 Buffer:

Sodium Chloride 160 mM (9.35 g/L), Potassium Chloride, 4.5 mM (0.335 g/L), Glucose
10 mM (1.8 g/L), Magnesium Chloride (Anhydrous) 1 mM (0.095 g/L), Calcium
Chloride 2 mM (0.222 g/L), HEPES 10 mM (2.38 g/L) in water.

### Na/TMA C1 Bath 1 Buffer:

Sodium Chloride 96 mM (5.61 g/L), Potassium Chloride 4.5 mM (0.335 g/L),
Tetramethylammonium (TMA)-Cl 64 mM (7.01 g/L), Glucose 10 mM (1.8 g/L),
Magnesium Chloride (Anhydrous) 1 mM (0.095 g/L), Calcium Chloride 2 mM (0.222
g/L) HEPES 10 mM (2.38 g/L) in water.

### Hexyl Dye Solution (2× Concentration):

Bath 1 Buffer containing 0.5% β-cyclodextrin (made fresh prior to each use, Sigma #C₄₇₆₇), 8 μM CC2-DMPE and 2 μM DiSBAC₆(3). The solution was made by adding 10% Pluronic F127 stock equal to combined volumes of CC2-DMPE and DiSBAC₆(3). The order of preparation was first mix Pluronic and CC2-DMPE, then add DiSBAC₆(3), then while vortexing add Bath 1/β-Cyclodextrin.

Compound Loading Buffer (2× concentration): Na/TMA Cl Bath1 Buffer containing HS (omitted in experiments run in the absence of human serum (HS))50%, VABSC-1 1 mM, BSA 0.2 mg/ml (in Bath-1), KCl 9 mM, DMSO 0.625%.

### Assay Protocol (7 Key Steps):

1) To reach the final concentration in each well, 375 nL of each compound was pre-spotted (in neat DMSO) into polypropylene compound plates at 240× desired final concentration from an intermediate stock concentration of 0.075 mM, in an 11 point dose response, 3-fold dilution, resulting in a top dose of 300 nM final concentration in the cell plate. Vehicle control (neat DMSO), and positive control (an established Na_{V}1.8 inhibitor, 25 μM final in assay in DMSO) were added manually to the outermost columns of each plate respectively. The compound plate was backfilled with 45 μL per well of Compound Loading Buffer resulting in a 240 fold dilution of compound following a 1:1 transfer of compound into the cell plate (see Step 6). Final DMSO concentration for all wells in the assay was 0.625% (0.75% DMSO was supplemented to the Compound Loading Buffer for a final DMSO concentration of 0.625%). This assay dilution protocol was adjusted to enable a higher dose range to be tested in the presence of HS or if the final assay volume was altered.
2) Hexyl Dye Solution was prepared.
3) Cell plates were prepared. On the day of the assay, the media was aspirated, and the cells were washed three times with 80 μL of Bath-1 buffer, maintaining 25 μL residual volume in each well.
4) 25 μL per well of Hexyl Dye Solution was dispensed into the cell plates. The cells were incubated for 20 minutes at room temperature or ambient conditions in darkness.
5) 45 μL per well of Compound Loading Buffer was dispensed into compound plates.
6) The cell plates were washed three times with 80 μL per well of Bath-1 Buffer, leaving 25 L of residual volume. Then 25 μL per well from compound plate was transferred to each cell plate. The mixture was incubated for 30 minutes at room temperature/ambient conditions.
7) The cell plate containing compound was read on E-VIPR using the current-controlled amplifier to deliver stimulation wave pulses using a symmetrical biphasic waveform. The user-programmed electrical stimulus protocols were 1.25-4 Amps and 4 millisecond pulse width (dependent on electrode composition) were delivered at 10 Hz for 10 seconds. A pre-stimulus recording was performed for each well for 0.5 seconds to obtain the un-stimulated intensities baseline. The stimulatory waveform was followed by 0.5 seconds of post-stimulation recording to examine the relaxation to the resting state. All E-VIPR responses were measured at 200 Hz acquisition rate.

### Data Analysis:

Data were analyzed and reported as normalized ratios of emission intensities measured in the 460 nm and 580 nm channels. The response as a function of time was reported as the ratios obtained using the following formula:

$$R ( t ) = \frac{( {intensity}_{460 nm} )}{( {intensity}_{580 nm} )}$$

The data were further reduced (i.e. normalized) by calculating the initial (Rᵢ) and final (R_{f}) ratios. These were the average ratio values during part or all of the pre-stimulation period and during sample points during the stimulation period. The fluorescence ratio (R_{f}/Rᵢ) was then calculated and reported as a function of time.

Control responses were obtained by performing assays in the presence of the positive control, and in the absence of pharmacological agents (DMSO vehicle negative control). Responses to the negative (N) and positive (P) controls were calculated as above. The compound antagonist % activity A was then defined as:

$$A = \frac{X - N}{P - N} \times 100$$

where X is the ratio response of the test compound (i.e. the maximum amplitude of the ratio response or number of action potential peaks, at the beginning of the pulse train in the presence of test compound). Using this analysis protocol, dose response curves were plotted and IC₅₀ values were generated for various compounds of the present invention as reported below.

Compounds having a measured IC₅₀ value less than 0.01 μM in the E-VIPR Assay described above include: 10.

Compounds having a measured IC₅₀ value less than 0.1 μM and greater than or equal to 0.01 μM in the E-VIPR Assay described above include: 1, 4, 5, 6, 7, 9, 11, 12, 14, 17, 21, 22, 23, 24, and 25.

Compounds having a measured IC₅₀ value less than 1 μM and greater than or equal to 0.1 μM in the E-VIPR Assay described above include: 2, 3, 8, and 26.

Compounds having a measured IC₅₀ value greater than or equal to 1 μM in the E-VIPR Assay described above include: 13, 15, 16, 18, 19, and 20.

Many modifications and variations of the embodiments described herein may be made without departing from the scope, as is apparent to those skilled in the art. The specific embodiments described herein are offered by way of example only.

1. A compound of formula (I) 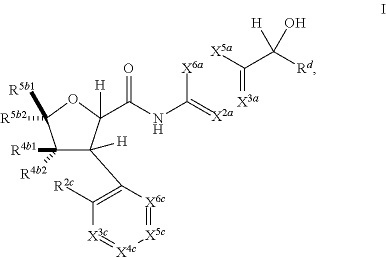
or a pharmaceutically acceptable salt thereof, wherein:
X^{2a} is N, N⁺—O⁻, or C—R^{2a};
X^{3a} is N or N⁺—O⁻;
X^{5a} is N, N⁺—O⁻, or C—R^{5a};
X^{6a} is N, N⁺—O⁻, or C—R^{6a};
R^{d} is (CH₂)ₘ(CHR^{e})ₙ(CH₂)ₚH;
m, n, and p are each independently 0 or 1;
R^{e} is H, OH, halo, C₁-C₆ alkoxy, or C₁-C₆ haloalkoxy;
R^{2a} and R^{6a} are each independently H, halo, C₁-C₆ alkyl, or C₁-C₆ haloalkyl;
R^{5a} is H, halo, CH₂OH, C₁-C₆ alkyl, C₁-C₆ haloalkyl, or R^{5a} and R^{d} form a CH₂CH₂ chain linking the C atoms to which R^{5a} and R^{d} are attached, wherein the CH₂ group that is bound to the C atom to which R^{5a} is attached may be replaced with O;
R^{4b1} and R^{4b2} are each independently H, C₁-C₆ alkyl, C₃-C₆ cycloalkyl, or C₁-C₆ haloalkyl;
R^{5b1} and R^{5b2} are each independently H, C₁-C₆ alkyl, C₃-C₆ cycloalkyl, or C₁-C₆ haloalkyl;
X^{3c} is N or C—R^{3c};
X^{4c} is N or C—R^{4c};
X^{5c} is N or C—R^{5c};
X^{6c} is N or C—R^{6c};
R^{2c} is H, OH, halo, C₁-C₆ alkyl, C₂-C₆ alkenyl, C₁-C₆ haloalkyl, C₁-C₆ alkoxy, C₁-C₆ haloalkoxy, O—CH₂—C(R^{2c1})(R^{2c2})(R^{2c3}), O—CH(R^{2c4})(R^{2c5}), or -L¹-L²-(C₃-C₆ cycloalkyl), wherein said cycloalkyl is optionally substituted with 1-2 halo;
R^{2c1} and R^{2c2} are each independently H or C₁-C₆ alkyl, or R^{2c1} and R^{2c2} together with the C atom to which they are attached form C═O;
R^{2c3} is OH, C₁-C₆ alkoxy, C₁-C₆ haloalkoxy, or N(R^{2c6})(R^{2c7}); or R^{2c2} and R^{2c3} together with the C atom to which they are attached form a 3-7 membered heterocycloalkyl;
R^{2c4} and R^{2c5} together with the C atom to which they are attached form a 3-7 membered heterocycloalkyl;
R^{2c6} and R^{2c7} are each C₁-C₆ alkyl, or R^{2c6} and R^{2c7} together with the N atom to which they are attached form a 3-8 membered heterocycloalkyl;
L¹ is a bond or O;
L² is a bond or C₁-C₆ alkylene;
R^{3c} is H, halo, C₁-C₆ alkyl, or C₁-C₆ haloalkyl; or X^{3c} is C—R^{3c}, and R^{2c} and R^{3c}, together with the carbon atoms to which they are attached, form a ring of formula: 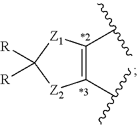
Z₁ and Z₂ are each independently O or CH₂;
each R is independently H or halo;
R^{4c} is H, halo, C₁-C₆ alkyl, C₁-C₆ haloalkyl, C₁-C₆ alkoxy, or C₁-C₆ haloalkoxy;
R^{5c} is H, halo, C₁-C₆ alkyl, or C₁-C₆ haloalkyl; and
R^{6c} is H, halo, C₁-C₆ alkyl, or C₁-C₆ haloalkyl;
provided that no more than two of X^{2a}, X^{3a}, X^{5a}, and X^{6a} are N or N⁺—O⁻; and
provided that no more than one of X³, X^{4c}, X^{5c}, and X^{6c} is N; and
provided that:
R^{1a} and R^{d} form a CH₂CH₂ chain linking the C atoms to which R^{a} and R^{d} are attached, wherein the CH₂ group that is bound to the C atom to which R^{a} is attached may be replaced with O; or
R^{2c} is O—CH₂—C(R^{2c1})(R^{2c2})(R^{2c3}) or O—CH(R^{2c4})(R^{2c}).

2. The compound of claim 1, wherein the compound has formula (I-A), (I-A-1), (I-A-2), (I-A-3), (I-B), (I-B-1), (I-B-2), (I-B-3), (I-C), (I-C-1), (I-C-2), (I-C-3), (I-D), (I-D-1), (I-D-2), (I-D-3), and (I-D-4): 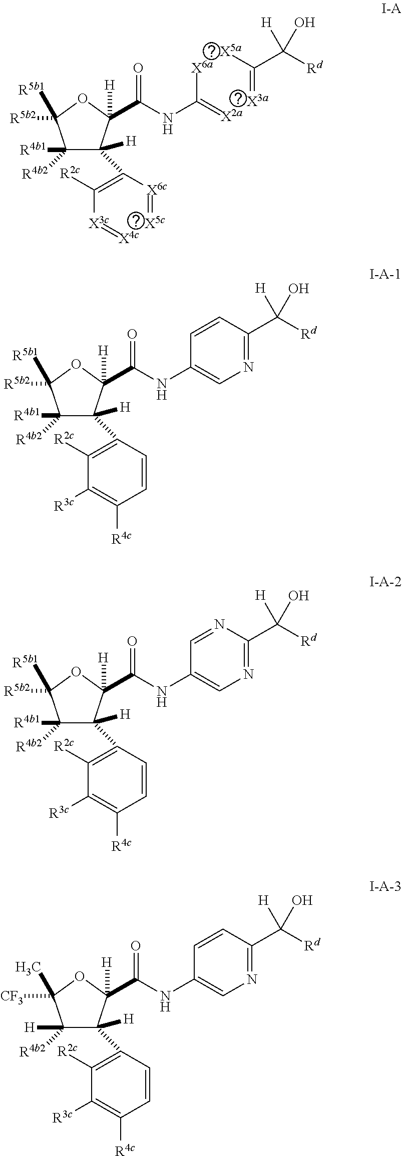 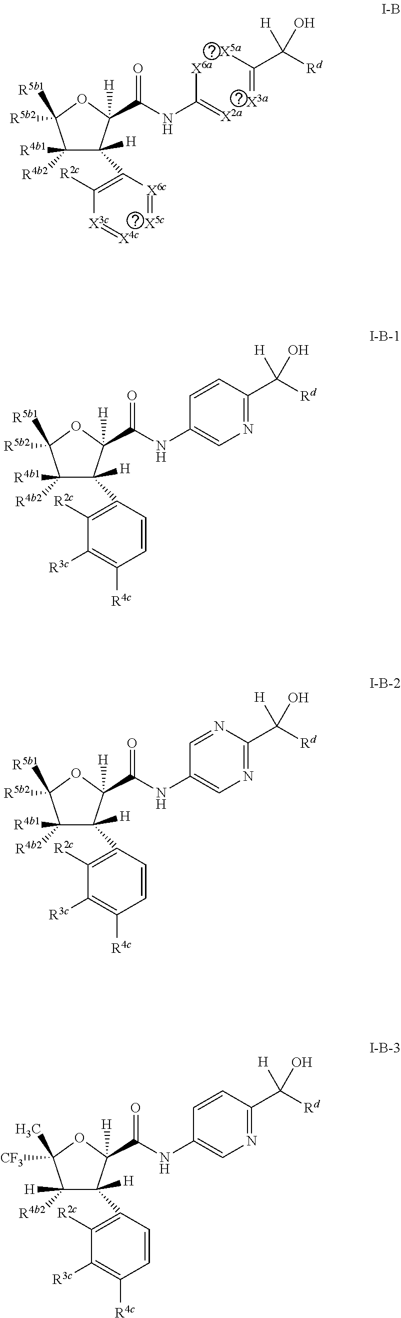 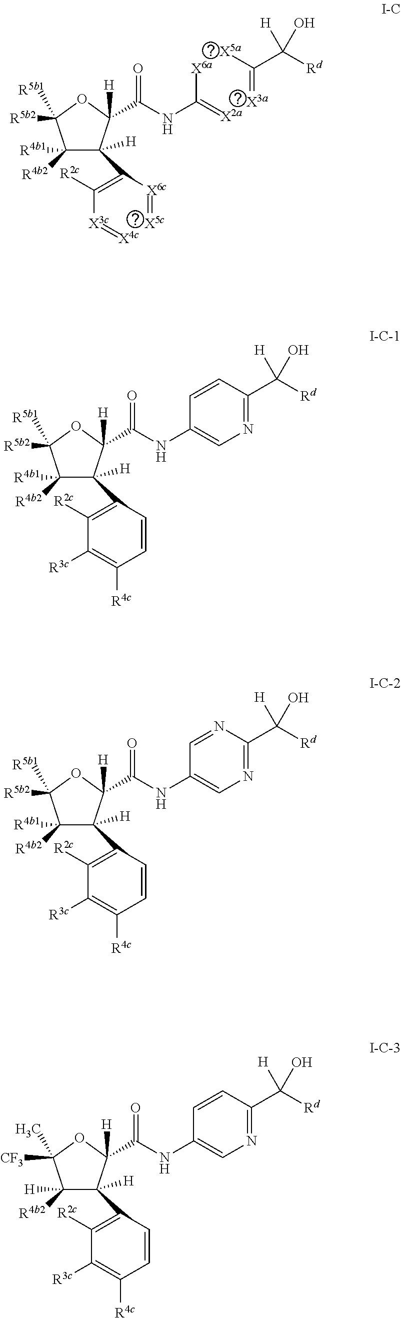 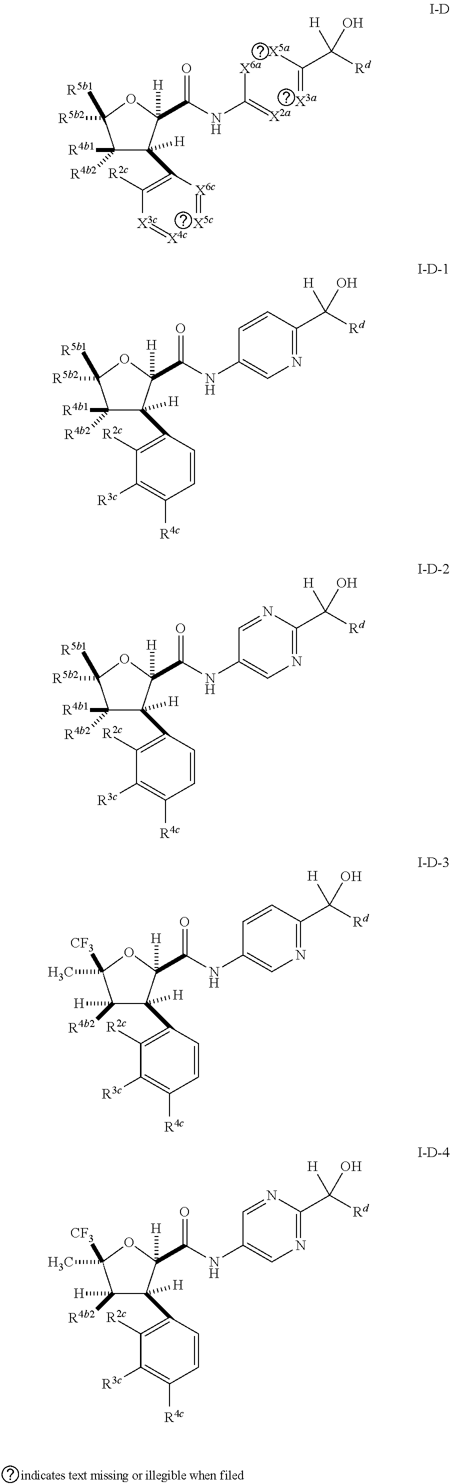
or a pharmaceutically acceptable salt thereof.

3-10. (canceled)

11. The compound of claim 1, or a pharmaceutically acceptable salt thereof, wherein X^{2a} is C—R^{2a}; and R^{2a} is H.

12. The compound of claim 1, or a pharmaceutically acceptable salt thereof, wherein X^{3a} is N.

13. The compound of claim 1, or a pharmaceutically acceptable salt thereof, wherein X^{5a} is N; or X^{5a} is C—R^{5a}; and R^{5a} is H.

14. (canceled)

15. The compound of claim 1, or a pharmaceutically acceptable salt thereof, wherein X^{6a} is C—R^{6a}; and R^{6a} is H.

16. The compound of claim 1, or a pharmaceutically acceptable salt thereof, wherein R^{e} is H, OH, or C₁-C₆ alkoxy.

17. The compound of claim 1, or a pharmaceutically acceptable salt thereof, wherein:
R^{4b1} is H or C₁-C₆ alkyl, optionally CH₃; and/or
R^{4b2} is H or C₁-C₆ alkyl, optionally CH₃.

18. (canceled)

19. The compound of claim 1, or a pharmaceutically acceptable salt thereof, wherein:
R^{5b1} is C₁-C₆ alkyl, optionally CH₃, or C₁-C₆ haloalkyl, optionally CF₃; and/or
R^{5b2} C₁-C₆ alkyl, optionally CH₃, or C₁-C₆ haloalkyl optionally CF₃.

20. (canceled)

21. The compound of claim 1, or a pharmaceutically acceptable salt thereof, wherein R^{2c} is C₁-C₆ alkoxy, optionally O—CH₂—C(R^{2c1})(R^{2c2})(R^{2c3}) or O—CH(R^{2c4})(R^{2c5}).

22. The compound of claim 1, or a pharmaceutically acceptable salt thereof, wherein X^{3c} is C—R^{3c}; and R^{3c} is halo, optionally F, or C₁-C₆ alkyl, optionally CH₃.

23. The compound of claim 1, or a pharmaceutically acceptable salt thereof, wherein X^{4c} is C—R^{4c}; and wherein R^{4c} is halo, optionally F.

24. The compound of claim 1, or a pharmaceutically acceptable salt thereof, wherein X^{5c} is C—R^{5c}; and wherein R^{e} is H.

25. The compound of claim 1, or a pharmaceutically acceptable salt thereof, wherein X^{6c} is C—R^{6c}; and wherein R^{6c} is H.

26. A compound selected from: 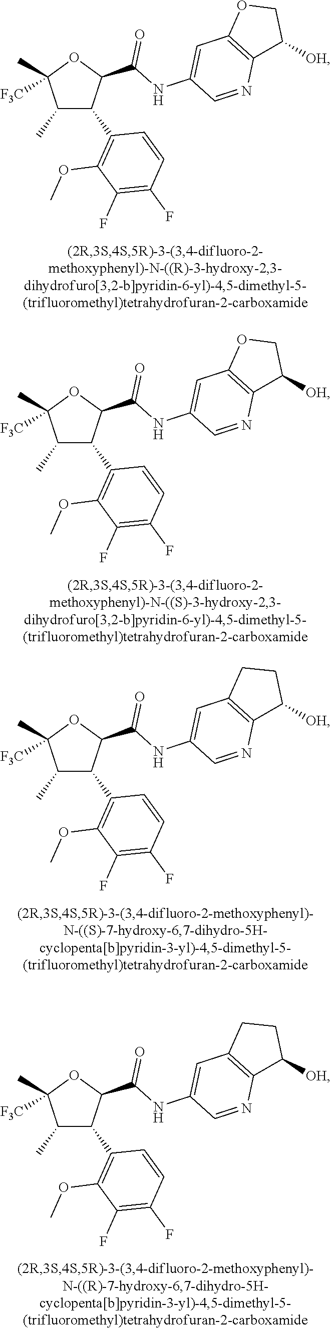 [SMILES: COc1c([C@H]2[C@H](C(=O)Nc3cnc4c(c3)OC[C@@H]4O)O[C@@](C)(C(F)(F)F)[C@H]2C)ccc(F)c1F ; COc1c([C@H]2[C@H](C(=O)Nc3cnc4c(c3)OC[C@H]4O)O[C@@](C)(C(F)(F)F)[C@H]2C)ccc(F)c1F ; COc1c([C@H]2[C@H](C(=O)Nc3cnc4c(c3)CC[C@@H]4O)O[C@@](C)(C(F)(F)F)[C@H]2C)ccc(F)c1F ; COc1c([C@H]2[C@H](C(=O)Nc3cnc4c(c3)CC[C@H]4O)O[C@@](C)(C(F)(F)F)[C@H]2C)ccc(F)c1F] 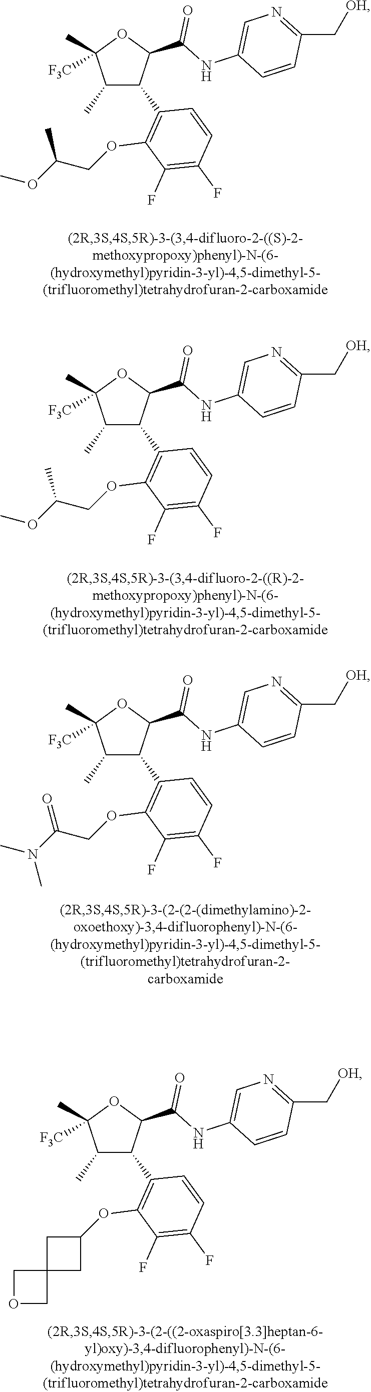 [SMILES: CO[C@@H](C)COc1c([C@H]2[C@H](C(=O)Nc3ccc(CO)nc3)O[C@@](C)(C(F)(F)F)[C@H]2C)ccc(F)c1F ; CO[C@H](C)COc1c([C@H]2[C@H](C(=O)Nc3ccc(CO)nc3)O[C@@](C)(C(F)(F)F)[C@H]2C)ccc(F)c1F ; C[C@H]1[C@@H](c2ccc(F)c(F)c2OCC(=O)N(C)C)[C@H](C(=O)Nc2ccc(CO)nc2)O[C@@]1(C)C(F)(F)F ; C[C@H]1[C@@H](c2ccc(F)c(F)c2OC2CC3(COC3)C2)[C@H](C(=O)Nc2ccc(CO)nc2)O[C@@]1(C)C(F)(F)F] 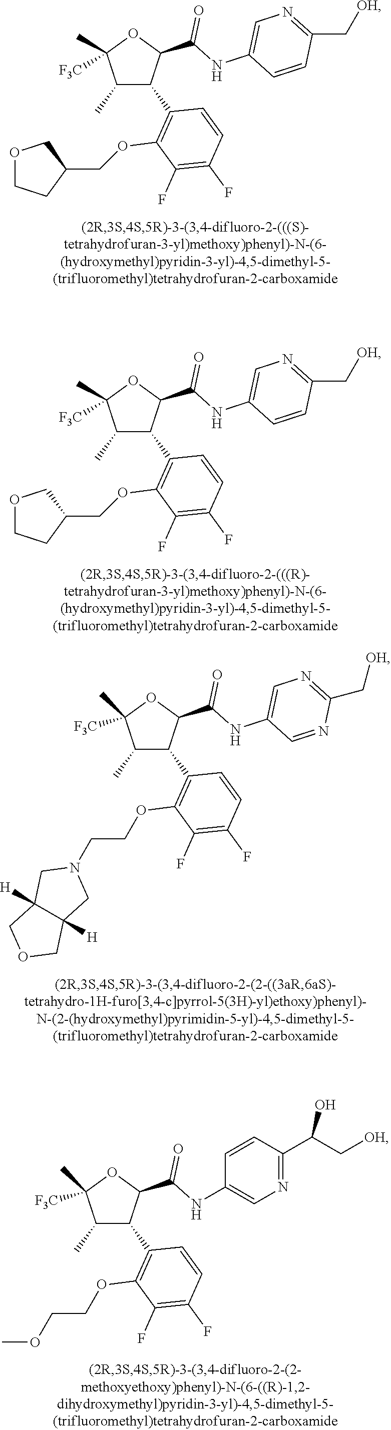 [SMILES: C[C@H]1[C@@H](c2ccc(F)c(F)c2OC[C@H]2CCOC2)[C@H](C(=O)Nc2ccc(CO)nc2)O[C@@]1(C)C(F)(F)F ; C[C@H]1[C@@H](c2ccc(F)c(F)c2OC[C@@H]2CCOC2)[C@H](C(=O)Nc2ccc(CO)nc2)O[C@@]1(C)C(F)(F)F ; C[C@H]1[C@@H](c2ccc(F)c(F)c2OCCN2C[C@H]3COC[C@H]3C2)[C@H](C(=O)Nc2cnc(CO)nc2)O[C@@]1(C)C(F)(F)F ; COCCOc1c([C@H]2[C@H](C(=O)Nc3ccc([C@@H](O)CO)nc3)O[C@@](C)(C(F)(F)F)[C@H]2C)ccc(F)c1F] 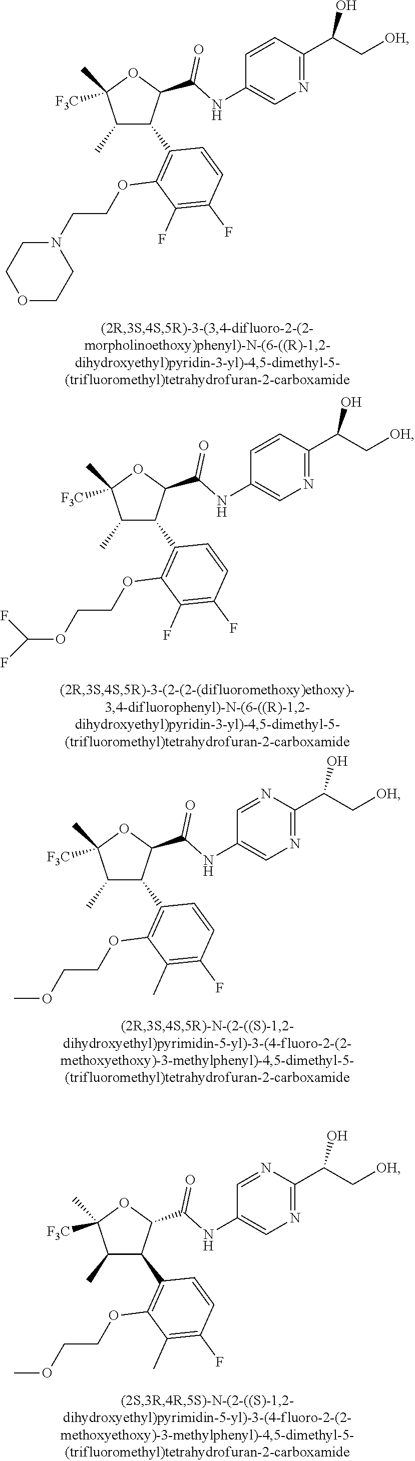 [SMILES: C[C@H]1[C@@H](c2ccc(F)c(F)c2OCCN2CCOCC2)[C@H](C(=O)Nc2ccc([C@@H](O)CO)nc2)O[C@@]1(C)C(F)(F)F ; C[C@H]1[C@@H](c2ccc(F)c(F)c2OCCOC(F)F)[C@H](C(=O)Nc2ccc([C@@H](O)CO)nc2)O[C@@]1(C)C(F)(F)F ; COCCOc1c([C@H]2[C@H](C(=O)Nc3cnc([C@H](O)CO)nc3)O[C@@](C)(C(F)(F)F)[C@H]2C)ccc(F)c1C ; COCCOc1c([C@@H]2[C@@H](C(=O)Nc3cnc([C@H](O)CO)nc3)O[C@](C)(C(F)(F)F)[C@@H]2C)ccc(F)c1C] 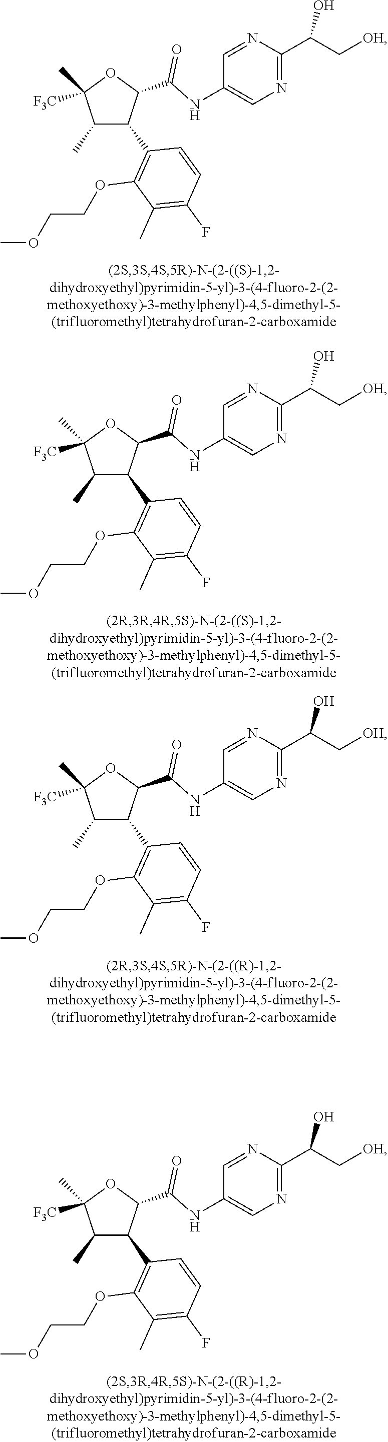 [SMILES: COCCOc1c([C@H]2[C@@H](C(=O)Nc3cnc([C@H](O)CO)nc3)O[C@@](C)(C(F)(F)F)[C@H]2C)ccc(F)c1C ; COCCOc1c([C@H]2[C@@H](C)[C@@](C)(C(F)(F)F)O[C@H]2C(=O)Nc2cnc([C@H](O)CO)nc2)ccc(F)c1C ; COCCOc1c([C@H]2[C@H](C(=O)Nc3cnc([C@@H](O)CO)nc3)O[C@@](C)(C(F)(F)F)[C@H]2C)ccc(F)c1C ; COCCOc1c([C@@H]2[C@@H](C(=O)Nc3cnc([C@@H](O)CO)nc3)O[C@](C)(C(F)(F)F)[C@@H]2C)ccc(F)c1C] 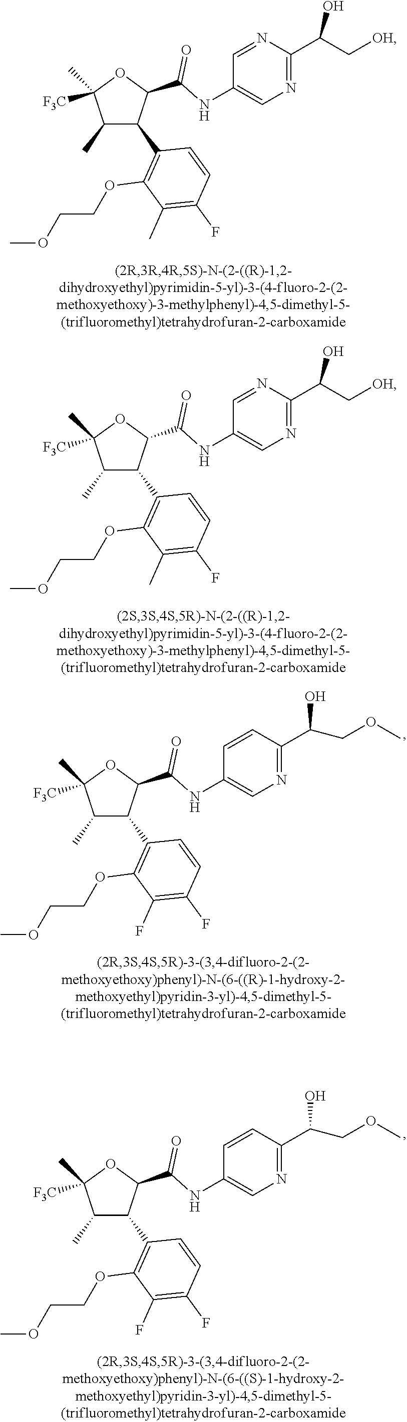 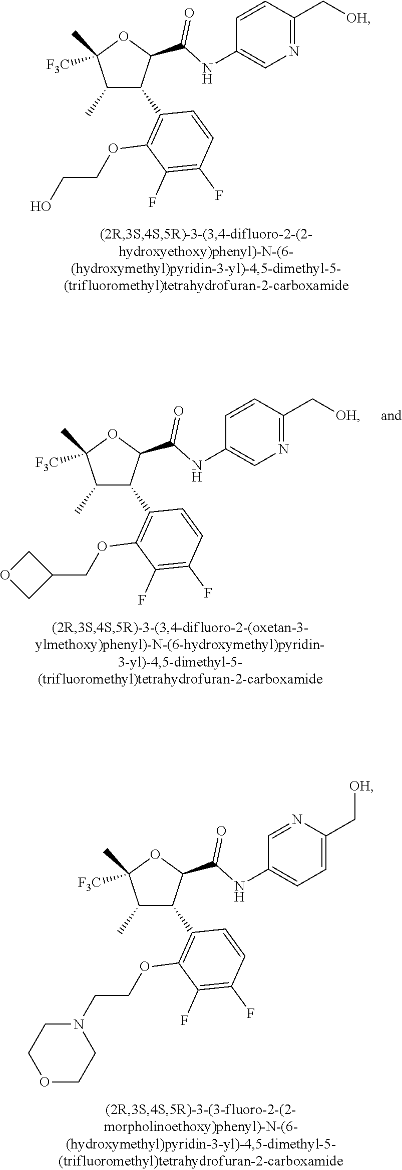 [SMILES: C[C@H]1[C@@H](c2ccc(F)c(F)c2OCCO)[C@H](C(=O)Nc2ccc(CO)nc2)O[C@@]1(C)C(F)(F)F ; C[C@H]1[C@@H](c2ccc(F)c(F)c2OCC2COC2)[C@H](C(=O)Nc2ccc(CO)nc2)O[C@@]1(C)C(F)(F)F ; C[C@H]1[C@@H](c2ccc(F)c(F)c2OCCN2CCOCC2)[C@H](C(=O)Nc2ccc(CO)nc2)O[C@@]1(C)C(F)(F)F]
or a pharmaceutically acceptable salt thereof.

27. The compound of claim 1 in non-salt form.

28. (canceled)

29. A pharmaceutical composition comprising the compound of claim 1, or a pharmaceutically acceptable salt thereof, and one or more pharmaceutically acceptable carriers or vehicles.

30. A method of inhibiting a voltage-gated sodium channel in a subject comprising administering to the subject the compound of claim 1, or a pharmaceutically acceptable salt thereof,
optionally wherein the voltage-gated sodium channel is Na_{V}1.8.

31. (canceled)

32. A method of treating or lessening the severity in a subject of chronic pain, gut pain, neuropathic pain, musculoskeletal pain, acute pain, inflammatory pain, cancer pain, idiopathic pain, postsurgical pain, visceral pain, multiple sclerosis, Charcot-Marie-Tooth syndrome, incontinence, pathological cough, or cardiac arrhythmia comprising administering to the subject an effective amount of the compound of claim 1, or a pharmaceutically acceptable salt thereof.

33. The method of claim 32, where the method comprises treating or lessening the severity in the subject of one or more of neuropathic pain, optionally one or more of post-herpetic neuralgia, small-fiber neuropathy, idiopathic small-fiber neuropathy, or diabetic neuropathy, optionally diabetic peripheral neuropathy; musculoskeletal pain, optionally osteoarthritis pain; acute pain, acute post-operative pain; postsurgical pain, optionally one or more of bunionectomy pain, abdominoplasty pain, or heriorrhaphy pain; or visceral pain.

34. (canceled)

35. The method of claim 32, wherein said subject is treated with one or more additional therapeutic agents administered concurrently with, prior to, or subsequent to treatment with the compound, pharmaceutically acceptable salt, or pharmaceutical composition.

36. (canceled)

37. A method of treating or lessening the severity in a subject of chronic pain, gut pain, neuropathic pain, musculoskeletal pain, acute pain, inflammatory pain, cancer pain, idiopathic pain, postsurgical pain, visceral pain, multiple sclerosis, Charcot-Marie-Tooth syndrome, incontinence, pathological cough, or cardiac arrhythmia comprising administering to the subject an effective amount of the compound of claim 26, or a pharmaceutically acceptable salt thereof.
